(19) United States
(12) Patent Application Publication
(10) Pub. No.: US 2012/0149662 A1
(43) Pub. Date: Jun. 14, 2012

(54) Heterocyclic Compounds as Janus Kinase Inhibitors

(75) Inventors: Yarlagadda S. Babu, Durham, NC (US); Pravin L. Kotian, Durham, NC (US); V. Satish Kumar, Durham, NC (US); Minwan Wu, Durham, NC (US); Tsu-Hsing Lin, Durham, NC (US)

(73) Assignee: BioCryst Pharmaceuticals, Inc., Durham, NC (US)

(21) Appl. No.: 13/392,383

(22) PCT Filed: Aug. 27, 2010
(86) PCT No.: PCT/US10/46999

§ 371 Date: Feb. 24, 2012

Related U.S. Application Data
(60) Provisional application No. 61/237,546, filed on Aug. 27, 2009.
(60) Provisional application No. 61/313,583, filed on Mar. 12, 2010.

(51) Int. Cl. A61K 31/695; A61K 31/53; C07D 487/04; A61K 31/519; C07F 7/10; A61P 35/00; A61K 31/5025

(52) U.S. Cl. 514/63; 514/210.18; 514/210.21; 514/243; 514/248; 514/265.1; 544/184; 544/229; 544/236; 544/280

(57) ABSTRACT

The invention provides compounds of formula (I):

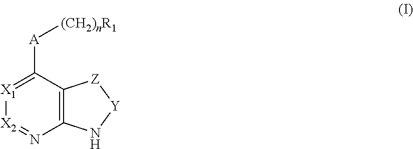

or a salt thereof as described herein. The invention also provides pharmaceutical compositions comprising a compound of formula (I), processes for preparing compounds of formula (I), intermediates useful for preparing compounds of formula (I) and therapeutic methods for suppressing an immune response or treating cancer or a hematologic malignancy using compounds of formula (I).

### CROSS-REFERENCE TO RELATED APPLICATIONS

This patent application claims the benefit of priority of U.S. application Ser. No. 61/237,546, filed Aug. 27, 2009 and U.S. application Ser. No. 61/313,583, filed Mar. 12, 2010 which applications are herein incorporated by reference.

### BACKGROUND OF THE INVENTION

Janus kinase 3 (JAK3) is a cytoplasmic protein tyrosine kinase associated with the common gamma chain (γc), which is an integral component of various cytokine receptors (Elizabeth Kudlacz et al., American Journal of Transplantation, 2004, 4, 51-57).

While effective in the prevention of transplant rejection, commonly used immunosuppressants, such as calcineurin inhibitors, possess a number of significant dose-limiting toxicities, thereby prompting a search for agents with novel mechanisms of action. The inhibition of JAK3 represents an attractive strategy for immunosuppression based upon its limited tissue distribution, lack of constitutive activation and the evidence for its role in immune cell function.

JAK3 is a viable target for immunosuppression and transplant rejection. JAK3 specific inhibitors may also be useful for treatment of hematologic and other malignancies that involve pathologic Jak activation.

Currently, there is a need for compounds, compositions and methods that are useful for treating diseases and conditions associated with pathologic JAK activation.

### SUMMARY OF THE INVENTION

In one embodiment, the invention provides a compound of the invention which is a

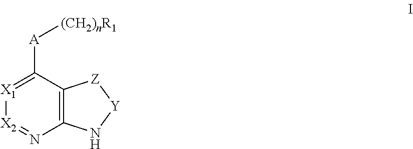

wherein:

A is CR₂R₃, NR₃, O or S; or when R₁ is other than H, A can also be absent;

X₁ is N or CR₄;

X₂ is N or CR₅;

Y is CR₆R₇, C═O or C═S, and Z is CR₈R₉, NR₁₀, O, S, C═O, C═S;

or Y is O, S or NR₁₁, and Z is CR₁₂R₁₃, C═O or C═S;

or Y is CR₆ and Z is CR₈ when X₁ is N or CR₄ and X₂ is N;

the bond represented by-is a single bond; or when X₁ is N or CR₄, X₂ is N, Y is CR₆ and Z is CR₈ the bond represented by-is a double bond;

n is 0 or 1;

R₁ is H, alkyl, halogen, cycloalkyl, heterocycle, heteroaryl, aryl or a bridged ring group; wherein any aryl or heteroaryl of R₁ is optionally substituted with one or more (e.g. 1, 2, 3, 4 or 5) Rₐ groups; and wherein any alkyl, cycloalkyl, heterocycle or bridged ring group of R₁ is optionally substituted with one or more (e.g. 1, 2, 3, 4 or 5) groups selected from Rₐ, oxo and ═NOR_{z}; or R₁ is halogen when A is CR₂R₃ or absent; or R₁ is —Oalkyl when A is CR₂R₃, NR₃ or absent; wherein —Oalkyl is optionally substituted with one or more (e.g. 1, 2, 3, 4 or 5) groups selected from Rₐ, oxo and ═NOR_{z};

R₂ is H, alkyl or cycloalkyl;

R₃ is H, CN, —C(O)alkyl, —C(O)alkenyl, —C(O)alkynyl, —C(O)cycloalkyl, —C(O)aryl, —C(═O)C(═O)NHlower alkyl, —CONR_{b}R_{c}, alkyl, alkenyl, heterocycle, heteroaryl or aryl; wherein any aryl, —C(O)aryl or heteroaryl of R₃ is optionally substituted with one or more (e.g. 1, 2, 3, 4 or 5) R_{d} groups; and wherein any alkyl, alkenyl, heterocycle, —C(O)alkyl, —C(O)alkenyl, —C(O)alkynyl, —C(O)cycloalkyl or —C(═O)C(═O)NHlower alkyl of R₃ is optionally substituted with one or more groups (e.g. 1, 2, 3, 4 or 5) selected from R_{d}, oxo and ═NOR_{z}; and R₄ is H, halogen, alkyl, cycloalkyl, alkenyl, alkynyl, aryl, heteroaryl, heterocycle, NO₂, CN, OH, —ORₑ, —NR_{f}R_{g}, N₃, —SH, —SRₑ, —C(O)alkyl, —C(O)alkenyl, —C(O)alkynyl, —C(O)cycloalkyl, —C(O)aryl, —C(O)heteroaryl, —C(O)heterocycle, —C(O)ORₕ, —C(O)NR_{f}R_{g}, —C(═NR_{f})NR_{f}R_{g}, —NR_{f}CORₑ, —NR_{f}C(O)ORₑ, —NR_{f}S(O)₂Rₑ, —NR_{f}CONR_{f}R_{g}, —OC(O)NR_{f}R_{g}, —S(O)Rₑ, —S(O)NR_{f}R_{g}, —S(O)₂Rₑ, —S(O)₂OH, —S(O)₂NR_{f}R_{g} or —C(═O)C(═O)NHlower alkyl; wherein any aryl, heteroaryl, —C(O)aryl or —C(O)heteroaryl of R₄ is optionally substituted with one or more (e.g. 1, 2, 3, 4 or 5) Rᵢ groups; and wherein any alkyl, cycloalkyl, alkenyl, alkynyl, heterocycle, —C(O)alkyl, —C(O)alkenyl, —C(O)alkynyl, —C(O)cycloalkyl, —C(O)heterocycle or —C(═O)C(═O)NHlower alkyl of R₄ is optionally substituted with one or more (e.g. 1, 2, 3, 4 or 5) groups selected from Rᵢ, oxo and ═NOR_{z};

or R₃ and R₄ together with the atoms to which they are attached form a five-membered heterocycle or a five-membered heteroaryl; wherein the five-membered heterocycle is optionally substituted with one or more groups (e.g. 1 or 2) selected from oxo or alkyl; and wherein the five-membered heteroaryl is optionally substituted with —OR₁₆ or —NHR₁₇;

R₅ is H, halogen, alkyl, cycloalkyl, alkenyl, alkynyl, aryl, heteroaryl, heterocycle, NO₂, CN, —OH, —ORⱼ, —NRₖRₘ, N₃, SH, —SRⱼ, —C(O)Rₙ, —C(O)ORₙ, —C(O)NRₖRₘ, —C(═NRₖ)NRₖRₘ, —NRₖCORⱼ, —NRₖC(O)ORⱼ, —NRₖS(O)₂Rⱼ, —NRₖCONRₖRₘ, —OC(O)NRₖRₘ, —S(O)Rⱼ, —S(O)NRₖRₘ, —S(O)₂Rⱼ, —S(O)₂OH, or —S(O)₂NRₖRₘ; wherein any aryl or heteroaryl of R₅ is optionally substituted with one or more (e.g. 1, 2, 3, 4 or 5) Rₚ groups; and wherein any alkyl, cycloalkyl, alkenyl, alkynyl or heterocycle of R₅ is optionally substituted with one or more groups selected from Rₚ, oxo and ═NOR_{z};

R₆ is H, OH, —CN, NO₂, CO₂R_{q}, —C(O)R_{q}, —NR_{q}COR_{q}, —NR_{q}Rᵣ, halogen, lower alkyl, CONR_{q}Rᵣ, or alkenyl; wherein lower alkyl or alkenyl is optionally substituted with one or more (e.g. 1, 2, 3, 4 or 5) Rₛ groups;

R₇ is H, OH, NO₂, CO₂H, —NR_{q}Rᵣ, halogen or lower alkyl; which lower alkyl is optionally substituted with one or more (e.g. 1, 2, 3, 4 or 5) Rₛ groups;

R₈ is H, OH, —CN, NO₂, CO₂R_{q}, —C(O)R_{q}, —NR_{q}COR_{q}, —NR_{q}Rᵣ, halogen, lower alkyl, CONR_{q}Rᵣ, or alkenyl; wherein lower alkyl or alkenyl is optionally substituted with one or more (e.g. 1, 2, 3, 4 or 5) Rₛ groups;

R₉ is H, OH, NO₂, CO₂H, —NR_{q}Rᵣ, halogen or lower alkyl; which lower alkyl is optionally substituted with one or more (e.g. 1, 2, 3, 4 or 5) Rₛ groups;

R₁₀ is H or alkyl;

R₁₁is H or alkyl;

R_{z2} is H or alkyl;

R₁₃ is H or alkyl;

R₁₆ is H or alkyl;

R₁₇ is H, —C(O)alkyl, —C(O)alkenyl, —C(O)alkynyl, —C(O)cycloalkyl, —C(O)aryl, —C(O)heteroaryl, —C(O)heterocycle, or —C(═O)C(═O)NHR₁₈;

R₁₈ is lower alkyl or cycloalkyl; wherein lower alkyl or cycloalkyl is optionally substituted with one or more (e.g. 1, 2 or 3) —Olower alkyl;

each Rₐ is independently selected from halogen, aryl, heteroaryl, heterocycle, alkyl, alkenyl, alkynyl, cycloalkyl, OH, CN, —OR_{z}, —Oaryl, —Oheterocycle, —Oheteroaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2}, —C(O)heterocycle, —C(O)aryl, —C(O)heteroaryl and —C(O)C(O)R_{z}; wherein any aryl, heteroaryl, —Oaryl, —Oheteroaryl, —Saryl, —Sheteroaryl, —S(O)aryl, —S(O)heteroaryl, —S(O)₂aryl, —S(O)₂heteroaryl, —NHCOaryl, —NHCOheteroaryl, —NHS(O)₂aryl, —C(O)aryl or —C(O)heteroaryl of Rₐ is optionally substituted with one or more (e.g. 1, 2, 3, 4 or 5) R_{y} groups; and wherein any heterocycle, —Oheterocycle, alkyl, alkenyl, alkynyl, cycloalkyl or —C(O)heterocycle of Rₐ is optionally substituted with one or more (e.g. 1, 2, 3, 4 or 5) groups selected from R_{y}, oxo, ═NOR_{z}, ═NOH and ═CR_{z3}R_{z4};

R_{b} and R_{c} are each independently selected from H, alkyl, alkenyl, alkynyl, cycloalkyl, heterocycle, aryl and heteroaryl; or R_{b} and R_{c} together with the nitrogen to which they are attached form a pyrrolidino, piperidino, piperazino, azetidino, morpholino, or thiomorpholino;

each R_{d} is independently selected from halogen, aryl, heteroaryl, heterocycle, R_{z}, OH, CN, —OR_{z}, —Oaryl, —OC(O)R_{z}, —OC(O)NR_{z1}R_{z2}, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{Z}; wherein any aryl, heteroaryl, heterocycle, —Oaryl, —Saryl, —Sheteroaryl, —S(O)aryl, —S(O)heteroaryl, —S(O)₂aryl, —S(O)₂heteroaryl, —NHCOaryl, —NHCOheteroaryl or —NHS(O)₂aryl of R_{d} is optionally substituted with one or more (e.g. 1, 2, 3, 4 or 5) R_{y} groups;

each Rₑ is independently alkyl, alkenyl, alkynyl, cycloalkyl, heterocycle, heteroaryl or aryl;

R_{f} and R_{g} are each independently selected from H, alkyl, alkenyl, alkynyl, cycloalkyl, heterocycle, aryl and heteroaryl; or R_{f} and R_{g} together with the nitrogen to which they are attached form a pyrrolidino, piperidino, piperazino, azetidino, morpholino, or thiomorpholino;

each Rᵢ, is independently H, alkyl, alkenyl, alkynyl, cycloalkyl, heterocycle, heteroaryl or aryl;

each Rᵢ is independently selected from halogen, aryl, heteroaryl, heterocycle, R_{z}, OH, CN, —Oaryl, —OC(O)R_{z}, —OC(O)NR_{z1}R_{z2}, SH, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z}; wherein any aryl, heteroaryl, heterocycle, —Oaryl, —Saryl, —Sheteroaryl, —S(O)aryl, —S(O)heteroaryl, —S(O)₂aryl, —S(O)₂heteroaryl, —NHCOaryl or —NHCOheteroaryl of Rᵢ is optionally substituted with one or more (e.g. 1, 2, 3, 4 or 5) R_{y} groups;

each Rⱼ is independently alkyl, alkenyl, alkynyl, cycloalkyl, heterocycle, heteroaryl or aryl;

Rₖ and Rₘ, are each independently selected from H, alkyl, alkenyl, alkynyl, cycloalkyl, heterocycle, aryl and heteroaryl; or Rₖ and Rₘ, together with the nitrogen to which they are attached form a pyrrolidino, piperidino, piperazino, azetidino, morpholino, or thiomorpholino;

each Rₙ is independently H, alkyl, alkenyl, alkynyl, cycloalkyl, heterocycle, heteroaryl or aryl;

each Rₚ is independently selected from halogen, aryl, heteroaryl, heterocycle, R_{z}, OH, CN, —OR_{z}, —Oaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z}; wherein any aryl, heteroaryl, heterocycle, —Oaryl, —Saryl, —Sheteroaryl, —S(O)aryl, —S(O)heteroaryl, —S(O)₂aryl, —S(O)₂heteroaryl, —NHCOaryl, —NHCOheteroaryl or —NHS(O)₂aryl of Rₚ is optionally substituted with one or more (e.g. 1, 2, 3, 4 or 5) R_{y} groups;

R_{q} and Rᵣ, are each independently selected from H, alkyl, alkenyl, alkynyl, cycloalkyl, heterocycle and heteroaryl; or R_{q} and Rᵣ together with the nitrogen to which they are attached form a pyrrolidino, piperidino, piperazino, azetidino, morpholino, or thiomorpholino ring;

each Rₛ is independently selected from halogen, aryl, heteroaryl, heterocycle, R_{z}, OH, CN, —OR_{z}, —Oaryl, —OC(O)R_{z}, —OC(O)NR_{z1}R_{z2}, oxo, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, ═NOR_{z}, —CHO, —C(O)R_{z}, —C(O)OH, —C(O)OR_{z}, —C(O)NR_{z1}R_{z2} and —C(O)C(O)R_{z}; wherein any aryl, heteroaryl, heterocycle, —Oaryl, —Saryl, —Sheteroaryl, —S(O)aryl, —S(O)heteroaryl, —S(O)₂aryl, —S(O)₂heteroaryl, —NHCOaryl, —NHCOheteroaryl or —NHS(O)₂aryl of Rₛ is optionally substituted with one or more (e.g. 1, 2, 3, 4 or 5) R_{y} groups;

each Rₜ is independently selected from halogen, CF₃, —OCF₃, CN, OH, —NH₂, —Olower alkyl, —Oaryl, —NHlower alkyl, —N(lower alkyl)₂, —C(O)NHlower alkyl, —C(O)N(lower alkyl)₂, aryl, heterocycle and heteroaryl; wherein any aryl, —Oaryl, heteroaryl or heterocycle of Rₜ is optionally substituted with one or more (e.g. 1, 2 or 3) groups selected from aryl and alkyl; and wherein any —Olower alkyl, —NHlower alkyl, N(lower alkyl)₂, —C(O)NHlower alkyl or —C(O)N(lower alkyl)₂ of Rₜ is optionally substituted with one or more (e.g. 1 or 2) NH₂ groups;

each R_{y} is independently halogen, R_{z}, OH, CN, —OR_{z}, —Oaryl, —Oheteroaryl, —OC(O)R_{z}, —OC(O)OR_{z}, —OC(O)NR_{z1}R_{z2}, SH, SR_{z}, —Saryl, —Sheteroaryl, —S(O)R_{z}, —S(O)aryl, —S(O)heteroaryl, —S(O)₂OH, —S(O)₂R_{z}, —S(O)₂OR_{z}, —S(O)₂Oaryl, —OS(O)₂R_{z}, —S(O)₂aryl, —OS(O)₂aryl, —S(O)₂heteroaryl, —OS(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, CHO, —C(O)R_{z}, —C(O)OH, —C(O)OR_{z}, —C(O)Oaryl, —C(O)NR_{z1}R_{z2}, —C(O)aryl, —OC(O)aryl, —C(O)heteroaryl, —OC(O)heteroaryl, —C(O)C(O)R_{z}, —C(═NCN)NH₂, aryl, heterocycle or heteroaryl; wherein any —Oaryl, —Oheteroaryl, —Saryl, —Sheteroaryl, —S(O)aryl, —S(O)heteroaryl, —S(O)₂Oaryl, —S(O)₂aryl, —OS(O)₂aryl, —S(O)₂heteroaryl, —OS(O)₂heteroaryl, —NHCOaryl, —NHCOheteroaryl, —NHS(O)₂aryl, —C(O)Oaryl, —C(O)aryl, —OC(O)aryl, —C(O)heteroaryl, —OC(O)heteroaryl, aryl, or heteroaryl of R_{y} is optionally substituted with one or more (e.g. 1, 2, 3, 4 or 5) halogen, OH, SH, R_{z}, —OR_{z}, —SR_{z}, CN, —NR_{z1}R_{z2}, —NO₂, —CHO, —Oaryl, —Oheteroaryl, —C(O)R_{z}, —C(O)OR_{z}, —C(O)OH, —NHCOR_{z}, —NHS(O)₂R_{z}, —NHS(O)₂aryl, —C(O)NR_{z1}R_{z2}, —NHCONR_{z1}R_{z2}, —NHCOheteroaryl, —NHCOaryl, —NHC(O)OR_{z}, —(C₂-C₆)alkynyl, —S(O)R_{z}, —S(O)₂R_{z}, —S(O)aryl, —S(O)₂aryl, —S(O)₂NR_{z1}R_{z2}, —Saryl, —Sheteroaryl, aryl or heteroaryl; wherein —Oaryl, —Oheteroaryl, —NHS(O)₂aryl, —NHCOheteroaryl, —NHCOaryl, —S(O)aryl, —S(O)₂aryl, —Saryl, —Sheteroaryl, aryl or heteroaryl is optionally substituted with one or more (e.g. 1, 2, 3, 4 or 5) groups selected from halogen, CN, —CF₃, NO₂ and (C₁-C₃)alkyl; and wherein any heterocycle of R_{y} is optionally substituted with one or more (e.g. 1, 2, 3, 4 or 5) groups selected from halogen, CN, NO₂, oxo, OH, SH, R_{z}, —OR_{z}, —S(O)₂R_{z}, —S(O)₂aryl, —S(O)₂heteroaryl, —C(O)R_{z}, —C(O)aryl, —C(O)heteroaryl or heteroaryl; wherein —S(O)₂aryl, —S(O)₂heteroaryl, —C(O)aryl, —C(O)heteroaryl or heteroaryl is optionally substituted with one or more (e.g. 1, 2, 3, 4 or 5) groups selected from halogen, CN, —CF₃, NO₂ and (C₁-C₃)alkyl;

each R_{z} is independently lower alkyl or cycloalkyl; wherein any lower alkyl of R_{z} is optionally substituted with one or more (e.g. 1, 2 or 3) groups selected from halogen, CN, —SCN, OH, —NH₂, —Olower alkyl, —NHlower alkyl, —N(lower alkyl)₂, —C(O)NHlower alkyl, —C(O)N(lower alkyl)₂, —C(O)lower alkyl, heterocycle, cycloalkyl, aryl, heteroaryl, —S(O)₂aryl, —S(O)aryl, —Saryl, —Sheteroaryl, —Oaryl and —Oheteroaryl, wherein aryl, heterocycle, heteroaryl, —S(O)₂aryl, —S(O)aryl, —Saryl, —Sheteroaryl, —Oaryl or —Oheteroaryl is optionally substituted with one or more (e.g. 1, 2 or 3) lower alkyl, CN, —O(C₁-C₆)alkyl, NH₂, —NHheteroaryl or —NHS(O)₂(C₁-C₆)alkyl; and wherein any cycloalkyl of R_{z} is optionally substituted with one or more (e.g. 1, 2 or 3) groups selected from (C₁-C₆)alkyl, halogen, CN, OH, —NH₂, —Olower alkyl, —NHlower alkyl, —C(O)NHlower alkyl, —C(O)N(lower alkyl)₂, heterocycle, cycloalkyl, aryl and heteroaryl, wherein aryl, heterocycle or heteroaryl may be substituted with one or more (e.g. 1, 2 or 3) lower alkyl; and wherein (C₁-C₆)alkyl is optionally substituted with OH, NHC(O)aryl or —O(C₁-C₆)alkyl;

R_{z1} and R_{z2} are each independently selected from H, alkyl, alkenyl, alkynyl, lower cycloalkyl, aryl, heterocycle and heteroaryl; wherein any alkyl, alkenyl or alkynyl of R_{z1} or R_{z2} is optionally substituted with one or more (e.g. 1, 2 or 3) Rₜ or groups; and wherein any lower cycloalkyl, aryl, heterocycle or heteroaryl of R_{z1} or R_{z2} is optionally substituted with one or more (e.g. 1, 2 or 3) groups selected from Rₜ or (C₁-C₆)alkyl; or R_{z1} and R_{z2} together with the nitrogen to which they are attached form a cyclic amino; wherein the cyclic amino is optionally substituted with one or more (e.g. 1, 2 or 3) groups selected from Rₜ, oxo and alkyl; and

R_{z3} and R_{z4} are each independently selected from H and CN; or R_{z3} and R_{z4} together with the atom to which they are attached form a cycloalkyl; or a salt thereof.

The invention also provides a pharmaceutical composition comprising a compound of formula I or a pharmaceutically acceptable salt thereof, in combination with a pharmaceutically acceptable diluent or carrier.

The invention also provides method for treating a disease or condition associated with pathologic JAK activation (e.g. a cancer, a hematologic malignancy or other malignancy) in a mammal (e.g. a human), comprising administering a compound of formula I, or a pharmaceutically acceptable salt thereof, to the mammal.

The invention also provides a compound of formula I, or a pharmaceutically acceptable salt thereof, for use in the prophylactic or therapeutic treatment of a disease or condition associated with pathologic JAK activation (e.g. a cancer, a hematologic malignancy or other malignancy).

The invention also provides a compound of formula I, or a pharmaceutically acceptable salt thereof for use in medical therapy (e.g. for use in treating a disease or condition associated with pathologic JAK activation such as cancer, a hematologic malignancy or other malignancy).

The invention also provides a compound of formula I or a pharmaceutically acceptable salt thereof for the manufacture of a medicament for the treatment of a disease or condition associated with pathologic JAK activation (e.g. a cancer, a hematologic malignancy or other malignancy) in a mammal (e.g. a human).

The invention also provides a method for suppressing an immune response in a mammal (e.g. a human), comprising administering a compound of formula I, or a pharmaceutically acceptable salt thereof, to the mammal.

The invention also provides a compound of formula I, or a pharmaceutically acceptable salt thereof, for use in the prophylactic or therapeutic suppression of an immune response.

The invention also provides the use of a compound of formula I, or a pharmaceutically acceptable salt thereof for the manufacture of a medicament for suppressing an immune response in a mammal (e.g. a human).

The invention also provides novel processes and novel intermediates disclosed herein that are useful for preparing compounds of formula I or salts thereof, for example, those described in schemes 1-79.

### DETAILED DESCRIPTION OF THE INVENTION

### Definitions

The term “alkyl” as used herein refers to alkyl groups having from 1 to 10 carbon atoms which are straight or branched monovalent groups.

The term “lower alkyl” as used herein refers to alkyl groups having from 1 to 6 carbon atoms which are straight or branched monovalent groups (i.e. (C₁-C₆)alkyl). This term is exemplified by groups such as methyl, ethyl, n-propyl, iso-propyl, n-butyl, t-butyl, isobutyl, n-pentyl, neopentyl and the like

The terms “alkenyl” or “alkene” as used herein refers to an alkenyl group having from 2 to 10 carbon atoms which are straight or branched monovalent groups and having at least one double bond. Such groups are exemplified by vinyl(ethen-1-yl), allyl, 1-propenyl, 2-propenyl(allyl), 1-methylethen-1-yl, 1-buten-1-yl, 2-buten-1-yl, 3-buten-1-yl, 1-methyl-1-propen-1-yl, 2-methyl-1-propen-1-yl, 1-methyl-2-propen-1-yl, and 2-methyl-2-propen-1-yl, preferably 1-methyl-2-propen-1-yl and the like.

The term “alkynyl” or “alkyne” as used herein refers to an alkynyl group having from 2-10 carbon atoms which are straight or branched monovalent groups and having at least one triple bond. Such groups are exemplified by, but not limited to ethyn-1-yl, propyn-1-yl, propyn-2-yl, 1-methylprop-2-yn-1-yl, butyn-1-yl, butyn-2-yl, butyn-3-yl, and the like.

The term “halogen” as used herein refers to fluoro, chloro, bromo and iodo. In one embodiment halogen is preferably fluoro.

The term “cycloalkyl” as used herein refers to saturated or partially unsaturated cyclic hydrocarbon ring systems, such as those containing 1 to 3 rings and 3 to 8 carbons per ring wherein multiple ring cycloalkyls can have fused and spiro bonds to one another but not bridging bonds. Therefore, cycloalkyl does not include bridged cyclic hydrocarbons as defined below. Exemplary groups include but are not limited to cyclopropyl, cyclobutyl, cyclopentyl, cyclohexyl, cycloheptyl, cyclooctyl, cyclobutenyl, cyclohexenyl, cyclooctadienyl, decahydronaphthalene and spiro[4.5]decane.

The term “lower cycloalkyl” as used herein refers to a cycloalkyl containing 1 ring and 3-6 carbon atoms (i.e. (C₃-C₆)cycloalkyl). Exemplary groups include cyclopropyl, cyclobutyl, cyclopentyl and cyclohexyl.

The term “aryl” as used herein refers to a monovalent aromatic cyclic group of from 6 to 14 carbon atoms having a single ring (e.g. phenyl) or multiple condensed rings (e.g. naphthyl or anthryl) wherein the condensed rings may be aromatic, saturated or partially saturated provided that at least one of the condensed rings is aromatic. Exemplary aryls include, but are not limited to, phenyl, indanyl naphthyl, 1,2-dihydronaphthyl and 1,2,3,4-tetrahydronaphthyl.

The term “heteroaryl” as used herein refers to a group of from 1 to 10 carbon atoms and 1 to 4 heteroatoms selected from the group consisting of oxygen, nitrogen and sulfur in the ring. The sulfur and nitrogen heteroatoms atoms may also be present in their oxidized forms. Such heteroaryl groups can have a single aromatic ring with at least one heteroatom (e.g. pyridyl, pyrimidinyl or furyl) or multiple condensed rings (e.g. indolizinyl or benzothienyl) wherein all of the condensed rings may or may not be aromatic and/or contain a heteroatom provided that at least one of the condensed rings is aromatic with at least one heteroatom. Exemplary heteroaryl groups include, but are not limited to pyridyl, pyrrolyl, pyrazinyl, pyrimidinyl, pyridazinyl, pyrazolyl, thienyl, indolyl, thiophenyl, imidazolyl, oxazolyl, thiazolyl, furyl, oxadiazolyl, thiadiazolyl, quinolyl, isoquinolyl, benzothiazolyl, benzoxazolyl, indazolyl, indolyl, quinoxalyl, quinazolyl, 5,6,7,8-tetrahydroisoquinoline and the like.

The term “heterocycle” or “heterocyclic” or “heterocycloalkyl” refers to a group of from 1 to 10 carbon atoms and 1 to 4 heteroatoms selected from the group consisting of oxygen, nitrogen and sulfur in the ring. The sulfur and nitrogen heteroatoms atoms may also be present in their oxidized forms. Such heterocycle groups include a single saturated or partially unsaturated ring with at least one heteroatom (e.g. azetidinyl or piperidinyl). Heterocycle groups also include multiple condensed rings wherein the condensed rings may be aryl, cycloalkyl or heterocycle provided that at least one of the condensed rings is a heterocycle (i.e. a saturated or partially unsaturated ring with at least one heteroatom). Heterocycles do not included aza-bridged cyclic hydrocarbons as defined below. Heterocycles include aziridinyl, azetidinyl, pyrrolizinyl, piperidinyl, homopiperidinyl, morpholinyl, thiomorpholinyl, piperazinyl, tetrahydrofuranyl, tetrahydrothiophenyl, dihydrooxazolyl, tetrahydropyranyl, tetrahydrothiopyranyl, 1,2,3,4-tetrahydroquinolyl, 1,2,3,4-tetrahydroisoquinolyl, benzoxazinyl and dihydrooxazolyl.

The term “cyclic amino” as used herein is a subgroup of heterocycloalkyls and refers to a monovalent 3-membered to 8-membered saturated or partially unsaturated, single, nonaromatic ring which has at least one nitrogen atom, and may have one or more identical or different hetero atoms selected from the group consisting of nitrogen, oxygen, and sulfur wherein the nitrogen or sulfur atoms may be oxidized. Aza-bridged cyclic hydrocarbons are excluded. Cyclic amino includes but is not limited to values such as aziridino, azetidino, pyrrolidino, piperidino, homopiperidino, morpholino, thiomorpholino, and piperazino.

The term “bridged ring group” includes “bridged cyclic hydrocarbon” and “aza-bridged cyclic hydrocarbon.”

The term “bridged cyclic hydrocarbon” is a saturated or partially unsaturated, bicyclic or polycyclic bridged hydrocarbon group having two or three C₃-C₁₀ cycloalkyl rings and at least one bridging group. Bicyclic or polycyclic C₄-C₁₆ bridged hydrocarbon groups are particularly preferable. Bridged cyclic hydrocarbon ring systems include but are not limited to cyclo[2.1.1]hexyl, bicyclo[2.2.1]heptyl, bicyclo[2.2.2]octyl, bicyclo[4.3.1]decyl, bicyclo[3.3.1]nonyl, bornyl, bornenyl, norbornyl, norbornenyl, 6,6-dimethylbicyclo[3.1.1]heptyl, tricyclobutyl, and adamantyl. In one embodiment bridged cyclic hydrocarbon is adamantyl or bicyclo[2.2.1]heptyl.

The term “aza-bridged cyclic hydrocarbon” is a saturated or partially unsaturated, bicyclic or polycyclic bridged hydrocarbon group having two or three rings in which at least one of the atoms is a nitrogen atom. In one embodiment the aza-bridged cyclic hydrocarbon is a bicyclic or polycyclic C₄-C₁₆ aza-bridged cyclic hydrocarbon group. Aza-bridged cyclic hydrocarbons include but are not limited to ring systems such as azanorbornyl, quinuclidinyl, isoquinuclidinyl, tropanyl, 8-azabicyclo[3.2.1]octanyl, azabicyclo[2.2.1]heptanyl, 2-azabicyclo[3.2.1]octanyl, azabicyclo[3.2.2]nonanyl, azabicyclo[3.3.0]nonanyl, and azabicyclo[3.3.1]nonanyl. In one embodiment aza-bridged cyclic hydrocarbon is preferably 8-azabicyclo[3.2.1]octanyl or 2-oxa-5-azabicyclo[2.2.1]hept-5-yl.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartrate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Specific values listed below for radicals, substituents, and ranges, are for illustration only; they do not exclude other defined values or other values within defined ranges for the radicals and substituents. The specific values listed below are specific values for compounds of formula I as well as compounds of formula Ia, Ia1, Ia2, Ia3, Ia4, Ia5, Ib, Ib1, Ib2, Ib3, Ib4, Ib5, Ic, Ic1, Ic2, Ic3, Ic4, Ic5, Id, Id1, Id2, Id3, Id4, Id5, Id6, Id7, Id8, Id9, Id10, Ie, Ie1, Ie2, Ie3, Ie4, Ie5, Ie6, Ie7, Ie8, Ie9, Ie10, Ie11, Ie12, Ie13, Ie14, Ie15, Ie16, Ie17, Ie18, Ie19, Ie20, Ie21, Ie22, Ie23, Ie24, Ie25, Ie26, or Ie27.

A specific compound of formula I is a compound of formula Ia, Ia1, Ia2, Ia3, Ia4 or Ia5:

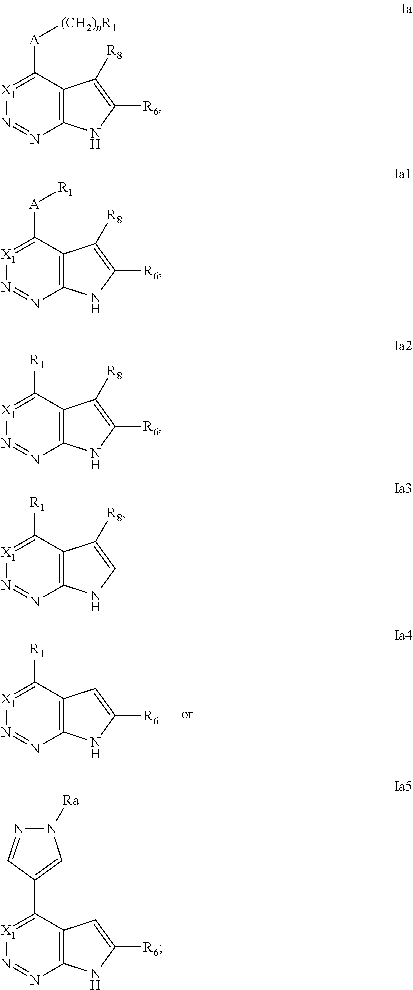

or a salt thereof.

Another specific compound of formula I is a compound of formula Ib, Ib1, Ib2, Ib3, Ib4 or Ib5:

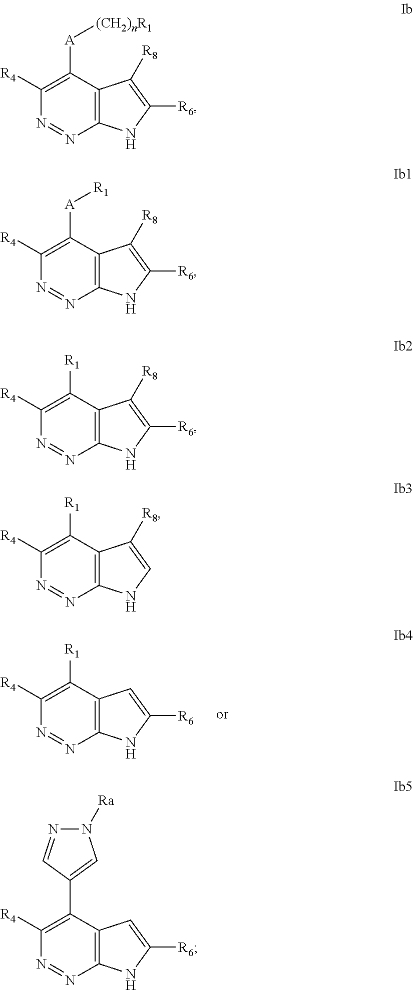

or a salt thereof.

Another specific compound of formula I is a compound of formula Ic, Ic1, Ic2, Ic3, Ic4 or Ic5:

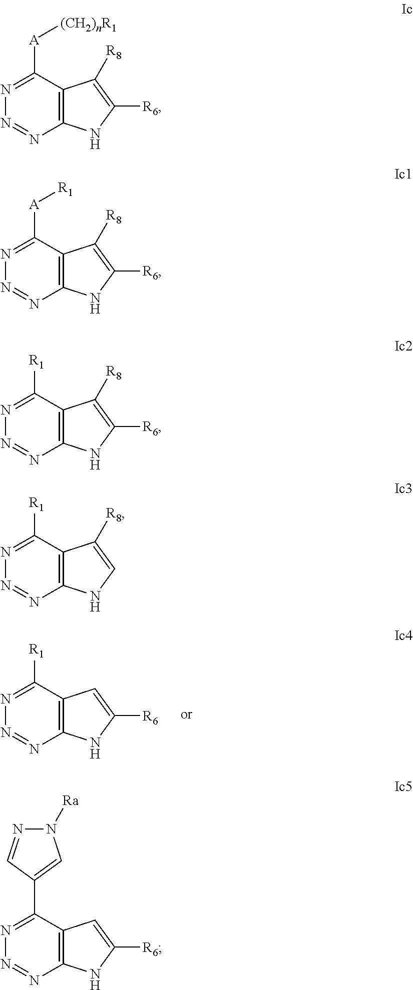

or a salt thereof.

Another specific compound of formula I is a compound of formula Id1, Id2, Id3, Id4, Id5, Id6, Id7, Id8, Id9 or Id10:

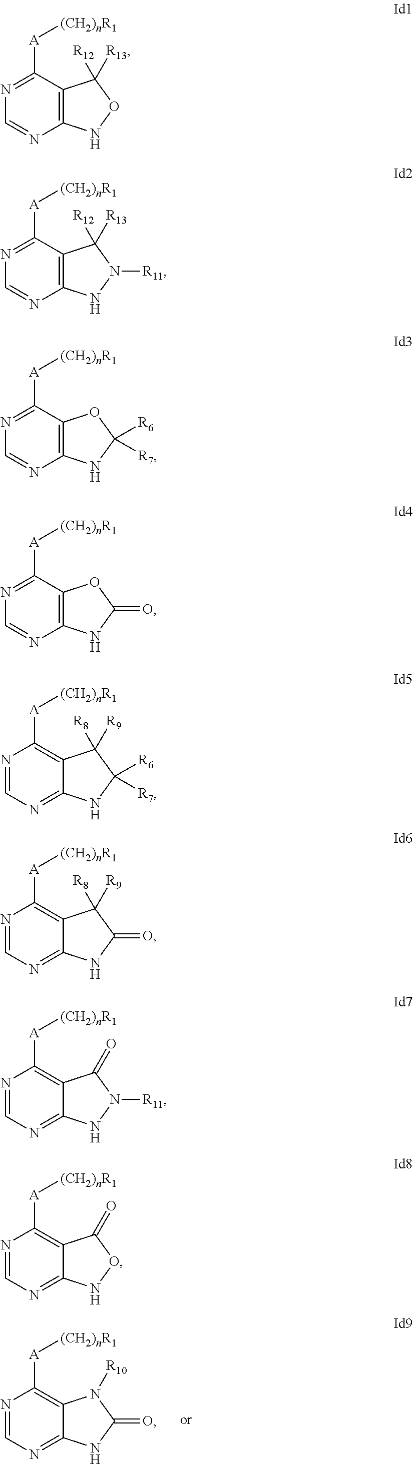

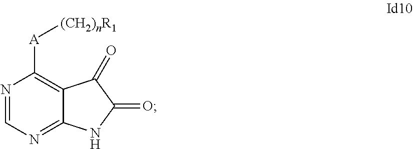

or a salt thereof.

Another specific compound of formula I is a compound of formula Ie, Ie1, Ie2, Ie3, Ie4, Ie5, Ie6, Ie7, Ie8, Ie9, Ie10, Ie11, Ie12, Ie13, Ie14, Ie15, Ie16, Ie17, Ie18, Ie19, Ie20, Ie21, Ie22, Ie23, Ie24, Ie25, Ie26, or Ie27:

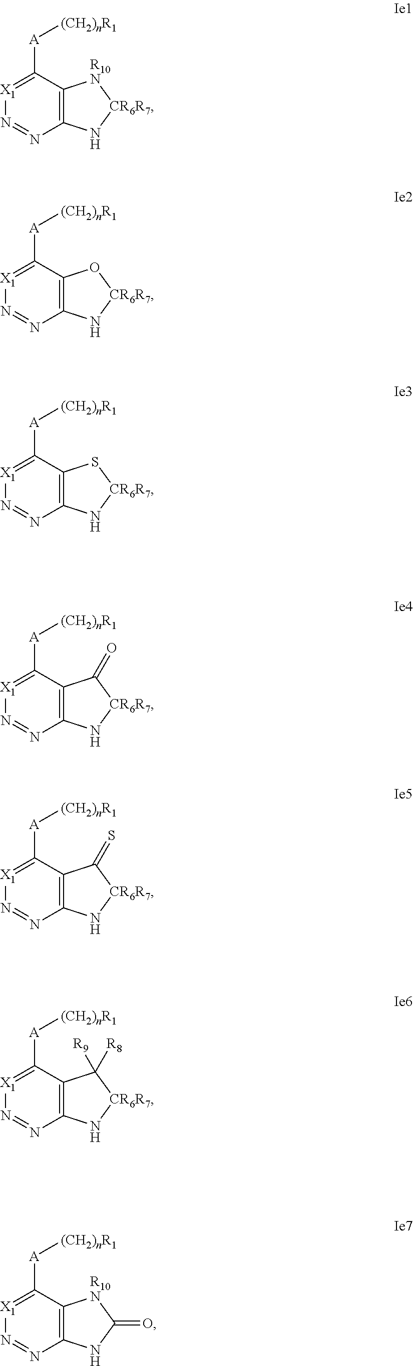

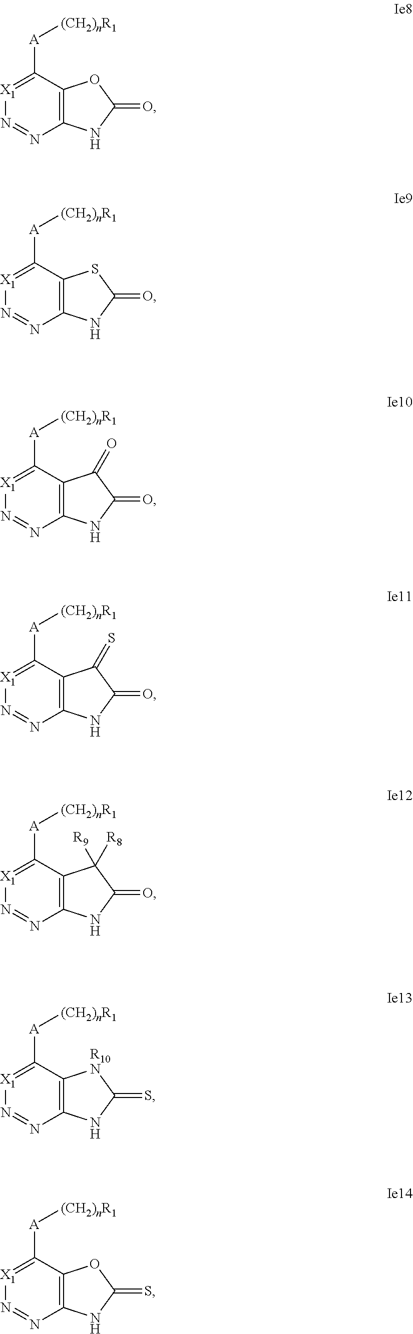

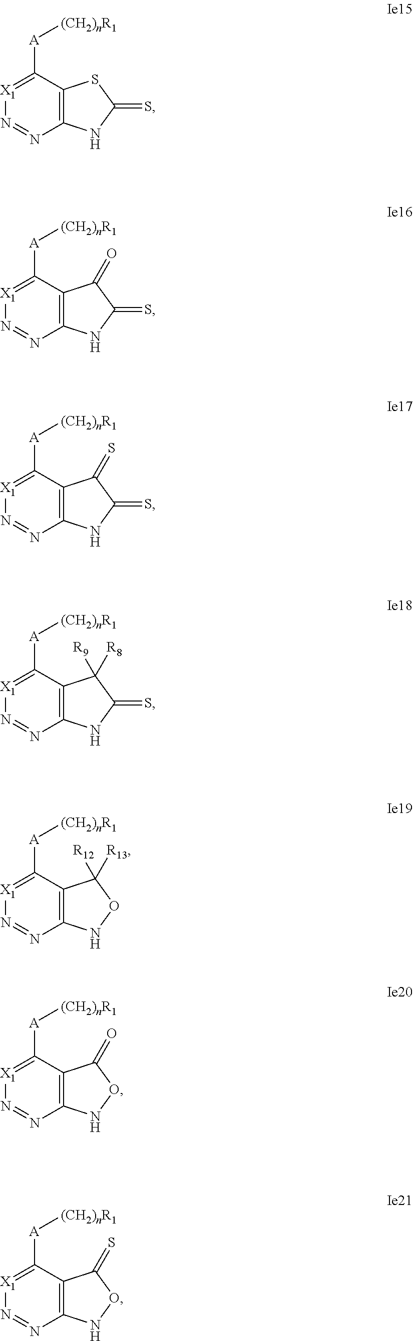

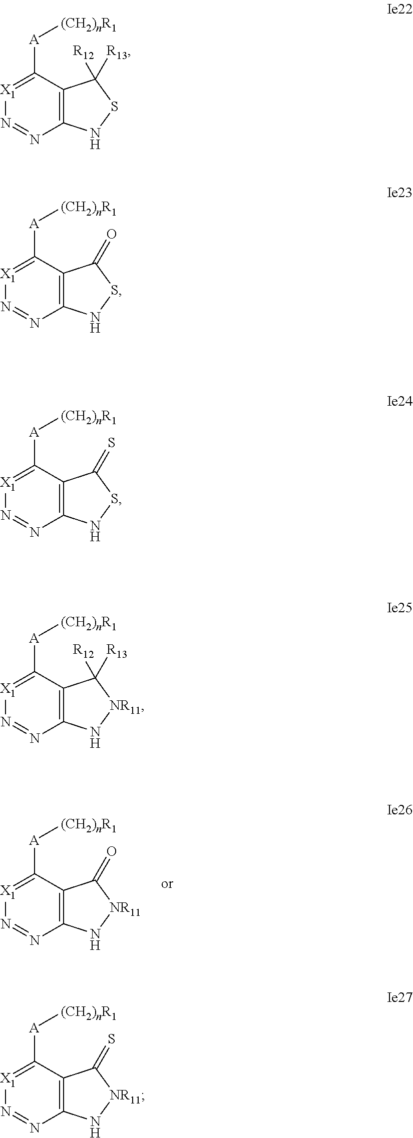

or a salt thereof.

In one embodiment, the invention provides a compound of the invention which is a compound of formula I:

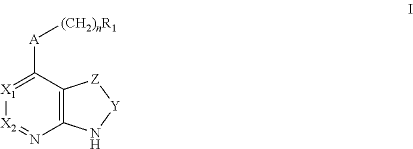

wherein:

A is CR₂R₃, NR₃, O or S;

X₁ is N or CR₄;

X₂ is N or CR₅;

Y is CR₆R₇, C═O or C═S, and Z is CR₈R₉, NR₁₀, O, S, C═O, C═S; or Y is O, S or NR₁₁; and Z is CR₁₂R₁₃, C═O or C═S; or Y is CR₆ and Z is CR₈ when X₁ is N or CR₄ and X₂ is N;

the bond represented by-is a single bond; or when X₁ is N or CR₄, X₂ is N, Y is CR₆ and Z is CR₈ the bond represented by-is a double bond;

n is 0 or 1;

R₁ is H, alkyl, cycloalkyl, heterocycle, heteroaryl, aryl, —Oalkyl or a bridged ring group wherein any aryl or heteroaryl of R₁ may be optionally substituted with one or more (e.g. 1, 2, 3, 4 or 5) Rₐ groups and wherein any alkyl, cycloalkyl, heterocycle or bridged ring group of R₁ may be optionally substituted with one or more (e.g. 1, 2, 3, 4 or 5) groups selected from Rₐ, oxo and ═NOR_{z};

R₂ is H, alkyl or cycloalkyl;

R₃ is H, CN, —C(O)alkyl, —C(O)alkenyl, —C(O)alkynyl, —C(O)cycloalkyl, —C(O)aryl, —C(═O)C(═O)NHlower alkyl, —CONRₕR_{c}, alkyl, alkenyl, heterocycle, or heteroaryl, wherein any aryl or heteroaryl of R₃ may be optionally substituted with one or more (e.g. 1, 2, 3, 4 or 5) R_{d} groups and wherein any alkyl, alkenyl, alkynyl, cycloalkyl, heterocycle or lower alkyl of R₃ may be optionally substituted with one or more (e.g. 1, 2, 3, 4 or 5) groups selected from R_{d}, oxo and ═NOR_{z}; and R₄ is H, halogen, alkyl, cycloalkyl, alkenyl, alkynyl, aryl, heteroaryl, heterocycle, NO₂, CN, OH, —ORₑ, —NR_{f}R_{g}, N₃, —SH, —SRₑ, —C(O)alkyl, —C(O)alkenyl, —C(O)alkynyl, —C(O)cycloalkyl, —C(O)aryl, —C(O)heteroaryl, —C(O)heterocycle, —C(O)ORₕ, —C(O)NR_{f}R_{g}, —C(═NR_{f})NR_{f}R_{g}, —NR_{f}CORₑ, —NR_{f}C(O)ORₑ, —NR_{f}S(O)₂Rₑ, —NR/CONR_{f}R_{g}, —OC(O)NR_{f}R_{g}, —S(O)Rₑ, —S(O)NR_{f}R_{g}, —S(O)₂Rₑ, —S(O)₂OH, —S(O)₂NR_{f}R_{g} or —C(═O)C(═O)NHlower alkyl wherein any aryl or heteroaryl of R₄ may be optionally substituted with one or more (e.g. 1, 2, 3, 4 or 5) Rᵢ, groups and wherein any alkyl, lower alkyl, cycloalkyl, alkenyl, alkynyl or heterocycle of R₄ may be optionally substituted with one or more (e.g. 1, 2, 3, 4 or 5) groups selected from Rᵢ, oxo and ═NOR_{z};

or R₃ and R₄ together with the atoms to which they are attached form a five-membered heterocycle or a five-membered heteroaryl wherein the five-membered heterocycle is optionally substituted with one or more (e.g. 1 or 2) groups selected from oxo or alkyl and wherein the five-membered heteroaryl is optionally substituted with —OR₁₆ or —NHR₁₇;

R₅ is H, halogen, alkyl, cycloalkyl, alkenyl, alkynyl, aryl, heteroaryl, heterocycle, NO₂, CN, —OH, —ORⱼ, —NRₖRₘ, N₃, SH, —SRⱼ, —C(O)Rₙ, —C(O)ORₙ, —C(O)NRₖRₘ, —C(═NRₖ)NRₖRₘ, —NRₖCORⱼ, —NRₖC(O)ORⱼ, —NR_{b}S(O)₂Rⱼ, —NRₖCONRₖRₘ, —OC(O)NRₖRₘ, —S(O)Rⱼ, —S(O)NRₖRₘ, —S(O)₂Rⱼ, —S(O)₂OH, or —S(O)₂NRₖRₘ wherein any aryl or heteroaryl of R₅ may be optionally substituted with one or more (e.g. 1, 2, 3, 4 or 5) Rₚ groups and wherein any alkyl, cycloalkyl, alkenyl, alkynyl or heterocycle of R₅ may be optionally substituted with one or more (e.g. 1, 2, 3, 4 or 5) groups selected from Rₚ, oxo and ═NOR_{z};

R₆ is H, OH, NO₂, CO₂H, —NR_{q}Rᵣ, halogen or lower alkyl which lower alkyl is optionally substituted with one or more (e.g. 1, 2, 3, 4 or 5) Rₛ groups;

R₇ is H, OH, NO₂, CO₂H, —NR_{q}Rᵣ, halogen or lower alkyl which lower alkyl is optionally substituted with one or more (e.g. 1, 2, 3, 4 or 5) Rₛ groups;

R₈ is H, OH, NO₂, CO₂H, halogen or lower alkyl which lower alkyl is optionally substituted with one or more (e.g. 1, 2, 3, 4 or 5) Rₛ groups;

R₉ is H, OH, NO₂, CO₂H, —NR_{q}Rᵣ, halogen or lower alkyl which lower alkyl is optionally substituted with one or more (e.g. 1, 2, 3, 4 or 5) Rₛ groups;

R₁₀ is H or alkyl;

R₁₁ is alkyl;

R₁₂ is H or alkyl;

R₁₃ is H or alkyl;

R₁₆ is H or alkyl;

R₁₇ is H, —C(O)alkyl, —C(O)alkenyl, —C(O)alkynyl, —C(O)cycloalkyl, —C(O)aryl, —C(O)heteroaryl, —C(O)heterocycle, or —C(═O)C(═O)NHR₁₈;

R₁₈ is lower alkyl or cycloalkyl wherein lower alkyl or cycloalkyl may be substituted with one or more (e.g. 1, 2 or 3) —Olower alkyl;

each Rₐ is independently selected from halogen, aryl, heteroaryl, heterocycle, R_{z}, OH, CN, —OR_{z}, —Oaryl, —Oheterocycle, —Oheteroaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2}, —C(O)heterocycle, —C(O)heteroaryl and —C(O)C(O)R_{z}, and wherein any aryl, heteroaryl, or heterocycle of Rₐ may be optionally substituted with one or more (e.g. 1, 2, 3, 4 or 5) R_{y} groups;

R_{b} and R_{c} are each independently selected from H, alkyl, alkenyl, alkynyl, cycloalkyl, heterocycle and heteroaryl; or R_{b} and R_{c} together with the nitrogen to which they are attached form a pyrrolidino, piperidino, piperazino, azetidino, morpholino, or thiomorpholino;

each R_{d} is independently selected from halogen, aryl, heteroaryl, heterocycle, R_{z}, OH, CN, —OR_{z}, —Oaryl, —OC(O)R_{z}, —OC(O)NR_{z1}R_{z2}, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z}, and wherein any aryl of R_{d} may be optionally substituted with one or more (e.g. 1, 2, 3, 4 or 5) R_{y} groups;

each Rₑ is independently alkyl, alkenyl, alkynyl, cycloalkyl, heterocycle, heteroaryl or aryl;

R_{f} and R_{g} are each independently selected from H, alkyl, alkenyl, alkynyl, cycloalkyl, heterocycle and heteroaryl; or R_{f} and R_{g} together with the nitrogen to which they are attached form a pyrrolidino, piperidino, piperazino, azetidino, morpholino, or thiomorpholino;

each Rₕ is independently H, alkyl, alkenyl, alkynyl, cycloalkyl, heterocycle, heteroaryl or aryl;

each Rᵢ is independently selected from halogen, aryl, heteroaryl, heterocycle, R_{z}, OH, CN, —OR_{z}, —Oaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z} and wherein any aryl of Rᵢ, may be optionally substituted with one or more (e.g. 1, 2, 3, 4 or 5) R_{y} groups;

each Rⱼ is independently alkyl, alkenyl, alkynyl, cycloalkyl, heterocycle, heteroaryl or aryl;

Rₖ and Rₘ are each independently selected from H, alkyl, alkenyl, alkynyl, cycloalkyl, heterocycle and heteroaryl; or Rₖ and Rₘ together with the nitrogen to which they are attached form a pyrrolidino, piperidino, piperazino, azetidino, morpholino, or thiomorpholino;

each Rₙ is independently H, alkyl, alkenyl, alkynyl, cycloalkyl, heterocycle, heteroaryl or aryl;

each Rₚ is independently selected from halogen, aryl, heteroaryl, heterocycle, R_{I}, OH, CN, —OR_{z}, —Oaryl, —OC(O)R_{z}, —OC(O)NR_{z1}R_{z2}, SH, —SR_{I}, —Saryl, —Sheteroaryl, —S(O)R_{I}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z}, and wherein any aryl of Rₚ may be optionally substituted with one or more (e.g. 1, 2, 3, 4 or 5) R_{y} groups;

R_{q} and Rᵣ are each independently selected from H, alkyl, alkenyl, alkynyl, cycloalkyl, heterocycle and heteroaryl; or R_{q} and Rᵣ together with the nitrogen to which they are attached form a pyrrolidino, piperidino, piperazino, azetidino, morpholino, or thiomorpholino ring;

each Rₛ is independently selected from halogen, aryl, heteroaryl, heterocycle, R_{z}, OH, CN, —OR_{z}, —Oaryl, —OC(O)R_{z}, —OC(O)NR_{z1}R_{z2}, oxo, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, ═NOR_{z}, —CHO, —C(O)R_{z}, —C(O)OH, —C(O)OR_{z}, —C(O)NR_{z1}R_{z2} and —C(O)C(O)R_{z} wherein any aryl of Rₛ may be optionally substituted with one or more (e.g. 1, 2, 3, 4 or 5) R_{y} groups;

each Rₜ is independently selected from halogen, CN, OH, —Olower alkyl, —NHlower alkyl, —C(O)NHlower alkyl, —C(O)N(lower alkyl)₂, heterocycle and heteroaryl wherein any heterocycle of Rₜ may be substituted with one or more (e.g. 1, 2 or 3) lower alkyl;

each R_{y} is independently halogen, aryl, R_{z}, OH, CN, OR_{z}, —Oaryl, —Oheteroaryl, —OC(O)R_{z}, —OC(O)NR_{z1}R_{z2}, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, CHO, —C(O)R_{z}, —C(O)OH, —C(O)OR_{z}, —C(O)NR_{z1}R_{z2}, —C(O)C(O)R_{z}; heterocycle or heteroaryl;

each R_{z} is independently lower alkyl or lower cycloalkyl wherein lower alkyl or lower cycloalkyl may be optionally substituted with one or more (e.g. 1, 2 or 3) groups selected from halogen, CN, OH, —Olower alkyl, —NHlower alkyl, —C(O)NHlower alkyl, —C(O)N(lower alkyl)₂, heterocycle and heteroaryl wherein heterocycle may be substituted with one or more (e.g. 1, 2 or 3) lower alkyl; and

R_{z1} and R_{z2} are each independently selected from H, lower alkyl, alkenyl, alkynyl, lower cycloalkyl, heterocycle and heteroaryl, wherein lower alkyl or lower cycloalkyl may be optionally substituted with one or more (e.g. 1, 2 or 3) Rₜ groups; or R_{z1} and R_{z2} together with the nitrogen to which they are attached form a cyclic amino;

or a salt thereof.

In another embodiment, the invention provides a compound of the invention which is a compound of formula I:

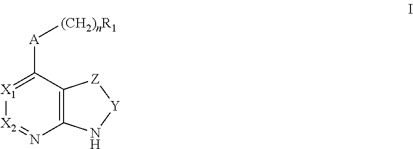

wherein:

A is CR₂R₃, NR₃, O or S; or when R₁ is other than H, A can also be absent;

X₁ is N or CR₄;

X₂ is N or CR₅;

Y is CR₆R₇, C═O or C═S, and Z is CR₈R₉, NR₁₀, O, S, C═O, C═S; or Y is O, S or NR₁₁; and Z is CR₁₂R₁₃, C═O or C═S; or Y is CR₆ and Z is CR₈ when X₁ is N or CR₄ and X₂ is N;

the bond represented by-is a single bond; or when X₁ is N or CR₄, X₂ is N, Y is CR₆ and Z is CR₈ the bond represented by-is a double bond;

n is 0 or 1;

R₁ is H, alkyl, halogen, cycloalkyl, heterocycle, heteroaryl, aryl, —Oalkyl or a bridged ring group wherein any aryl or heteroaryl of R₁ may be optionally substituted with one or more (e.g. 1, 2, 3, 4 or 5) Rₐ groups and wherein any alkyl, cycloalkyl, heterocycle or bridged ring group of R₁ may be optionally substituted with one or more (e.g. 1, 2, 3, 4 or 5) groups selected from Rₐ, oxo and ═NOR_{z};

R₂ is H, alkyl or cycloalkyl;

R₃ is H, CN, —C(O)alkyl, —C(O)alkenyl, —C(O)alkynyl, —C(O)cycloalkyl, —C(O)aryl, —C(═O)C(═O)NHlower alkyl, —CONR_{b}R_{c}, alkyl, alkenyl, heterocycle, heteroaryl, or absent wherein any aryl or heteroaryl of R₃ may be optionally substituted with one or more (e.g. 1, 2, 3, 4 or 5) R_{d} groups and wherein any alkyl, alkenyl, alkynyl, cycloalkyl, heterocycle or lower alkyl of R₃ may be optionally substituted with one or more groups (e.g. 1, 2, 3, 4 or 5) selected from R_{d}, oxo and ═NOR_{z}; and R₄ is H, halogen, alkyl, cycloalkyl, alkenyl, alkynyl, aryl, heteroaryl, heterocycle, NO₂, CN, OH, —ORₑ, —NR_{f}R_{g}, N₃, —SH, —SRₑ, —C(O)alkyl, —C(O)alkenyl, —C(O)alkynyl, —C(O)cycloalkyl, —C(O)aryl, —C(O)heteroaryl, —C(O)heterocycle, —C(O)ORₕ, —C(O)NR_{f}R_{g}, —C(═NR_{f})NR_{f}R_{g}, —NR_{f}CORₑ, —NR_{f}C(O)ORₑ, —NR_{f}S(O)₂Rₑ, —NR_{f}CONR_{f}R_{g}, —OC(O)NR_{f}R_{g}, —S(O)Rₑ, —S(O)NR_{f}R_{g}, —S(O)₂Rₑ, —S(O)₂OH, —S(O)₂NR_{f}R_{g} or —C(═O)C(═O)NHlower alkyl wherein any aryl or heteroaryl of R₄ may be optionally substituted with one or more (e.g. 1, 2, 3, 4 or 5) Rᵢ groups and wherein any alkyl, lower alkyl, cycloalkyl, alkenyl, alkynyl or heterocycle of R₄ may be optionally substituted with one or more (e.g. 1, 2, 3, 4 or 5) groups selected from R oxo and ═NOR_{z}; or R₃ and R₄ together with the atoms to which they are attached form a five-membered heterocycle or a five-membered heteroaryl wherein the five-membered heterocycle is optionally substituted with one or more groups (e.g. 1 or 2) selected from oxo or alkyl and wherein the five-membered heteroaryl is optionally substituted with —OR₁₆ or —NHR₁₇;

R₅ is H, halogen, alkyl, cycloalkyl, alkenyl, alkynyl, aryl, heteroaryl, heterocycle, NO₂, CN, —OH, —ORⱼ, —NRₖRₘ, N₃, SH, —SRⱼ, —C(O)Rₙ, —C(O)ORₙ, —C(O)NRₖRₘ, —C(═NRₖ)NRₖRₘ, —NRₖCORⱼ, —NRₖC(O)ORⱼ, —NR_{b}S(O)₂Rⱼ, —NRₖCONRₖRₘ, —OC(O)NRₖRₘ, —S(O)Rⱼ, —S(O)NRₖRⱼ, —S(O)₂Rⱼ, —S(O)₂OH, or —S(O)₂NRₖRₘ, wherein any aryl or heteroaryl of R₅ may be optionally substituted with one or more (e.g. 1, 2, 3, 4 or 5) Rₚ groups and wherein any alkyl, cycloalkyl, alkenyl, alkynyl or heterocycle of R₅ may be optionally substituted with one or more groups selected from Rₚ, oxo and ═NOR_{z};

R₆ is H, OH, NO₂, CO₂H, —NR_{q}Rᵣ, halogen or lower alkyl which lower alkyl is optionally substituted with one or more (e.g. 1, 2, 3, 4 or 5) Rₛ groups;

R₇ is H, OH, NO₂, CO₂H, —NR_{q}Rᵣ, halogen or lower alkyl which lower alkyl is optionally substituted with one or more (e.g. 1, 2, 3, 4 or 5) Rₛ groups;

R₈ is H, OH, NO₂, CO₂H, —NR_{q}Rᵣ, halogen or lower alkyl which lower alkyl is optionally substituted with one or more (e.g. 1, 2, 3, 4 or 5) Rₛ groups;

R₉ is H, OH, NO₂, CO₂H, —NR_{q}Rᵣ, halogen or lower alkyl which lower alkyl is optionally substituted with one or more (e.g. 1, 2, 3, 4 or 5) Rₛ groups;

R₁₀ is H or alkyl;

R₁₁ is alkyl;

R₁₂ is H or alkyl;

R₁₃ is H or alkyl;

R₁₆ is H or alkyl;

R₁₇ is H, —C(O)alkyl, —C(O)alkenyl, —C(O)alkynyl, —C(O)cycloalkyl, —C(O)aryl, —C(O)heteroaryl, —C(O)heterocycle, or —C(═O)C(═O)NHR₁₈;

R₁₈ is lower alkyl or cycloalkyl wherein lower alkyl or cycloalkyl may be substituted with one or more —Olower alkyl;

each Rₐ is independently selected from halogen, aryl, heteroaryl, heterocycle, —(C₁-C₆)alkyl, —(C₃-C₆)cycloalkyl, OH, CN, —OR_{z}, —Oaryl, —Oheterocycle, —Oheteroaryl, —OC(O)R_{z}, —OC(O)NR_{z1}R_{z2}, SH, —SR_{z}, —Saryl, —Sheteroaryl, —S(O)R₂, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2}, —C(O)heterocycle, —C(O)heteroaryl and —C(O)C(O)R_{z}, and wherein any aryl, heteroaryl, heterocycle, alkyl or cycloalkyl of Rₐ may be optionally substituted with one or more (e.g. 1, 2, 3, 4 or 5) R_{y} groups;

R_{b} and R_{c} are each independently selected from H, alkyl, alkenyl, alkynyl, cycloalkyl, heterocycle and heteroaryl; or R_{b} and R_{c} together with the nitrogen to which they are attached form a pyrrolidino, piperidino, piperazino, azetidino, morpholino, or thiomorpholino;

each R_{d} is independently selected from halogen, aryl, heteroaryl, heterocycle, R_{z}, OH, CN, —OR_{z}, —Oaryl, —OC(O)R_{z}, —OC(O)NR_{z1}R_{z2}, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z} and wherein any aryl of R_{d} may be optionally substituted with one or more (e.g. 1, 2, 3, 4 or 5) R_{y} groups;

each Rₑ is independently alkyl, alkenyl, alkynyl, cycloalkyl, heterocycle, heteroaryl or aryl;

R_{f} and R_{g} are each independently selected from H, alkyl, alkenyl, alkynyl, cycloalkyl, heterocycle and heteroaryl; or R_{f} and R_{g} together with the nitrogen to which they are attached form a pyrrolidino, piperidino, piperazino, azetidino, morpholino, or thiomorpholino;

each Rₕ is independently H, alkyl, alkenyl, alkynyl, cycloalkyl, heterocycle, heteroaryl or aryl;

each Rⱼ is independently selected from halogen, aryl, heteroaryl, heterocycle, R_{z}, OH, CN, —OR_{z}, —Oaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z} and wherein any aryl of Rₚ may be optionally substituted with one or more (e.g. 1, 2, 3, 4 or 5) R_{y} groups;

each Rⱼ is independently alkyl, alkenyl, alkynyl, cycloalkyl, heterocycle, heteroaryl or aryl;

Rₖ and Rₘ are each independently selected from H, alkyl, alkenyl, alkynyl, cycloalkyl, heterocycle and heteroaryl; or Rₖ and Rₘ together with the nitrogen to which they are attached form a pyrrolidino, piperidino, piperazino, azetidino, morpholino, or thiomorpholino;

each Rₙ is independently H, alkyl, alkenyl, alkynyl, cycloalkyl, heterocycle, heteroaryl or aryl;

each Rₚ is independently selected from halogen, aryl, heteroaryl, heterocycle, R_{z}, OH, CN, —OR_{z}, —Oaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z} and wherein any aryl of Rₚ may be optionally substituted with one or more (e.g. 1, 2, 3, 4 or 5) R_{y} groups;

R_{q} and Rᵣ are each independently selected from H, alkyl, alkenyl, alkynyl, cycloalkyl, heterocycle and heteroaryl; or R_{q} and Rᵣ together with the nitrogen to which they are attached form a pyrrolidino, piperidino, piperazino, azetidino, morpholino, or thiomorpholino ring;

each Rₛ is independently selected from halogen, aryl, heteroaryl, heterocycle, R_{z}, OH, CN, —OR_{z}, —Oaryl, —OC(O)R_{z}, —OC(O)NR_{z1}R_{z2}, oxo, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, ═NOR_{z}, —CHO, —C(O)R_{z}, —C(O)OH, —C(O)OR_{z}, —C(O)NR_{z1}R_{z2} and —C(O)C(O)R_{z} wherein any aryl of Rₛ may be optionally substituted with one or more (e.g. 1, 2, 3, 4 or 5) R_{y} groups;

each Rₜ is independently selected from halogen, CN, OH, —NH₂, —Olower alkyl, —NHlower alkyl, —C(O)NHlower alkyl, —C(O)N(lower alkyl)₂, heterocycle and heteroaryl wherein any heterocycle of Rₜ may be substituted with one or more lower (e.g. 1, 2 or 3) alkyl;

each R_{y} is independently halogen, aryl, R_{z}, OH, CN, OR_{z}, —Oaryl, —Oheteroaryl, —OC(O)R_{z}, —OC(O)NR_{z1}R_{z2}, SH, SR_{z}, —Saryl, —Sheteroaryl, —S(O)R_{z}, —S(O)aryl, —S(O)heteroaryl, —S(O)₂OH, —S(O)₂R_{z}, —OS(O)₂R_{z}, —S(O)₂aryl, —OS(O)₂aryl, —S(O)₂heteroaryl, —OS(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, CHO, —C(O)R_{z}, —C(O)OH, —C(O)OR_{z}, —C(O)NR_{z1}R_{z2}, —C(O)aryl, —OC(O)aryl, —C(O)heteroaryl, —OC(O)heteroaryl, —C(O)C(O)R_{z}, aryl, heterocycle or heteroaryl wherein any aryl or hetereoaryl of R_{y} is optionally substituted with one or more (e.g. 1, 2, 3, 4 or 5) halogen, (C₁-C₃)alkyl, CF₃, —O(C₁-C₆)alkyl, CN, —OCH₂CN, NR_{z1}R_{z2}, —NO₂, —CHO, —Oaryl, —OCF₃, —C(O)OR_{z}, —C(O)OH, aryl, —NHCOR_{z}, —NHS(O)₂R_{z}, —C(O)NR_{z1}R_{z2}, —NHCONR_{z1}R_{z2}, —NHCOheteroaryl, —NHC(O)OR_{z}, —(C₂-C₆)alkynyl, —Saryl or heteroaryl wherein heteroaryl is optionally substituted with (C₁-C₃)alkyl and wherein any heterocycle of R_{y} is optionally substituted with one or more R_{z}, —S(O)₂R_{z}, —S(O)₂aryl, —S(O)₂heteroaryl, —C(O)R_{z}, —C(O)aryl, —C(O)heteroaryl or heteroaryl wherein aryl or hetereoaryl is optionally substituted with one or more (e.g. 1, 2 or 3) halogen or (C₁-C₃)alkyl;

each R_{z} is independently lower alkyl or lower cycloalkyl wherein lower alkyl or lower cycloalkyl may be optionally substituted with one or more (e.g. 1, 2 or 3) groups selected from halogen, CN, OH, —NH₂, —Olower alkyl, —NHlower alkyl, —C(O)NHlower alkyl, —C(O)N(lower alkyl)₂, heterocycle, cycloalkyl and heteroaryl wherein heterocycle may be substituted with one or more (e.g. 1, 2 or 3) lower alkyl; and

R_{z1} and R_{z2} are each independently selected from H, lower alkyl, alkenyl, alkynyl, lower cycloalkyl, heterocycle and heteroaryl, wherein lower alkyl or lower cycloalkyl may be optionally substituted with one or more (e.g. 1, 2 or 3) Rₜ groups; or R_{z1} and R_{z2} together with the nitrogen to which they are attached form a cyclic amino;

or a salt thereof

A specific value for A is NR₃.

Another specific value for A is O.

A specific group of compounds of formula I are compounds wherein A is absent.

Another specific group of compounds of formula I are compounds wherein A is absent and n is 0.

A specific value for X₁ is CR₄.

Another specific value for X₁ is N.

A specific value for X₂ is CR₅.

Another specific value for X₂ is N.

A specific group of compounds of formula I are compounds wherein X₁ is N and X₂ is CR₅.

A specific group of compounds of formula I are compounds wherein X₁ is N and X₂ is N.

A specific group of compounds of formula I are compounds wherein X₁ is CR₄ and X₂ is N.

A specific group of compounds of formula I are compounds wherein X₁ is CR₄ and X₂ is CR₅.

A specific group of compounds of formula I are compounds wherein R₃ is H, CN, —C(O)alkyl, —C(O)alkenyl, —C(O)alkynyl, —C(O)cycloalkyl, —C(O)aryl, —C(═O)C(═O)NHlower alkyl, —CONR_{b}R_{c}, alkyl, alkenyl, heterocycle, or heteroaryl; and R₄ is H, halogen, alkyl, cycloalkyl, alkenyl, alkynyl, aryl, heteroaryl, heterocycle, NO₂, CN, OH, —ORₑ, —NR_{f}R_{g}, N₃, —SH, —SRₑ, —C(O)alkyl, —C(O)alkenyl, —C(O)alkynyl, —C(O)cycloalkyl, —C(O)aryl, —C(O)heteroaryl, —C(O)heterocycle, —C(O)ORₕ, —C(O)NR_{f}R_{g}, —C(═NR_{f})NR_{f}R_{g}, —NR_{f}CORₑ, —NR_{f}C(O)ORₑ, —NR_{f}S(O)₂Rₑ, —NR_{f}CONR_{f}R_{g}, —OC(O)NR_{f}R_{g}, —S(O)Rₑ, —S(O)NR_{f}R_{g}, —S(O)₂Rₑ, —S(O)₂OH, —S(O)₂NR_{f}R_{g} or —C(═O)C(═O)NHlower alkyl.

Another specific group of compounds of formula I are compounds wherein R₃ is H, CN, —C(O)alkyl, —C(O)alkenyl, —C(O)alkynyl, —C(O)cycloalkyl, —C(O)aryl, —C(═O)C(═O)NHlower alkyl, —CONR_{b}R_{c}, alkyl, alkenyl, heterocycle, or heteroaryl; wherein any aryl or heteroaryl of R₃ may be optionally substituted with one or more R_{d} groups and wherein any alkyl, alkenyl, alkynyl, cycloalkyl, heterocycle or lower alkyl of R₃ may be optionally substituted with one or more groups selected from R_{d}, oxo and ═NOR_{z}; and R₄ is H, halogen, alkyl, cycloalkyl, alkenyl, alkynyl, aryl, heteroaryl, heterocycle, NO₂, CN, OH, —ORₑ, —NR_{f}R_{g}, N₃, —SH, —SRₑ, —C(O)alkyl, —C(O)alkenyl, —C(O)alkynyl, —C(O)cycloalkyl, —C(O)aryl, —C(O)heteroaryl, —C(O)heterocycle, —C(O)ORₕ, —C(O)NR_{f}R_{g}, —C(═NR_{f})NR_{f}R_{g}, —NR_{f}CORₑ, —NR_{f}C(O)ORₑ, —NR_{f}S(O)₂Rₑ, —NR_{f}CONR_{f}R_{g}, —OC(O)NR_{f}R_{g}, —S(O)Rₑ, —S(O)NR_{f}R_{g}, —S(O)₂Rₑ, —S(O)₂OH, —S(O)₂NR_{f}R_{g} or —C(═O)C(═O)NHlower alkyl; wherein any aryl or heteroaryl of R₄ may be optionally substituted with one or more Rᵢ groups and wherein any alkyl, lower alkyl, cycloalkyl, alkenyl, alkynyl or heterocycle of R₄ may be optionally substituted with one or more groups selected from Rᵢ, oxo and ═NOR_{z}.

A specific value for R₄ is H, heteroaryl, heterocycle or —C(O)NR_{f}R_{g}; wherein heteroaryl is optionally substituted with one or more Rᵢ groups; and wherein heterocycle is optionally substituted with one or more groups selected from Rᵢ, oxo and ═NOR_{z};

Another specific value for R₄ is heteroaryl, heterocycle or —C(O)NR_{f}R_{g}.

Another specific value for R₄ is —C(O)NR_{f}R_{g}.

Another specific value for R₄ is —CONH₂.

Another specific value for R₄ is heteroaryl.

Another specific value for R₄ is:

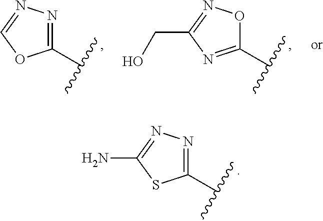

Another specific value for R₄ is H.

A specific value for R₃ is alkyl or H.

Another specific value for R₃ is CH₃ or H.

Another specific value for R₃ is H.

A specific group of compounds of formula I are compounds wherein R₃ and R₄ together with the atoms to which they are attached form a five-membered heterocycle or a five-membered heteroaryl wherein the five-membered heterocycle is optionally substituted with one or more groups selected from oxo and alkyl and wherein the five-membered heteroaryl is optionally substituted with —OR₁₆ or —NHR₁₇.

Another specific group of compounds of formula I are compounds wherein R₄ and R₃ together are —N(R₁₄)C(O)—, —C(O)N(R₁₅)—, —C(OR₁₆)═N— or —C(NHR₁₇)═N— wherein R₁₄ is H or alkyl and R₁₅ is H or alkyl.

Another specific group of compounds of formula I are compounds wherein R₄ and R₃ together are —N(R₁₄)C(O)—.

Another specific group of compounds of formula I are compounds wherein R₄ and R₃ together are —C(NHR₁₇)═N—.

Another specific group of compounds of formula I are compounds wherein R₄ and R₃ together are —C(O)N(R₁₅)—.

Another specific group of compounds of formula I are compounds wherein R₄ and R₃ together are —C(OR₁₆)═N—.

A specific value for R₅ is H.

A specific group of compounds of formula I are compounds of the formula:

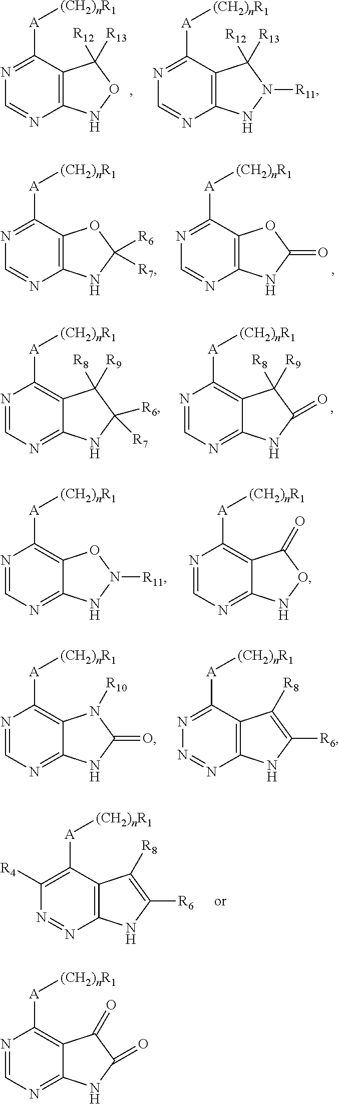

or a salt thereof.

A specific value for R₆ is H.

A specific value for R₇ is H.

A specific value for R₈ is H.

Another specific value for R₈ is CONR_{q}Rᵣ,

Another specific value for R₈ is CONH₂.

A specific value for R₉ is H.

A specific group of compounds are compounds wherein R₇ is H and R₉ is H.

A specific value for R₁₀ is H.

A specific value for R₁₁ is alkyl.

A specific value for R₁₂ is H.

A specific value for R₁₃ is H.

A specific value for n is 0.

Another specific value for n is 1.

A specific value for R₁ is alkyl, cycloalkyl, aryl, heterocycle, heteroaryl or bridged ring group.

Another specific value for R₁ is H.

Another specific value for R₁ is alkyl, cycloalkyl, aryl, heterocycle, heteroaryl or bridged ring group; wherein any aryl or heteroaryl of R₁ is optionally substituted with one or more Rₐ groups and wherein any alkyl, cycloalkyl, heterocycle or bridged ring group of R₁ is optionally substituted with one or more groups selected from Rₐ, oxo and ═NOR_{z.}

Another specific value for R₁ is cycloalkyl, aryl, heterocycle, heteroaryl or bridged ring group.

Another specific value for R₁ is cycloalkyl, aryl, heterocycle, heteroaryl or bridged ring group; wherein any aryl or heteroaryl of R₁ is optionally substituted with one or more Rₐ groups and wherein any alkyl, cycloalkyl, heterocycle or bridged ring group of R₁ is optionally substituted with one or more groups selected from Rₐ, oxo and ═NOR_{z.}

Another specific value for R₁ is bridged ring group.

Another specific value for R₁ is bridged ring group; wherein any bridged ring group of R₁ is optionally substituted with one or more groups selected from Rₐ, oxo and ═NOR_{z}.

Another specific value for R₁ is bridged cyclic hydrocarbon.

Another specific value for R₁ is bridged cyclic hydrocarbon; wherein any bridged cyclic hydrocarbon of R₁ is optionally substituted with one or more groups selected from Rₐ, oxo and ═NOR_{z.}

Another specific value for R₁ is aza-bridged cyclic hydrocarbon.

Another specific value for R₁ is aza-bridged cyclic hydrocarbon; wherein any aza-bridged cyclic hydrocarbon of R₁ is optionally substituted with one or more groups selected from Rₐ, oxo and ═NOR_{z.}

Another specific value for R₁ is adamantyl or 8-azabicyclo[3.2.1]octanyl.

Another specific value for R₁ is adamantyl or 8-azabicyclo[3.2.1]octanyl; wherein adamantyl or 8-azabicyclo[3.2.1]octanyl is optionally substituted with one or more groups selected from Rₐ, oxo and ═NOR_{z.}

Another specific value for R₁ is adamantyl or 8-azabicyclo[3.2.1]octanyl substituted with one or more —OH.

Another specific value for R₁ is heteroaryl.

Another specific value for R₁ is heteroaryl; wherein any heteroaryl of R₁ is optionally substituted with one or more Rₐ groups.

Another specific value for R₁ is pyrrolyl, thienyl, benzothienyl, furyl, benzofuranyl, thiazolyl, oxazolyl, pyrazolyl, imidazolyl or oxadiazolyl.

Another specific value for R₁ is pyrrolyl, thienyl, benzothienyl, furyl, benzofuranyl, thiazolyl, oxazolyl, pyrazolyl, imidazolyl or oxadiazolyl; each optionally substituted with one or more Rₐ groups.

Another specific group value for R₁ is pyrrolyl, thienyl, benzothienyl, furyl, benzofuranyl, thiazolyl, oxazolyl, pyrazolyl, imidazolyl or oxadiazolyl each substituted with one or more Rₐ groups.

Another specific value for R₁ is:

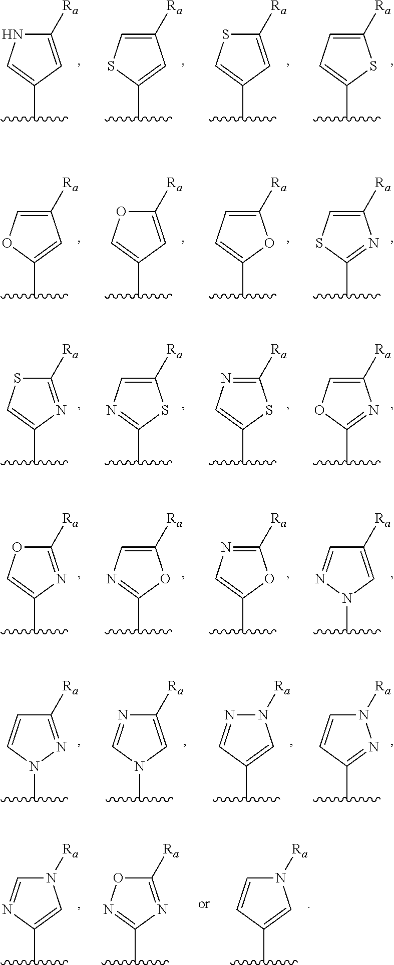

Another specific value for R₁ is halogen.

Another specific value for R₁ is pyrrolyl or pyrazolyl; each substituted with one or more Rₐ groups.

Another specific value for R₁ is:

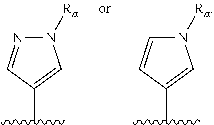

Another specific value for R₁ is aryl; wherein aryl is optionally substituted with one or more Rₐ groups.

Another specific value for R₁ is aryl; wherein aryl is substituted with one or more Rₐ groups.

Another specific value for R₁ is phenyl; wherein phenyl is substituted with one or more Rₐ groups.

Another specific value for R₁ is heterocycle; wherein any heterocycle of R₁ is optionally substituted with one or more Rₐ groups.

Another specific value for R₁ is piperidinyl; wherein piperidinyl is optionally substituted with one or more Rₐ groups.

A specific group of compounds of formula I are compounds wherein R₁ is piperidinyl; wherein piperidinyl is optionally substituted with one or more groups independently selected from alkyl and —C(O)R_{z}; wherein alkyl is optionally substituted with one or more groups selected from R_{y}, oxo, ═NOR_{z}, ═NOH and ═CR_{z3}R_{z4}.

A specific group of compounds of formula I are compounds wherein R₁ is halogen, n is 0 and A is absent.

A specific value for Rₐ is heterocycle, (C₁-C₆)alkyl or (C₃-C₆)cycloalkyl.

Another specific value for Rₐ is heterocycle, (C₁-C₆)alkyl or (C₃-C₆)cycloalkyl; wherein any heterocycle, (C₁-C₆)alkyl, or (C₃-C₆)cycloalkyl of Rₐ is substituted with one or more R_{y} groups.

Another specific value for Rₐ is oxetanyl, tetrahydrofuranyl, oxiranyl, tetrahydropryanyl, azetidinyl, aziridinyl, piperidinyl, pyrrolidinyl, cyclopropyl, cyclobutyl, cyclopentyl, cyclohexyl, ethyl or propyl; each of which is substituted with one or more R_{y} groups.

A specific group of compounds of formula I are compounds wherein Rₐ is substituted with one or more R_{y} groups.

Another specific value for Rₐ is alkyl, cycloalkyl, heterocycle or —C(O)NR_{z1}R_{z2}; wherein any heterocycle, alkyl or cycloalkyl of Rₐ is optionally substituted with one or more groups selected from R_{y} oxo, ═NOR_{z}, ═NOH and ═CR_{z3}R_{z4}.

Another specific value for Rₐ is alkyl, cycloalkyl, heterocycle or —NR_{z1}R_{z2}; wherein any heterocycle, alkyl or cycloalkyl of Rₐ is optionally substituted with one or more R_{y} groups.

Another specific value for Rₐ is ethyl, propyl, butyl, pentyl, cyclopropyl, cyclobutyl, cyclopentyl, cyclohexyl, cycloheptyl, oxetanyl, tetrahydrofuranyl, oxiranyl, tetrahydropranyl, azetidinyl, aziridinyl, piperidinyl, pyrrolidinyl or —NR_{z1}R_{z2}; wherein ethyl, propyl, butyl, pentyl, cyclopropyl, cyclobutyl, cyclopentyl, cyclohexyl, cycloheptyl, oxetanyl, tetrahydrofuranyl, oxiranyl, tetrahydropranyl, azetidinyl, aziridinyl, piperidinyl or pyrrolidinyl are each optionally substituted with one or more R_{y} groups.

Another specific value for Rₐ is ethyl, propyl, cyclobutyl, cyclopentyl, cyclohexyl, oxetanyl or azetidinyl; each optionally substituted with one or more R_{y} groups.

Another specific value for Rₐ is:

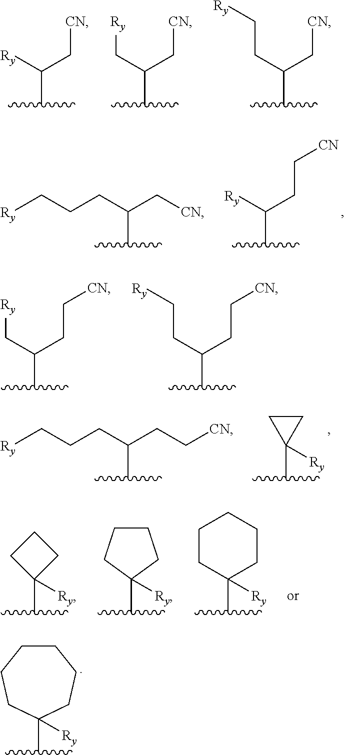

Another specific value for Rₐ is heteroaryl, heterocycle, alkyl, OH, CN, —OR_{z}, —Oheterocycle, —Oheteroaryl, —S(O)₂NR_{z1}R_{z2}, —C(O)R_{z}, —C(O)NR_{z1}R_{z2}, —C(O)heterocycle and —C(O)heteroaryl; wherein any heteroaryl, —Oheteroaryl or —C(O)heteroaryl of Rₐ is optionally substituted with one or more R_{y} groups; and wherein any heterocycle, —Oheterocycle, alkyl or —C(O)heterocycle of Rₐ is optionally substituted with one or more groups selected from R_{y}, oxo, ═NOR_{z}, ═NOH and —CR_{z3}R_{z4};

A specific value for R_{y} is R_{z}, OH, CN, OR_{z}, —Oheteroaryl, —OC(O)R_{z}, —S(O)₂R_{z}, —OS(O)₂R_{z}, —S(O)₂aryl, —OS(O)₂aryl, —S(O)₂heteroaryl, —OS(O)₂heteroaryl, —C(O)R_{z}, —C(O)aryl, —OC(O)aryl, —C(O)heteroaryl, —OC(O)heteroaryl, aryl, heterocycle or heteroaryl; wherein any aryl or hetereoaryl of R_{y} is optionally substituted with one or more halogen, (C₁-C₃)alkyl, CF₃, —O(C₁-C₃)alkyl, CN, —OCH₂CN, NR_{z1}R_{z2}, —NO₂, —CHO, —Oaryl, —OCF₃, —C(O)OR_{z}, —C(O)OH, aryl, —NHCOR_{z}, —NHS(O)₂R_{z}, —C(O)NR_{z1}R_{z2}, —NHCONR_{z1}R_{z2}, —NHCOheteroaryl, —NHC(O)OR_{z}, —(C₂-C₆)alkynyl, —Saryl or heteroaryl wherein heteroaryl is optionally substituted with (C₁-C₃)alkyl and wherein any heterocycle of R_{y} is optionally substituted with one or more R_{z}, —S(O)₂R_{z}, —S(O)₂aryl, —S(O)₂heteroaryl, —C(O)R_{z}, —C(O)aryl, —C(O)heteroaryl or heteroaryl wherein aryl or hetereoaryl is optionally substituted with one or more halogen or (C₁-C₃)alkyl.

Another specific value for R_{y} is R_{z}, OH, CN, —S(O)₂R_{z}, —C(O)OR_{z}, heterocycle or aryl; wherein any aryl of R_{y} is optionally substituted with one or more halogen, OH, SH, —OR_{z}, —SR_{z}, CN, —NR_{z1}R_{z2}, —NO₂, —CHO, —Oaryl, —Oheteroaryl, —C(O)R_{z}, —C(O)OR_{z}, —C(O)OH, —NHCOR_{z}, —NHS(O)₂R_{z}, —NHS(O)₂aryl, —C(O)NR_{z1}R_{z2}, —NHCONR_{z1}R_{z2}, —NHCOheteroaryl, —NHCOaryl, —NHC(O)OR_{z}, —(C₂-C₆)alkynyl, —S(O)R_{z}—S(O)₂R_{z}, —S(O)aryl, —S(O)₂aryl, —S(O)₂NR_{z1}R_{z2}, —Saryl, —Sheteroaryl, aryl or heteroaryl; wherein —Oaryl, —Oheteroaryl, —NHS(O)₂aryl, —NHCOheteroaryl, —NHCOaryl, —S(O)aryl, —S(O)₂aryl, —Saryl, —Sheteroaryl, aryl or heteroaryl is optionally substituted with one or more groups selected from halogen, CN, —CF₃, NO₂ and (C₁-C₃)alkyl; and wherein any heterocycle of R_{y} is optionally substituted with one or more groups selected from halogen, CN, NO₂, oxo, OH, SH, R_{z}, —OR_{z}, —S(O)₂R_{z}, —S(O)₂aryl, —S(O)₂heteroaryl, —C(O)R_{z}, —C(O)aryl, —C(O)heteroaryl or heteroaryl; wherein —S(O)₂aryl, —S(O)₂heteroaryl, —C(O)aryl, —C(O)heteroaryl or heteroaryl is optionally substituted with one or more groups selected from halogen, CN, —CF₃, NO₂ and (C₁-C₃)alkyl.

Another specific value for R_{y} is R_{z}, OH, CN, —OR_{z}, —C(O)R_{z}, —C(O)OR_{z} or aryl; wherein any aryl of R_{y} is optionally substituted with one or more halogen, OH, SH, R_{z}, —OR_{z}, —SR_{z}, CN, —NR_{z1}R_{z2}, —NO₂, —CHO, —Oaryl, —Oheteroaryl, —C(O)R_{z}, —C(O)OR_{z}, —C(O)OH, —NHCOR_{z}, —NHS(O)₂R_{z}, —NHS(O)₂aryl, —C(O)NR_{z1}R_{z2}, —NHCONR_{z1}R_{z2}, —NHCOheteroaryl, —NHCOaryl, —NHC(O)OR_{z}, —(C₂-C₆)alkynyl, —S(O)R_{z}, —S(O)₂R_{z}, —S(O)aryl, —S(O)₂aryl, —S(O)₂NR_{z1}R_{z2}, —Saryl, —Sheteroaryl, aryl or heteroaryl.

Another specific value for R_{y} is R_{z}, OH, CN, —OR_{z}, —C(O)R_{z}, —C(O)OR_{z} or aryl; wherein any aryl of R_{y} is optionally substituted with one or more OH.

Another specific value for R_{y} is R_{z}, OH, CN, —OR_{z}, —S(O)₂R_{z}, —C(O)OR_{z} or aryl; wherein any aryl of R_{y} is optionally substituted with one or more halogen, OH, SH, R_{z}, —OR_{z}, —SR_{z}, CN, —NR_{z1}R_{z2}, —NO₂, —CHO, —Oaryl, —Oheteroaryl, —C(O)R_{z}, —C(O)OR_{z}, —C(O)OH, —NHCOR_{z}, —NHS(O)₂R_{z}, —NHS(O)₂aryl, —C(O)NR_{z1}R_{z2}, —NHCONR_{z1}R_{z2}, —NHCOheteroaryl, —NHCOaryl, —NHC(O)OR_{z}, —(C₂-C₆)alkynyl, —S(O)R_{z}, —S(O)₂R_{z}, —S(O)aryl, —S(O)₂aryl, —S(O)₂NR_{z1}R_{z2}, —Saryl, —Sheteroaryl, aryl or heteroaryl.

Another specific value for R_{y} is R_{z}, OH, CN, S(O)₂R_{z}, —C(O)OR_{z} or aryl; wherein any aryl of R_{y} is optionally substituted with one or more OH.

A specific value for R_{z} is lower alkyl or cycloalkyl; wherein any lower alkyl of R_{z} is optionally substituted with one or more groups selected from CN and OH; and wherein any cycloalkyl of R_{z} is optionally substituted with one or more groups selected from CN and OH.

Another specific value for R_{z} is lower alkyl or cycloalkyl; wherein any lower alkyl of R_{z} is optionally substituted with one or more groups selected from halogen, CN and OH; and wherein any cycloalkyl of R_{z} is optionally substituted with one or more groups selected from halogen, CN and OH.

Another specific value for Rₐ is:

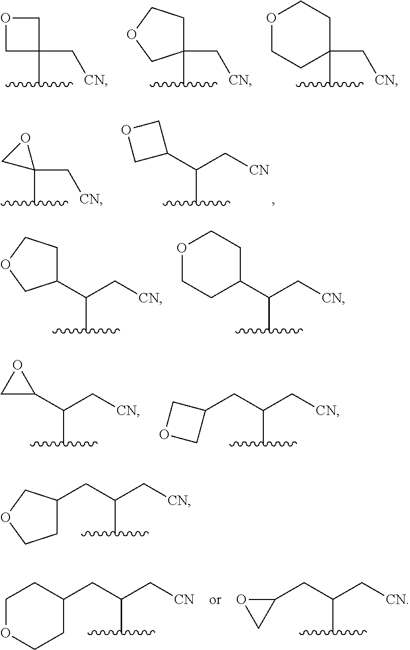

Another specific value for Rₐ is:

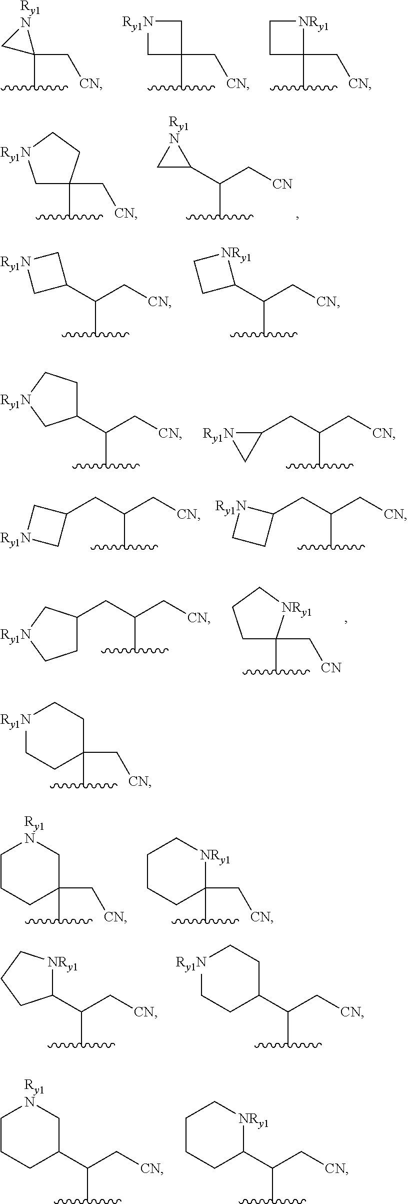

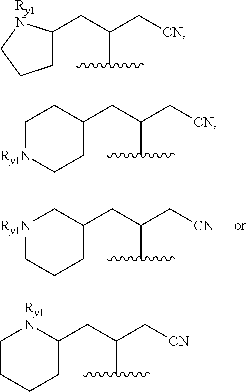

wherein each R_{y1} is independently R_{z}, —S(O)₂R_{z}, —S(O)₂aryl, —S(O)₂heteroaryl, —C(O)R_{z}, —C(O)aryl, —C(O)heteroaryl, or heteroaryl wherein any aryl or hetereoaryl of R_{y1} is optionally substituted with one or more halogen or (C₁-C₃)alkyl.

Another specific value for R_{y1} is H.

Another specific value for Rₐ is:

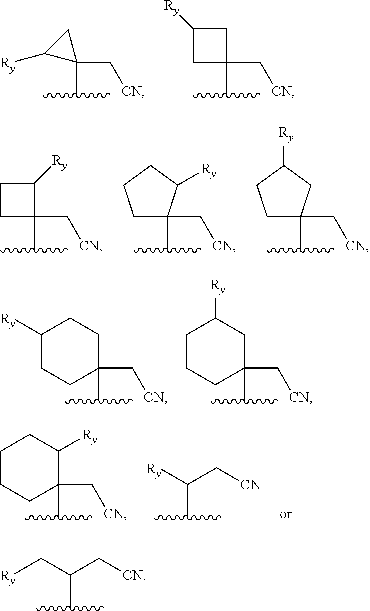

Another specific value for Rₐ is:

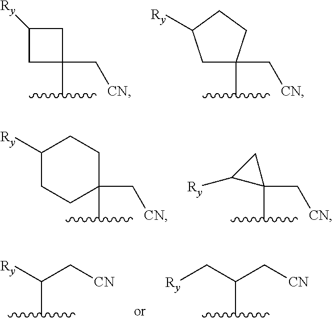

Another specific value for R_{y} is R_{z}, CN, OR_{z}, —Oheteroaryl, —OC(O)R_{z}, —S(O)₂R_{z}, —OS(O)₂R_{z}, —S(O)₂aryl, —OS(O)₂aryl, —S(O)₂heteroaryl, —OS(O)₂heteroaryl, —C(O)R_{z}, —C(O)aryl, —OC(O)aryl, —C(O)heteroaryl, —OC(O)heteroaryl, or heteroaryl wherein any aryl or hetereoaryl of R_{y} is optionally substituted with one or more halogen or (C₁-C₃)alkyl.

Another specific value for R_{y} is OH, CN, —CO₂R_{z}, aryl or heteroaryl wherein any aryl or hetereoaryl of R_{y} is optionally substituted with one or more halogen, (C₁-C₃)alkyl, CF₃, —O(C₁-C₃)alkyl, CN, —OCH₂CN, NR_{z1}R_{z2}, —NO₂, —CHO, —Oaryl, —OCF₃, —C(O)OR_{z}, —C(O)OH, aryl, —NHCOR_{z}, —NHS(O)₂R_{z}, —C(O)NR_{z1}R_{z2}, —NHCONR_{z1}R_{z2}, —NHCOheteroaryl, —NHC(O)OR_{z}, —(C₂-C₆)alkynyl, —Saryl or heteroaryl wherein heteroaryl is optionally substituted with (C₁-C₃)alkyl.

Another specific value for R_{y} is R_{z}.

Another specific value for Rₐ is:

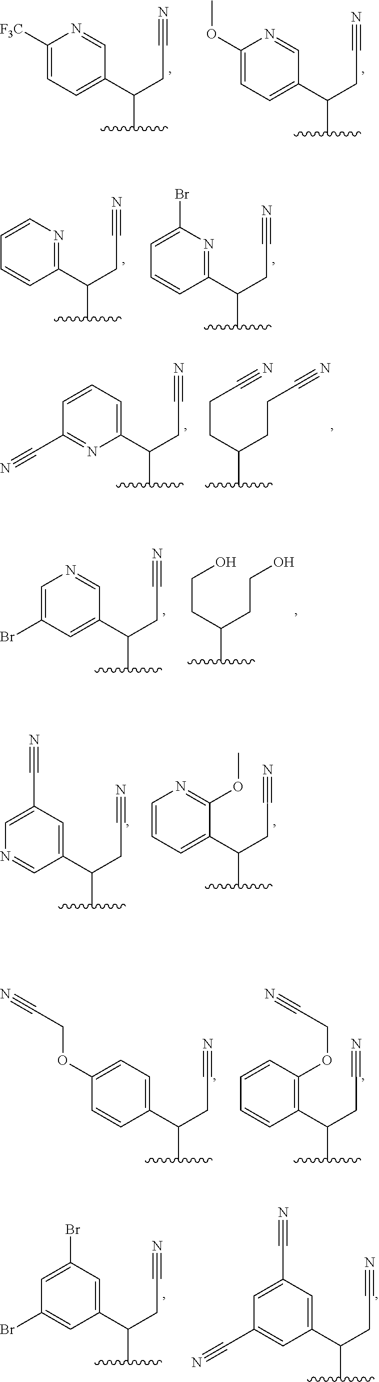

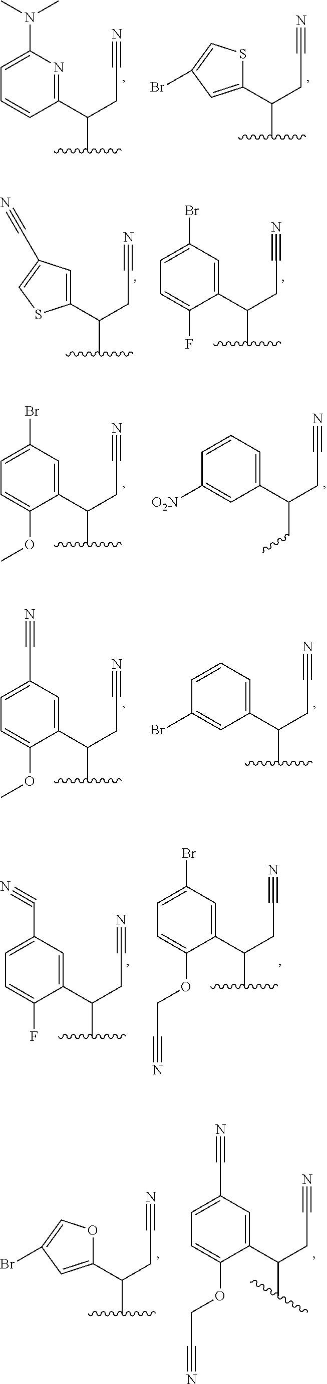

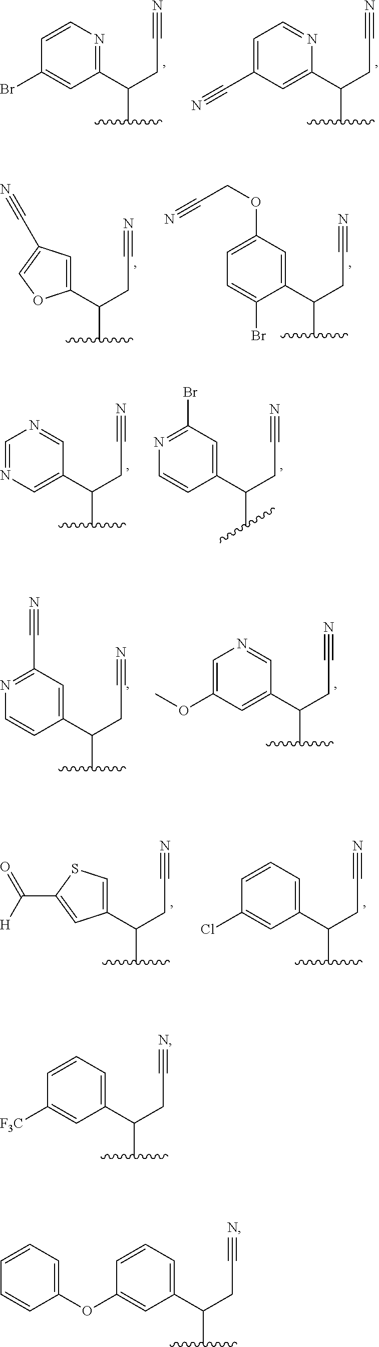

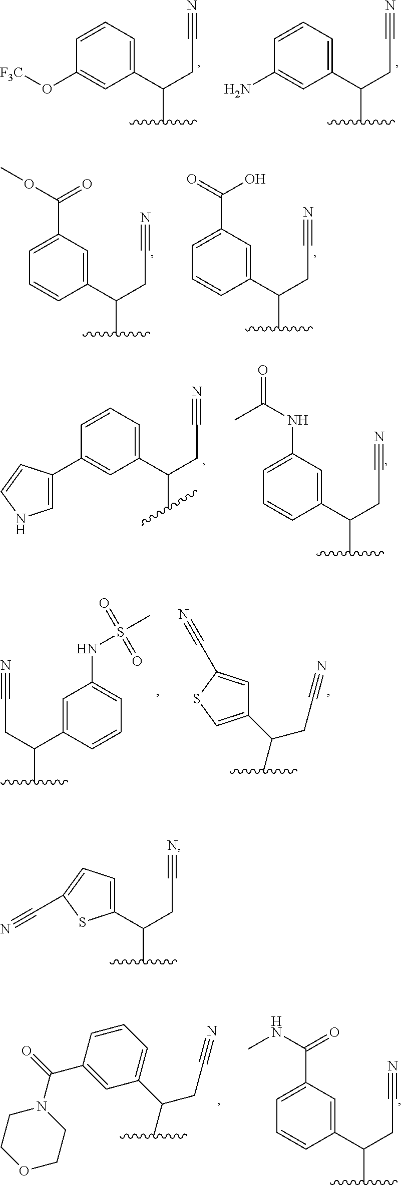

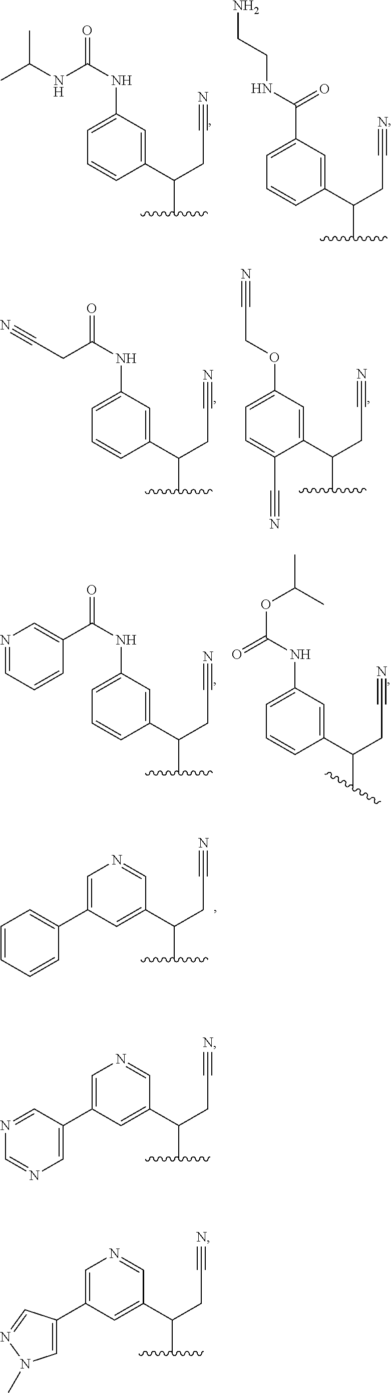

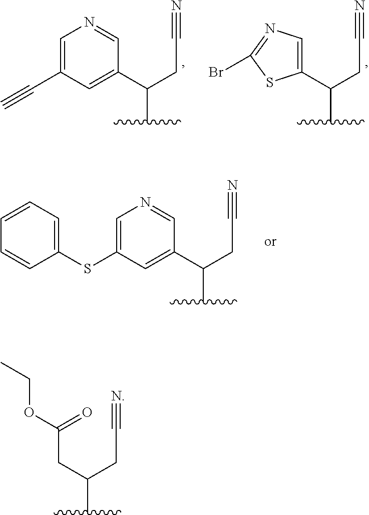

Another specific value for Rₐ is:

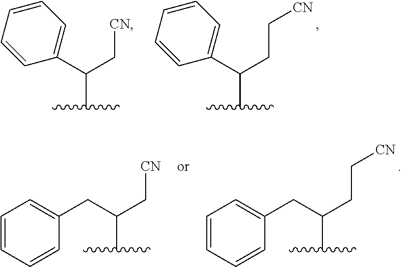

Another specific value for Rₐ is:

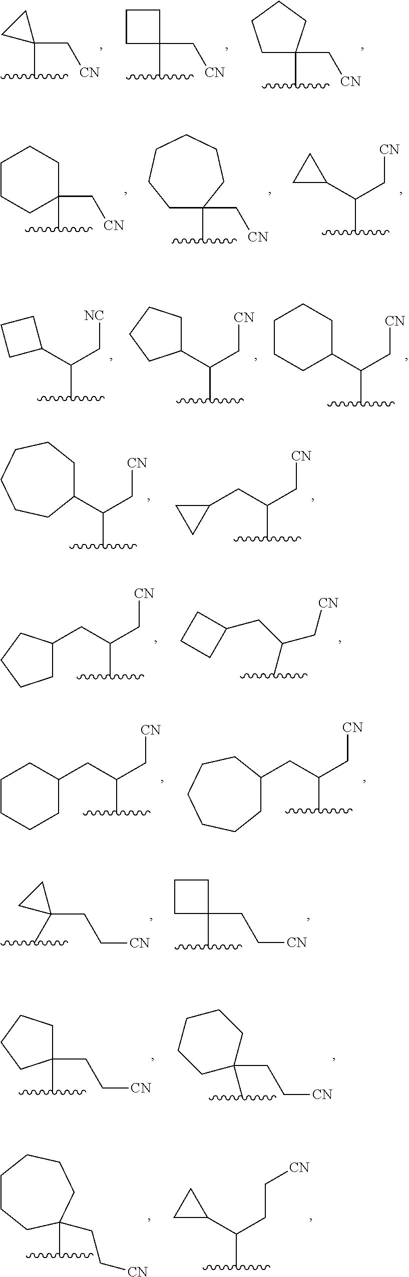

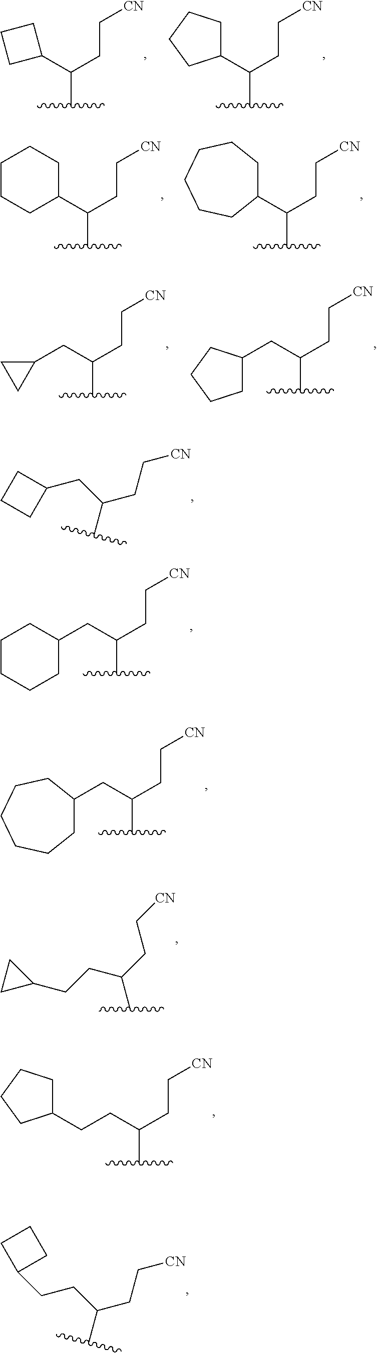

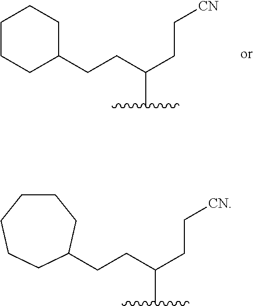

Another specific value for Rₐ is:

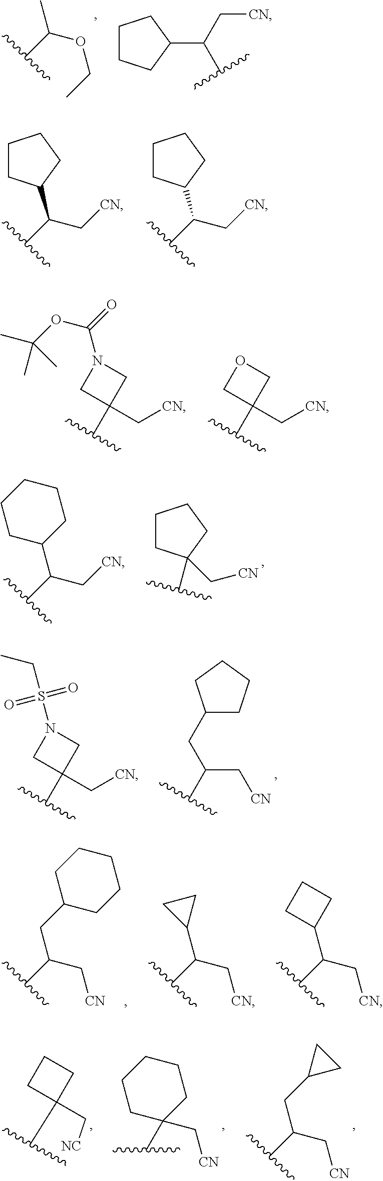

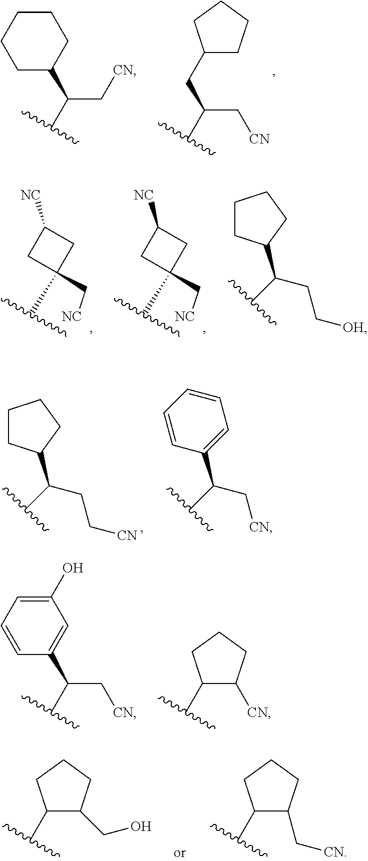

Another specific value for Rₐ is —NR_{z1}R₂.

Another specific value for Rₐ is:

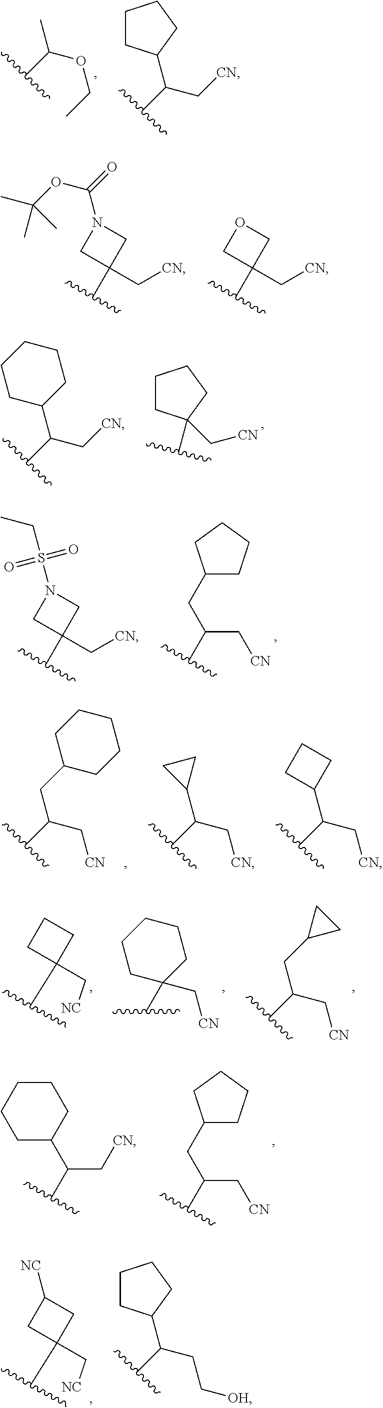

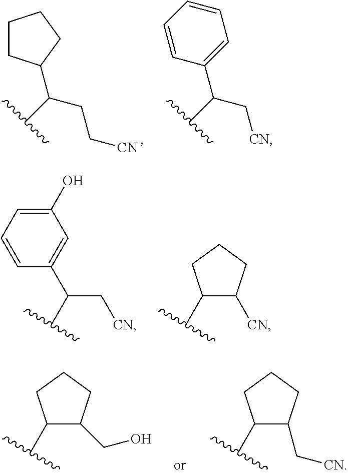

Another specific value for R₁ is:

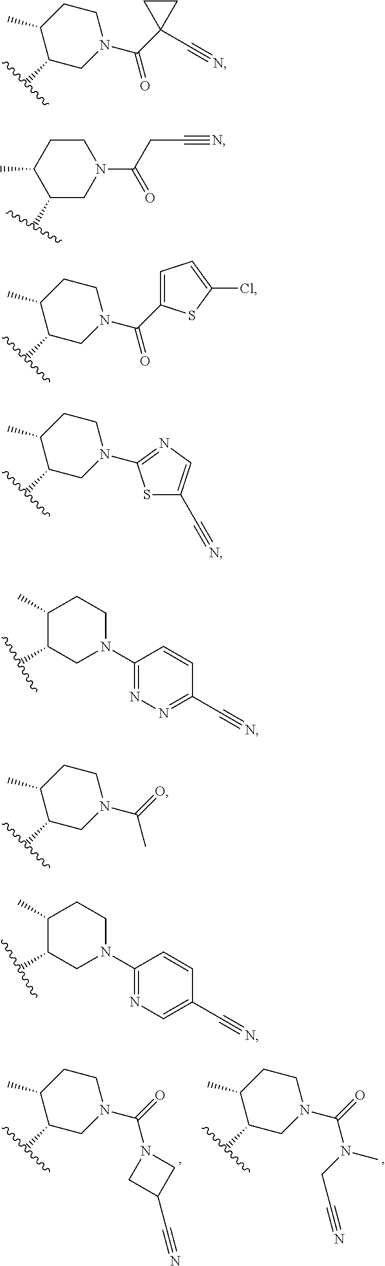

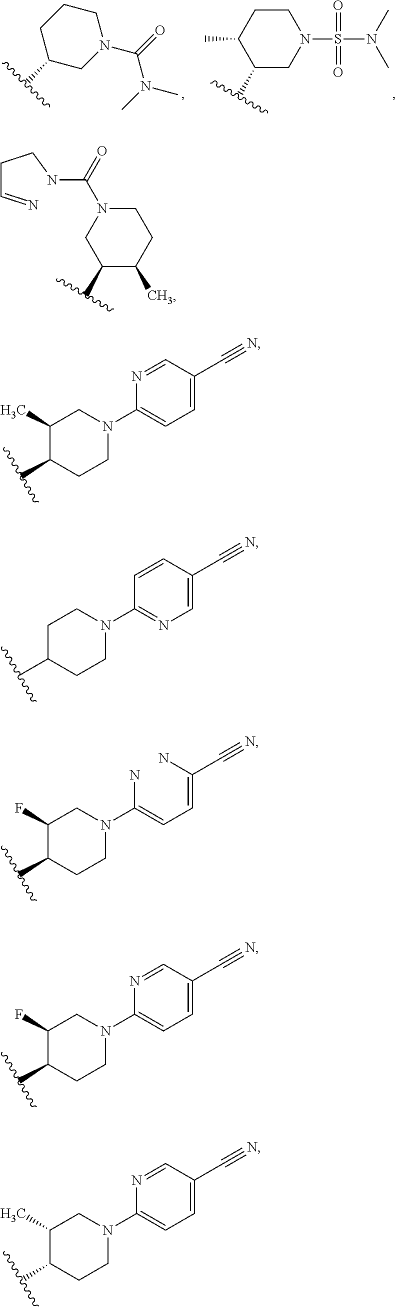

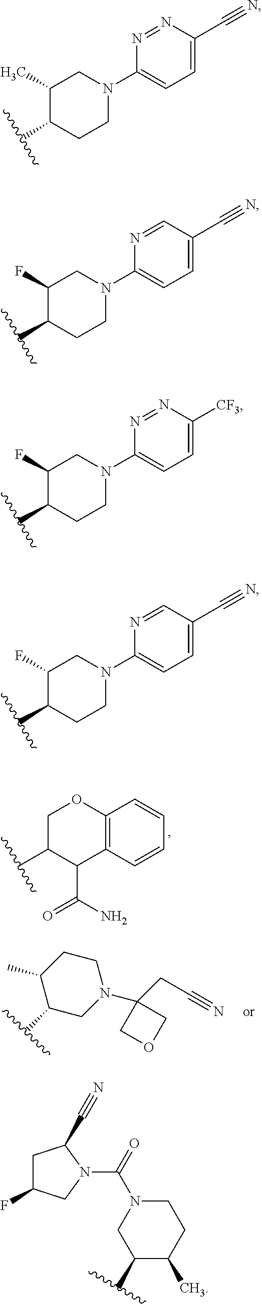

Another specific value for R₁ is:

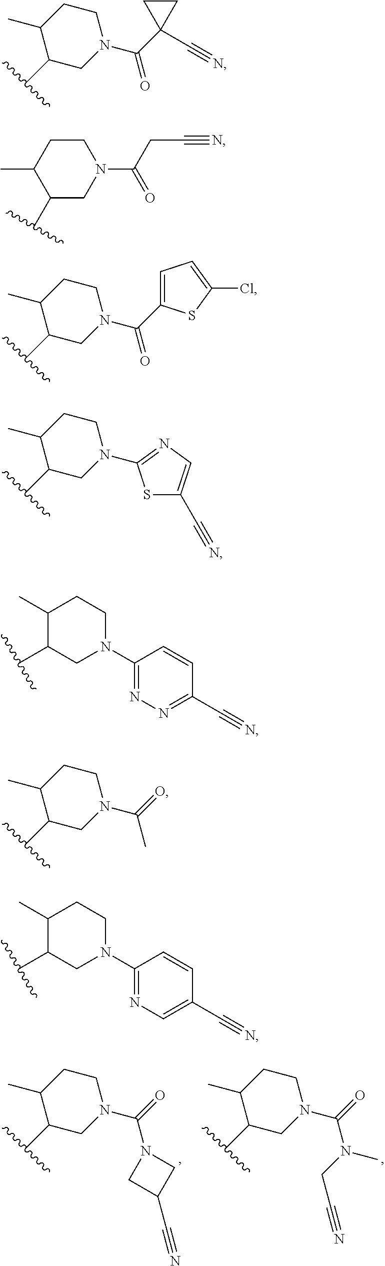

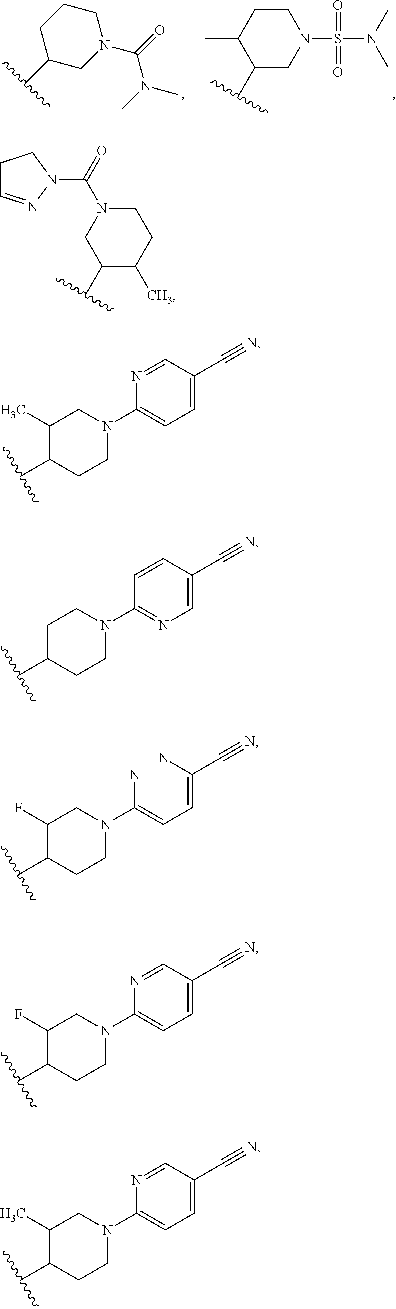

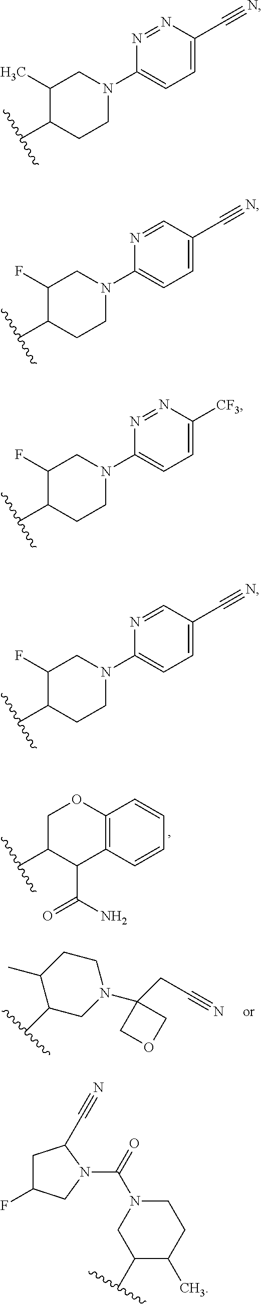

Another specific value for R₁ is:

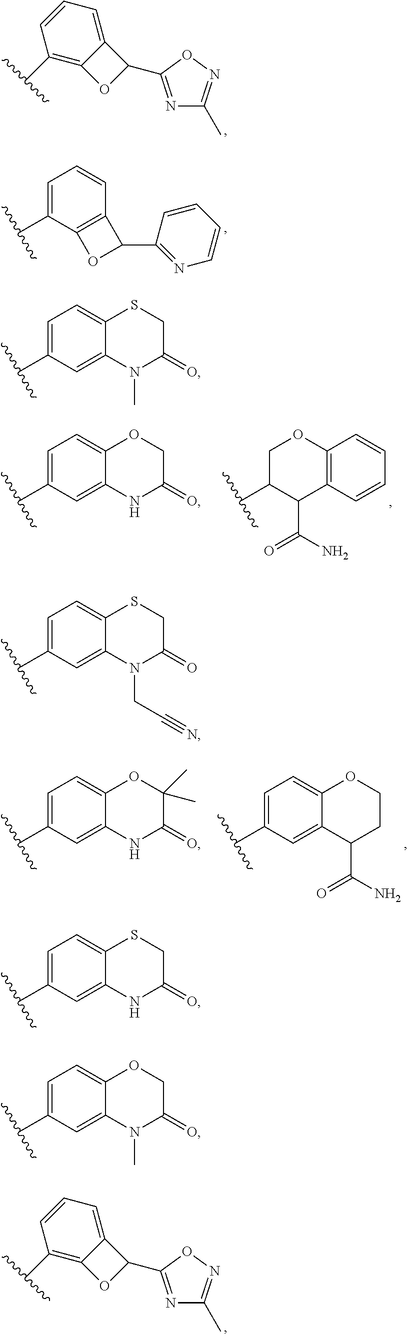

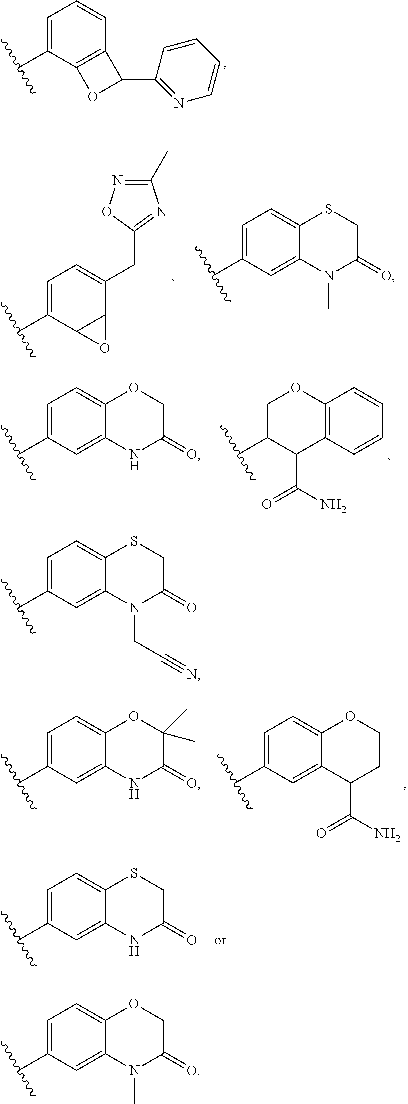

Another specific value for R₁ is:

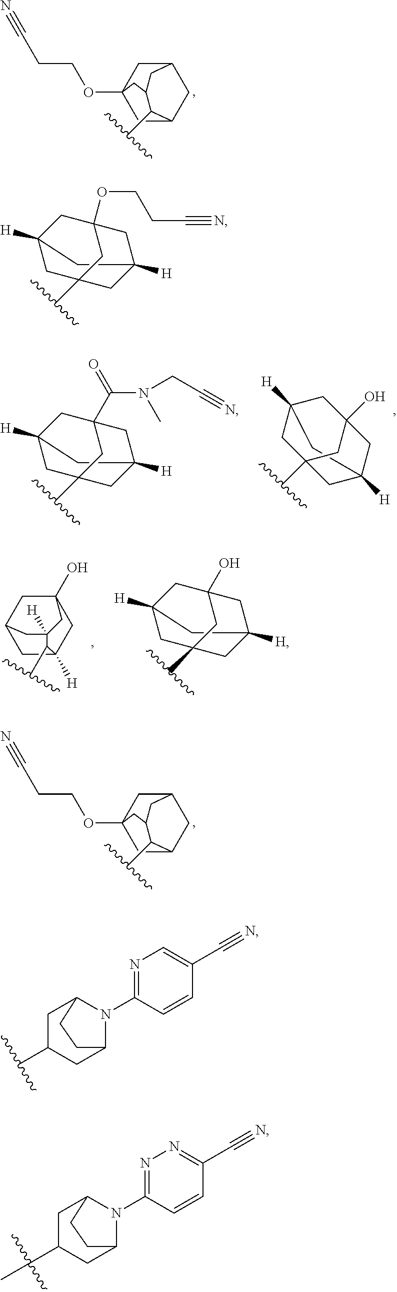

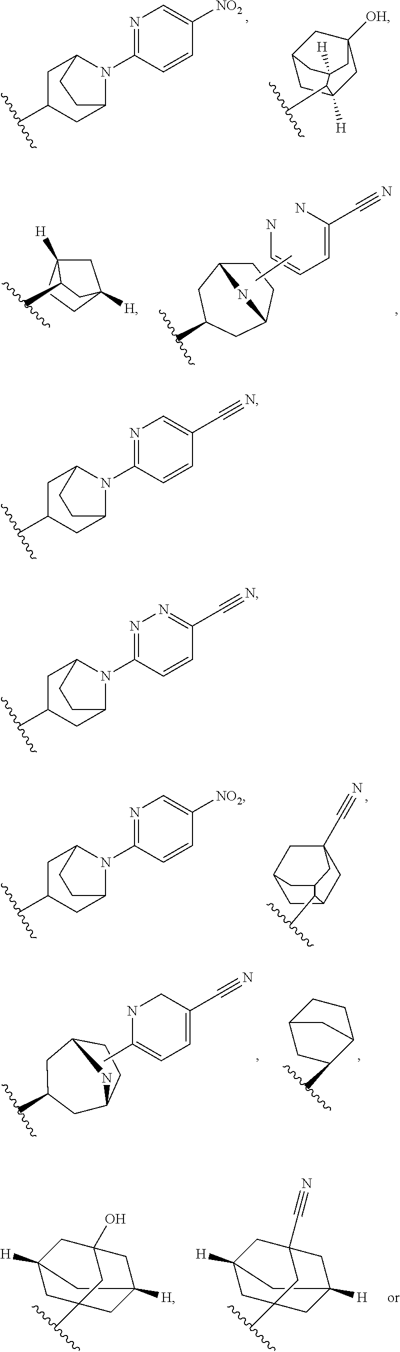

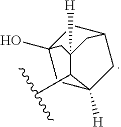

Another specific value for R₁ is:

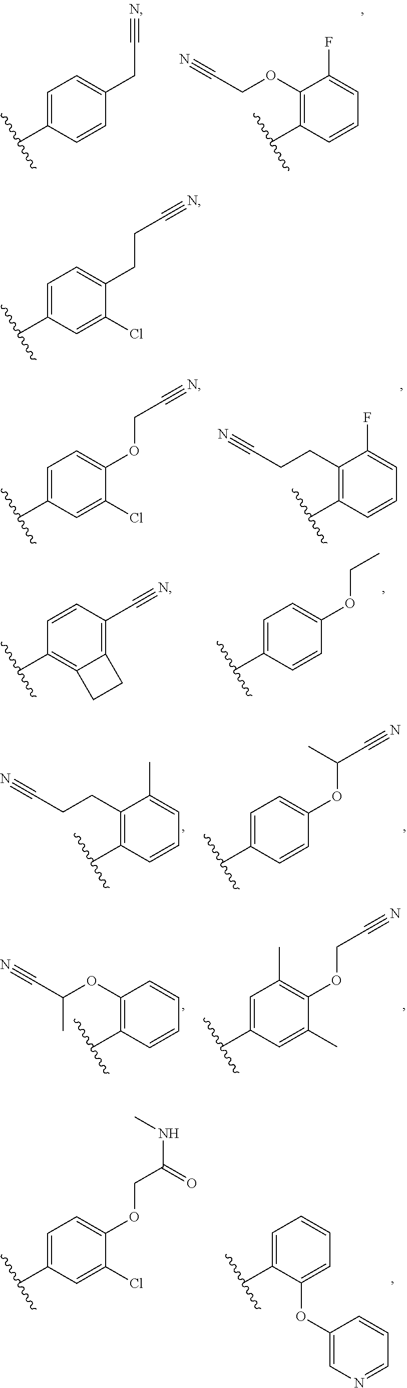

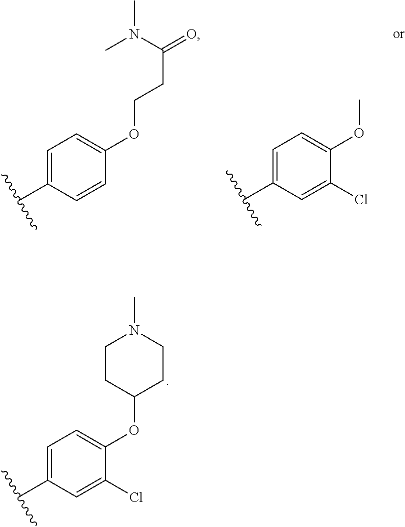

Another specific value for R₁ is:

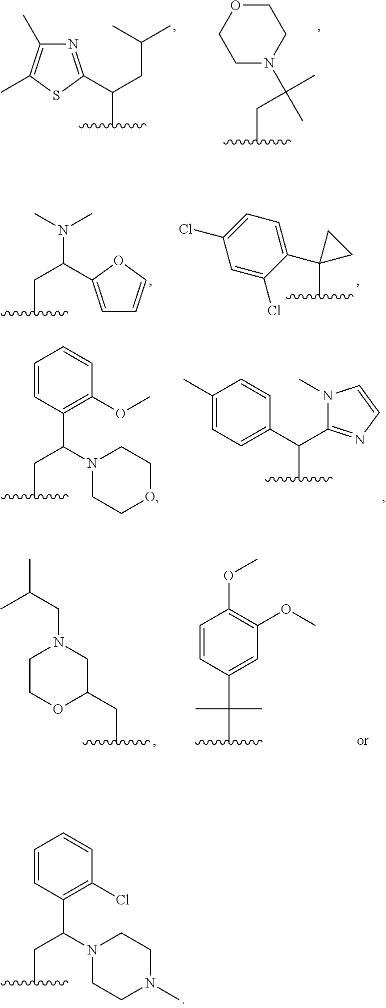

Another specific value for R₁ is:

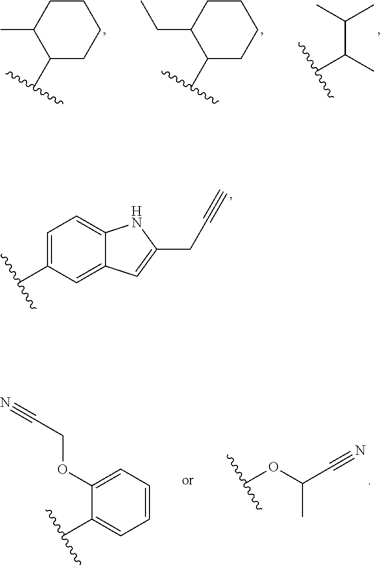

Another specific value for R₁ is:

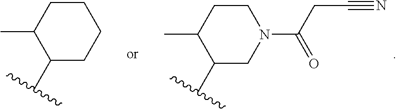

Another specific value for R₁ is:

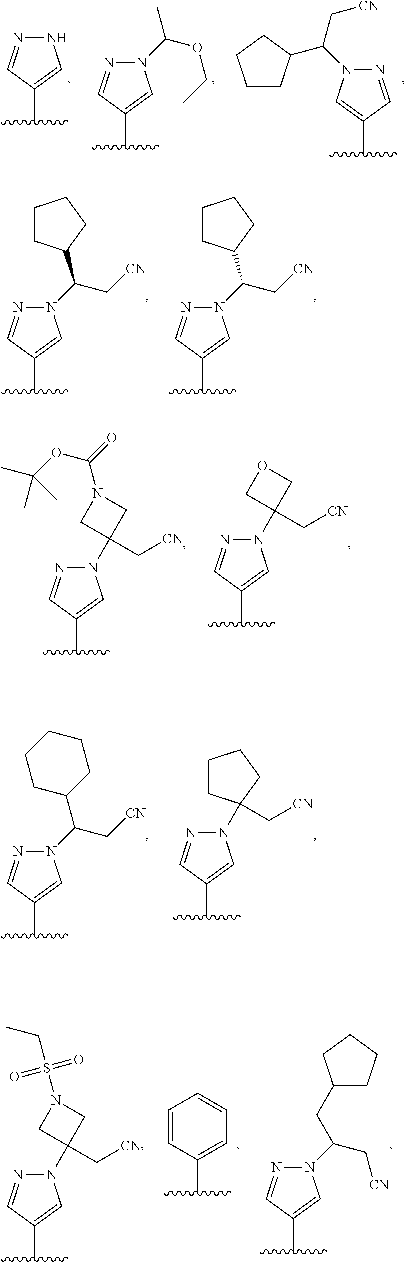

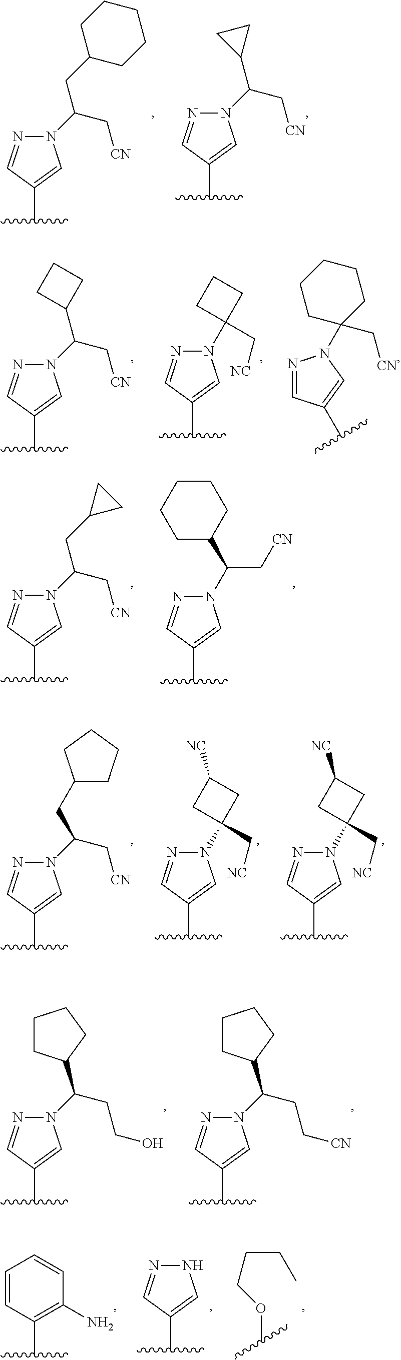

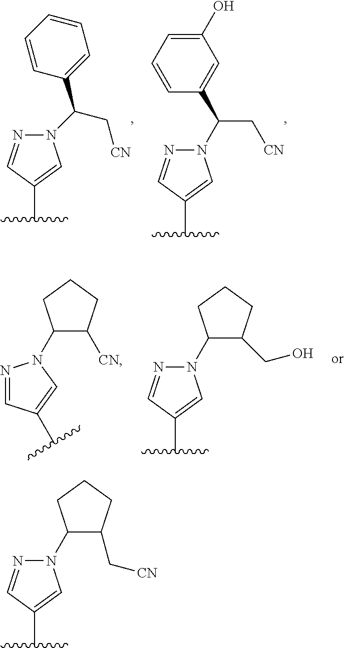

A specific group of compounds of formula I are compounds of formula:

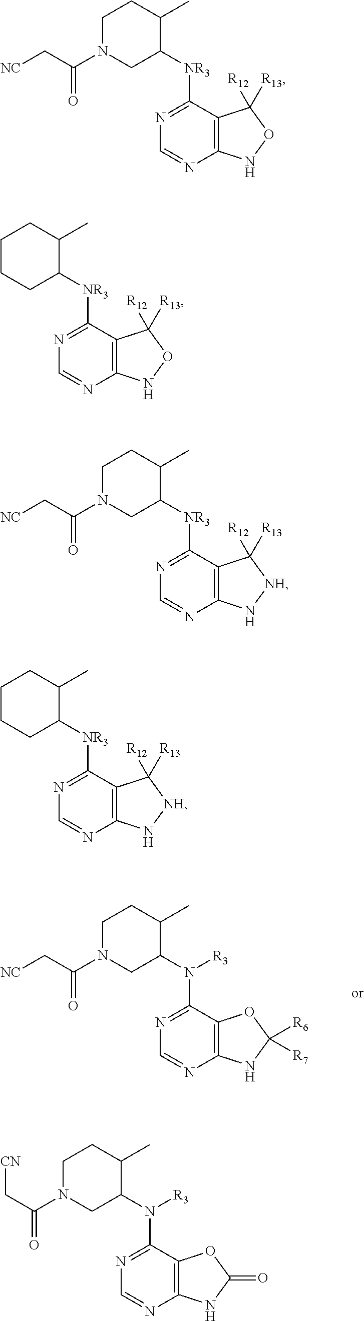

or a salt thereof.

Another specific group of compounds of formula I are compounds of formula:

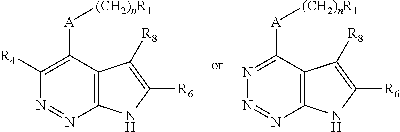

or a salt thereof.

In one embodiment of the invention, when X₁ is CR₄, X₂ is CR₅, Z is C═O and Y is O; then R₅ is H.

In another embodiment of the invention, when X₁ is CR₄, X₂ is CR₅, Z is C═O and Y is O; then R₅ is halogen, cycloalkyl, heteroaryl, heterocycle, NO₂, CN, —OH, —ORⱼ, —NRₖRₘ, N₃, SH, —SRⱼ, —C(O)Rₙ, —C(O)ORₙ, —C(O)NRₖRₘ, —C(═NRₖ)NRₖRₘ, —NRₖCON, —NRₖC(O)ORⱼ, —NR_{b}S(O)₂Rⱼ, —NRₖCONRₖRₘ, —OC(O)NRₖRₘ, —S(O)Rⱼ, —S(O)NRₖRₘ, —S(O)₂Rⱼ, —S(O)₂OH, or —S(O)₂NRₖRₘ; wherein any aryl or heteroaryl of R₅ is optionally substituted with one or more (e.g. 1, 2, 3, 4 or 5) Rₚ groups; and wherein any alkyl, cycloalkyl, alkenyl, alkynyl or heterocycle of R₅ is optionally substituted with one or more groups selected from Rₚ, oxo and ═NOR_{z};

In another embodiment of the invention when X₁ is N, X₂ is CR₅, Y is CR₆R₇ and Z is O; then R₅ is H.

In another embodiment of the invention when X₁ is N, X₂ is CR₅, Y is CR₆R₇ and Z is O; then R₅ is not —NRₖRₘ.

A specific compound of formula I is:

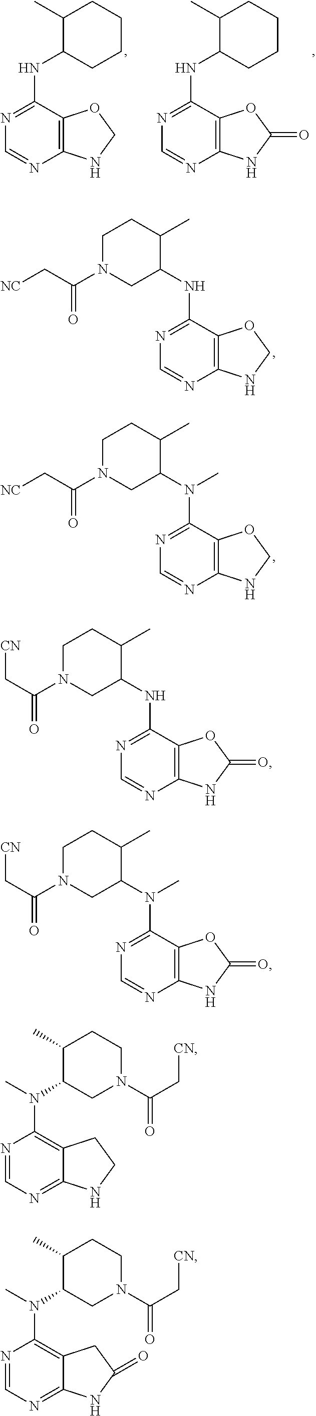

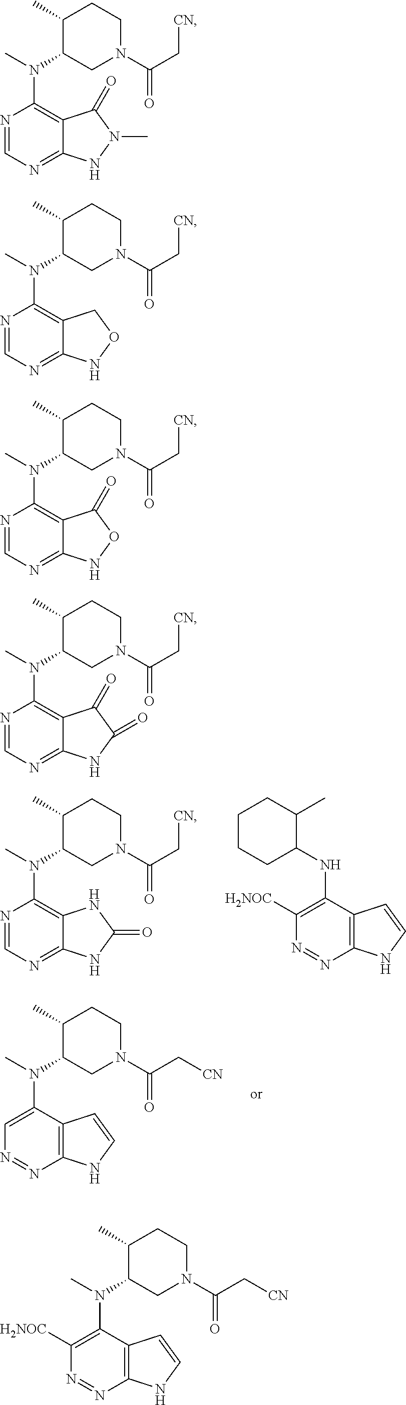

or a salt thereof.

Another specific compound of formula I is:

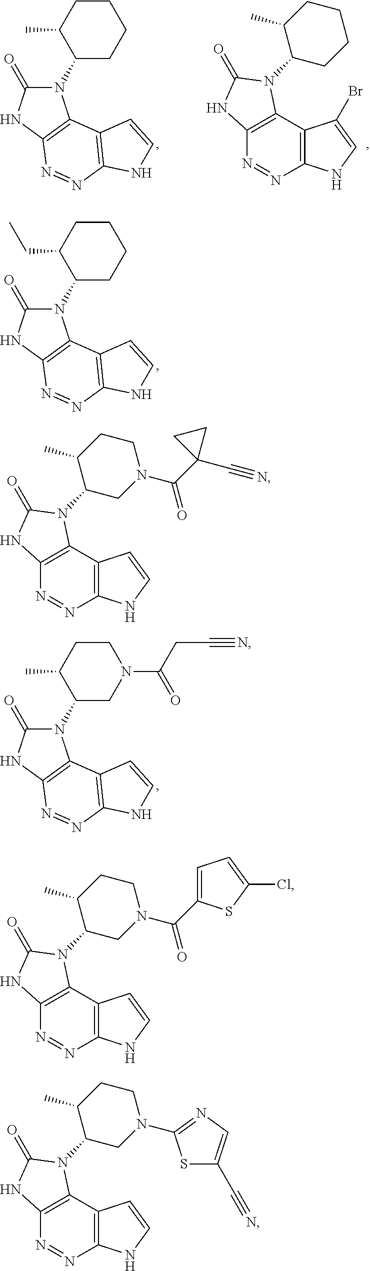

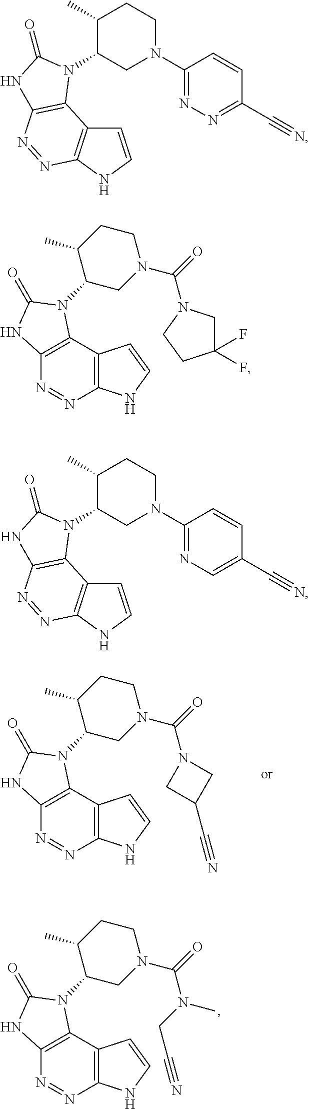

or a salt thereof.

Another specific compound of formula I is:

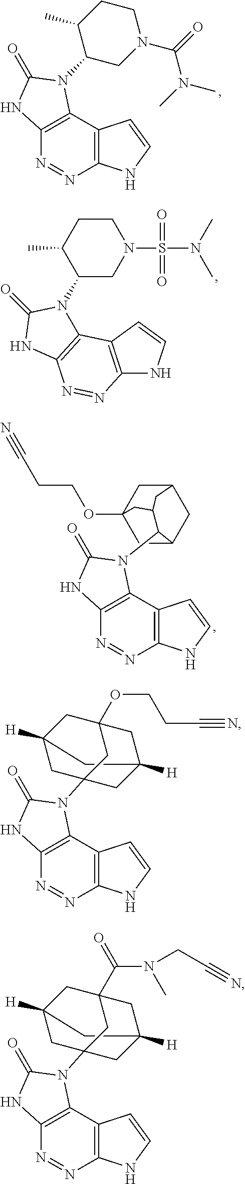

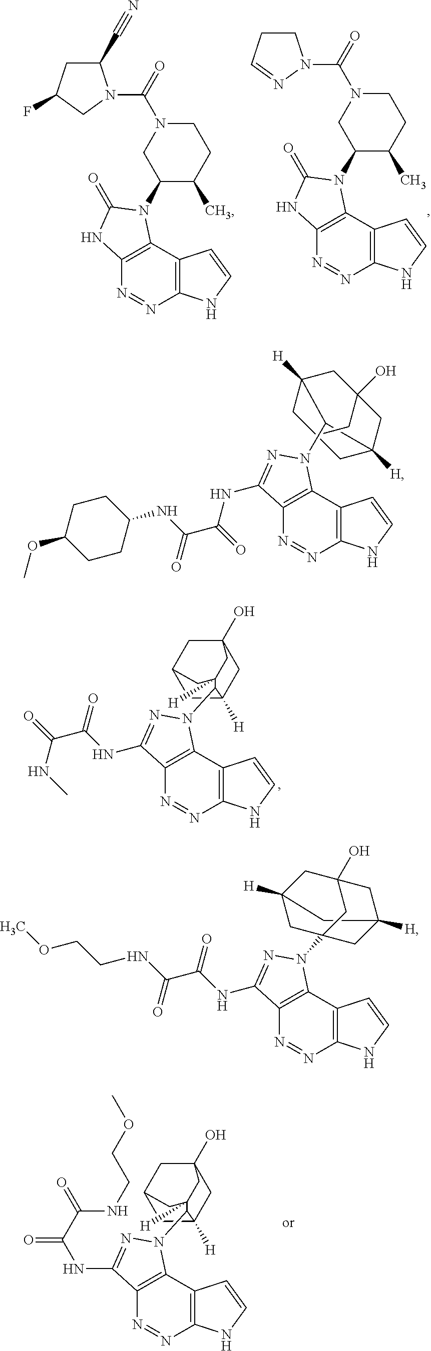

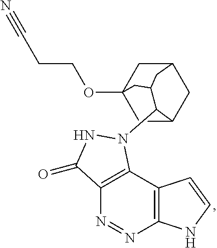

or a salt thereof.

Another specific compound of formula I is:

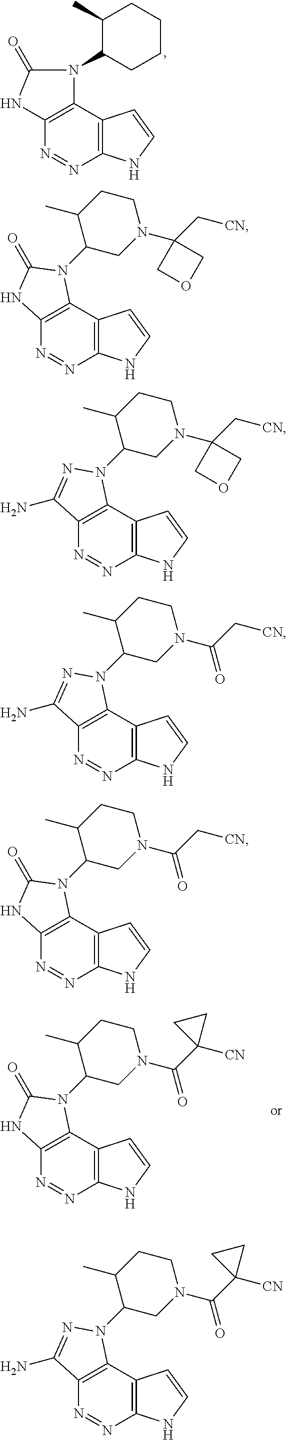

or a salt thereof.

Another specific compound of formula I is:

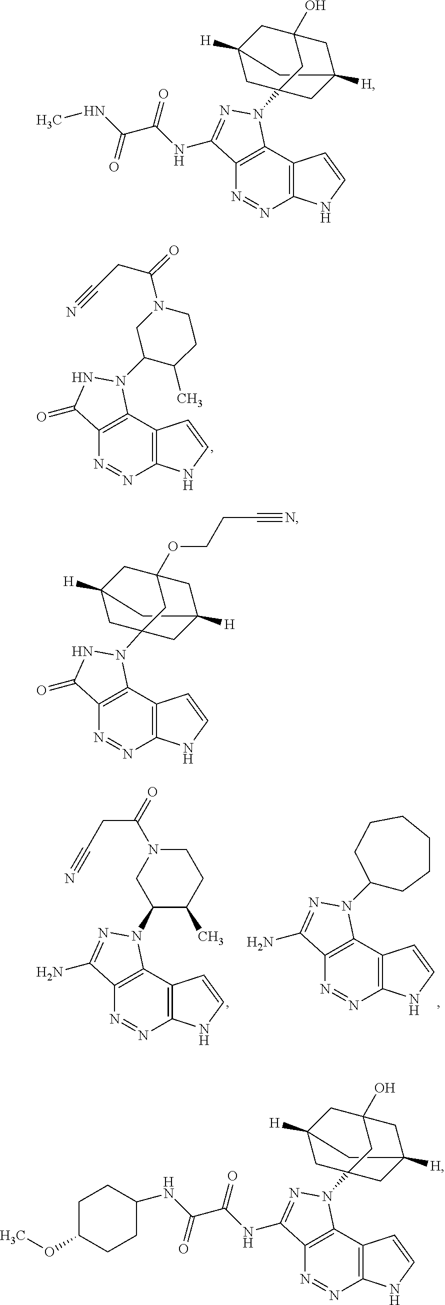

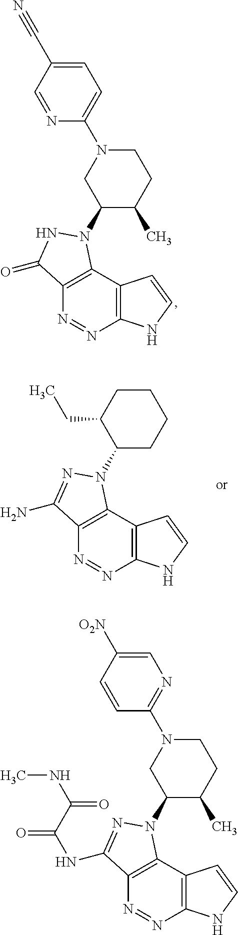

or a salt thereof.

Another specific compound of formula I is:

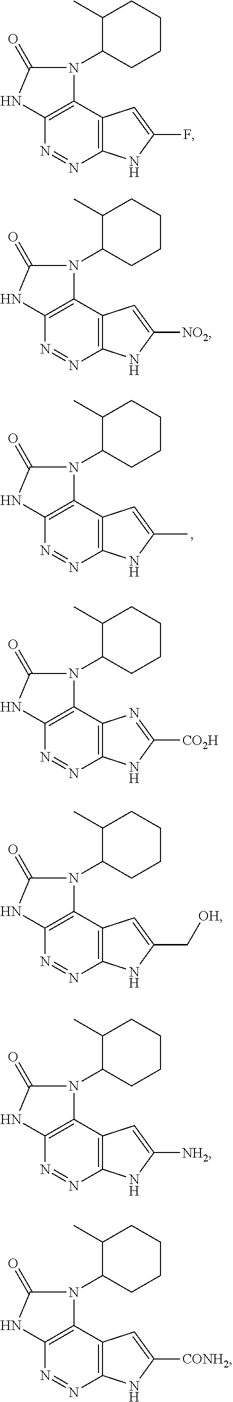

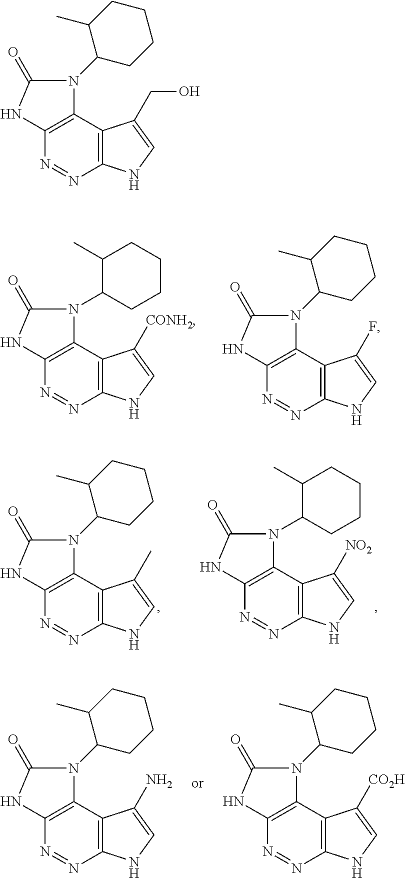

or a salt thereof.

Another specific compound of formula I is:

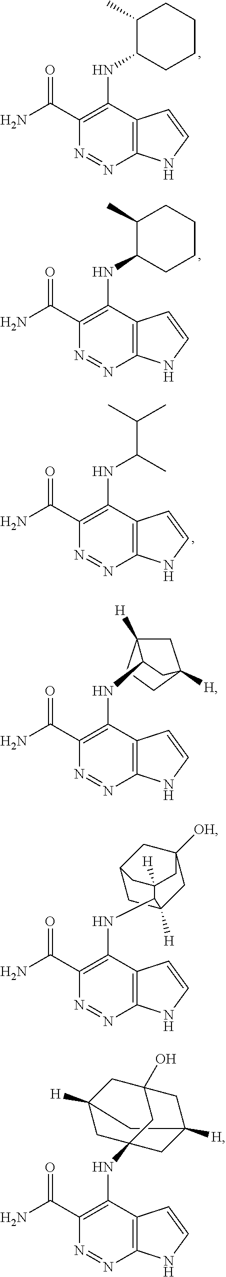

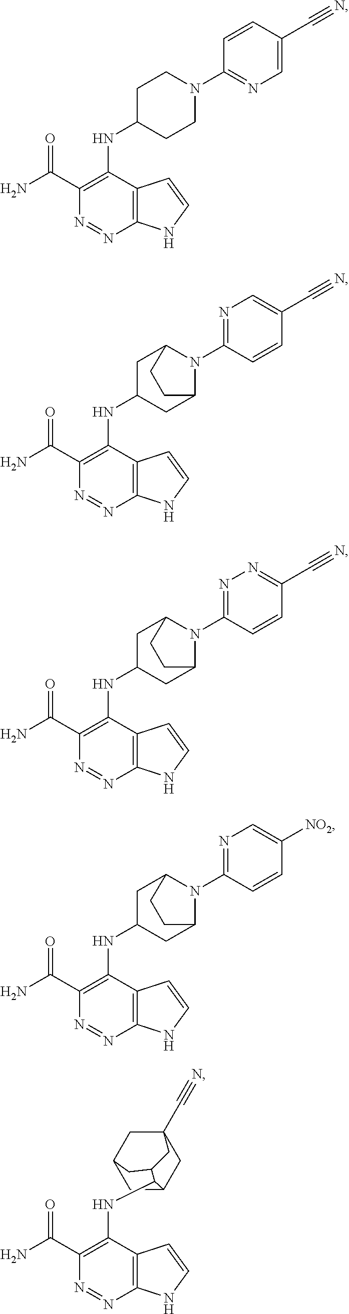

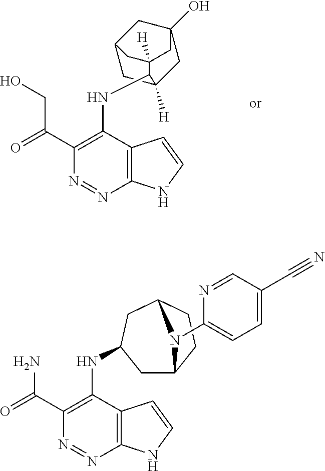

or a salt thereof.

Another specific compound of formula I is:

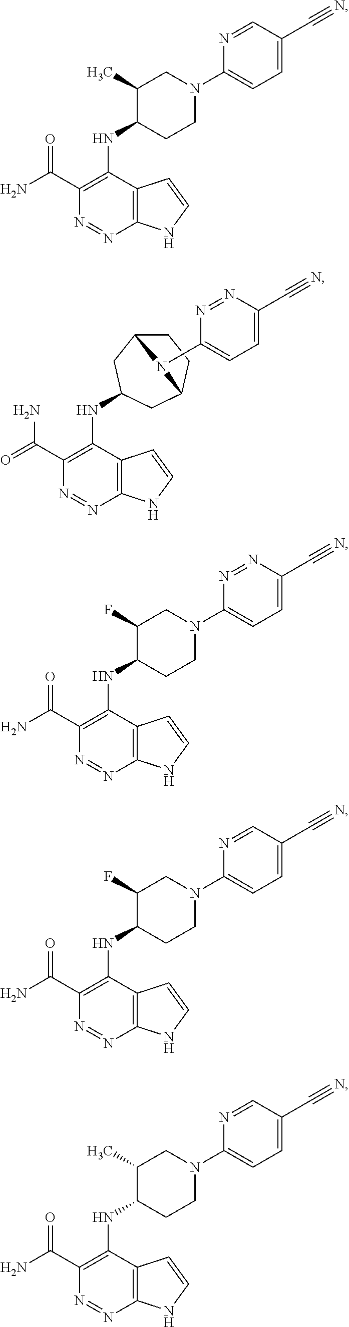

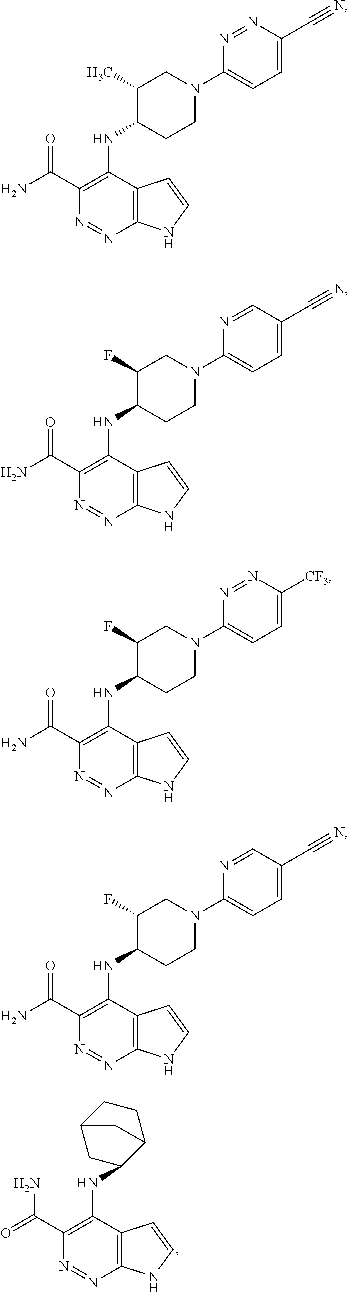

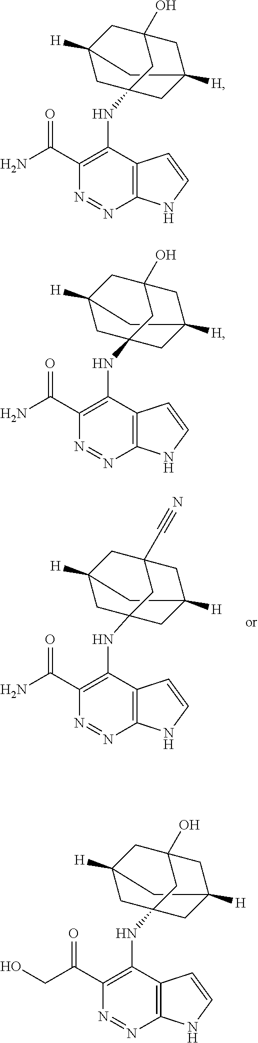

or a salt thereof.

Another specific compound of formula I is:

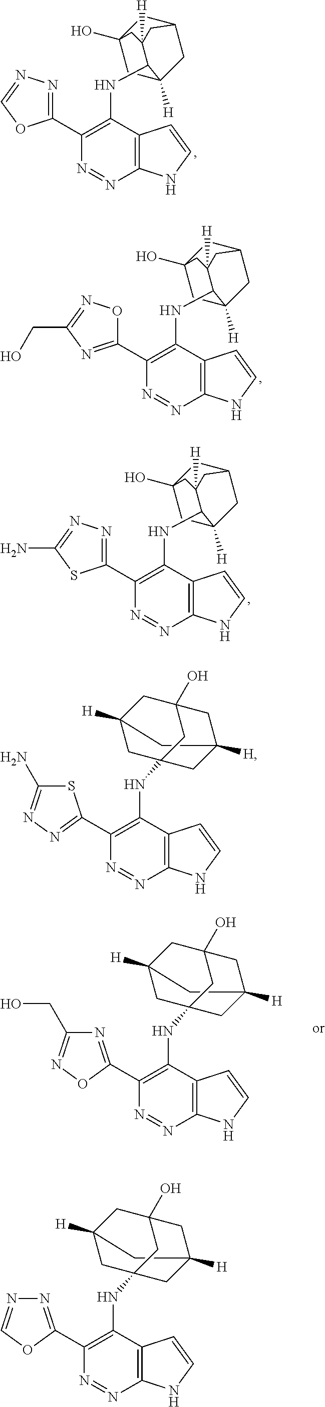

or a salt thereof.

Another specific compound of formula I is:

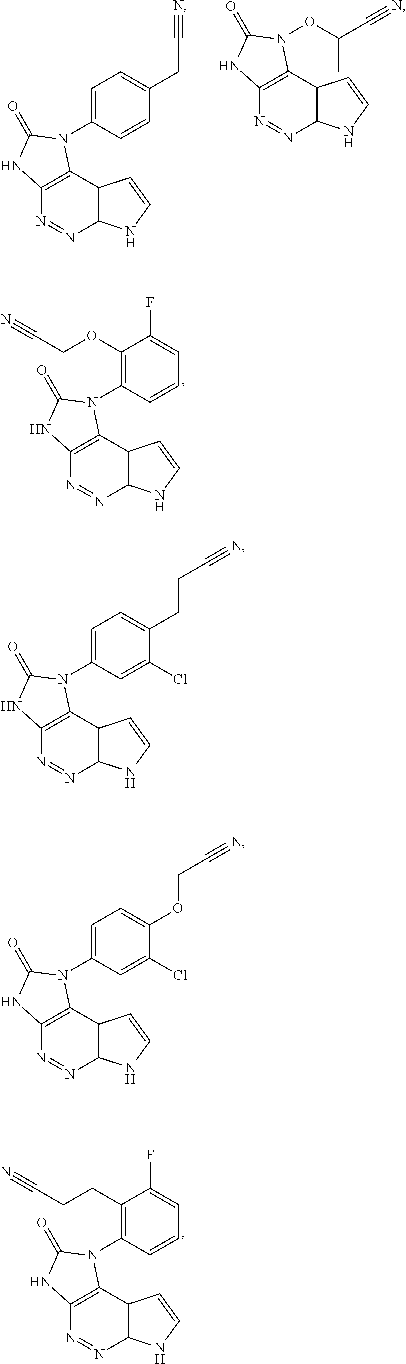

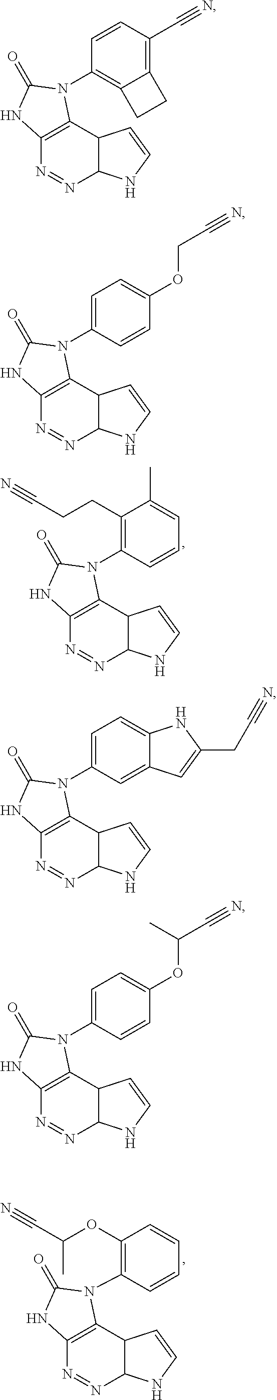

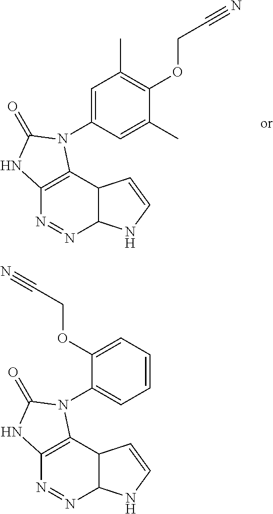

or a salt thereof.

Another specific compound of formula I is:

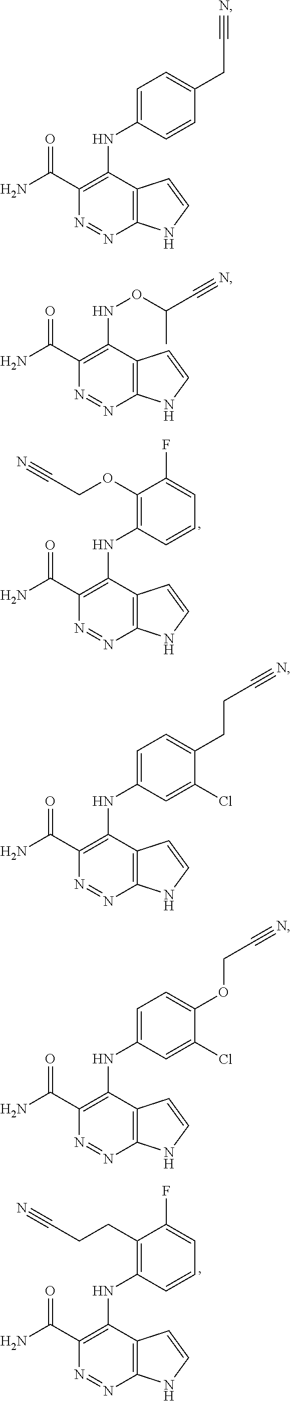

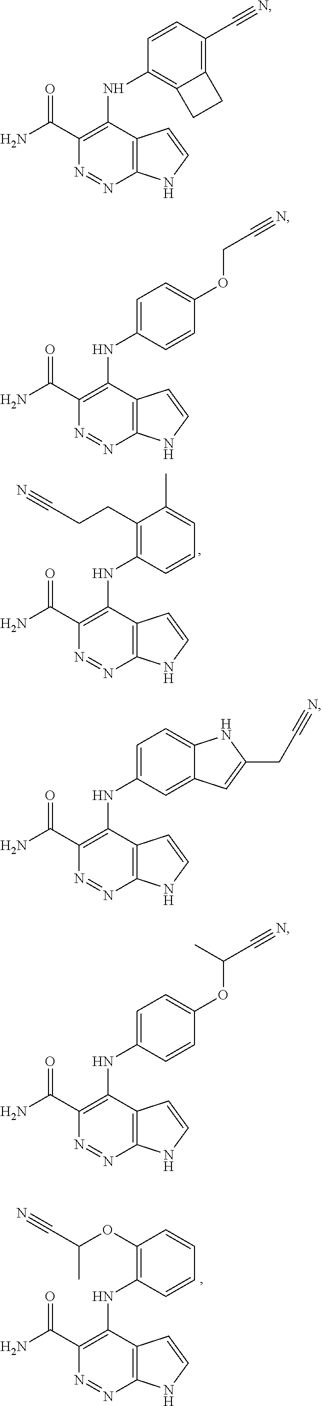

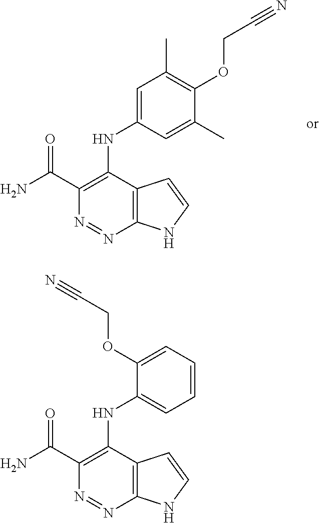

or a salt thereof.

Another specific compound of formula I is:

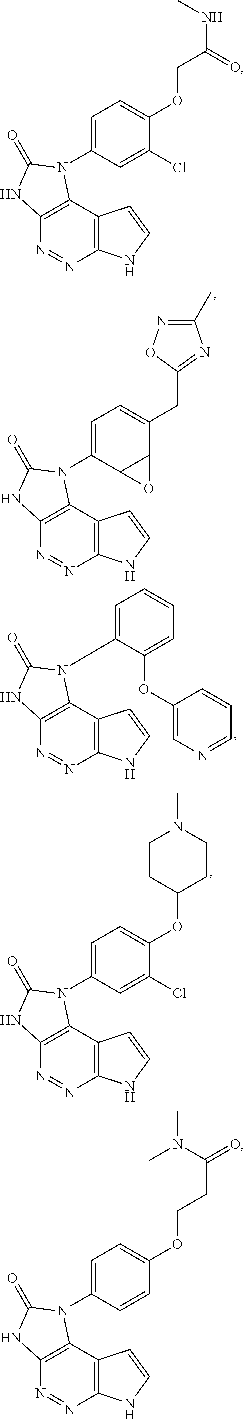

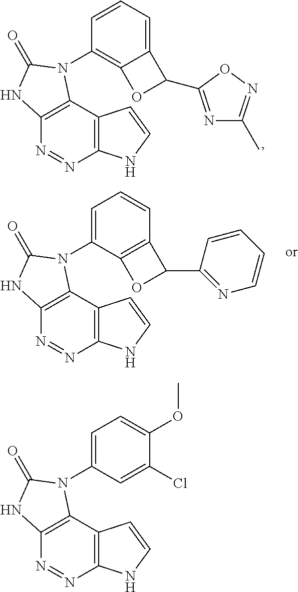

or a salt thereof.

Another specific compound of formula I is:

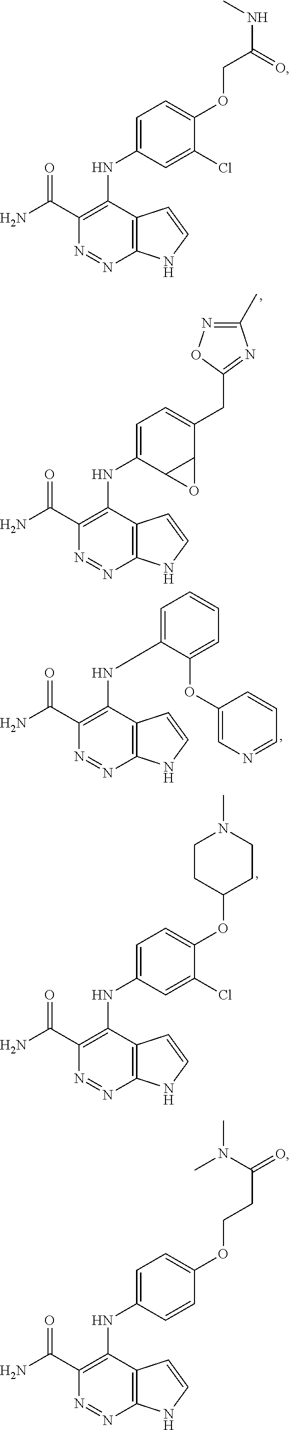

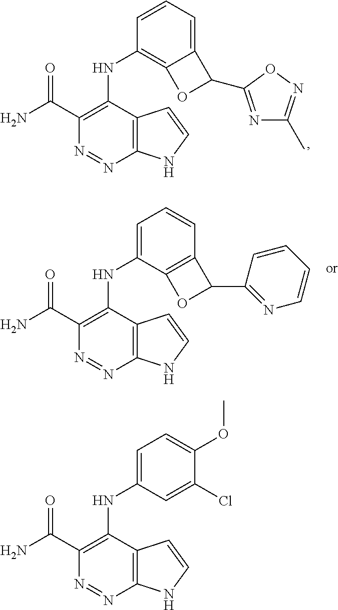

or a salt thereof.

Another specific compound of formula I is:

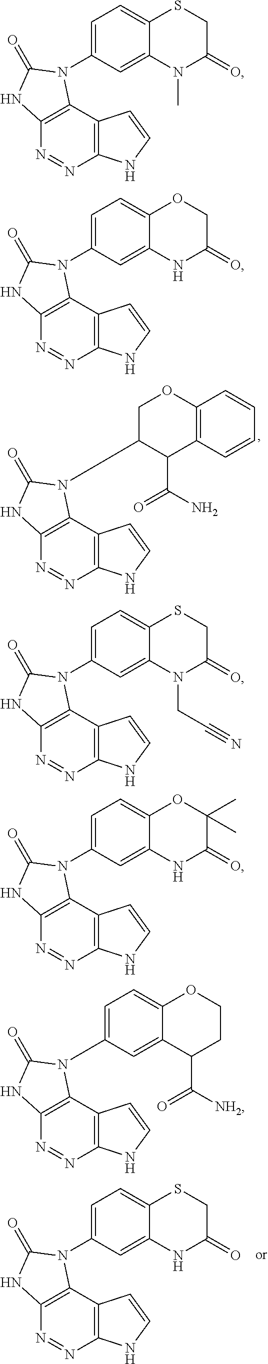

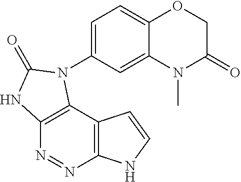

or a salt thereof.

Another specific compound of formula I is:

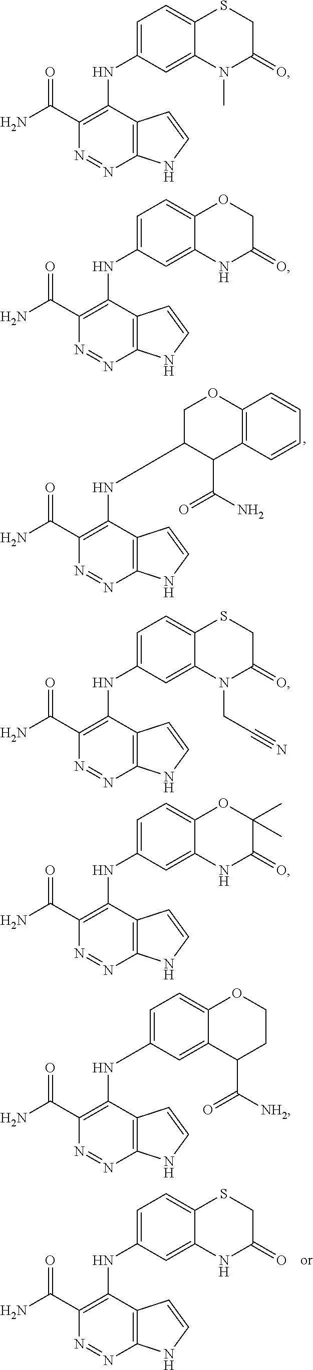

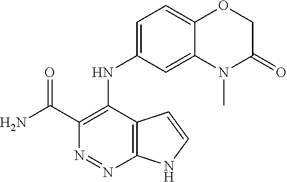

or a salt thereof.

Another specific compound of formula I is:

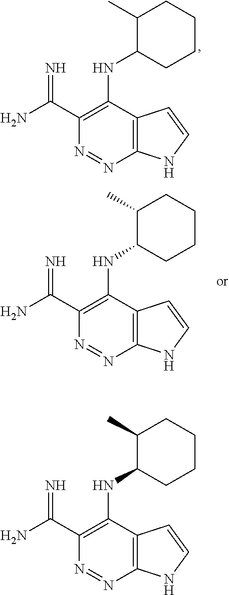

or a salt thereof.

Another specific compound of formula I is:

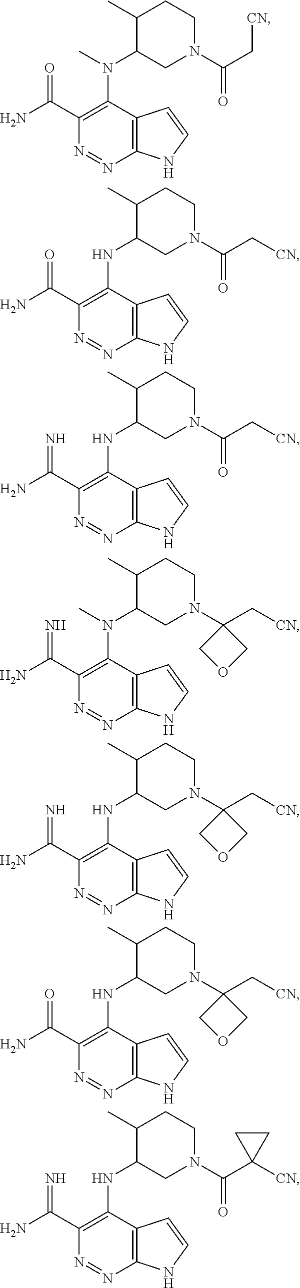

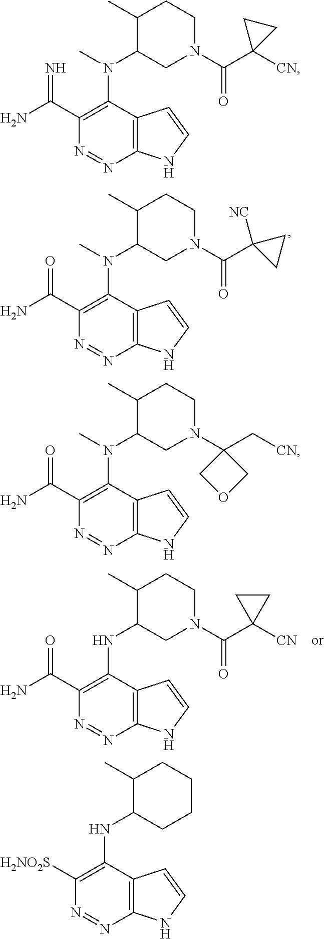

or a salt thereof.

Another specific compound of formula I is:

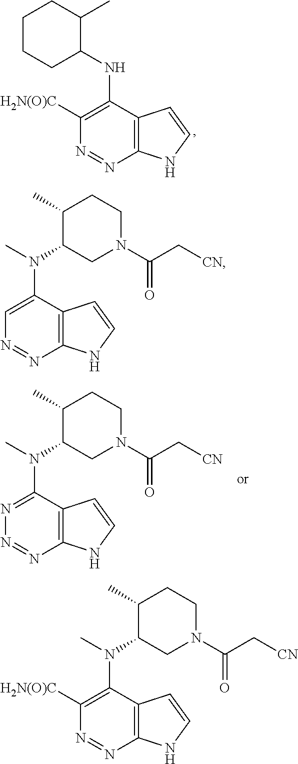

or a salt thereof.

Another specific compound of formula I is:

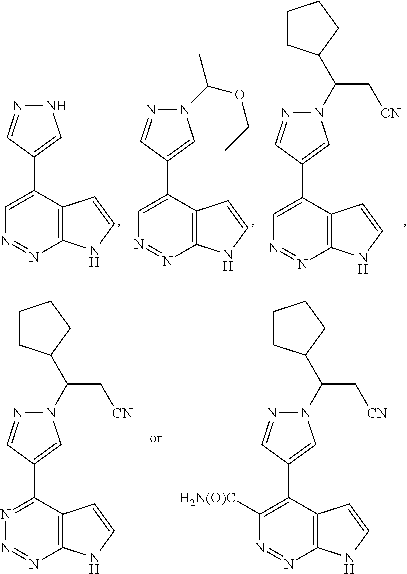

or a salt thereof.

Another specific compound of formula I is:

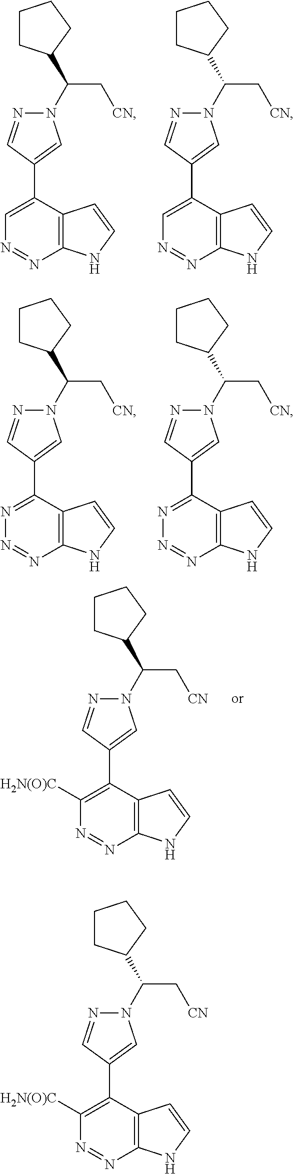

or a salt thereof.

Another specific compound of formula I is:

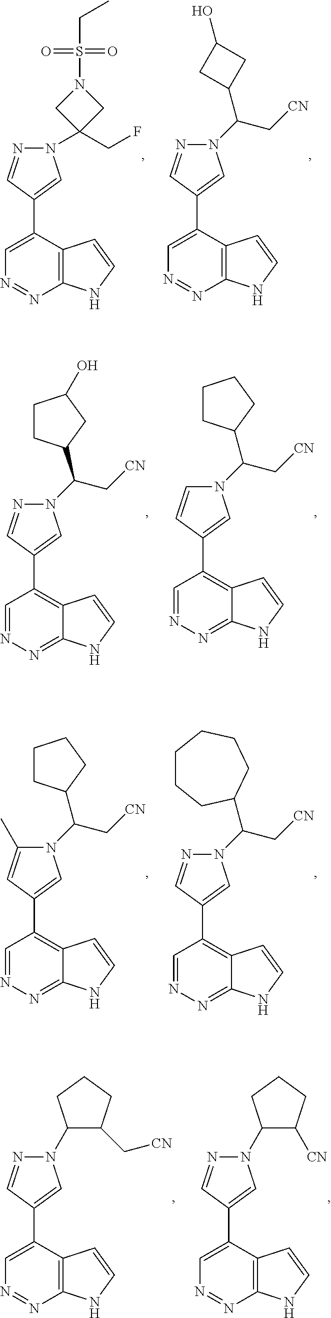

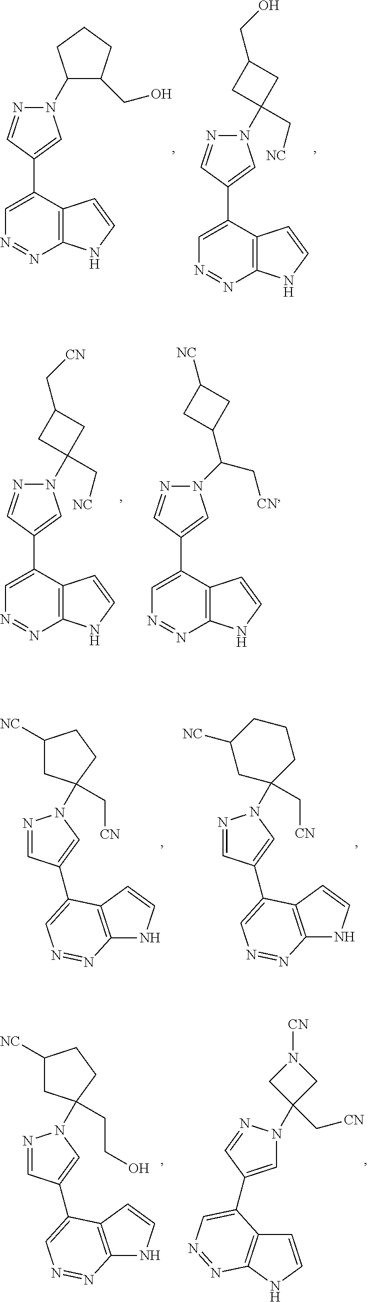

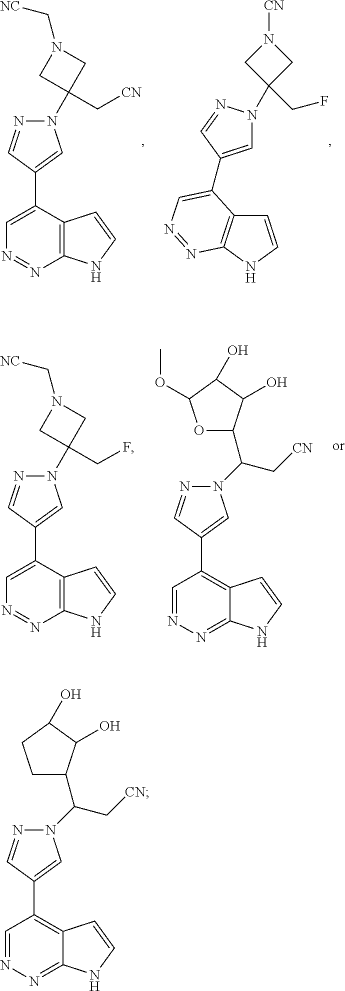

or a salt thereof.

Another specific compound of formula I is:
4-(1H-pyrazol-4-yl)-7H-pyrrolo[2,3-c]pyridazine;
4-(1-(1-ethoxyethyl)-1H-pyrazol-4-yl)-7H-pyrrolo[2,3-c]pyridazine;
3-(4-(7H-pyrrolo[2,3-c]pyridazin-4-yl)-1H-pyrazol-1-yl)-3-cyclopentylpropanenitrile;
(R)-3-(4-(7H-pyrrolo[2,3-c]pyridazin-4-yl)-1H-pyrazol-1-yl)-3-cyclopentyl propanenitrile;
(S)-3-(4-(7H-pyrrolo[2,3-c]pyridazin-4-yl)-1H-pyrazol-1-yl)-3-cyclopentyl propanenitrile;
tert-butyl 3-(4-(7H-pyrrolo[2,3-c]pyridazin-4-yl)-1H-pyrazol-1-yl)-3-(cyanomethyl)azetidine-1-carboxylate;
2-(3-(4-(7H-pyrrolo[2,3-c]pyridazin-4-yl)-1H-pyrazol-1-yl)oxetan-3-yl)acetonitrile;
3-(4-(7H-pyrrolo[2,3-c]pyridazin-4-yl)-1H-pyrazol-1-yl)-3-cyclohexylpropane nitrile;
2-(1-(4-(7H-pyrrolo[2,3-c]pyridazin-4-yl)-1H-pyrazol-1-yl)cyclopentyl)acetonitrile;
2-(3-(4-(7H-pyrrolo[2,3-c]pyridazin-4-yl)-1H-pyrazol-1-yl)-1-(ethylsulfonyl)azetidin-3-yl)acetonitrile;
4-phenyl-7H-pyrrolo[2,3-c]pyridazine;
3-(4-(7H-pyrrolo[2,3-c]pyridazin-4-yl)-1H-pyrazol-1-yl)-4-cyclopentylbutane nitrile;
3-(4-(7H-pyrrolo[2,3-c]pyridazin-4-yl)-1H-pyrazol-1-yl)-4-cyclohexylbutane nitrile;
3-(4-(7H-pyrrolo[2,3-c]pyridazin-4-yl)-1H-pyrazol-1-yl)-3-cyclopropylpropane nitrile;
3-(4-(7H-pyrrolo[2,3-c]pyridazin-4-yl)-1H-pyrazol-1-yl)-3-cyclobutylpropane nitrile;
2-(1-(4-(7H-pyrrolo[2,3-c]pyridazin-4-yl)-1H-pyrazol-1-yl)cyclobutyl)acetonitrile;
2-(1-(4-(7H-pyrrolo[2,3-c]pyridazin-4-yl)-1H-pyrazol-1-yl)cyclohexyl)acetonitrile;
3-(4-(7H-pyrrolo[2,3-c]pyridazin-4-yl)-1H-pyrazol-1-yl)-4-cyclopropylbutane nitrile;
(R)-3-(4-(7H-pyrrolo[2,3-c]pyridazin-4-yl)-1H-pyrazol-1-yl)-3-cyclohexylpropane nitrile;
(S)-3-(4-(7H-pyrrolo[2,3-c]pyridazin-4-yl)-1H-pyrazol-1-yl)-4-cyclopentylbutane nitrile;
(Z)-3-(4-(7H-pyrrolo[2,3-c]pyridazin-4-yl)-1H-pyrazol-1-yl)-3-(cyanomethyl)cyclobutanecarbonitrile;
(E)-3-(4-(7H-pyrrolo[2,3-c]pyridazin-4-yl)-1H-pyrazol-1-yl)-3-(cyanomethyl)cyclobutanecarbonitrile;
(R)-3-(4-(7H-pyrrolo[2,3-c]pyridazin-4-yl)-1H-pyrazol-1-yl)-3-cyclopentyl propan-1-ol;
(R)-4-(4-(7H-pyrrolo[2,3-c]pyridazin-4-yl)-1H-pyrazol-1-yl)-4-cyclopentylbutane nitrile;
2-(7H-pyrrolo[2,3-c]pyridazin-4-yl)aniline;
4-(1H-pyrrol-3-yl)-7H-pyrrolo[2,3-c]pyridazine;
(R)-3-(4-(7H-pyrrolo[2,3-c]pyridazin-4-yl)-1H-pyrazol-1-yl)-3-phenylpropane nitrile;
(R)-3-(4-(7H-pyrrolo[2,3-c]pyridazin-4-yl)-1H-pyrazol-1-yl)-3-(3-hydroxyphenyl);
4-hydroxy-7H-pyrrolo[2,3-d][1,2,3]triazine-5-carboxamide;
2-(4-(7H-pyrrolo[2,3-c]pyridazin-4-yl)-1H-pyrazol-1-yl)cyclopentane carbonitrile;
(2-(4-(7H-pyrrolo[2,3-c]pyridazin-4-yl)-1H-pyrazol-1-yl)cyclopentyl)methanol;
2-(2-(4-(7H-pyrrolo[2,3-c]pyridazin-4-yl)-1H-pyrazol-1-yl)cyclopentyl)acetonitrile; or
3-(4-methyl-3-(methyl(6-oxo-6,7-dihydro-5H-pyrrolo[2,3-d]pyrimidin-4-yl)amino)piperidin-1-yl)-3-oxopropanenitrile; or a salt thereof.

Another specific compound of formula I is:
(1R,2S)-2-(4-(7H-pyrrolo[2,3-c]pyridazin-4-yl)-1H-pyrazol-1-yl)cyclopentanecarbonitrile;
(1S,2S)-2-(4-(7H-pyrrolo[2,3-c]pyridazin-4-yl)-1,4-pyrazol-1-yl)cyclopentanecarbonitrile;
(1S,2R)-2-(4-(7H-pyrrolo[2,3-c]pyridazin-4-yl)-1H-pyrazol-1-yl)cyclopentanecarbonitrile;
(1R,2R)-2-(4-(7H-pyrrolo[2,3-c]pyridazin-4-yl)-1H-pyrazol-1-yl)cyclopentanecarbonitrile;
((1S,2S)-2-(4-(7H-pyrrolo[2,3-c]pyridazin-4-yl)-1H-pyrazol-1-yl)cyclopentyl)methanol;
((1R,2S)-2-(4-(7H-pyrrolo[2,3-c]pyridazin-4-yl)-1H-pyrazol-1-yl)cyclopentyl)methanol;
((1R,2R)-2-(4-(7H-pyrrolo[2,3-c]pyridazin-4-yl)-1H-pyrazol-1-yl)cyclopentyl)methanol;
((1S,2R)-2-(4-(7H-pyrrolo[2,3-c]pyridazin-4-yl)-1H-pyrazol-1-yl)cyclopentyl)methanol;
2-(2-(4-(7H-pyrrolo[2,3-c]pyridazin-4-yl)-1H-pyrazol-1-yl)cyclopentyl)acetonitrile;
2-((1R,2 S)-2-(4-(7H-pyrrolo[2,3-c]pyridazin-4-yl)-1H-pyrazol-1-yl)cyclopentyl)acetonitrile;
2-((1S,2S)-2-(4-(7H-pyrrolo[2,3-c]pyridazin-4-yl)-1H-pyrazol-1-yl)cyclopentyl)acetonitrile;
2-((1S,2R)-2-(4-(7H-pyrrolo[2,3-c]pyridazin-4-yl)-1H-pyrazol-1-yl)cyclopentyl)acetonitrile;
2-((1R,2R)-2-(4-(7H-pyrrolo[2,3-c]pyridazin-4-yl)-1H-pyrazol-1-yl)cyclopentyl)acetonitrile;
(S)-3-(4-(7H-pyrrolo[2,3-c]pyridazin-4-yl)-1H-pyrazol-1-yl)-3-cyclohexylpropanenitrile;
(R)-3-(4-(7H-pyrrolo[2,3-c]pyridazin-4-yl)-1H-pyrazol-1-yl)-4-cyclopentylbutanenitrile;
(S)-3-(4-(7H-pyrrolo[2,3-c]pyridazin-4-yl)-1H-pyrazol-1-yl)-4-cyclohexylbutanenitrile;
(R)-3-(4-(7H-pyrrolo[2,3-c]pyridazin-4-yl)-1H-pyrazol-1-yl)-4-cyclohexylbutanenitrile;
(R)-3-(4-(7H-pyrrolo[2,3-c]pyridazin-4-yl)-1H-pyrazol-1-yl)-3-cyclopropylpropanenitrile;
(S)-3-(4-(7H-pyrrolo[2,3-c]pyridazin-4-yl)-1H-pyrazol-1-yl)-3-cyclopropylpropanenitrile;
(R)-3-(4-(7H-pyrrolo[2,3-c]pyridazin-4-yl)-1H-pyrazol-1-yl)-3-cyclobutylpropanenitrile;
(S)-3-(4-(7H-pyrrolo[2,3-c]pyridazin-4-yl)-1H-pyrazol-1-yl)-3-cyclobutylpropanenitrile;
(R)-3-(4-(7H-pyrrolo[2,3-c]pyridazin-4-yl)-1H-pyrazol-1-yl)-4-cyclopropylbutanenitrile;
(S)-3-(4-(7H-pyrrolo[2,3-c]pyridazin-4-yl)-1H-pyrazol-1-yl)-4-cyclopropylbutanenitrile;
3-(4-(7H-pyrrolo[2,3-c]pyridazin-4-yl)-1H-pyrazol-1-yl)-3-cyclopentylpropan-1-ol;
(S)-3-(4-(7H-pyrrolo[2,3-c]pyridazin-4-yl)-1H-pyrazol-1-yl)-3-cyclopentylpropan-1-ol;
4-(4-(7H-pyrrolo[2,3-c]pyridazin-4-yl)-1H-pyrazol-1-yl)-4-cyclopentylbutanenitrile;
(S)-4-(4-(7H-pyrrolo[2,3-c]pyridazin-4-yl)-1H-pyrazol-1-yl)-4-cyclopentylbutanenitrile;
3-(4-(7H-pyrrolo[2,3-c]pyridazin-4-yl)-1H-pyrazol-1-yl)-3-phenylpropanenitrile;
(S)-3-(4-(7H-pyrrolo[2,3-c]pyridazin-4-yl)-1H-pyrazol-1-yl)-3-phenylpropanenitrile;
3-(4-(7H-pyrrolo[2,3-c]pyridazin-4-yl)-1H-pyrazol-1-yl)-3-(3-hydroxyphenyl)propanenitrile;
(S)-3-(4-(7H-pyrrolo[2,3-c]pyridazin-4-yl)-1H-pyrazol-1-yl)-3-(3-hydroxyphenyl)propanenitrile;
3-(4-(7H-pyrrolo[2,3-c]pyridazin-4-yl)-1H-pyrazol-1-yl)-3-(2-hydroxyphenyl)propanenitrile;
(S)-3-(4-(7H-pyrrolo[2,3-C]pyridazin-4-yl)-1H-pyrazol-1-yl)-3-(2-hydroxyphenyl)propanenitrile;
(R)-3-(4-(7H-pyrrolo[2,3-c]pyridazin-4-yl)-1H-pyrazol-1-yl)-3-(2-hydroxyphenyl)propanenitrile;
3-(4-(7H-pyrrolo[2,3-c]pyridazin-4-yl)-1H-pyrazol-1-yl)-3-(4-hydroxyphenyl)propanenitrile;
(S)-3-(4-(7H-pyrrolo[2,3-c]pyridazin-4-yl)-1H-pyrazol-1-yl)-3-(4-hydroxyphenyl)propanenitrile;
(R)-3-(4-(7H-pyrrolo[2,3-c]pyridazin-4-yl)-1H-pyrazol-1-yl)-3-(4-hydroxyphenyl)propanenitrile;
2-((1S,2S)-2-(4-(7H-pyrrolo[2,3-c]pyridazin-4-yl)-1H-pyrazol-1-yl)cyclopentyl)acetonitrile;
2-((1R,2S)-2-(4-(7H-pyrrolo[2,3-c]pyridazin-4-yl)-1H-pyrazol-1-yl)cyclopentyl)acetonitrile;
2-((1S,2R)-2-(4-(7H-pyrrolo[2,3-c]pyridazin-4-yl)-1H-pyrazol-1-yl)cyclopentyl)acetonitrile; or
2-((1R,2R)-2-(4-(7H-pyrrolo[2,3-c]pyridazin-4-yl)-1H-pyrazol-1-yl)cyclopentyl)acetonitrile; or a salt thereof.

In cases wherein n=0, R₁ is connected to NR₃, O or S by a carbon atom of R₁ (i.e. carbon linked).

Processes which can be used to prepare compounds of formula I and intermediates useful for preparing compounds of formula I are shown in Schemes 1-79.

### General Methods of Preparation of Invention Compounds:

Heterocycles and hetereoaryls can be prepared from know methods as reported in the literature (a. Ring system handbook, published by American Chemical Society edition 1993 and subsequent supplements. b. The Chemistry of Heterocyclic Compounds; Weissberger, A., Ed.; Wiley: New York, 1962. c. Nesynov, E. P.; Grekov, A. P. The chemistry of 1,3,4-oxadiazole derivatives. Russ. Chem. Rev. 1964, 33, 508-515. d. Advances in Heterocyclic Chemistry; Katritzky, A. R., Boulton, A. J., Eds.; Academic Press: New York, 1966. e. In Comprehensive Heterocyclic Chemistry; Potts, K. T., Ed.; Pergamon Press: Oxford, 1984. f. Eloy, F. A review of the chemistry of 1,2,4-oxadiazoles. Fortschr.Chem. Forsch. 1965, 4, pp 807-876. g. Adv. Heterocycl. Chem. 1976. h. Comprehensive Heterocyclic Chemistry; Potts, K. T., Ed.; Pergamon Press: Oxford, 1984.1. Chem. Rev. 1961 61, 87-127. j. 1,2,4-Triazoles; John Wiley & Sons: New York,1981; Vol 37). Some of the functional groups during the synthesis may need to be protected and subsequently deprotected. Examples of suitable protecting groups can be found in “Protective groups in organic synthesis” fourth edition edited by Greene and Wuts.

Schemes 1-3 outline methods to prepare compounds of formula I. Methods to prepare starting materials or intermediates of Schemes 1-3 and reaction conditions for performing the synthetic steps of Schemes 1-3 are known (for example see: Scheme 1: Kidwai, M.; Singhal, K. J. Heterocyclic Chem. 2007, 44, 1253-1257; Scheme 2: 1. Sazonov, N. V.; Safonova, T. S. Chem. of Heterocycclic Compounds, 1972, 8, 1163-1166, 2. Taylor, E. C.; Cheng, C. C. J. Org. Chem. 1960, 148-149. 3. Holy, A.; et al. J. Med. Chem. 2002, 45, 1918-1929).

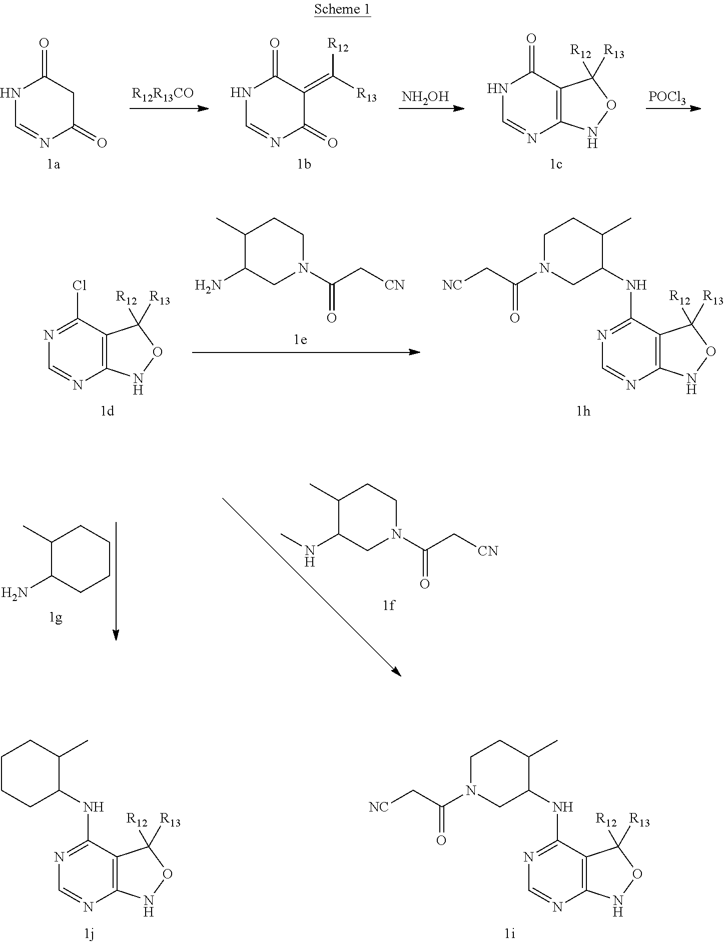

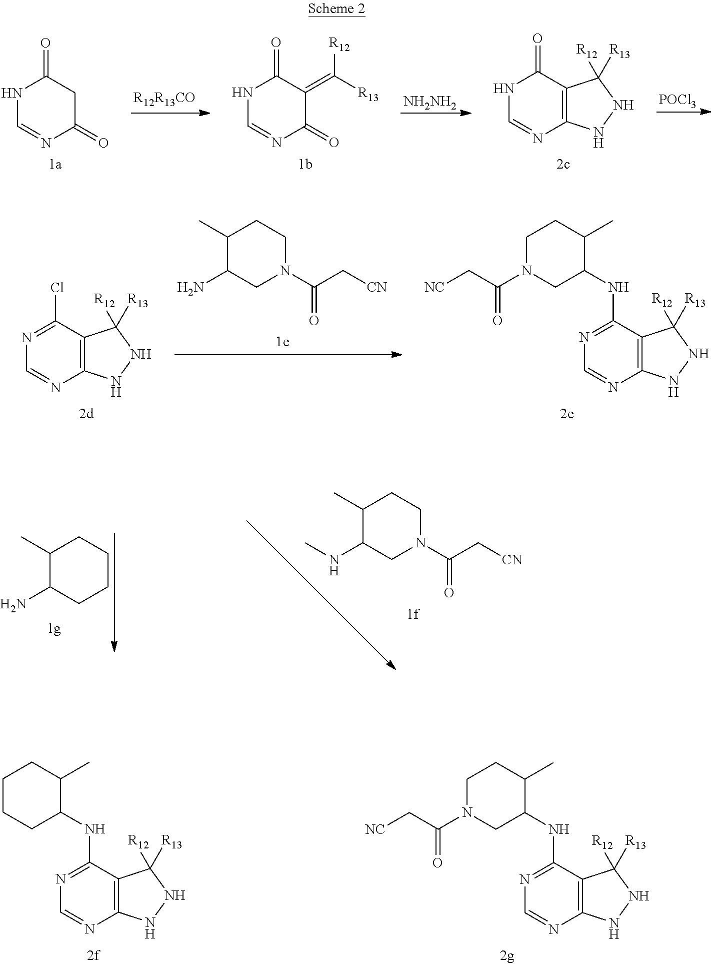

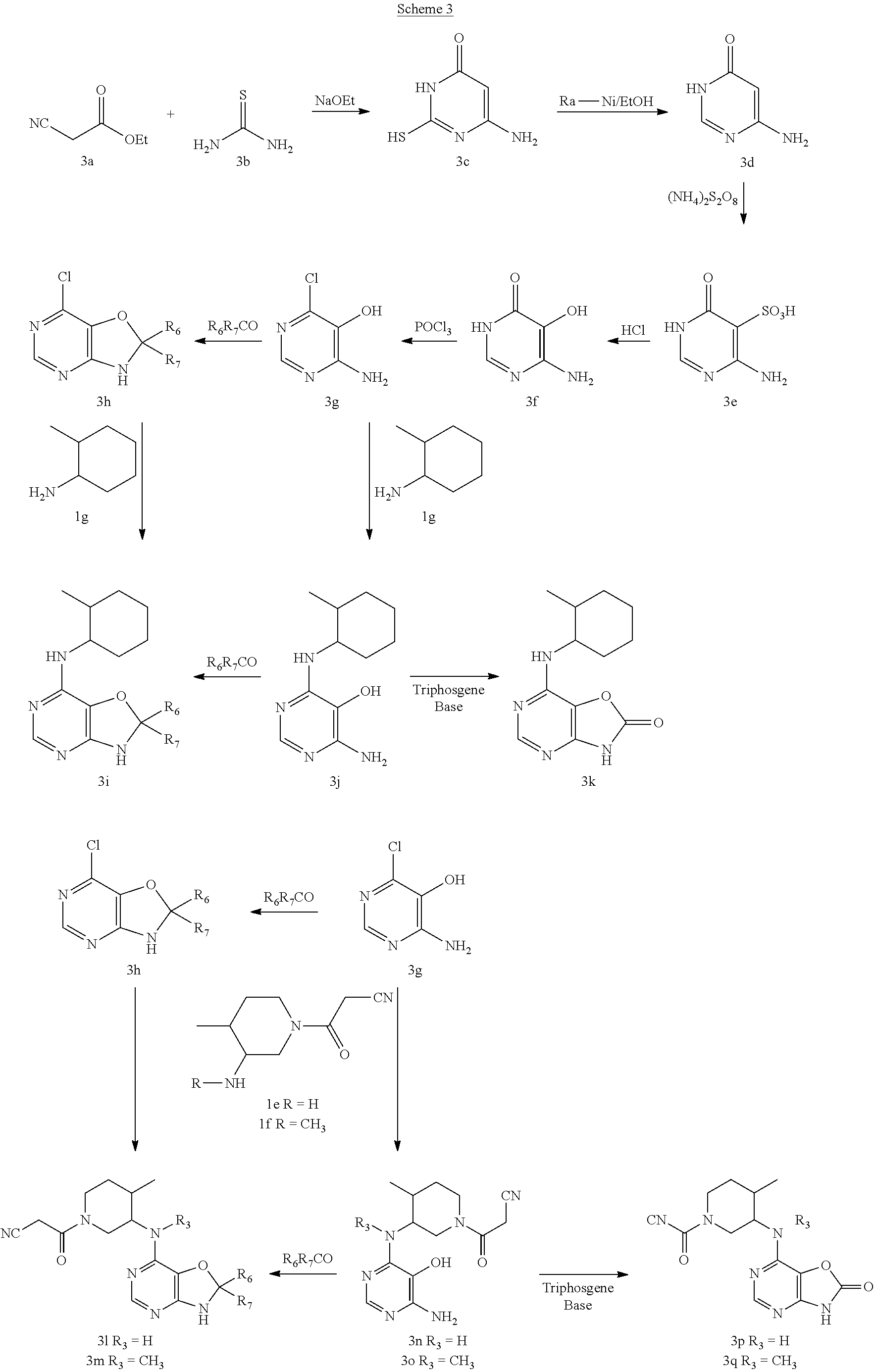

Schemes 4-8 outline methods to synthesize intermediates useful for preparing compounds of formula I. Methods to prepare starting materials or intermediates of Schemes 4-8 and reaction conditions for performing the synthetic steps of Schemes 4-8 are known (for example see: Scheme 4: 1. Ta-Shma, R.; et al. Tetrahedron, 2006, 62, 5469-5473. 2. Dirlam, J. P.; et al. J. Med. Chem. 1979, 22, 1118-1121).

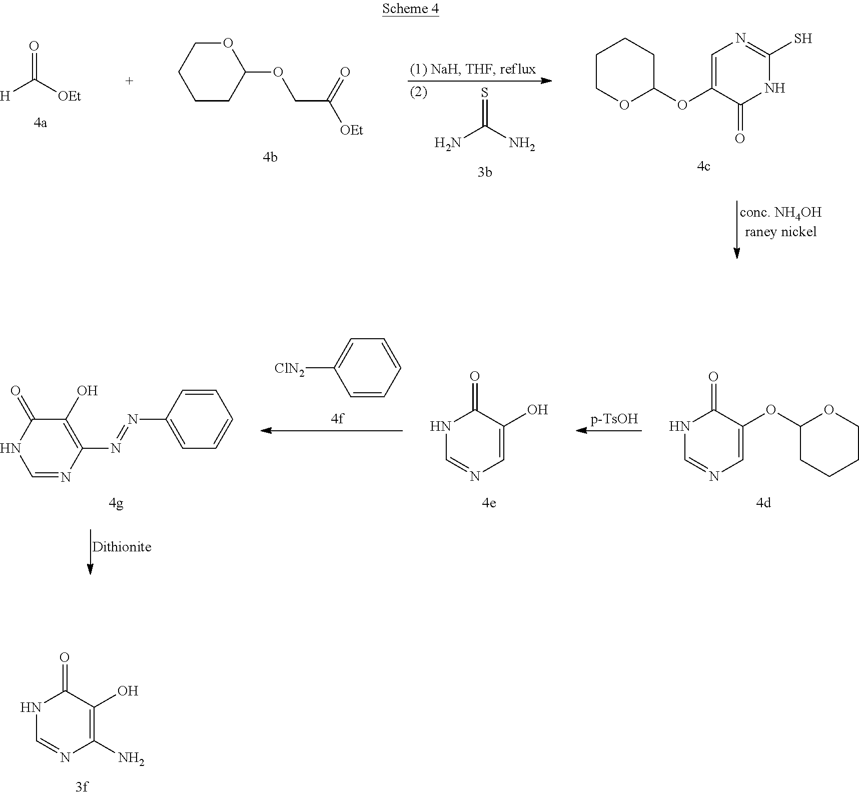

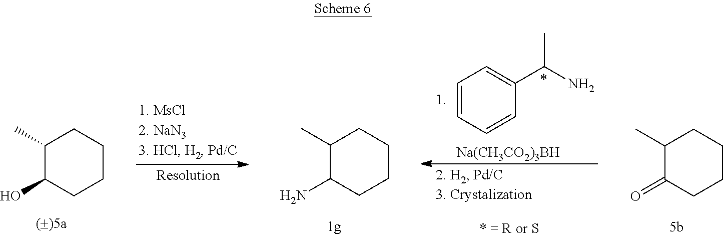

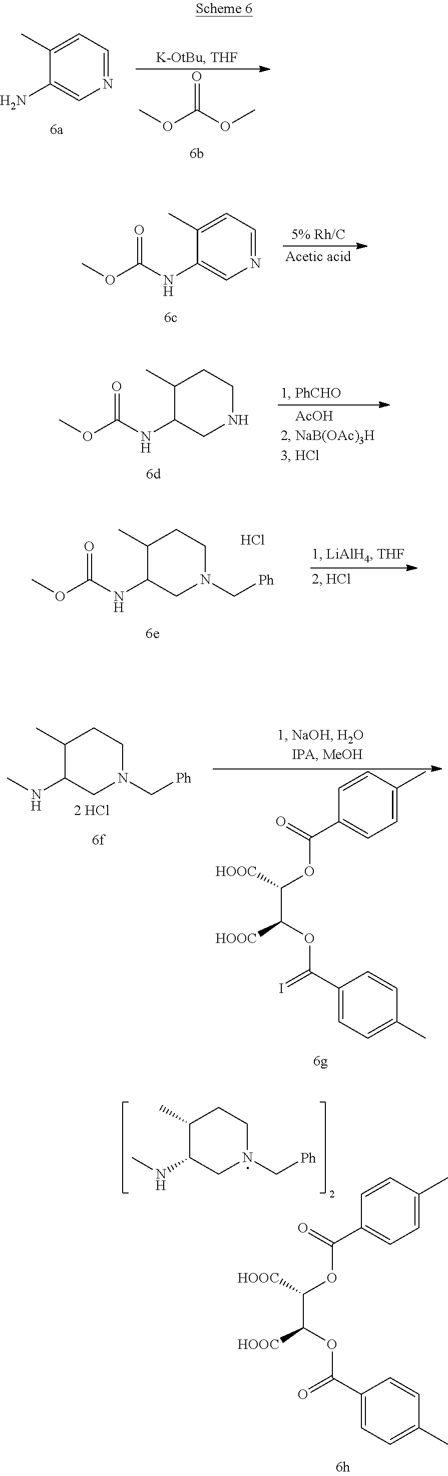

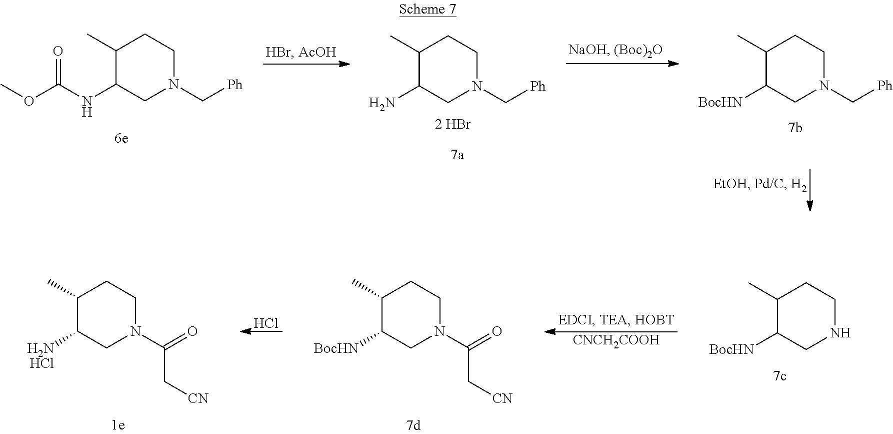

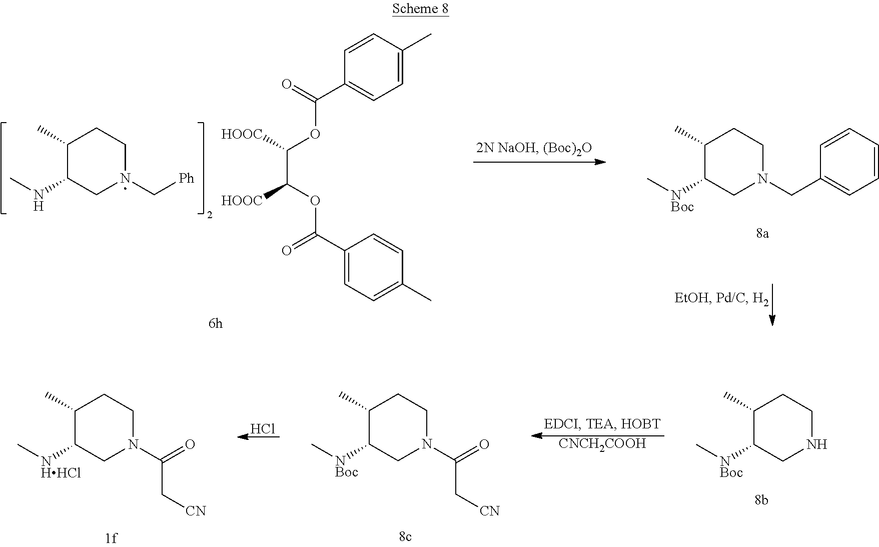

Schemes 9-16 outline methods to prepare compounds of formula I. Methods to prepare starting materials or intermediates of Schemes 9-16 and reaction conditions for performing the synthetic steps of Schemes 9-16 are known (for example see: Scheme 11: 1. WO 9413644 A1. 2. Revankar, Ganaphthi R.; Robins, Roland K. Journal of Heterocyclic Chemistry (1986), 23(6), 1869-78. 3. Anderson, Jack D.; Cottam, Howard B.; Larson, Steven B.; Nord, L. Dee; Revankar, Ganaphthi R.; Robins, Roland K. Journal of Heterocyclic Chemistry (1990), 27(2), 439-53); Scheme 12: WO 01/44211; Scheme 13: WO 2007125320; Scheme 14: Baraldi, Pier Giovanni et al., Tetrahedron (2002), 58(38), 7607-7611).

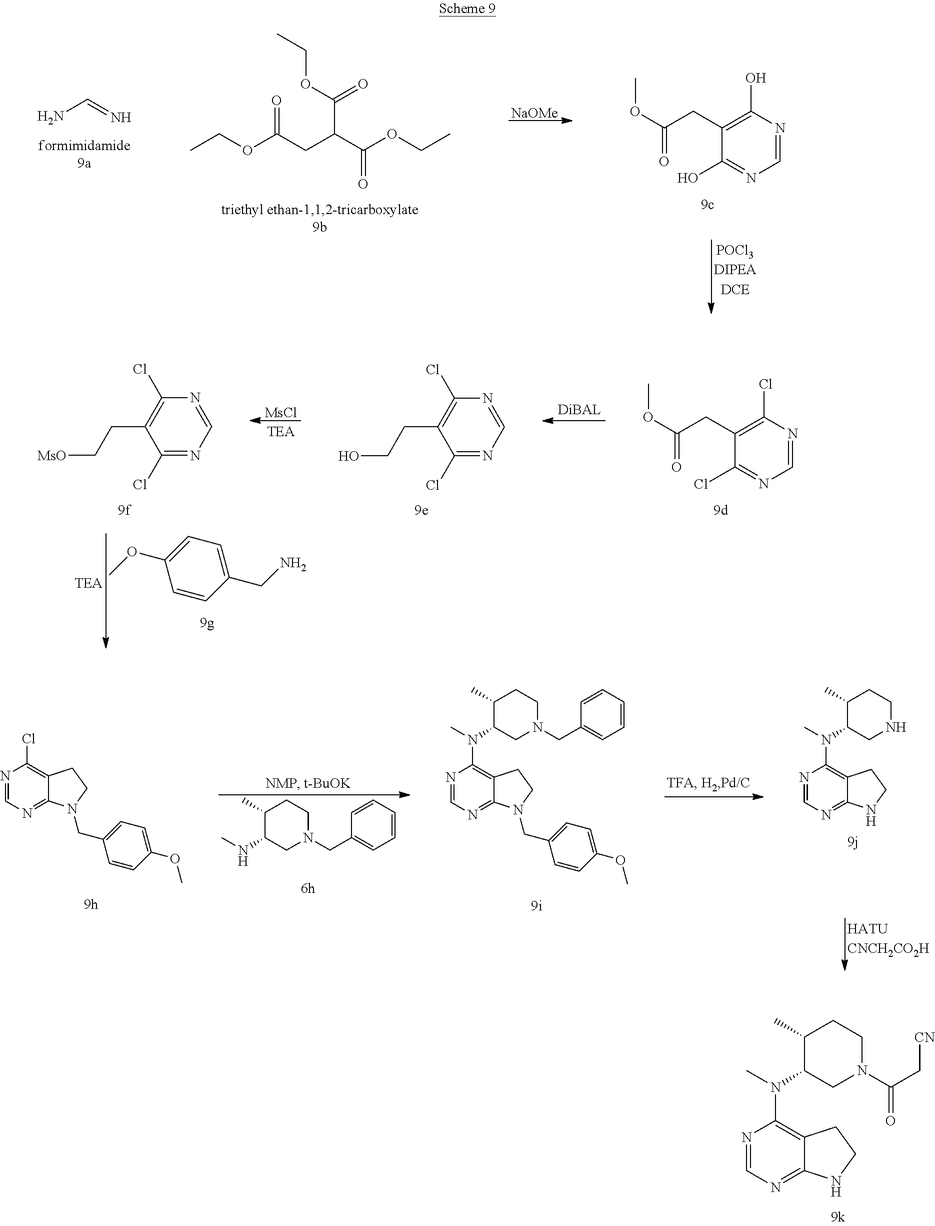

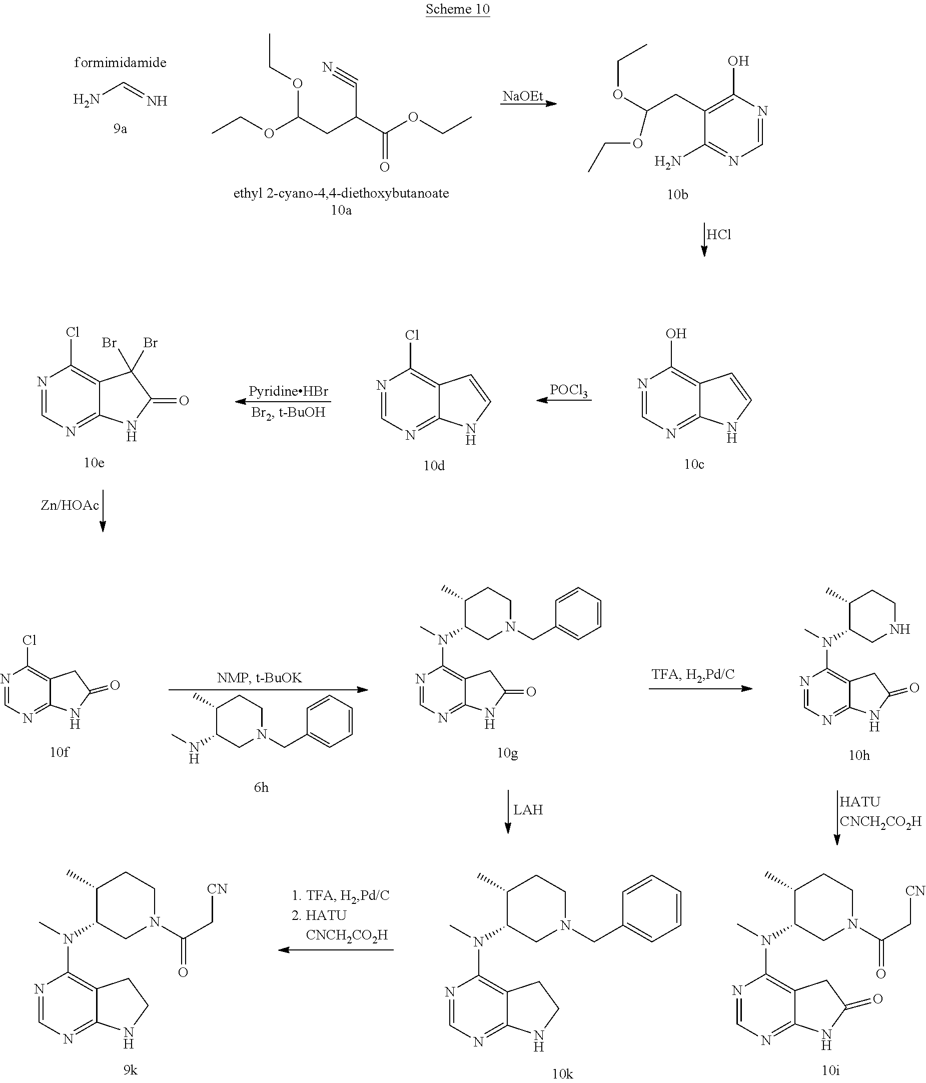

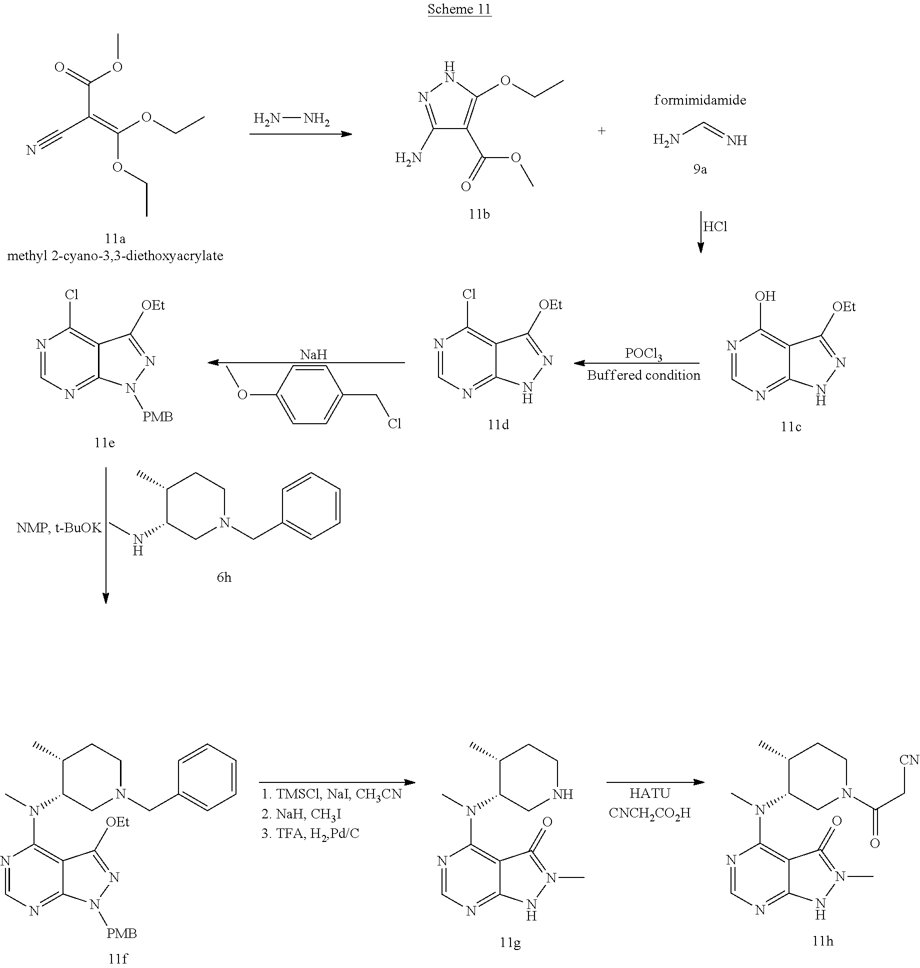

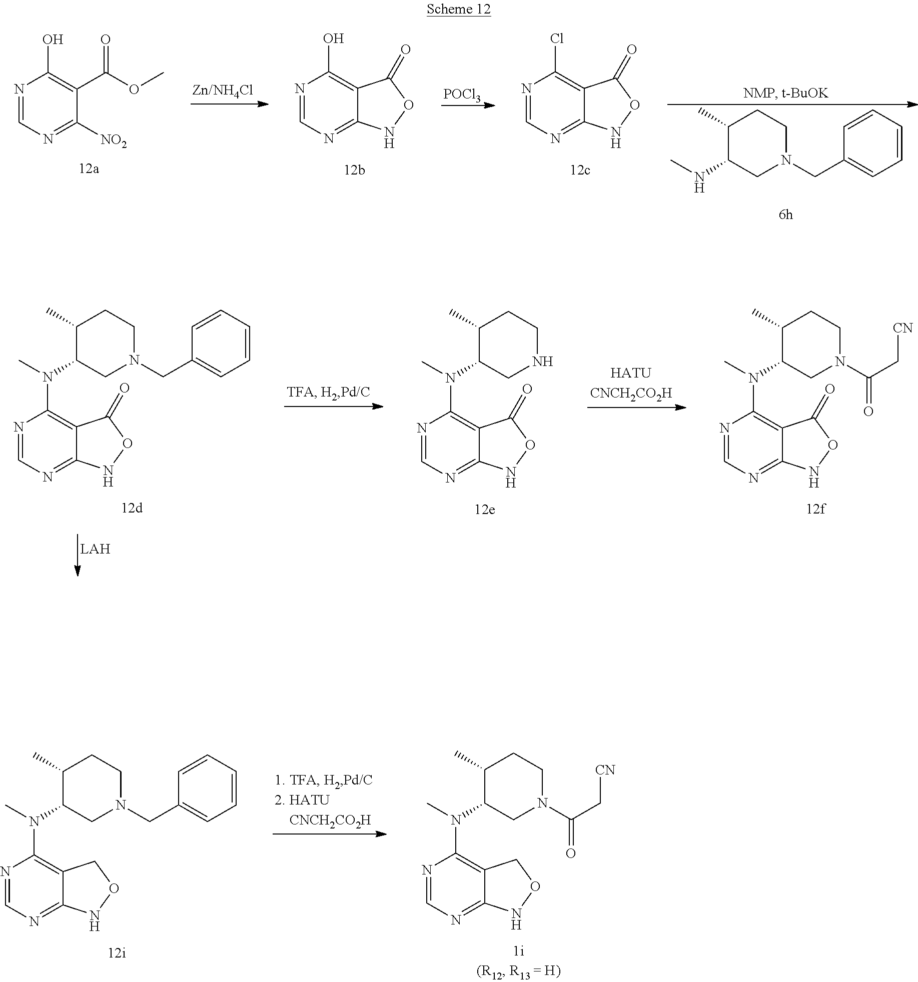

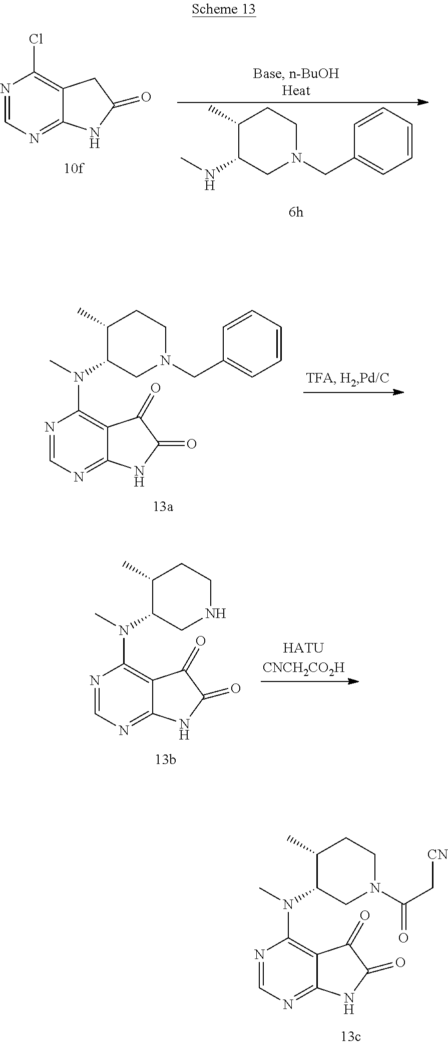

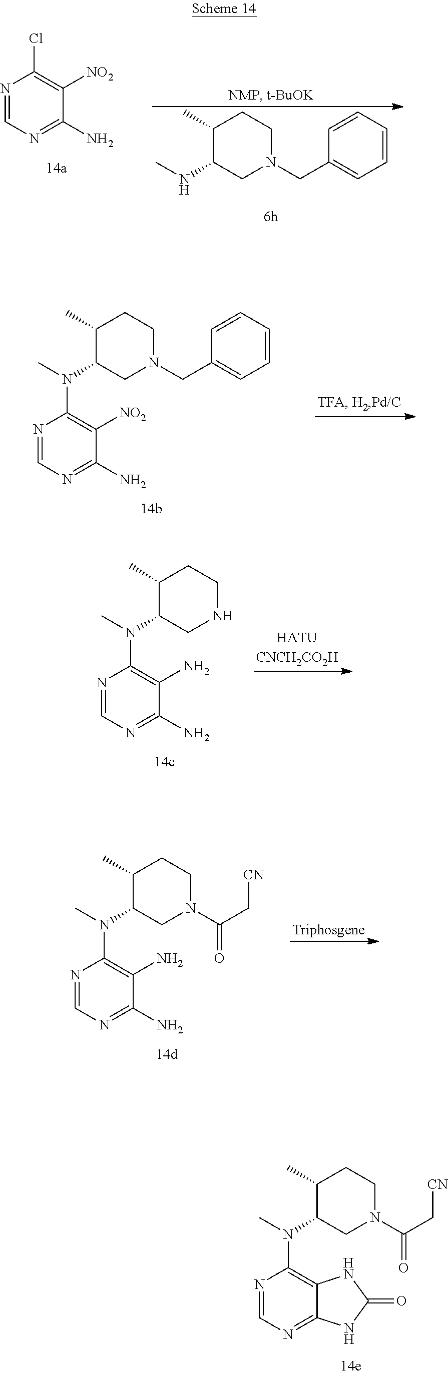

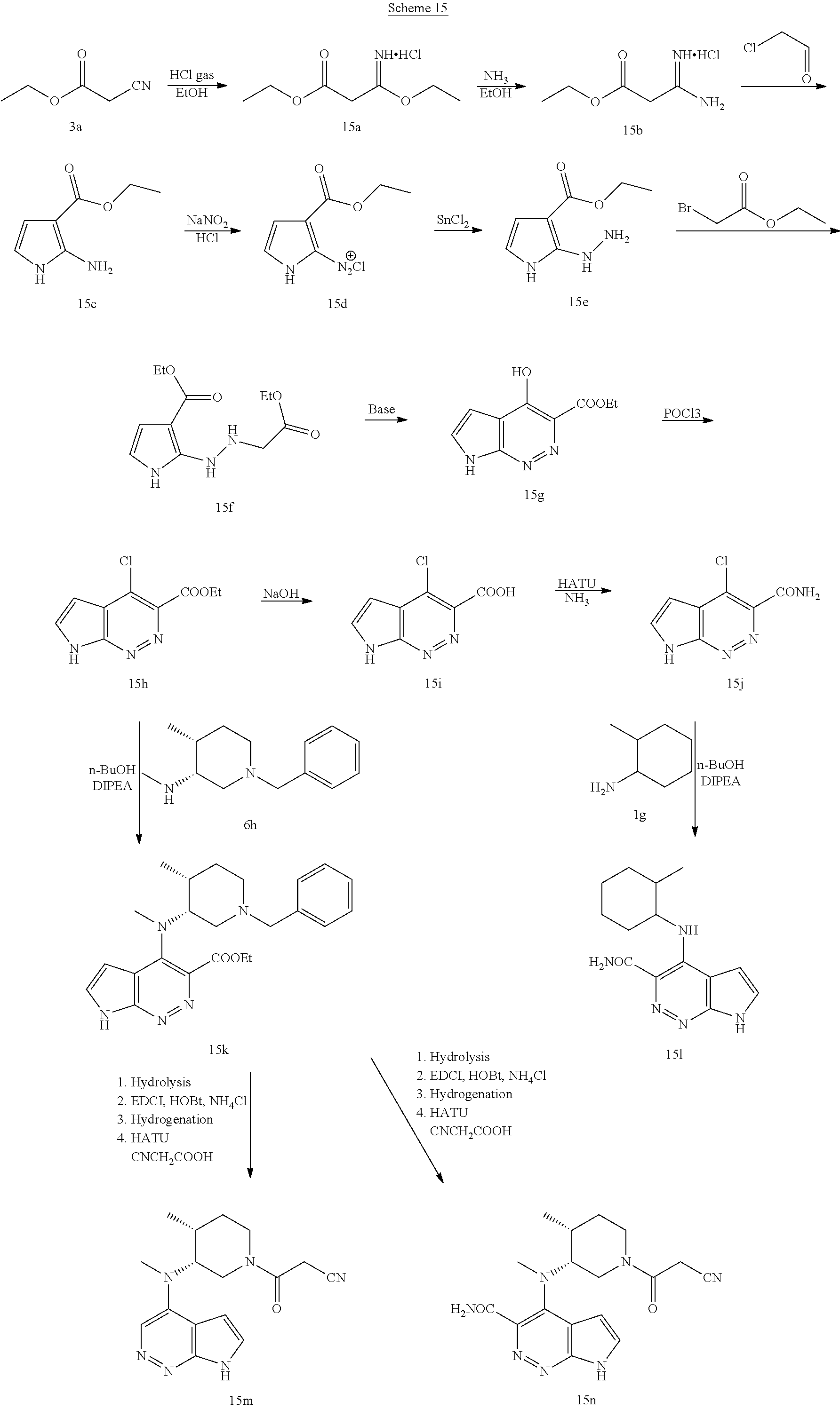

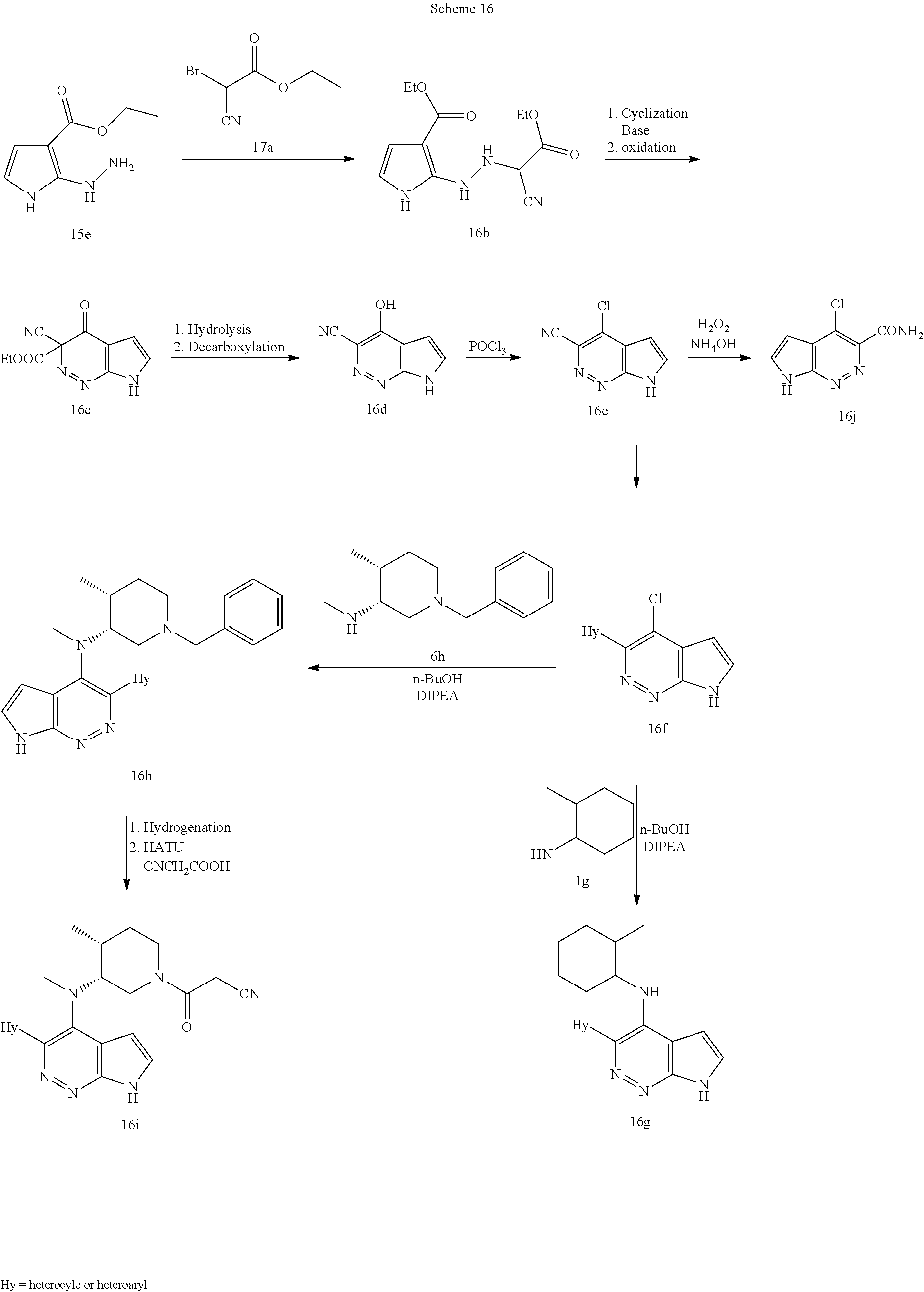

Schemes 17 and 18 outline methods to synthesize intermediates useful for preparing compounds of formula I. Methods to prepare starting materials or intermediates of Schemes 17 and 18 and reaction conditions for performing the synthetic steps of Schemes 17 and 18 are known (for example see: Scheme 17: 1. De Rosa, Michael; Issac, Roy P.; Houghton, Gregory, Tetrahedron Letters (1995), 36(51), 9261-4. 2. Turilli, Oreste; Gandino, Mario, Annali di Chimica (Rome, Italy) (1963), 53(11), 1687-96. 3. Youssef, Mohamed S. K.; El-Dean, Adel M. Kamal; Abbady, Mohamed S.; Hassan, Khairy M., Collection of Czechoslovak Chemical Communications (1991), 56(8), 1768-75. 4. Pattan, Shashikant R.; Ali, M. Shamrez; Pattan, Jayashri S.; Reddy, V. V. K., Indian Journal of Heterocyclic Chemistry (2004), 14(2), 157-158; Scheme 18: 1. Bray, Brian L.; Mathies, Peter H.; Naef, Reto; Solas, Dennis R.; Tidwell, Thomas T.; Artis, Dean R.; Muchowski, Joseph M; Journal of Organic Chemistry (1990), 55(26), 6317-28. 2. Gomez-Sanchez, Antonio; Maya, Ines; Hermosin, Isidro. Carbohydrate Research (1990), 200 167-80. 3. Cativiela, Carlos; Garcia, Jose I; Organic Preparations and Procedures International (1986), 18(4), 283-5. 4. Malona, John A.; Colbourne, Jessica M.; Frontier, Alison J. Organic Letters (2006), 8(24), 5661-5664.

### 5. Davies, James R.; Kane, Peter D.; Moody, Christopher J.; Slawin, Alexandra M. Z; Journal of Organic Chemistry (2005), 70(15), 5840-5851).

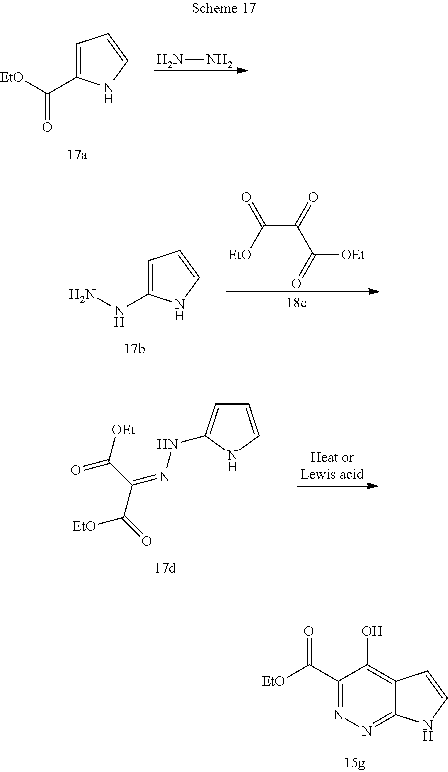

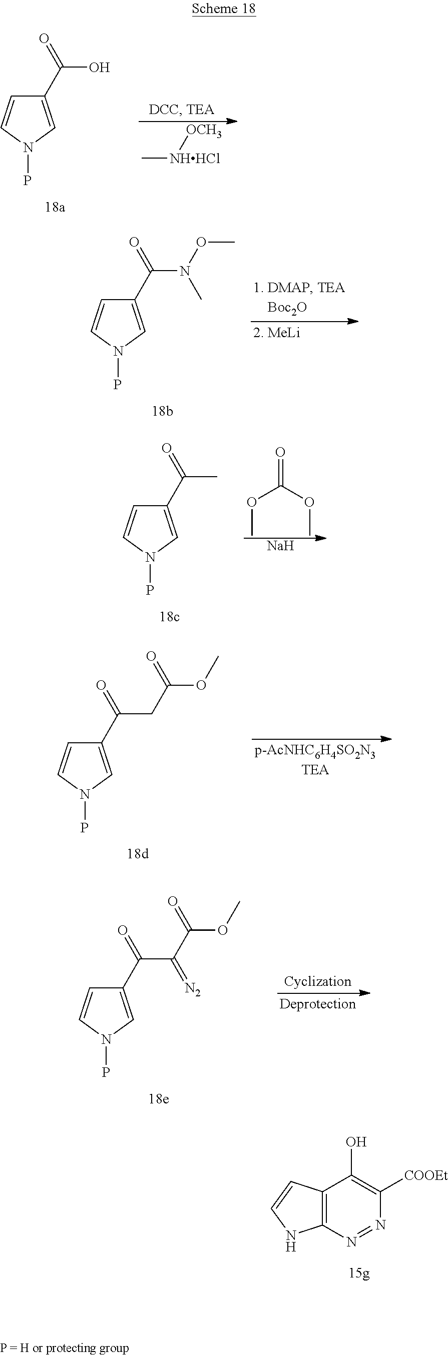

Schemes 19-21 outline methods to synthesize intermediates useful for preparing compounds of formula I. Methods to prepare starting materials or intermediates of Schemes 19-21 and reaction conditions for performing the synthetic steps of Schemes 19-21 are known (for example see: Scheme 19: 1. Morgentin, Remy; Jung, Frederic; Lamorlette, Maryannick; Maudet, Mickael; Menard, Morgan; Ple, Patrick; Pasquet, Georges; Renaud, Fabrice. Tetrahedron (2009), 65(4), 757-764. 2. Onnis, Valentina; De Logu, Alessandro; Cocco, Maria T.; Fadda, Roberta; Meleddu, Rita; Congiu, Cenzo. European Journal of Medicinal Chemistry (2009), 44(3), 1288-1295. Scheme 20:1. Bio, Matthew M.; Xu, Feng; Waters, Marjorie; Williams, J. Michael; Savary, Kimberly A.; Cowden, Cameron J.; Yang, Chunhua; Buck, Elizabeth; Song, Zhiguo J.; Tschaen, David M.; Volante, R. P.; Reamer, Robert A.; Grabowski, Edward J. J., Journal of Organic Chemistry (2004), 69(19), 6257-6266. 2. Lowen, Gregory T. (American Cyanamid Co., USA). U.S. (1991), 4 pp. CODEN: USXXAM U.S. Pat. No. 5,041,556 A U.S. Ser. No. 19/910,820 written in English. U.S. application Ser. Nos. 90-625739, 19/901,211. 3. Zepeda, L. Gerardo; Rojas-Gardida, Mirna; Morales-Rios, Martha S.; Joseph-Nathan, Pedro. Cent. Invest. Estud. Avanzados, Tetrahedron (1989), 45(20), 6439-48. 4. Aitken, Steven; Brooks, Gerald; Dabbs, Steven; Frydrych, Colin Henry; Howard, Steven; Hunt, Eric. PCT Int. Appl. (2002), 91 pp. CODEN: PIXXD2 WO 2002012199 A1 20020214. Scheme 2: 1. Migawa, Michael T.; Townsend, Leroy B; Journal of Organic Chemistry (2001), 66(14), 4776-4782. 2. Migawa, Michael T.; Townsend, Leroy B; Synthetic Communications (1999), 29(21), 3757-3772).

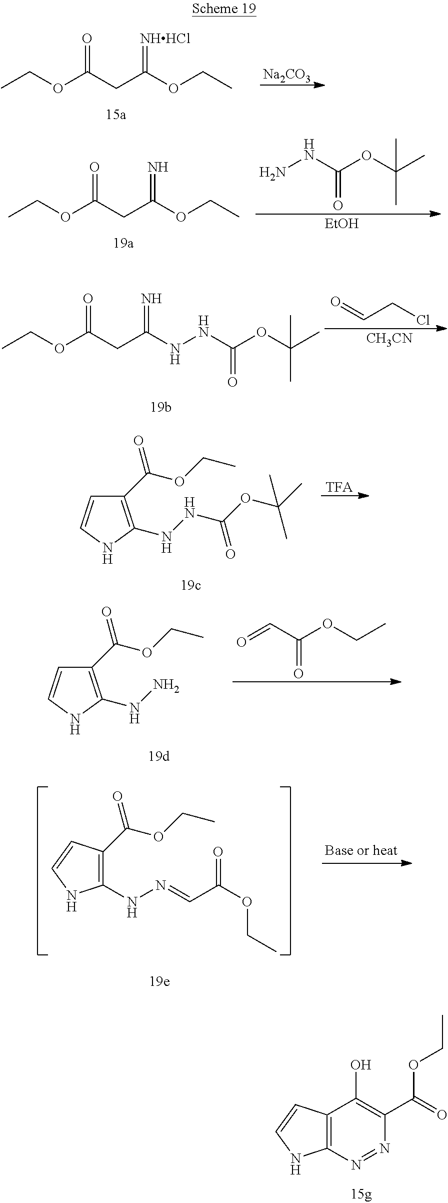

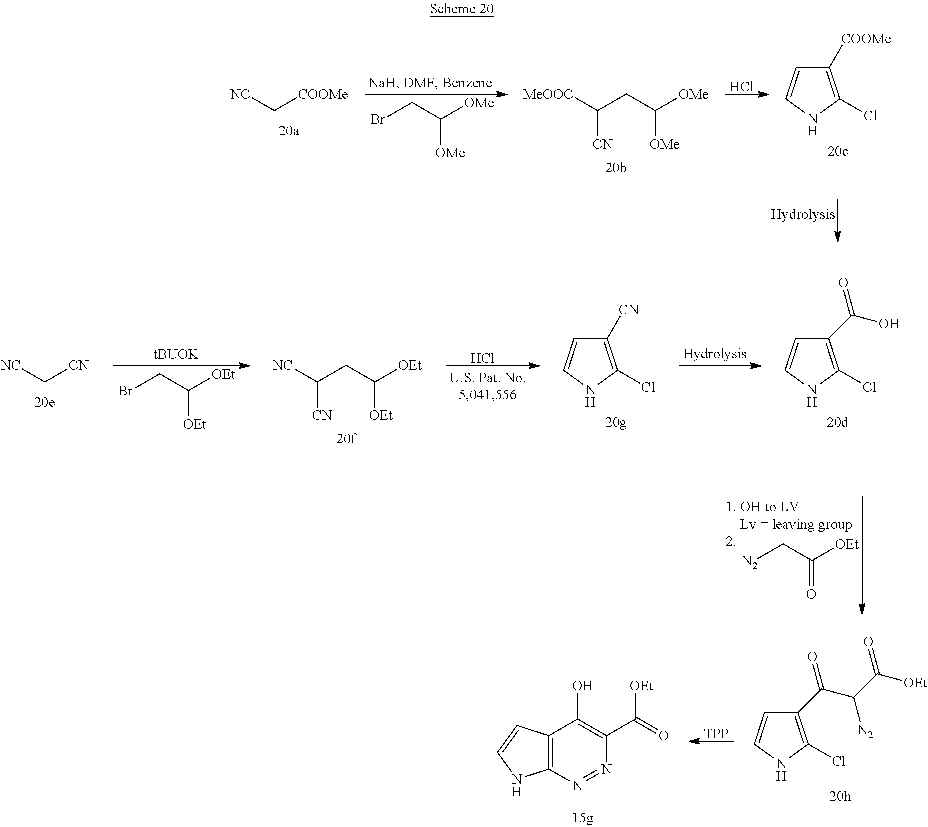

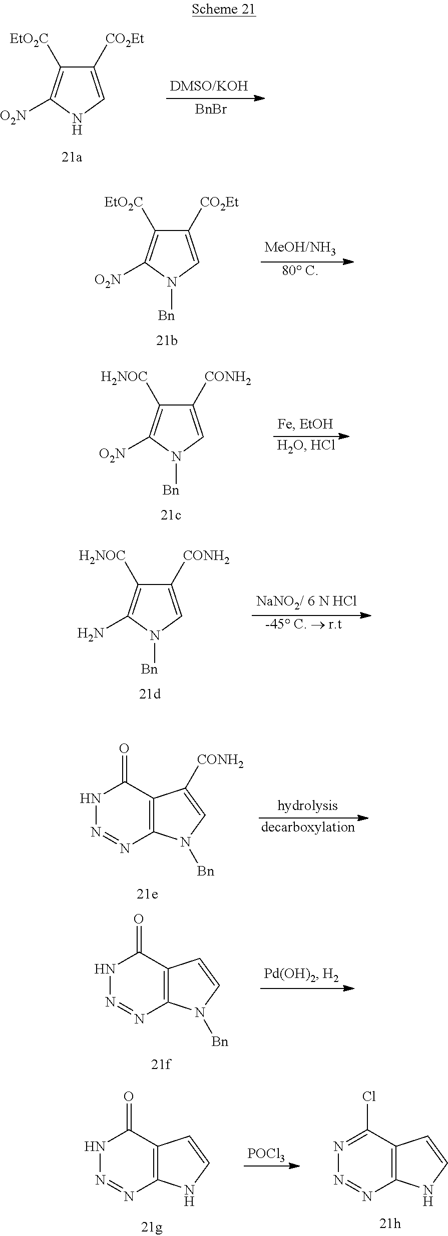

Scheme 22a outlines a general method that was used to prepare compounds of formula I while Schemes 22b and 23 depict alternative methods that can be used to prepare compounds of formula 1.

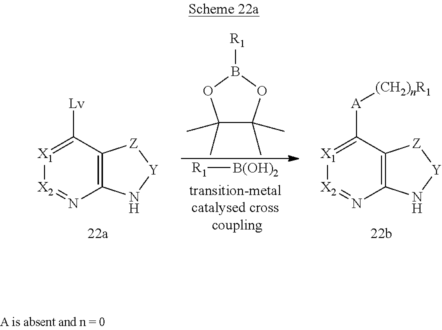

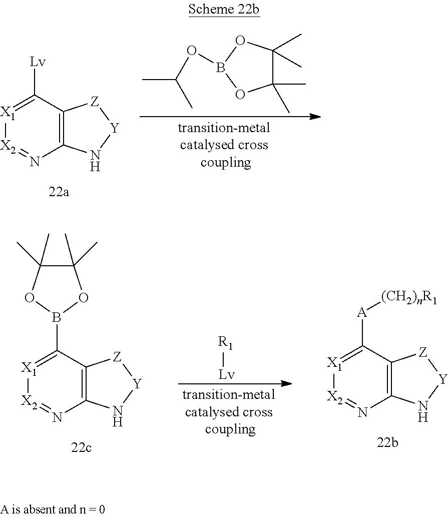

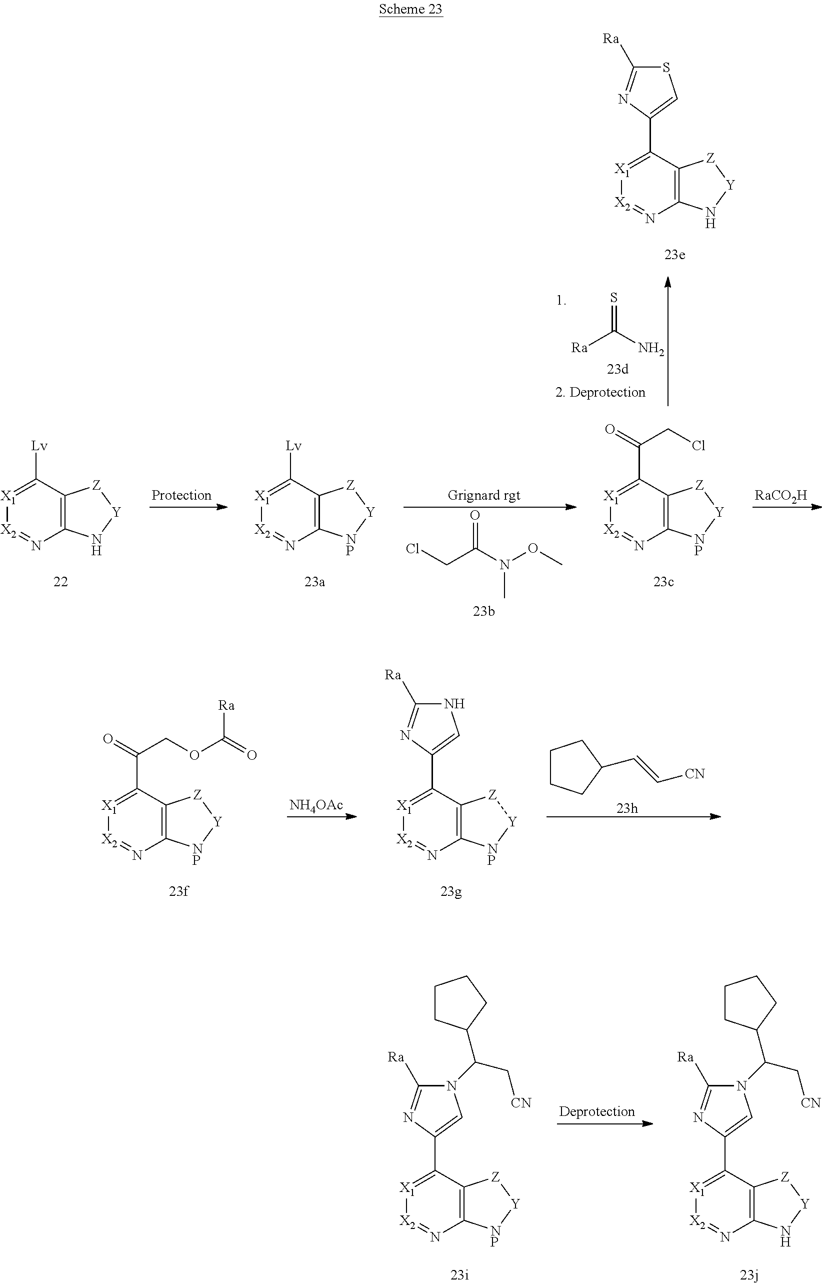

Schemes 24-25 outline methods which can be used to synthesize intermediates useful for preparing compounds of formula 1.

3-(Furan-2-yl)acrylaldehyde (24b) can be prepared from furan-2-carbaldehyde 24(a) according to procedures reported in the literature (for example see: 1. Valenta, Petr, et al., Organic Letters 2009, 11(10), 2117-2119. 1. McComsey, David F. et al., Encyclopedia of Reagents for Organic Synthesis (2001)3. Mahata, Pranab Kumar, et al., Synlett 2000, 9, 1345-1347. 4. Shapiro, Yu. M. et al., Khimiya Geterotsiklicheskikh Soedinenii 1993, 1, 25-8. 5. Bellassoued, Moncef, et al., Journal of Organic Chemistry 1993, 58(9), 2517-22. 6. Duhamel, L. et al., Journal of Organometallic Chemistry 1989, 363 (1-2), C4-C6. 7. Di Nunno, L., et al., Tetrahedron 1988, 44(12), 3639-44. 8. Duhamel, Lucette, et al., Organic Preparations and Procedures International 1986, 18(4), 219-26. 9. Bestmann, Hans Juergen, et al., Chemische Berichte 1982, 115(1), 161-71. 10. Bestmann, Hans Juergen, et al., Angewandte Chemie 1979, 91(9), 748.

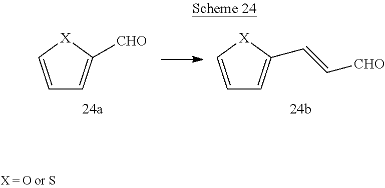

Furan-2-ylacrylaldehyde (25b) can be prepared from the appropriately substituted furan-2-carbaldehyde 25a according to the procedure reported in the literature (Mocelo, R.; Pustovarov, V. Esc. Quim., Univ. La Habana, Havana, Cuba. Revista sobre los Derivados de la Cana de Azucar (1976), 10(2), 3-9).

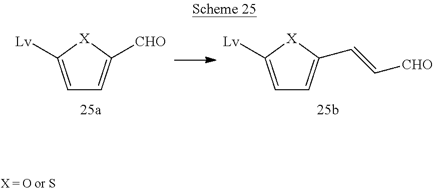

Schemes 26-30 outline methods which can be used to synthesize compounds of formula I. Methods to prepare starting materials or intermediates of Schemes 26-30 and reaction conditions for performing the synthetic steps of Schemes 26-30 are known (for example see: Scheme 26; Gotoh, Hiroaki, et al., Angewandte Chemie, International Edition 2006, 45(41), 6853-6856; Scheme 29; 1. WO2001023383, 2. JP07285931, 3. JP06345772 or 4. EP629626. Scheme 30; 1. Afshar, Davood Aghaei, et al., Journal of Chemical Research 2008, (9), 509-511; 2. Berthon-Gelloz, Guillaume, et al., Chemistry—A European Journal, 2009, 15(12), 2923-2931; 3. Sarma, C. R. et al., Indian Journal of Chemistry, Section B: Organic Chemistry Including Medicinal Chemistry (1989), 28B (11), 993-5.

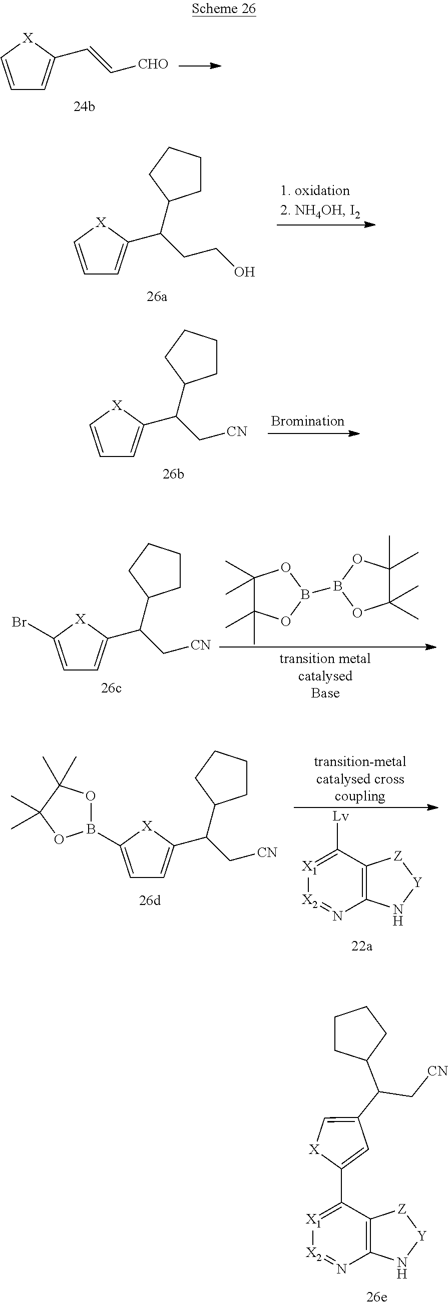

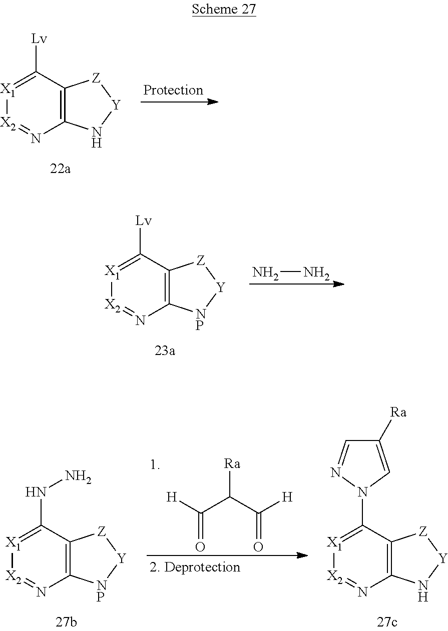

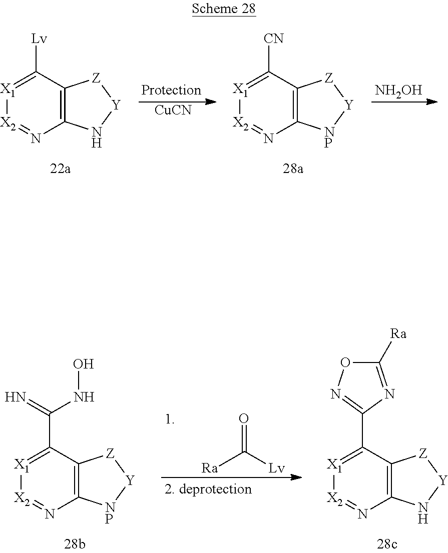

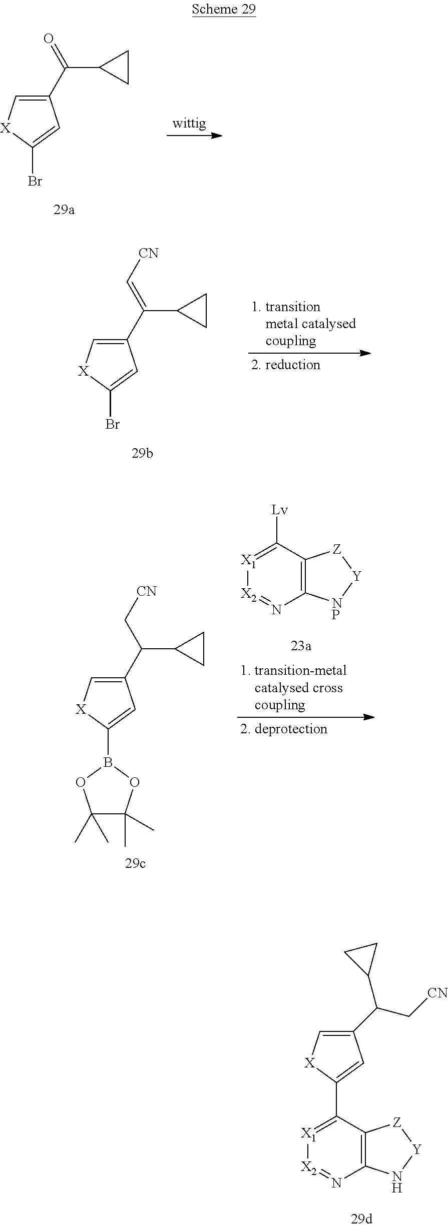

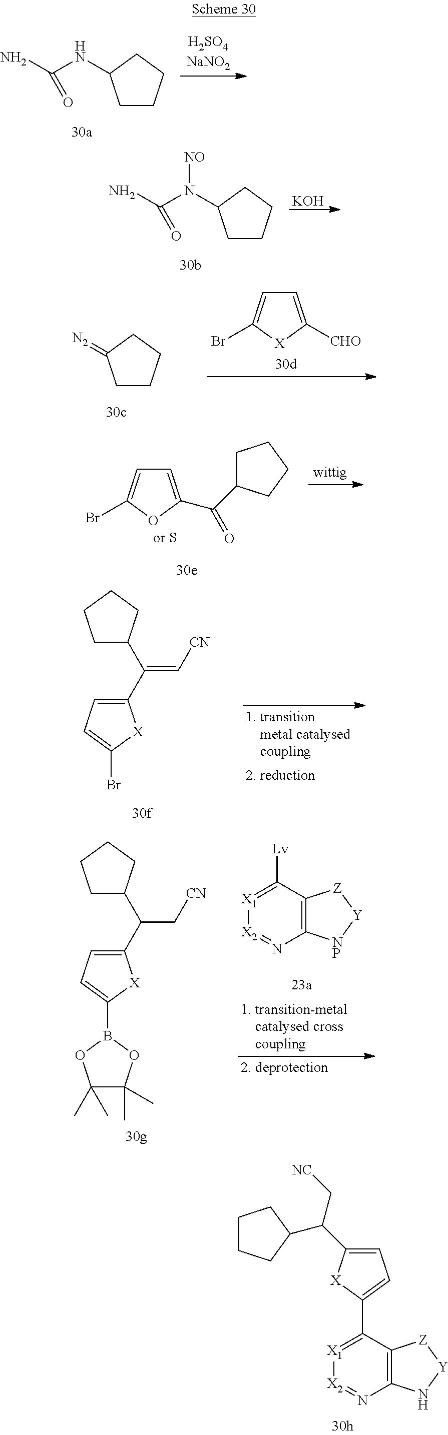

Schemes 31 and 32 depict synthetic routes that were used to prepare compounds of formula I as described in Examples 1 and 2.

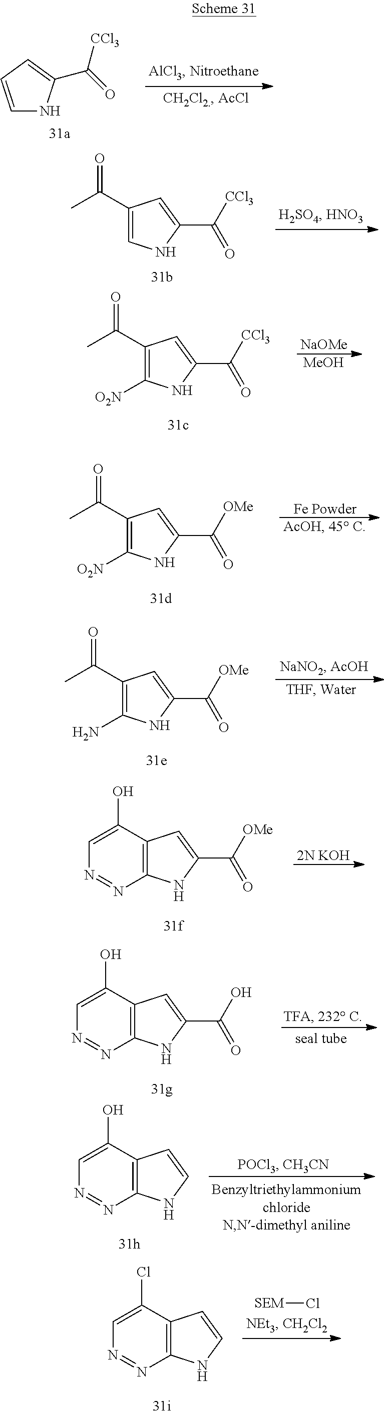

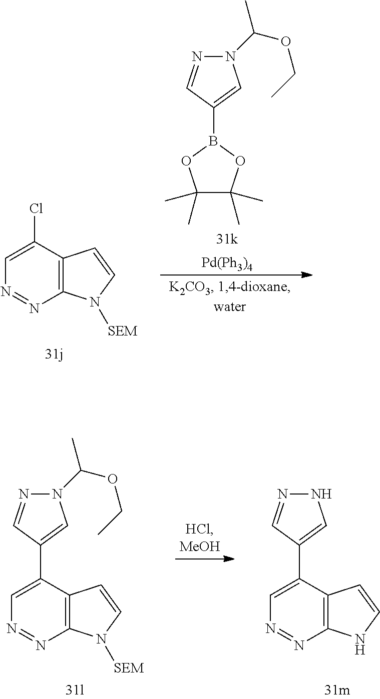

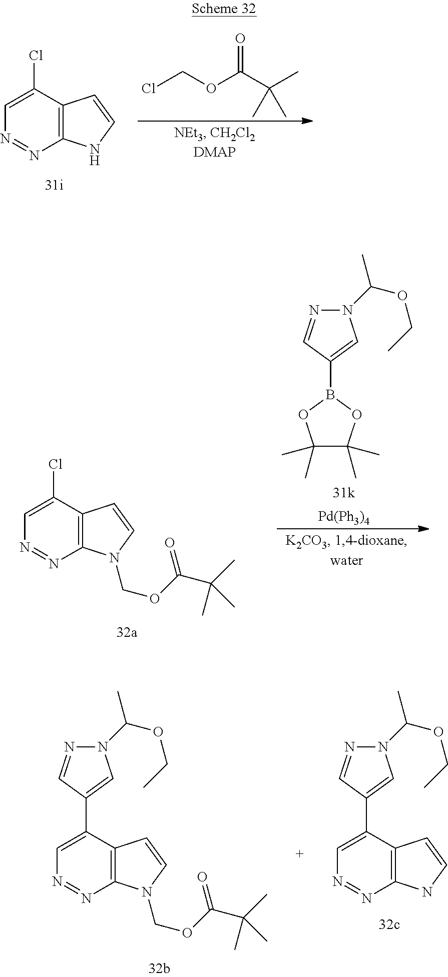

Scheme 33 outlines a method to synthesize an intermediate useful for preparing compounds of formula I. Methods to prepare starting materials and reaction conditions for performing the synthetic steps of Scheme 33 are known (for example see: 1. Sonoda, Miki, et al., Chemical & Pharmaceutical Bulletin 1982, 30(7), 2357-63. 2. Mohamed, Mosaad Sayed, et al., Acta Pharmaceutica (Zagreb, Croatia) 2009, 59(2), 145-158. 3. Ronan, Baptiste, et al., Fr. Demande 2006, 35 pp. FR 2881742 A1 20060811).

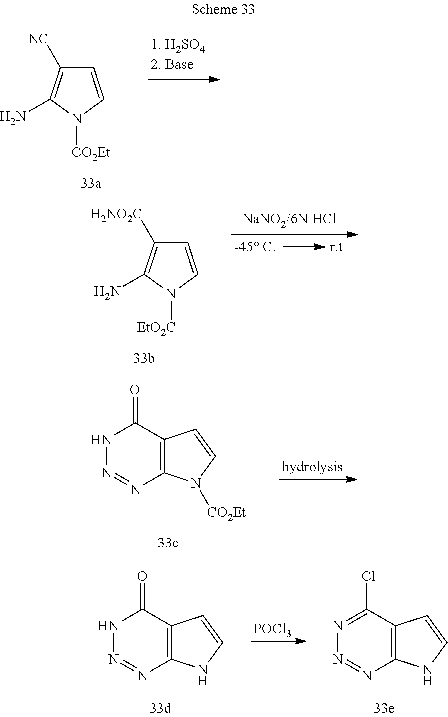

Scheme 34 outlines a method to synthesize compound 34j. Methods to prepare starting materials and reaction conditions for performing the synthetic steps of Scheme 34 are known (for example see 1. Choudary, Boyapati M., et al., Journal of Catalysis (2003), 218(1), 191-200. 2. Kim, Mary M., et al; Tetrahedron Letters (2008), 49(25), 4026-4028).

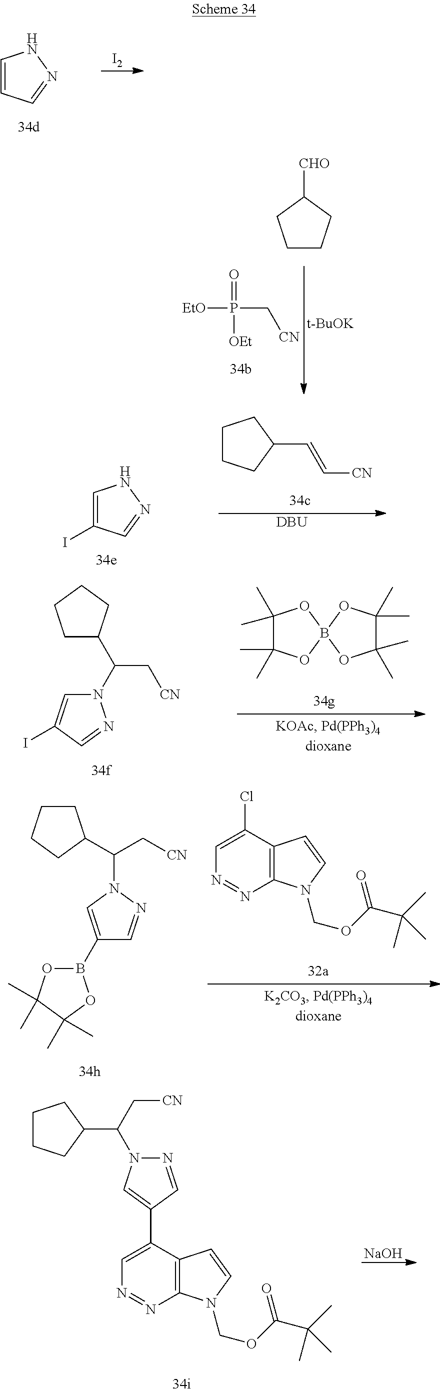

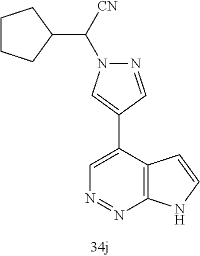

Schemes 35-79 outline methods that were used or can be used to prepare compounds of formula I or intermediates useful for preparing compounds of formula I.

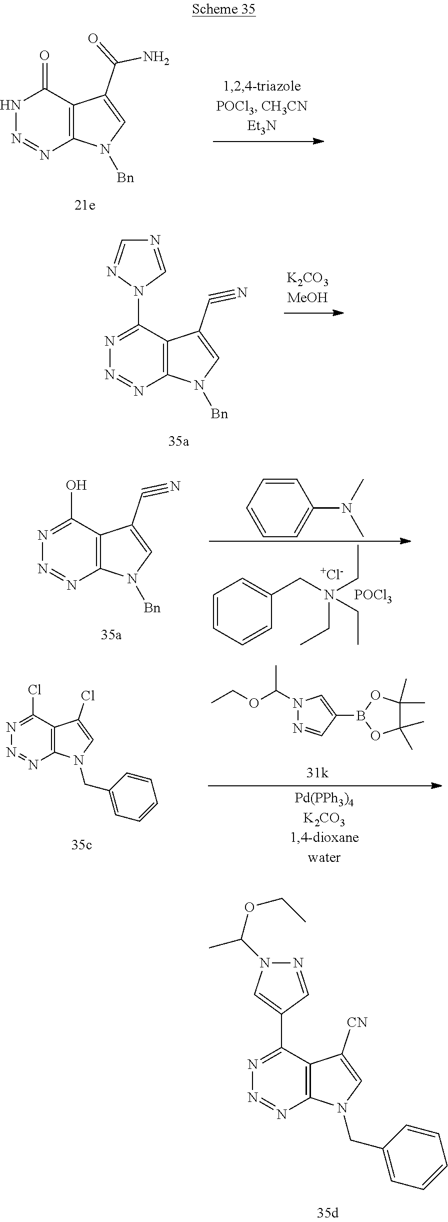

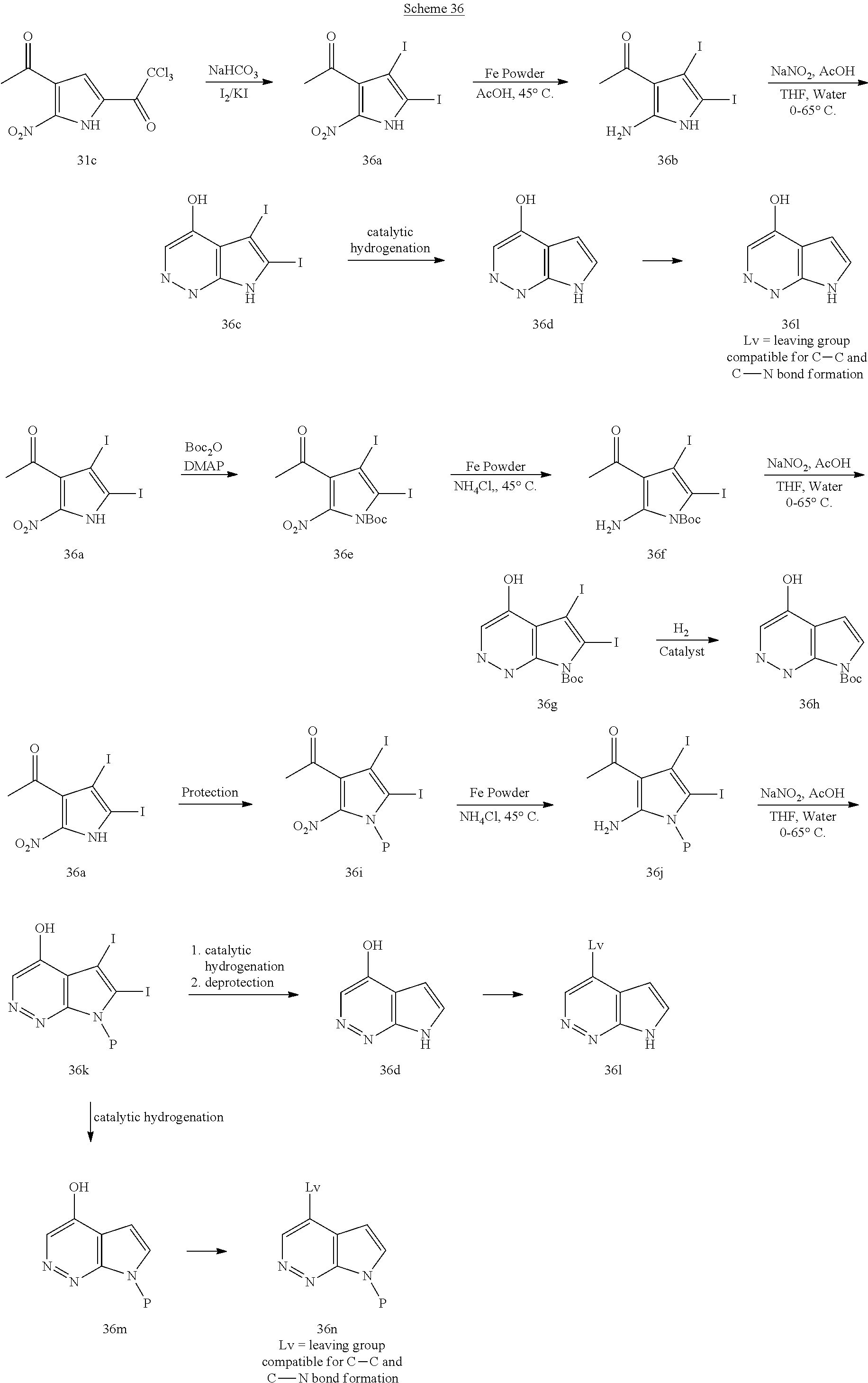

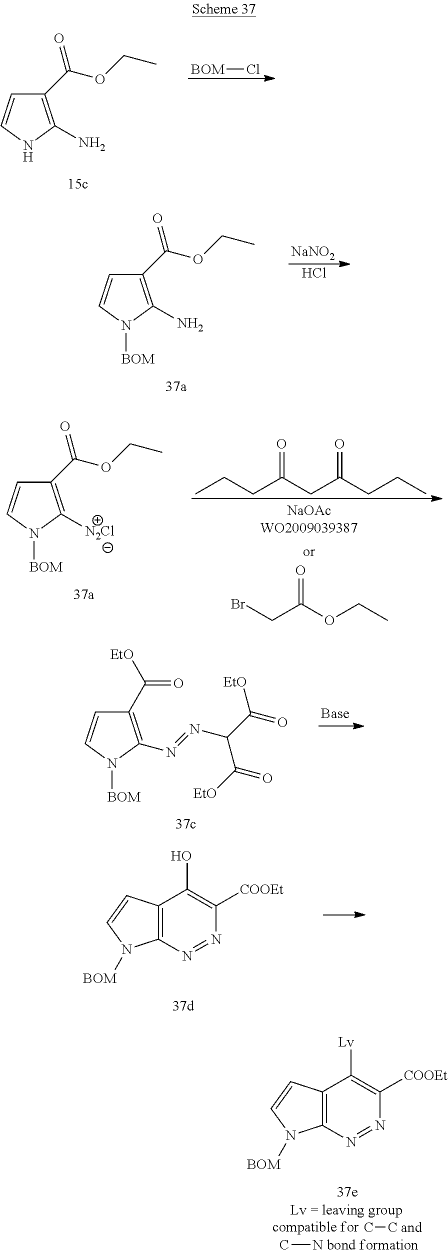

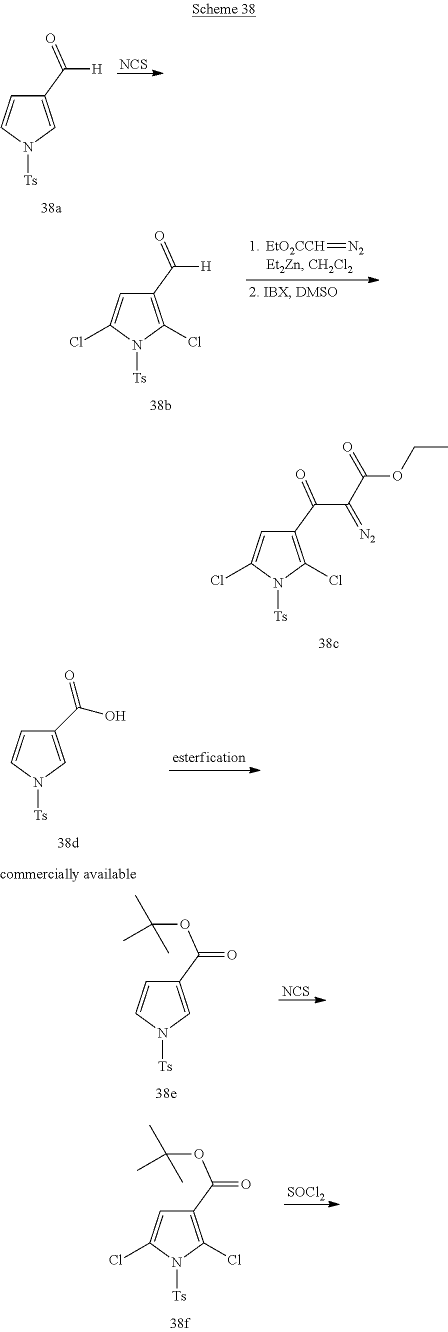

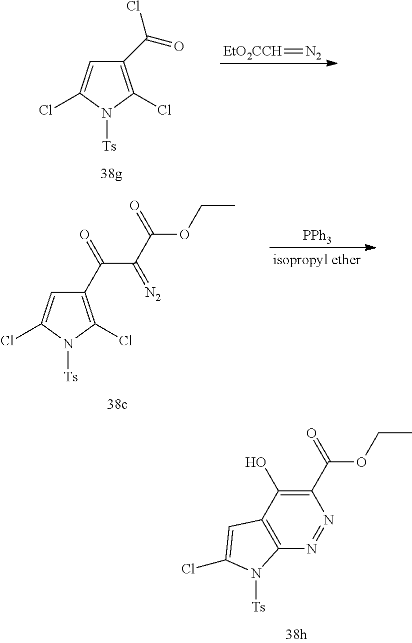

Chlorination of 1-tosyl-1H-pyrrole-3-carbaldehyde compound 38a furnished 2,5-dichloro-1-tosyl-1H-pyrrole-3-carbaldehyde 38b (as outlined by T. Ross Kelly and Rimma L. Moiseyeva J. Org. Chem. 1998, 63, 3147-3150). Compound 38b can be converted to ethyl 2-diazo-3-(2,5-dichloro-1-tosyl-1H-pyrrol-3-yl)-3-oxopropanoate 38c by two step process (James R. Davies, Peter D. Kane, Christopher J. Moody, and Alexandra M. Z. Slawin, J. Org. Chem. 2005, 70, 5840-5851). Compound 38c can also be prepared from commercially available 1-tosyl-1H-pyrrole-3-carboxylic acid 38d as outlined in Scheme 38. Trialkylphosphine or triphenylphosphine mediated cyclization affords ethyl 6-chloro-4-hydroxy-7-tosyl-7H-pyrrolo[2,3-c]pyridazine-3-carboxylate compound 38h from compound 38c (For examples of such cyclization see 1. Journal of Heterocyclic Chemistry, 24(1), 55-7; 1987; 2. Chemical & Pharmaceutical Bulletin, 38(12), 3211-17; 1990).

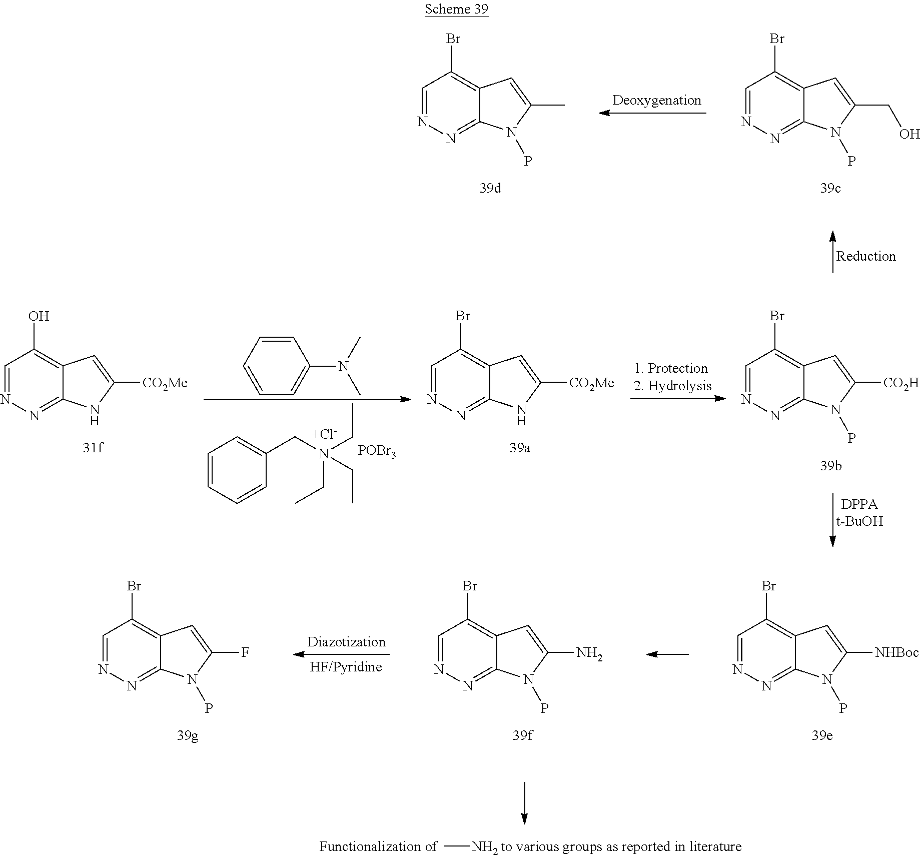

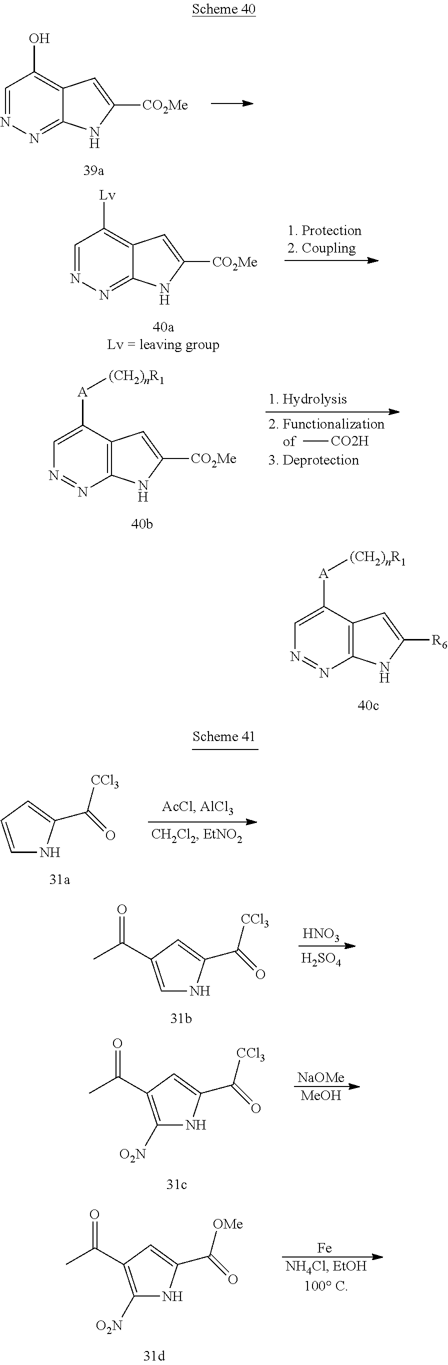

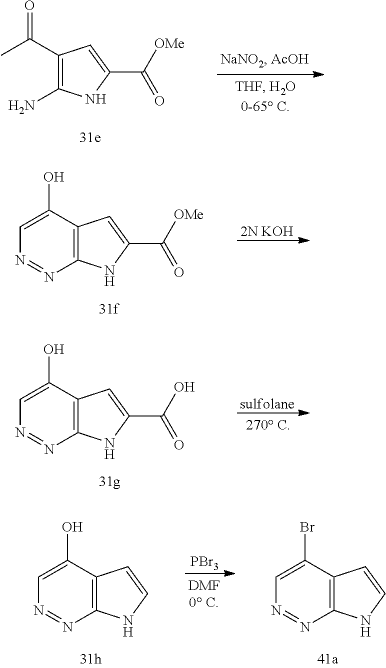

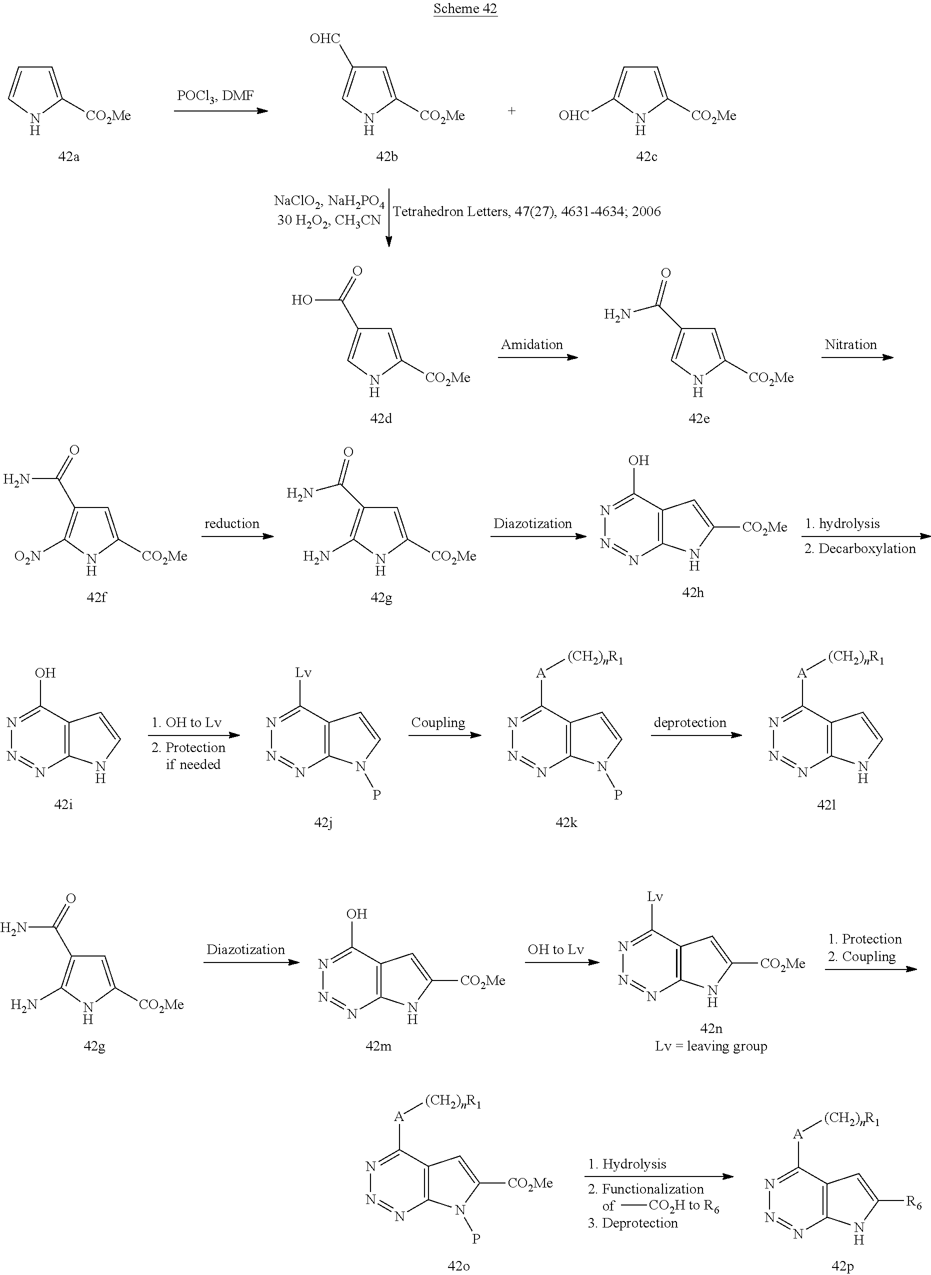

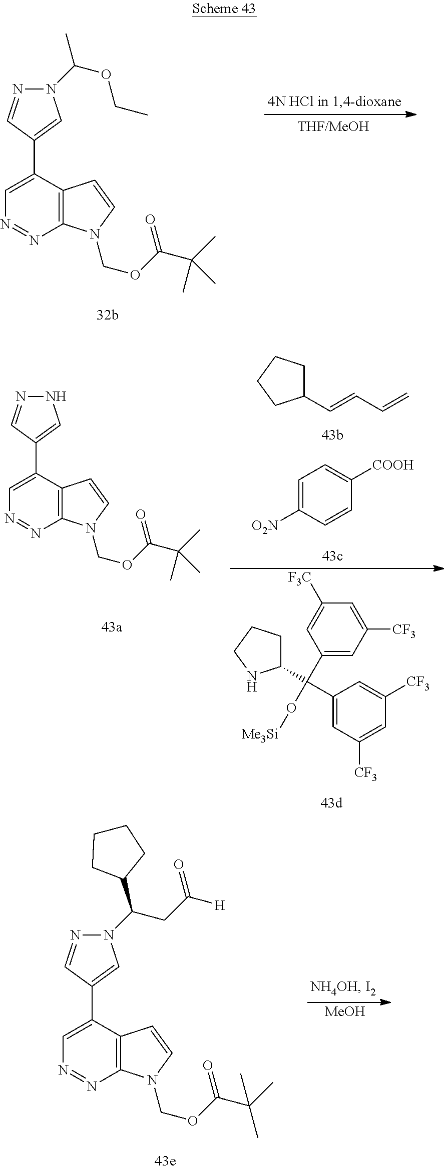

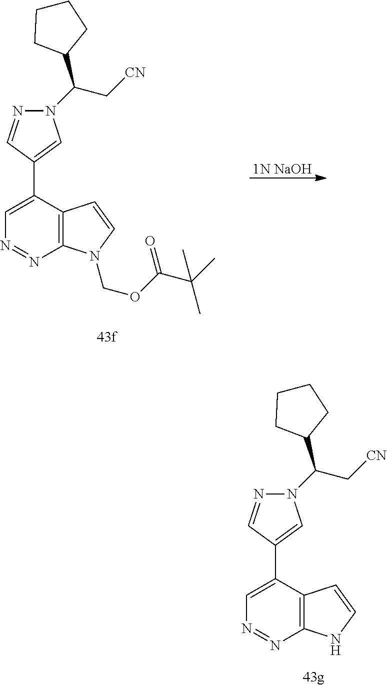

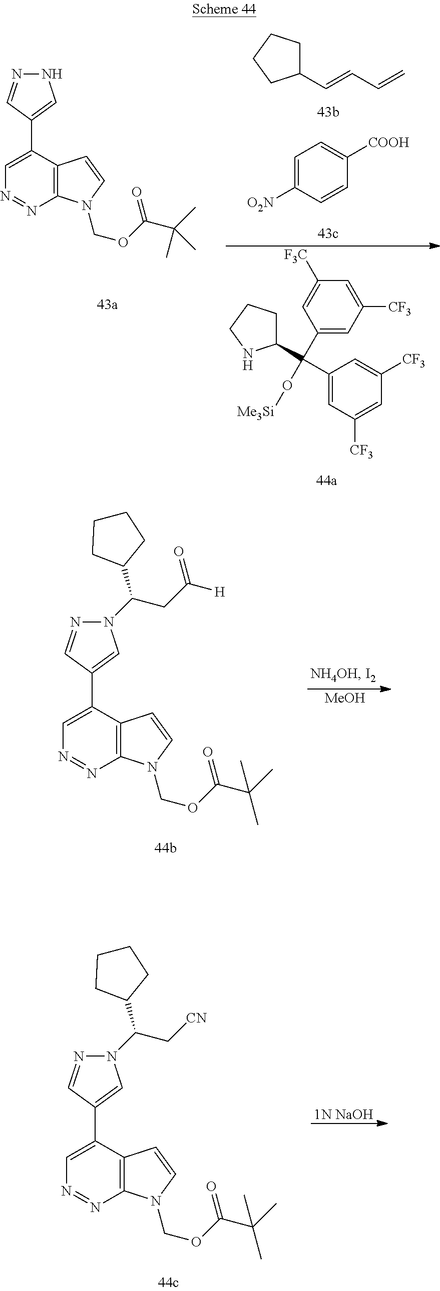

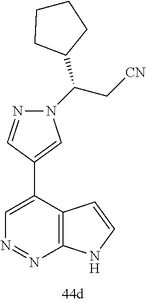

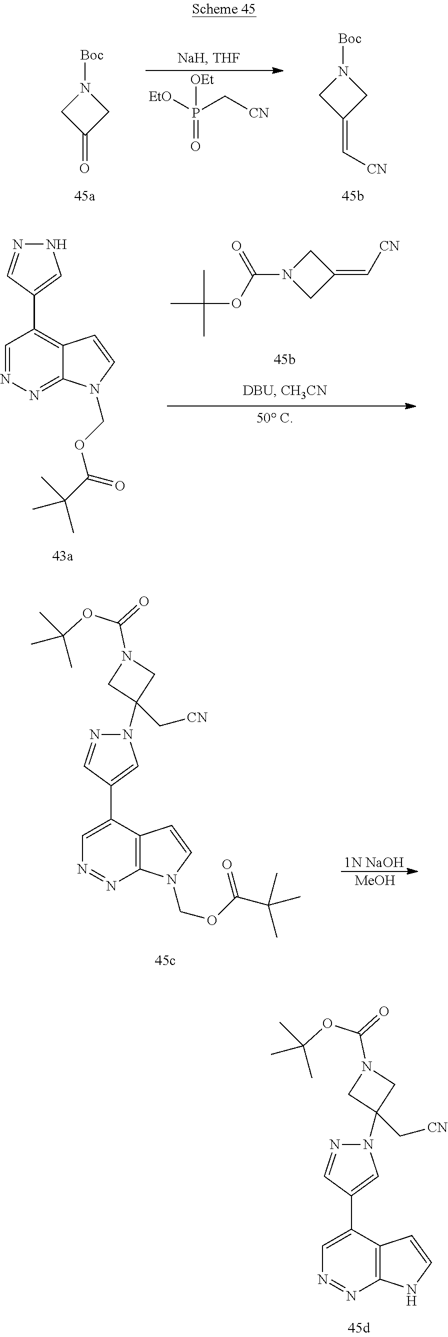

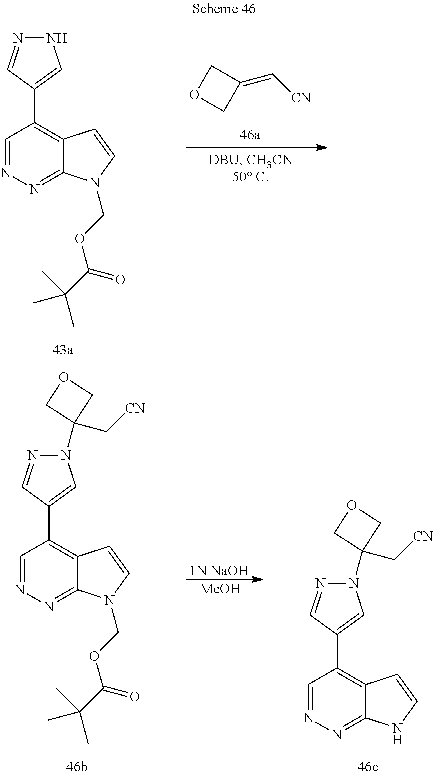

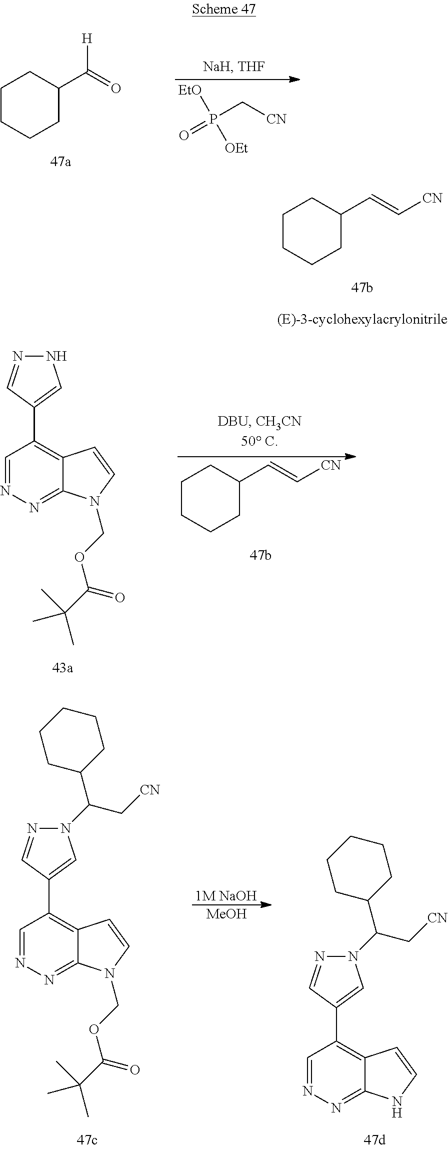

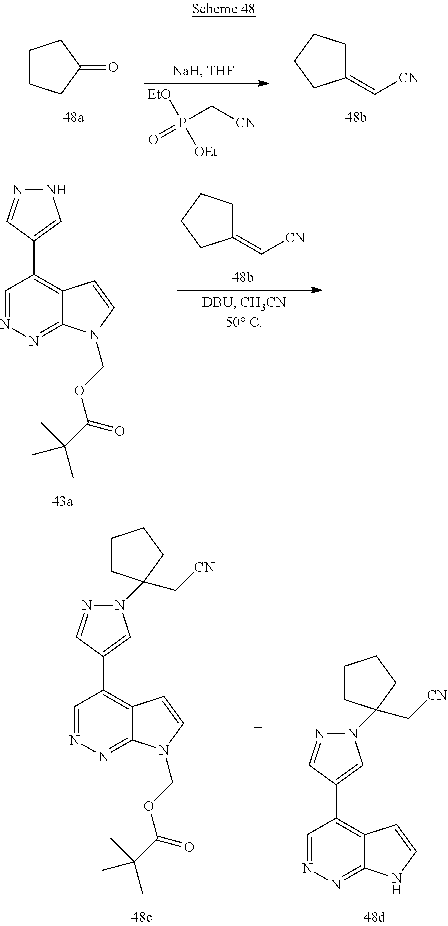

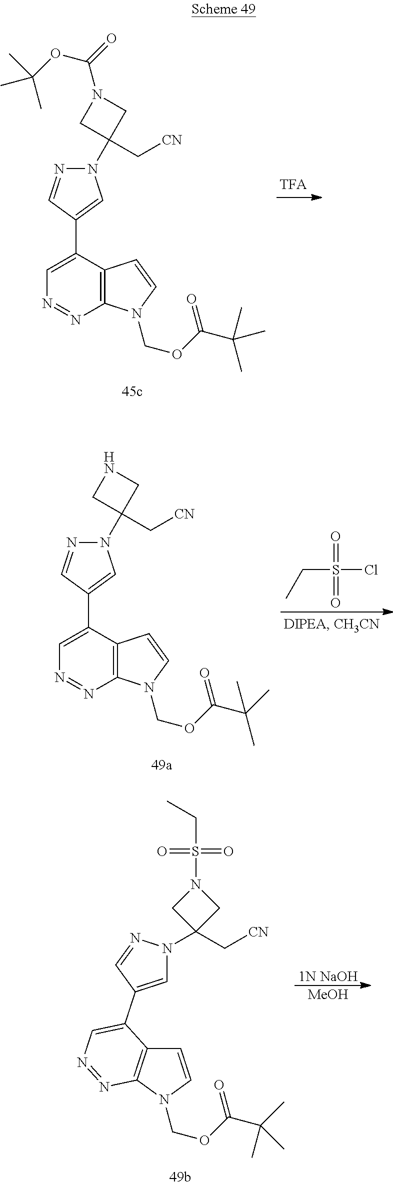

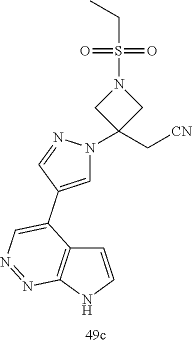

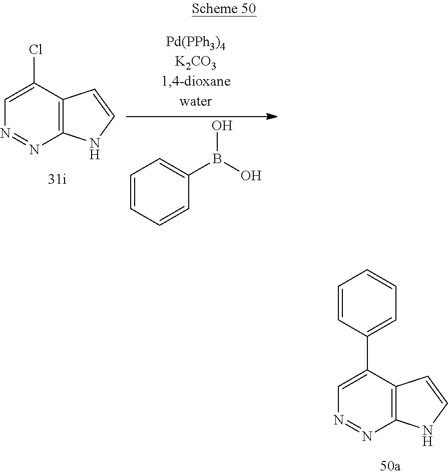

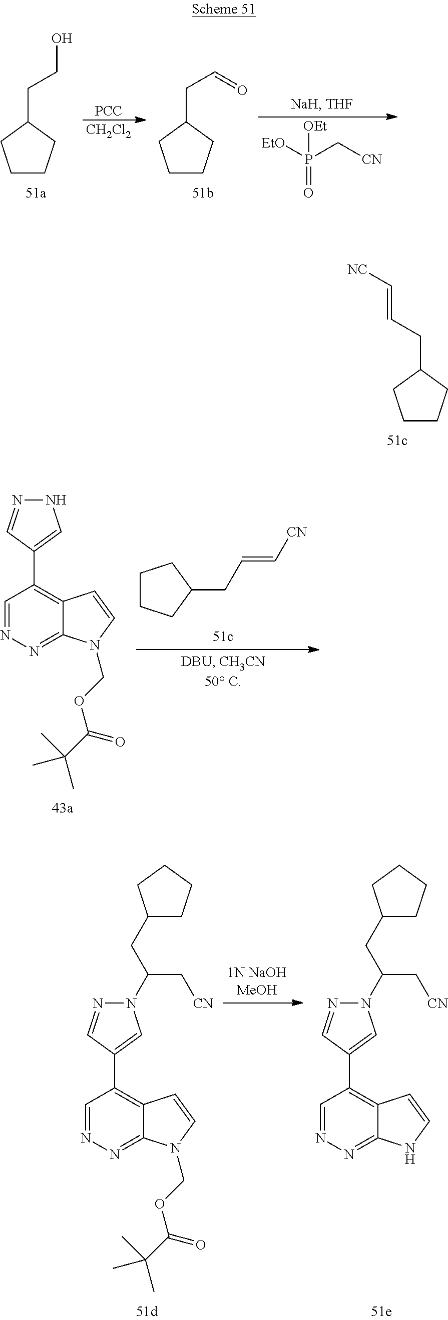

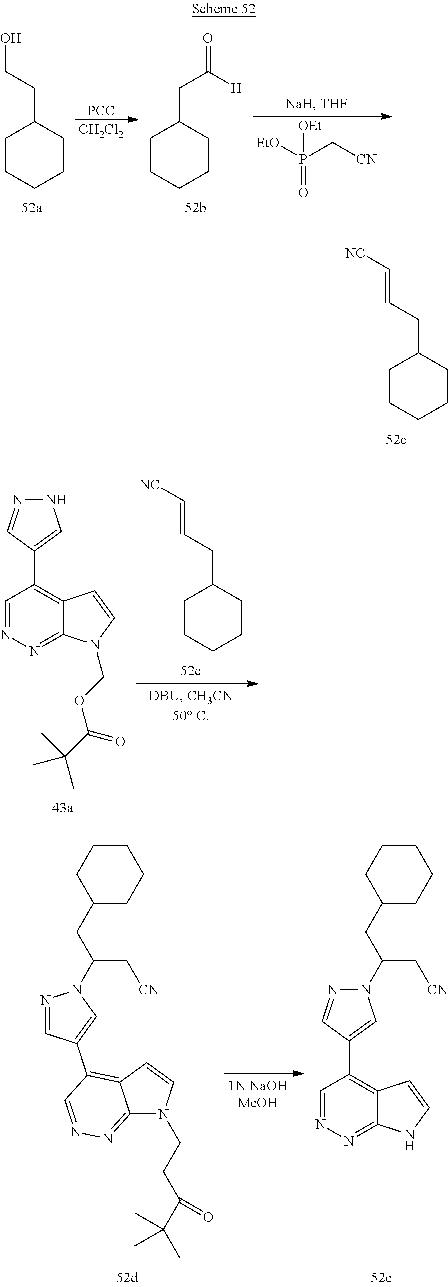

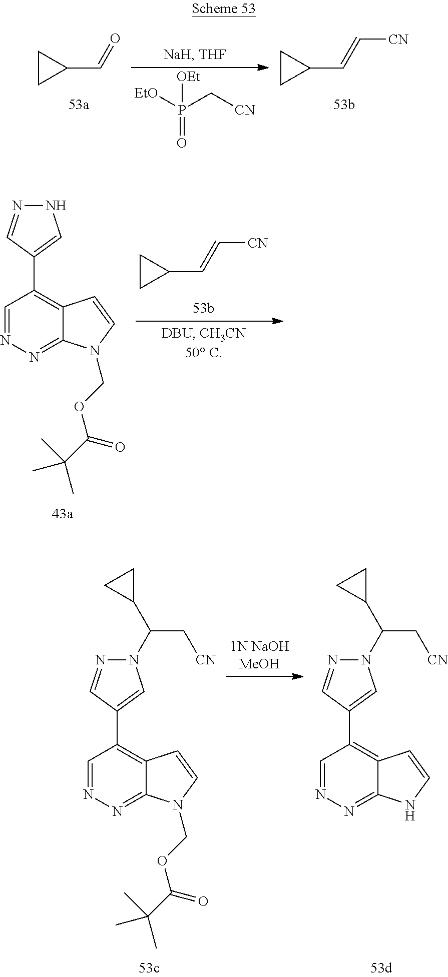

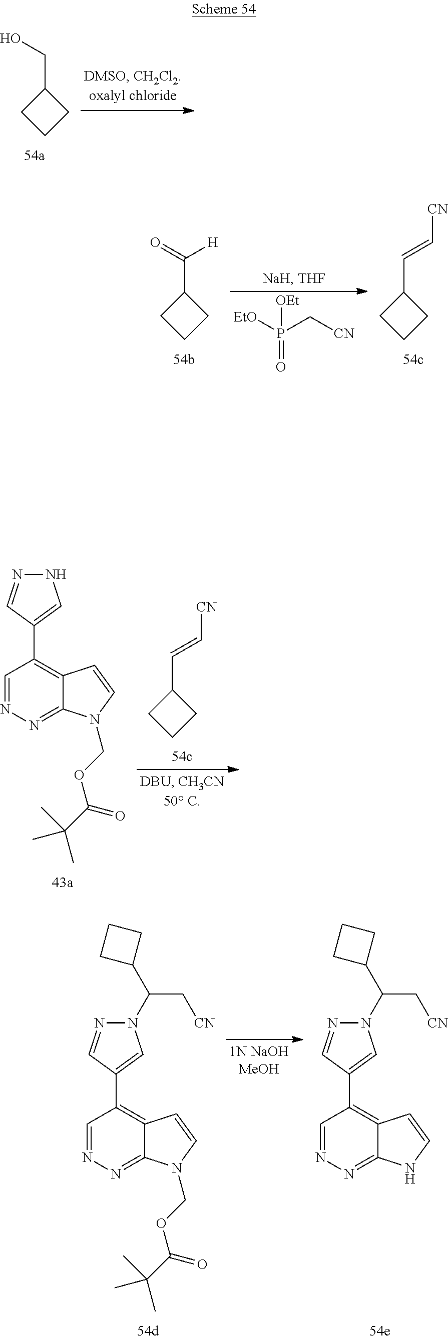

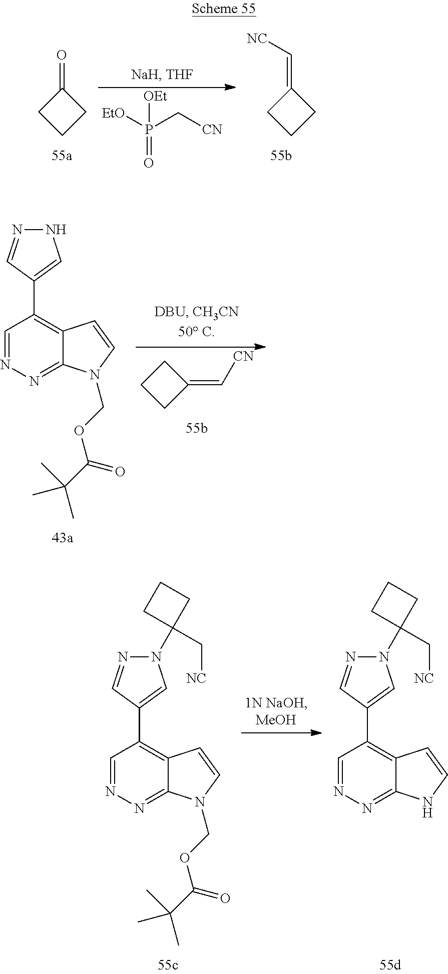

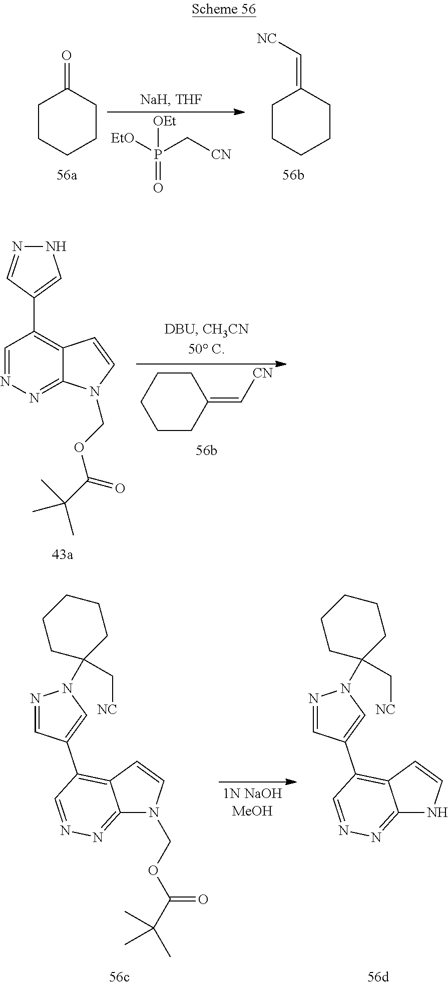

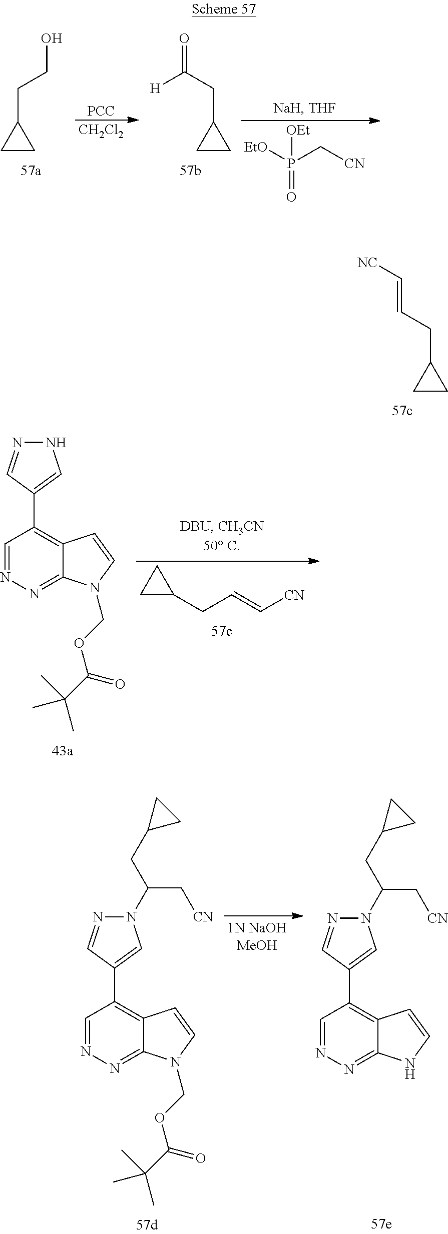

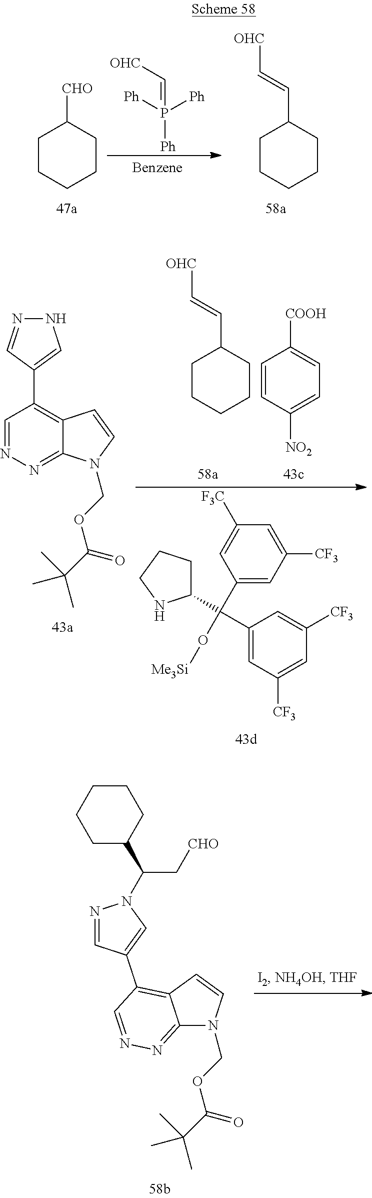

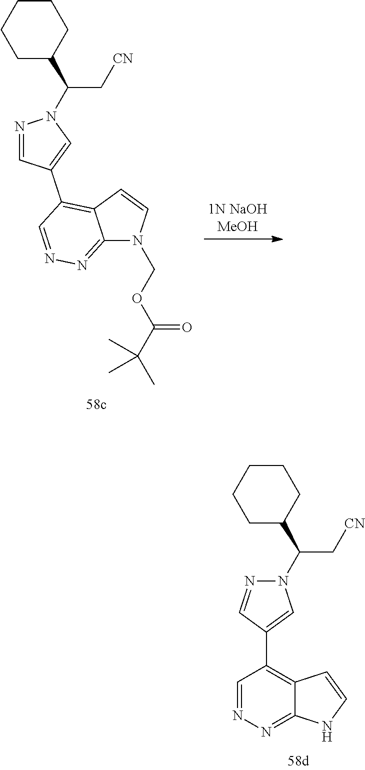

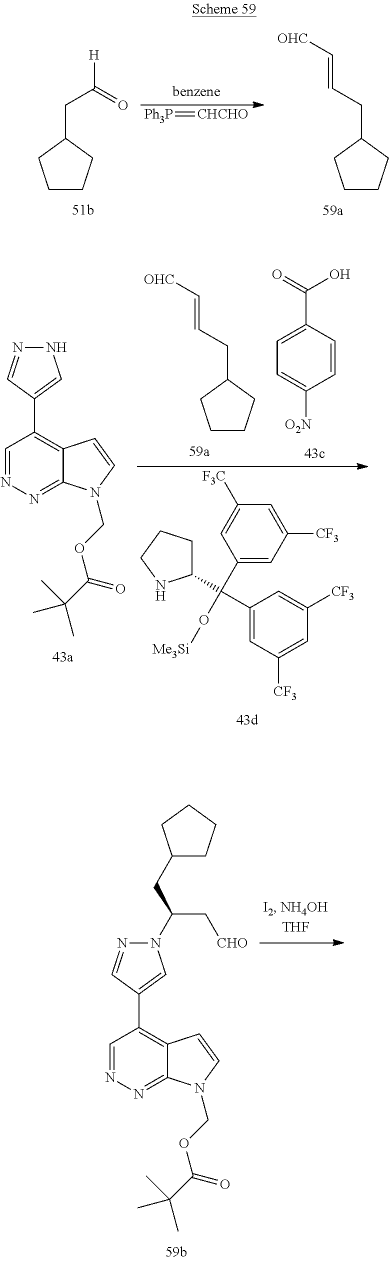

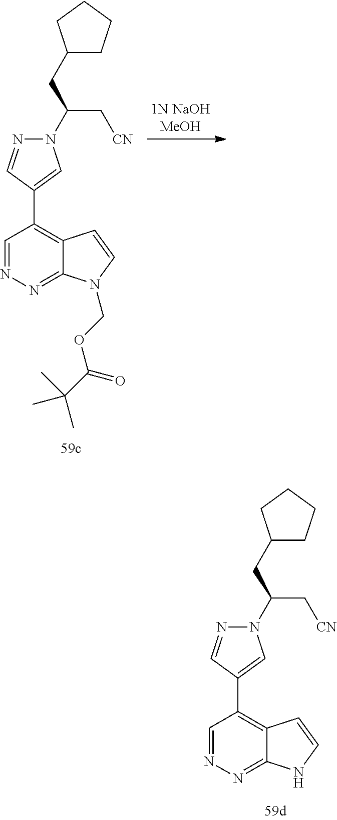

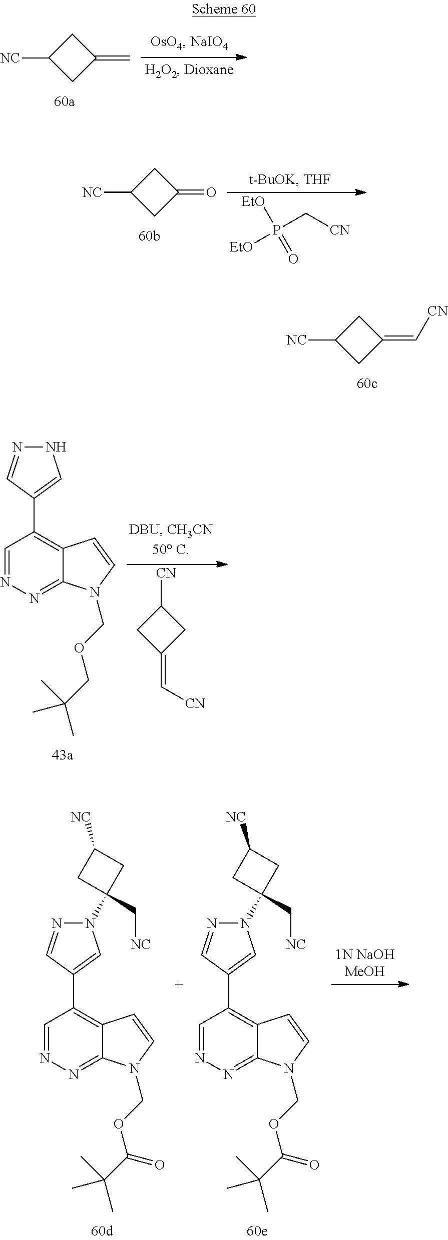

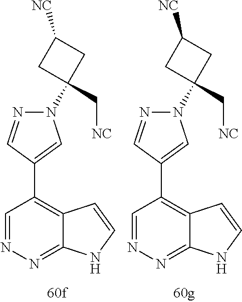

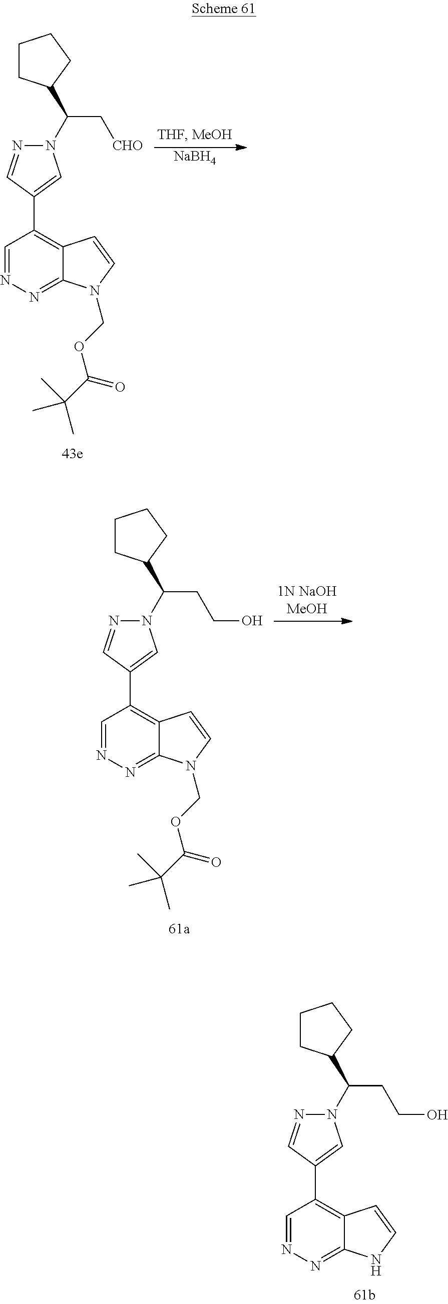

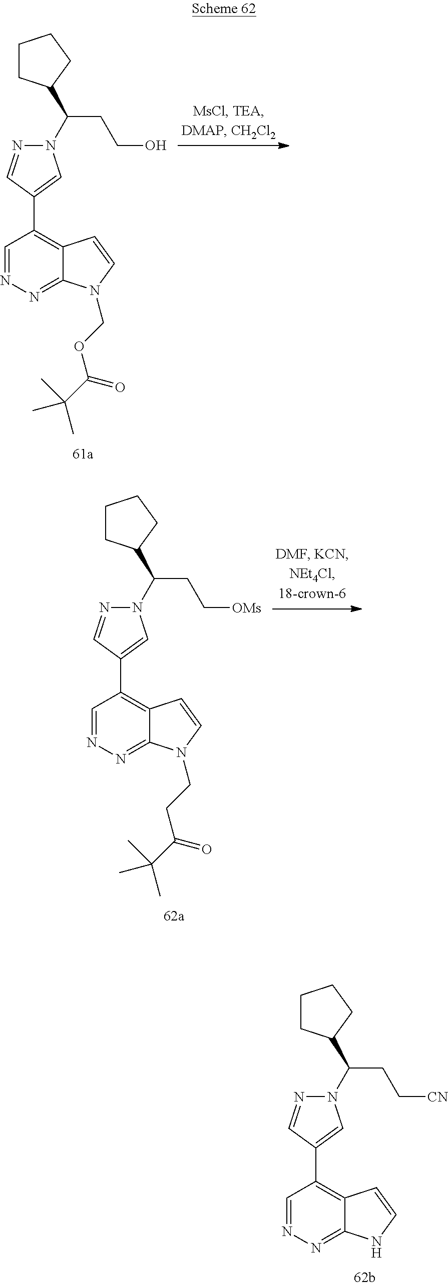

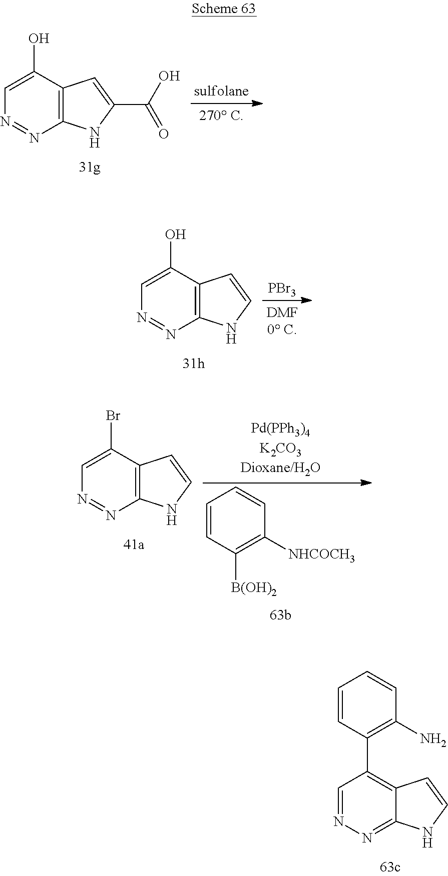

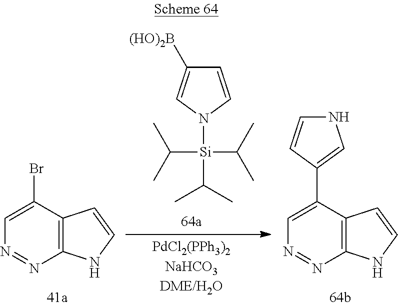

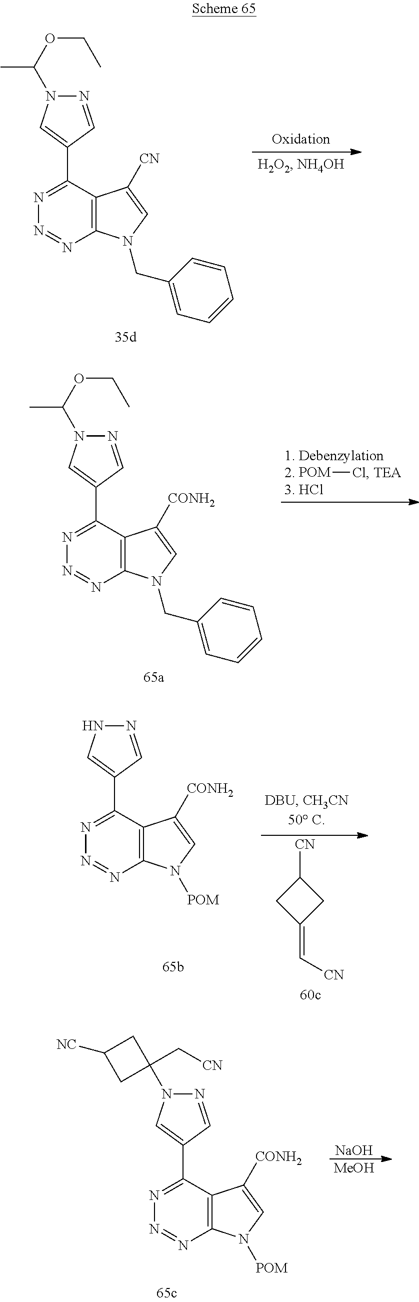

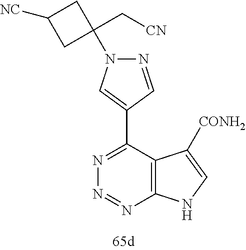

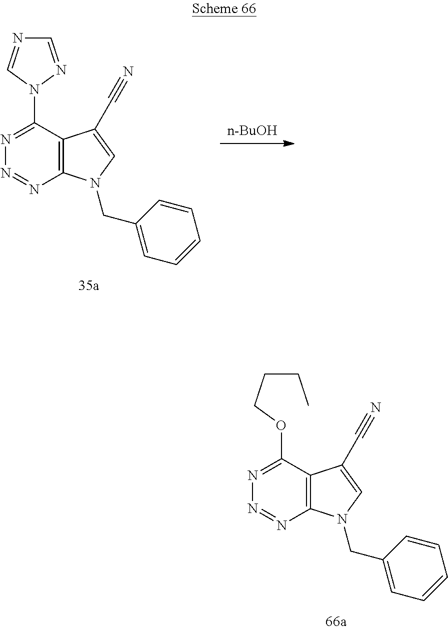

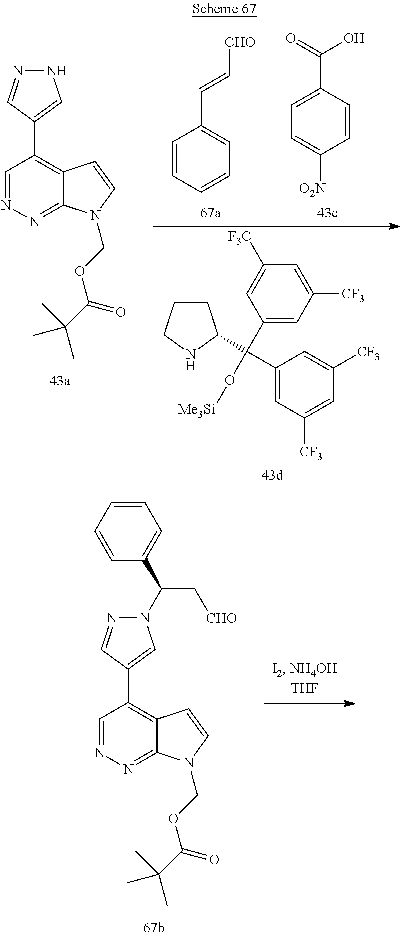

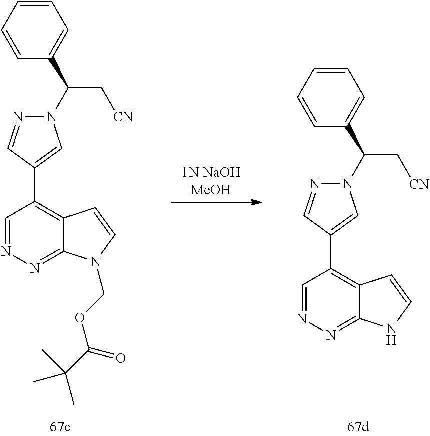

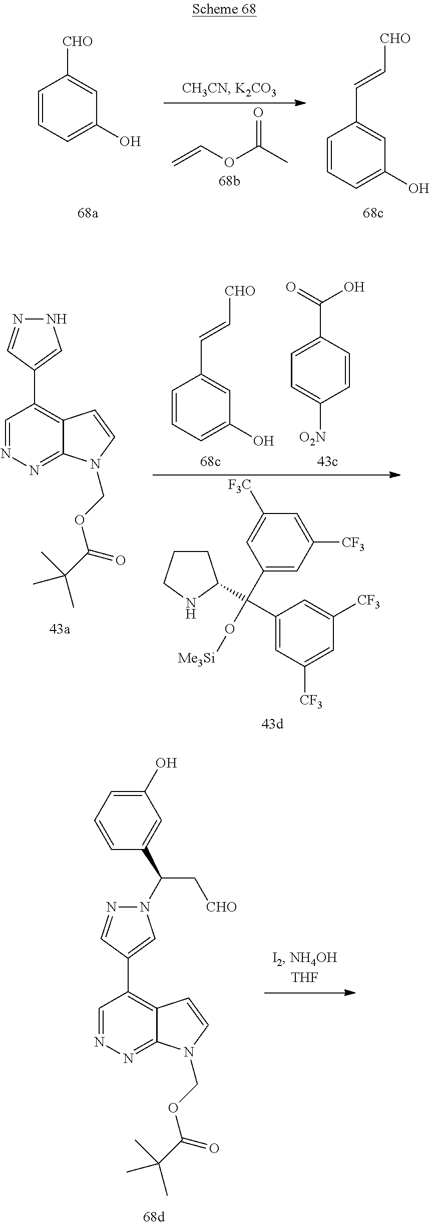

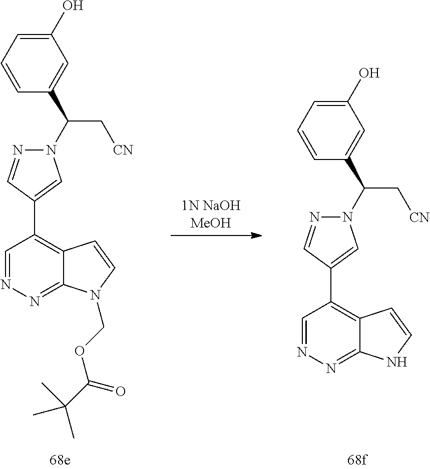

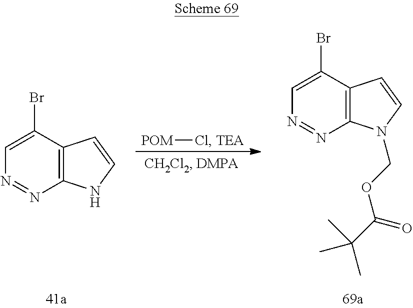

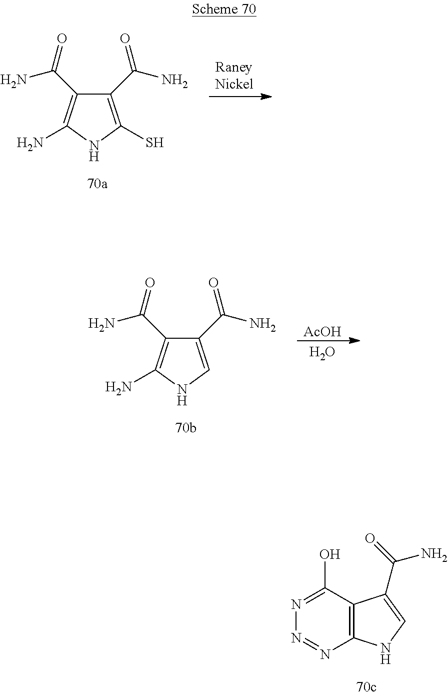

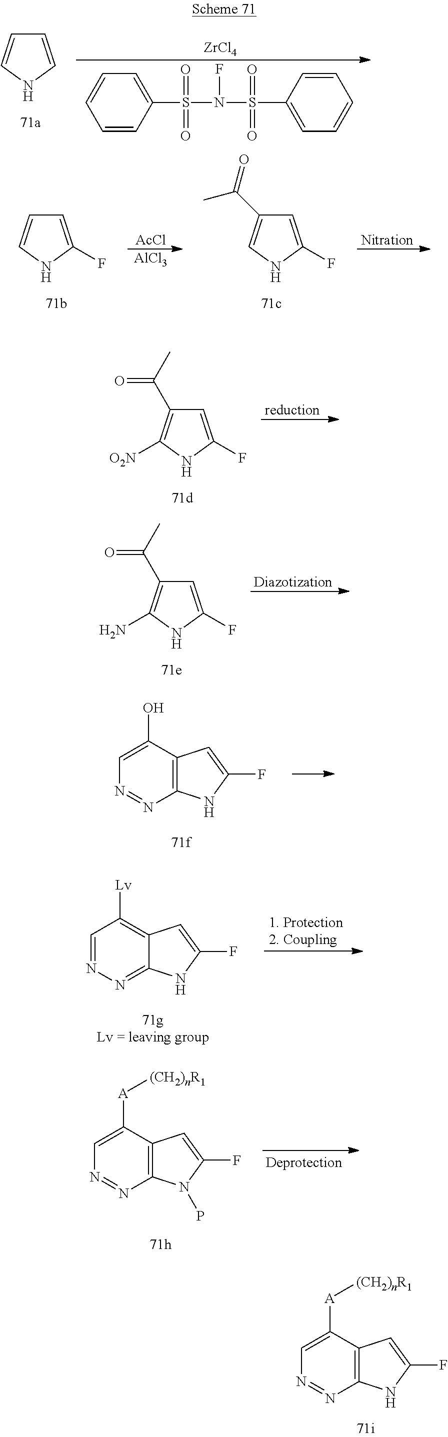

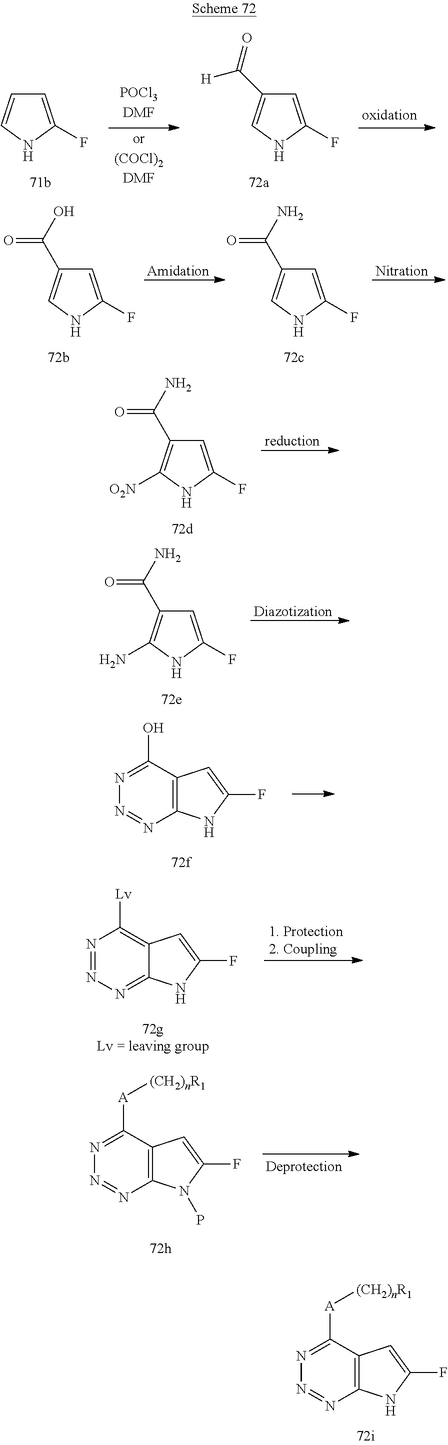

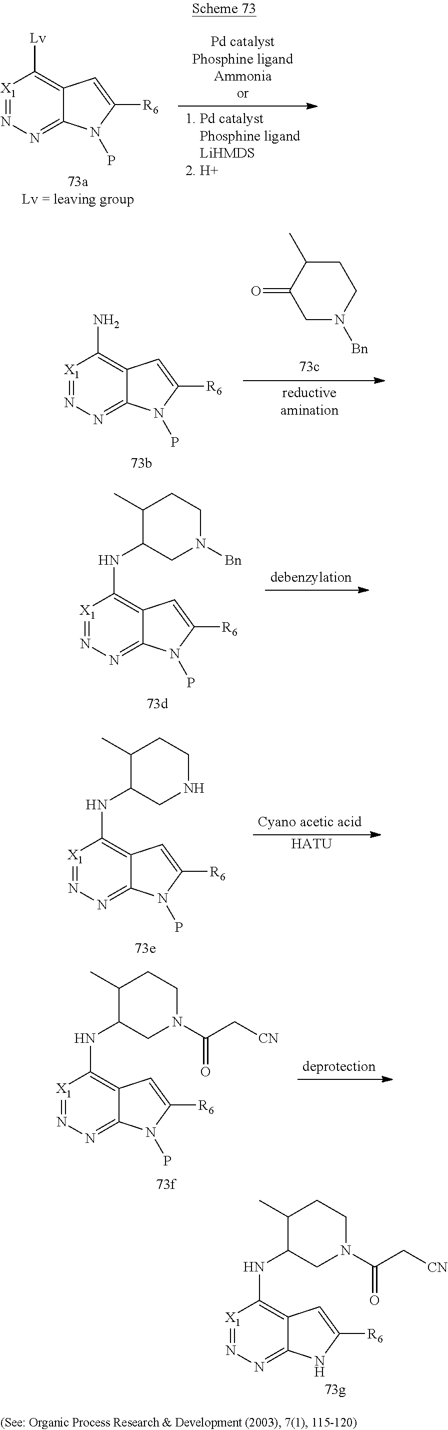

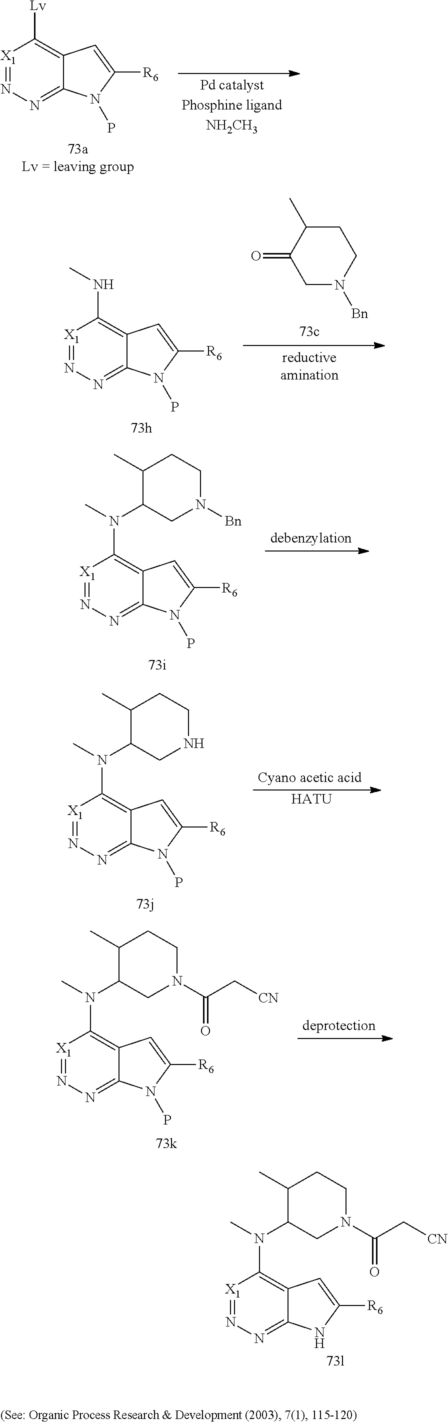

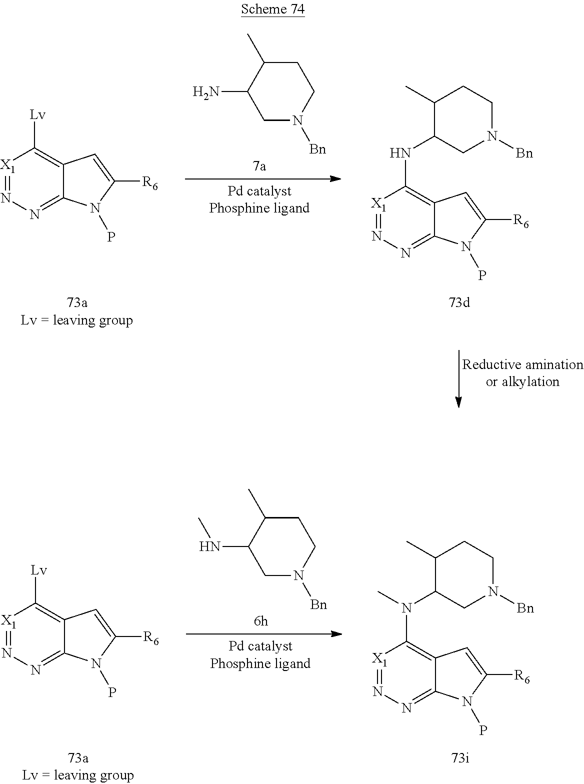

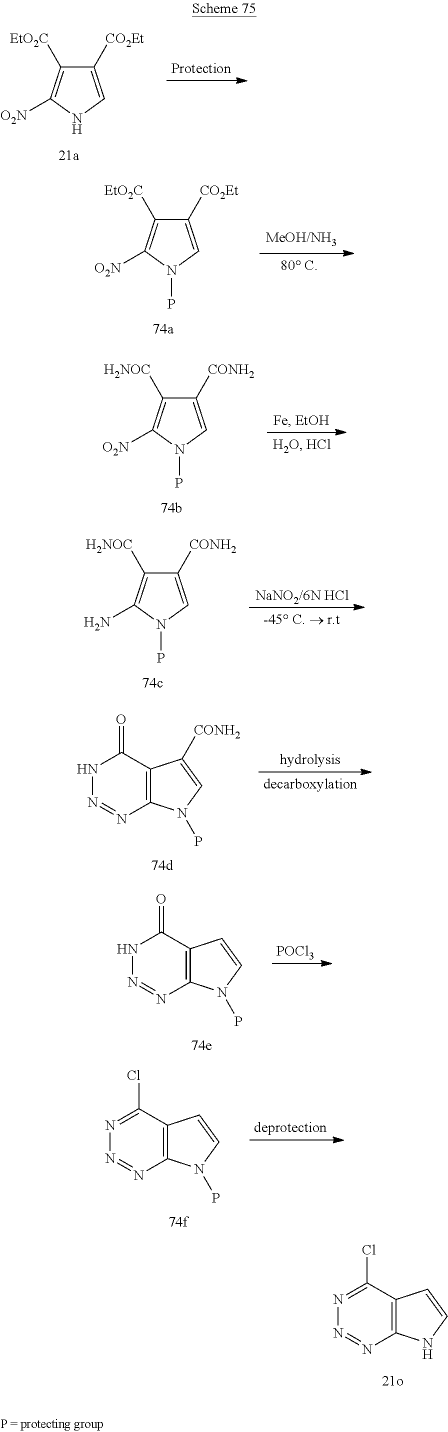

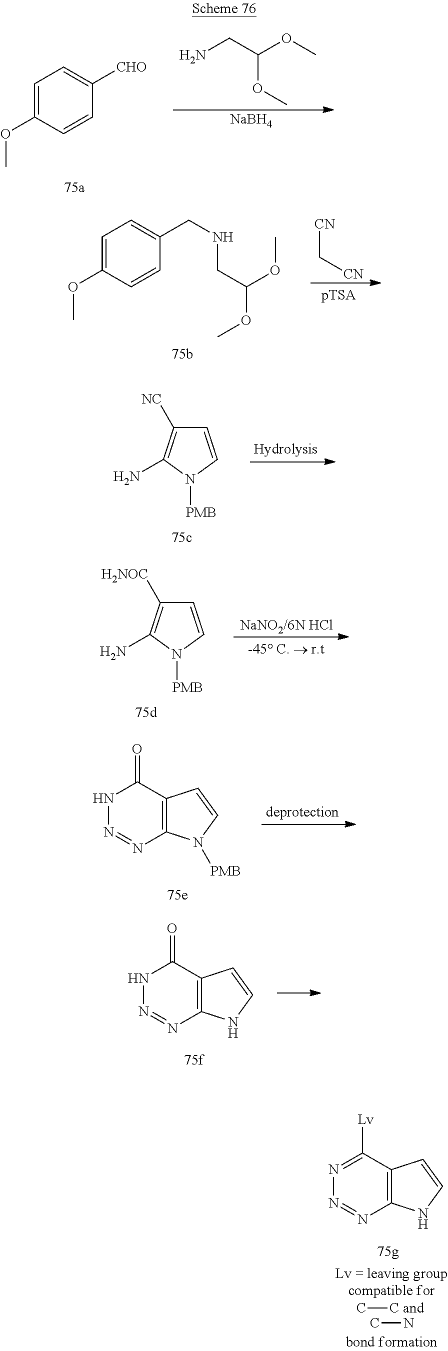

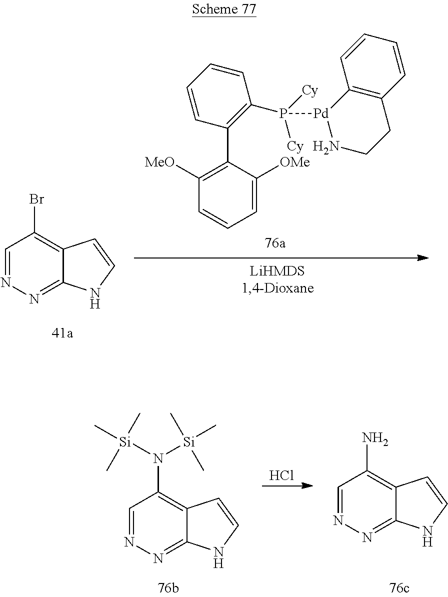

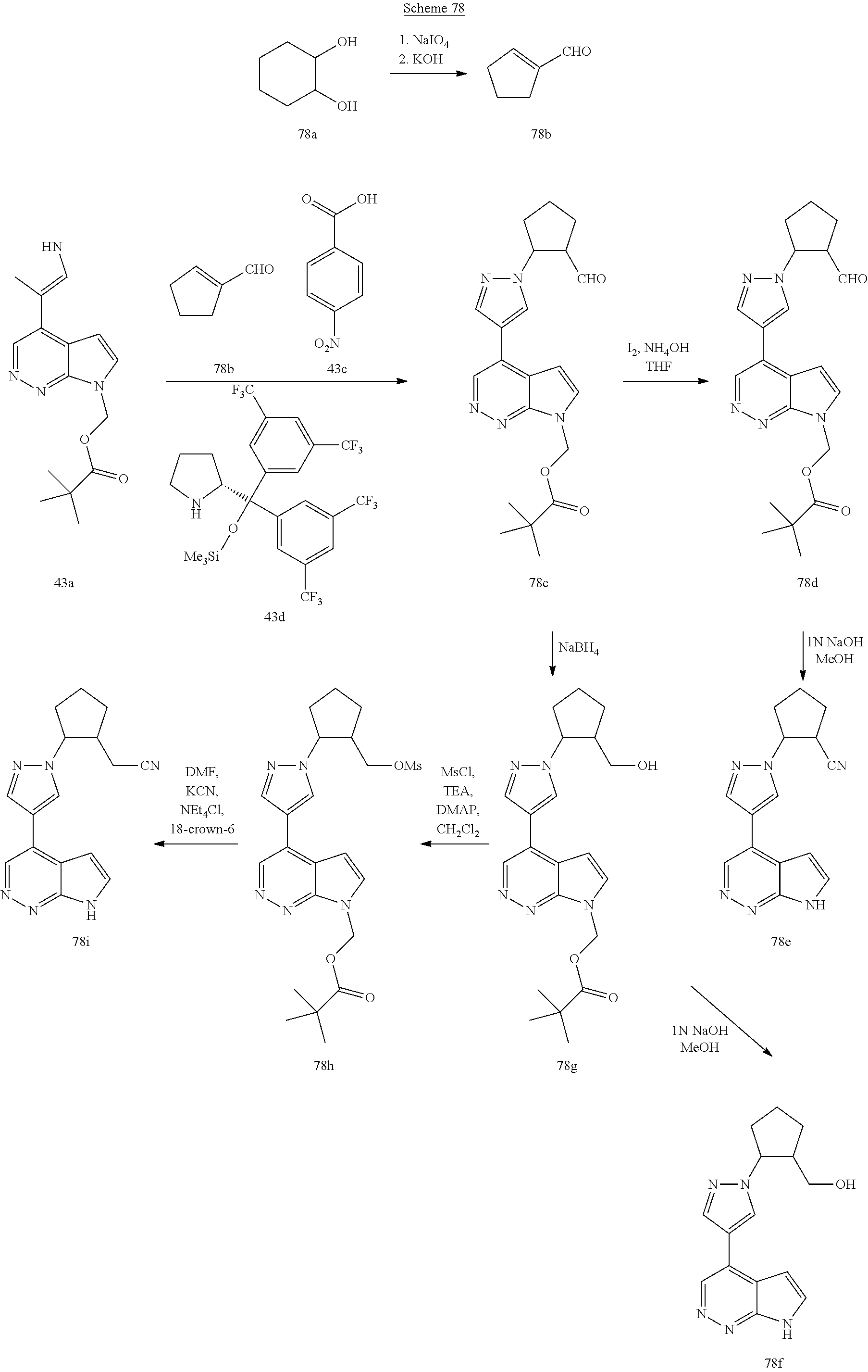

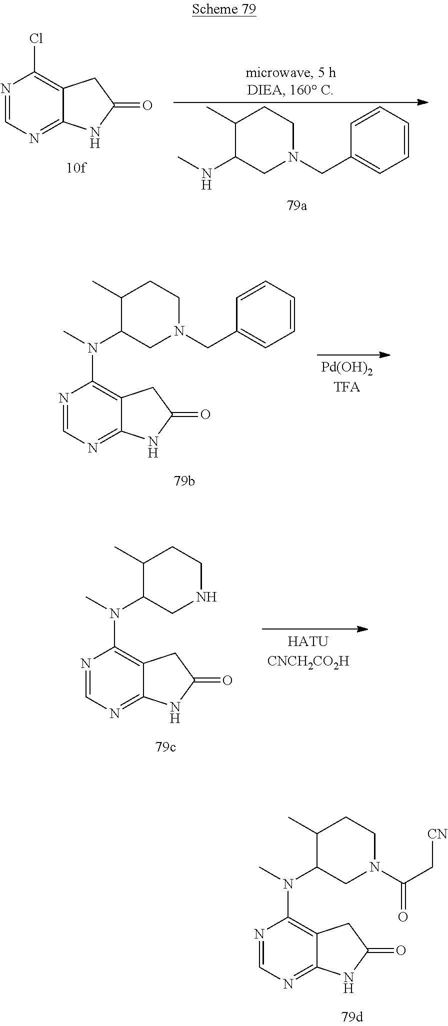

In one embodiment, the invention provides a method for preparing a salt of a compound of formula I, comprising reacting the compound of formula I with an acid under conditions suitable to provide the salt.

In one embodiment, the invention provides a method for preparing a pharmaceutical composition comprising a compound of formula I, or a pharmaceutically acceptable salt thereof, in combination with a pharmaceutically acceptable diluent or carrier, comprising combining the compound of formula I, or the pharmaceutically acceptable salt thereof, with the pharmaceutically acceptable diluent or carrier to provide the pharmaceutical composition.

The compounds of formula I can be formulated as pharmaceutical compositions and administered to a mammalian host, such as a human patient, in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following diluents and carriers: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula Ito the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents that are useful for immunosuppression. Accordingly, in one embodiment the invention also provides a composition comprising a compound of formula I, or a pharmaceutically acceptable salt thereof, at least one other therapeutic agent, and a pharmaceutically acceptable diluent or carrier. The invention also provides a kit comprising a compound of formula I, or a pharmaceutically acceptable salt thereof, at least one other therapeutic agent, packaging material, and instructions for administering the compound of formula I or the pharmaceutically acceptable salt thereof and the other therapeutic agent or agents to an animal to suppress an immune response in the animal.

Compounds of the invention may also be useful in the treatment of other diseases, conditions or disorders associated with the function of a kinase such as a Janus kinase (e.g. JAK1, JAK2 or TYK2) including the pathological activation of a kinase such as a Janus kinase (e.g. JAK1, JAK2 or TYK2). Accordingly, in one embodiment the invention provides a compound of formula I for the treatment of a kinase such as a Janus kinase (e.g. JAK1, JAK2 or TYK2) related disease, condition or disorder.

The ability of a compound of the invention to bind to JAK3 may be determined using pharmacological models which are well known to the art, or using Test A described below.

### Test A.

Inhibition constants (IC₅₀s) were determined against JAK3 (JH1domain-catalytic) kinase and other members of the JAK family. Assays were performed as described in Fabian et al. (2005) Nature Biotechnology, vol. 23, p. 329 and in Karaman et al. (2008) Nature Biotechnology, vol. 26, p. 127 Inhibition constants were determined using 11 point dose response curves which were performed in triplicate. Table 1 shown below lists compounds of the invention and their respective IC₅₀ values.

The ability of a compound of the invention to provide an immunomodulatory effect can also be determined using pharmacological models which are well known to the art. The ability of a compound of the invention to provide an anti-cancer effect can also be determined using pharmacological models which are well known to the art.

The invention will now be illustrated by the following non-limiting Examples.

### Example 1

### 4-(1H-pyrazol-4-yl)-7H-pyrrolo[2,3-c]pyridazine (31m)

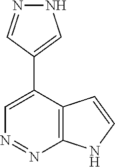

To a solution of 4-(1-(1-ethoxyethyl)-1H-pyrazol-4-yl)-7-((2-(trimethylsilypethoxy)methyl)-7H-pyrrolo[2,3-c]pyridazine 311 (34 mg, 0.087 mmol) in THF (2 mL) and methanol (2 mL) was added 4N HCl in 1,4-dioxane (1 mL) and stirred at room temperature overnight. The reaction mixture was concentrated in vacuum and the residue obtained was triturated with ether. The solid obtained was collected by filtration, washed with ether to give 4-(1H-pyrazol-4-yl)-7H-pyrrolo[2,3-c]pyridazine hydrochloride 31m (18 mg, 93%) as a yellow solid. ¹HNMR (300 MHz, DMSO).δ 13.85 (s, 1H), 9.65 (s, 1H), 8.79 (s, 2H), 8.68 (t, J=2.9, 1H), 7.54 (d, J=2.1, 1H). MS (ES⁺) 186.1 (M+1).

### Preparation of 4-(1-(1-ethoxyethyl)-1H-pyrazol-4-yl)-7-((2-(trimethylsilypethoxy)methyl)-7H-pyrrolo[2,3-c]pyridazine (311)

### Step 1:

To a stirred solution of 2,2,2-trichloro-1-(1H-pyrrol-2-yl)ethanone 31a (50 g, 235.33 mmol) in methylene chloride (250 mL) and nitroethane (250 mL) was added aluminum chloride (62.75 g, 470.67 mmol) and cooled to −30° C. To this cold solution acetyl chloride (23.09 g, 294.17 mmol) was added slowly over a period of 10 min. The reaction was stirred for additional 30 min poured into ice water (2000 mL) and extracted with ethyl acetate (3×500 mL), the ethyl acetate layers were combined washed with water (2×500 mL), brine (1×250 mL), dried and concentrated in vacuum. The residue was triturated with hexane (500 mL), and the solid obtained was collected by filtration to afford 1-(4-acetyl-1H-pyrrol-2-yl)-2,2,2-trichloroethanone 31b, (51.7 g, 86%) as a colorless solid. ¹HNMR (300 MHz, DMSO) δ 13.03 (s, 1H, D₂O exchangeable), 8.07 (s, 1H), 7.59 (s, 1H), 2.42 (s, 3H). MS (ES⁺¹) 253.7 (M−1).

### Step 2:

To a stirred solution of 1-(4-acetyl-1H-pyrrol-2-yl)-2,2,2-trichloroethanone 31b (50.0 g, 196.39 mmol) in conc. Sulfuric acid (400 mL) was added nitric acid (15.89 mL, 247.13 mmol, 70% solution) at 0° C. The reaction was stirred for 30 min and poured into ice water. The solid separated was collected by filtration and aqueous layer was extracted with ethyl acetate (2×1000 mL). The ethyl acetate layers were combined and the solid collected above was also dissolved in ethyl acetate extract. The ethyl acetate layer was washed with water (2×500 mL); brine (1×500 mL) dried and concentrated. The residue was triturated with hexanes (500 mL) and the solid obtained was collected by filtration to afford 1-(4-acetyl-5-nitro-1H-pyrrol-2-yl)-2,2,2-trichloroethanone 31c (54.5 g, 92.6%) as a colorless solid. ¹HNMR (300 MHz, DMSO) δ 11.27 (bs, 1H, D₂O exchangeable), 7.62-7.53 (m, 1H), 2.51 (s, 3H).

### Step 3:

To a stirred solution of 1-(4-acetyl-5-nitro-1H-pyrrol-2-yl)-2,2,2-trichloroethanone 31c (53.57 g, 180.09 mmol) from above step in methanol (200 mL) was added sodium methoxide (42.80 g, 198.09 mmol, 25% solution in methanol) at 20° C. The reaction was stirred at room temperature for 30 min and quenched carefully with a mixture of water and dilute HCl (200 mmol). The solid separated was collected by filtration. The filtrate was extracted with ethyl acetate (2×1000 mL). The solid collected was dissolved in combined ethyl acetate extracts and washed with water (2×500 mL), brine (1×250 mL), dried and concentrated in vacuum. The crude residue was purified by flash chromatography to furnish methyl 4-acetyl-5-amino-1H-pyrrole-2-carboxylate 31d (17.5 g pure and 4.25 g with small impurity). ¹HNMR (300 MHz, DMSO) δ 14.58 (s, 1H, D₂O exchangeable), 7.35-7.13 (m, 1H), 4.03-3.72 (m, 3H), 2.50 (s, 3H). MS (ES⁻¹) 211.0 (M−1).

### Step 4:

A solution of Methyl 4-acetyl-5-amino-1H-pyrrole-2-carboxylate 31d (10 g, 47.00 mmol) in acetic acid (60 mL) was heated at 45° C. To a homogenous solution iron powder (7.87 g, 55.85 mmol) was added and continued stirring at 45° C. After 30 min the reaction temperature reaches 100° C., and the reaction mixture becomes heterogeneous. The solid obtained was dissolved in 10% aq. ammonia in methanol (100 mL), filtered through celite and concentrated in vacuum. The residue obtained was purified by flash chromatography (silica gel, eluting with CMA 80 in chloroform 0 to 50%) to afford methyl 4-acetyl-5-amino-1H-pyrrole-2-carboxylate 31e (7.5 g. 87.5%) as a light brown solid. ¹HNMR (300 MHz, DMSO) δ 10.87 (s, 1H, D₂O exchangeable), 7.03 (d, J=2.4, 1H), 6.35 (s, 2H, D₂O exchangeable), 3.71 (s, 3H), 2.21 (s, 3H). MS (ES⁺¹) 183.2 (M+1)

### Step 5:

A solution of methyl 4-acetyl-5-amino-1H-pyrrole-2-carboxylate 31e (6.96 g, 38.20 mmol) in acetic acid (70 mL) and water (15 mL) was cooled to 18° C. and added a solution of sodium nitrite (3.95 g, 57.30 mmol) in water (10 mL) maintaining temperature below 20° C. The reaction mixture was stirred for 5 mins (TLC analysis shows disappearance of starting material) and warmed to 65° C. The reaction was stirred at 65° C. for 48 h and concentrated in vacuum. The residue was purified by flash chromatography (silica gel eluting with methanol in chloroform 0 to 15%) to furnish methyl 4-hydroxy-7H-pyrrolo[2,3-c]pyridazine-6-carboxylate 31f (2.5 g 33.8%) as brown solid. ¹HNMR (300 MHz, DMSO) δ 13.57 (s, 1H, D₂O exchangeable), 12.83 (s, 1H, D₂O exchangeable), 7.55 (s, 1H), 7.08 (s, 1H), 3.82 (s, 3H), MS (ES⁺¹) 194.1 (M+1), (ES⁻¹) 192.0 (M−1).

### Step 6:

Methyl 4-hydroxy-7H-pyrrolo[2,3-c]pyridazine-6-carboxylate 31f (0.376 g, 1.94 mmol was dissolved in potassium hydroxide solution (5.84 mL, 11.68 mmol) and heated at 70° C. for 30 mins The reaction was then cooled to 20° C. and neutralized with 3 N HCl. The solid obtained was collected by filtration and dried in vacuum to afford 4-hydroxy-7H-pyrrolo[2,3-c]pyridazine-6-carboxylic acid 31g (0.27 g, 77.6%) as a light brown solid. ¹HNMR (300 MHz, DMSO) δ 13.53 (s, 1H, D₂O exchangeable), 13.40-12.95 (m, 1H, D₂O exchangeable), 12.62 (s, 1H, D₂O exchangeable), 7.55 (s, 1H), 7.03 (s, 1H), MS (ES⁻¹) 178.0 (M−1).

### Step 7:

A solution of 4-hydroxy-7H-pyrrolo[2,3-c]pyridazine-6-carboxylic acid 31g (0.537 g, 3.00 mmol) dissolved in trifluoroacetic acid (12 mL) was added into a glass ampoule. The glass ampoule was sealed and heated at 230° C. for 48 h. The reaction mixture was cooled to room temperature and the contents of the ampoule was concentrated in vacuum to dryness to 7H-pyrrolo[2,3-c]pyridazin-4-ol 31h as a trifluoro acetate salt. The product obtained was pure enough to be taken to next step. ¹HNMR (300 MHz, DMSO) δ 8.85 (s, 1H), 8.27 (d, J=3.4, 1H), 7.12 (d, J=3.4, 1H). MS (ES⁺¹) 136.2 (M+1).

### Step 8:

To a solution of 7H-pyrrolo[2,3-c]pyridazin-4-ol 31h (3 mmol) from above reaction in acetonitrile (50 mL) and was added benzyltriethylammonium chloride (1.33 g, 4.50 mmol), N,N′-dimethylaniline (0.54 g, 4.50 mmol) and heated to 80° C. To the reaction mixture at 80° C. was added cautiously phosphorous oxy chloride (2.76 g, 18.0 mmol) and continued heating at 80° for 2 h. The reaction mixture was concentrated in vacuum to dryness and the residue obtained was quenched with ice water (10 mL). The pH of the mixture was adjusted to 7-8 using saturated aqueous sodium bicarbonate. The reaction mixture was diluted with ethyl acetate (50 mL) and filtered to remove insoluble solids. The organic layer was separated and the aqueous layer was extracted with ethyl acetate (2×50 mL). The organic layers were combined washed with water (2×20 mL), brine (1×20 mL), dried over MgSO₄ filtered and concentrated in vacuum to dryness. The crude residue obtained was purified by flash column chromatography [silica gel 12 g, eluting with a of (9:1) mixture ethyl acetate and methanol in hexanes (0 to 100%)] to furnish 4-chloro-7H-pyrrolo[2,3-c]pyridazine 31i (104 mg, 22.5% from acid) as a colorless solid. ¹HNMR (300 MHz, DMSO) δ 12.81 (d, J=29.3, 1H), 8.98 (s, 1H), 8.05 (dd, J=2.7, 3.3, 1H), 6.65 (dd, J=1.8, 3.4, 1H). MS (ES⁺¹) 154.1 (M+1).

### Step 9:

To a solution of 4-chloro-7H-pyrrolo[2,3-c]pyridazine 31i (0.168 g, 1.09 mmol) in methylene chloride (10 mL) was added triethyl amine (0.33 g, 3.28 mmol) and cooled to −10° C. To the cold reaction mixture was added (2-(chloromethoxy)ethyl)trimethylsilane (0.273 g, 1.64 mmol) and stirred in cold for 2 h. The reaction was then quenched by adding water (15 mL) and extracted with chloroform (3×25 mL). The chloroform layers were combined and washed with water (2×10 mL), brine (1×10 mL), dried and concentrated in vacuum. The residue obtained was purified by flash column chromatography[silica gel 12 g, eluting with (9:1) mixture of ethyl acetate and methanol in hexanes (0 to 50%)] to afford 4-chloro-7-((2-(trimethylsilypethoxy)methyl)-7H-pyrrolo[2,3-c]pyridazine 31j (0.52 mg, 16.8%) as a sticky brownish yellow syrup. ¹HNMR (300 MHz, DMSO) δ 8.81 (d, J=13.1, 1H), 8.31 (t, J=3.1, 1H), 6.91 (t, J=8.5, 1H), 6.31-6.19 (m, 2H), 3.92-3.74 (m, 2H), 1.04-0.89 (m, 2H), −0.00 (s, 9H).

### Step 10:

To a solution of 4-chloro-7-((2-(trimethylsilyl)ethoxy)methyl)-7H-pyrrolo[2,3-c]pyridazine 31j (48 mg, 0.16 mmol), in 1,4-dioxane (3 mL) was added 1-(1-ethoxyethyl)-4-(4,4,5,5-tetramethyl-1,3,2-dioxaborolan-2-yl)-1H-pyrazole 31k (commercially available, 45 mg, 0.16 mmol), water (1 mL), and K₂CO₃ (93 mg, 0.67 mmol). The reaction mixture was degassed by bubbling nitrogen for about 5 minutes and charged with tetrakis(triphenylphosphine) Pd(0) (7.8 mg, 0.0067 mmol). The reaction mixture was heated at 80° C. under nitrogen for 4 h cooled to room temperature and quenched with brine solution (15 mL). The aqueous layer was extracted with EtOAc (2×30 mL). The organic layers were combined washed with brine (10 mL), dried over MgSO₄, and concentrated in vacuo. The crude residue was purified by flash column chromatography (silica gel 12 g, eluting with (9:1) mixture of EtOAc and MeOH in hexane 0-20%) to afford 4-(1-(1-ethoxyethyl)-1H-pyrazol-4-yl)-74 (2-(trimethylsilyl)ethoxy)methyl)-7H-pyrrolo[2,3-c]pyridazine 311 (36 mg, 58&) as a dark brownish yellow oil. ¹HNMR (300 MHz, DMSO) δ 9.06 (s, 1H), 9.00 (s, 1H), 8.58 (s, 1H), 8.16 (d, J=2.5, 1H), 7.25 (d, J=2.5, 1H), 6.22 (s, 2H), 5.78 (q, J=6.0, 1H), 3.89-3.81 (m, 2H), 3.60 (dq, J=7.0, 9.6, 1H), 3.42-3.32 (m, 1H), 1.79 (d, J=6.0, 3H), 1.17 (t, J=7.0, 3H), 1.01-0.92 (m, 2H), 0.00 (s, 9H). MS (ES⁺) 388.1 (M+1).

### Example 2

### 4-(1-(1-ethoxyethyl)-1H-pyrazol-4-yl)-7H-pyrrolo[2,3-c]pyridazine (32c)

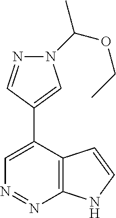

To a solution of (4-chloro-7H-pyrrolo[2,3-c]pyridazin-7-yl)methyl pivalate 32a (100 mg, 0.37 mmol), in 1,4-dioxane (4 mL) was added 1-(1-ethoxyethyl)-4-(4,4,5,5-tetramethyl-1,3,2-dioxaborolan-2-yl)-1H-pyrazole 31k (commercially available, 99 mg, 0.37 mmol), water (2 mL), and K₂CO₃ (93 mg, 0.67 mmol). The reaction mixture was degassed by bubbling nitrogen for about 5 minutes and charged with tetrakis(triphenylphosphine) Pd(0) (17 mg, 0.014 mmol). The reaction mixture was heated at 80° C. under nitrogen for 2 h cooled to room temperature and quenched with brine solution (10 mL). The aqueous layer was extracted with EtOAc (2×30 mL). The organic layers were combined washed with brine (10 mL), dried over MgSO₄, and concentrated in vacuo. The crude residue was purified by flash column chromatography (silica gel 12 g, eluting with (9:1) mixture of EtOAc and MeOH in hexane 0-20%) to afford (4-(1-(1-ethoxyethyl)-1H-pyrazol-4-yl)-7H-pyrrolo[2,3-c]pyridazin-7-yl)methyl pivalate 32b (32 mg, 23%) as a dark brownish yellow oil followed by 4-(1-(1-ethoxyethyl)-1H-pyrazol-4-yl)-7H-pyrrolo[2,3-c]pyridazine 32c (12 mg, 13%) as a colorless solid.

(4-(1-(1-ethoxyethyl)-1H-pyrazol-4-yl)-7H-pyrrolo[2,3-c]pyridazin-7-yl)methyl pivalate (32b): ¹HNMR (300 MHz, DMSO) δ 9.33 (d, J=6.3, 1H), 8.86 (s, 1H), 8.39 (s, 1H), 8.02 (d, J=3.7, 1H), 7.11 (d, J=3.7, 1H), 6.40 (s, 2H), 5.66 (q, J=6.0, 1H), 3.49 (dq, J=7.0, 9.6, 1H), 3.31-3.23 (m, 1H), 1.69 (d, J=6.0, 3H), 1.09 (s, 9H), 1.05 (t, J=7.0, 3H). MS (ES⁺) 372.0 (M+1), 743.0 (2M+1), 765.1 (2M+23); (ES⁻) 370.5 (M−1).

4-(1-(1-ethoxyethyl)-1H-pyrazol-4-yl)-7H-pyrrolo[2,3-c]pyridazine (32c): (12 mg, 13%) as a colorless solid. ¹HNMR (300 MHz, MeOD) S 9.10 (s, 1H), 8.62 (d, J=0.6, 1H), 8.28 (d, J=0.4, 1H), 7.85 (d, J=3.5, 1H), 6.94 (d, J=3.5, 1H), 5.67 (q, J=6.0, 1H), 3.55 (dq, J=7.0, 9.4, 1H), 3.43-3.33 (m, 1H), 1.75 (d, J=6.0, 3H), 1.17 (t, J=7.0, 3H). MS (ES⁺) 258.1 (M+1), 515.0 (2M+1), 537.0 (2M+23).

### Preparation of (4-chloro-7H-pyrrolo[2,3-c]pyridazin-7-yl)methyl pivalate 32a

To a solution of 4-chloro-7H-pyrrolo[2,3-c]pyridazine 31i (100 mg, 0.651 mmol) in methylene chloride (5 mL) was added triethyl amine (551 mg, 5.45 mmol) and cooled to −10° C., to the cold reaction mixture was added chloromethyl pivalate (348 mg, 2.24 mmol) and 4,4′-dimethylamino pyridine (5 mg) and heated at 50° C. overnight. The reaction was cooled to room temperature and quenched with water (15 mL) and extracted with chloroform (2×25 mL). The chloroform layers were combined and washed with water (2×10 mL), brine (1×10 mL), dried and concentrated in vacuum. The residue obtained was purified by flash column chromatography[silica gel 12 g, eluting with (9:1) mixture of ethyl acetate and methanol in hexanes (0 to 40%)] to afford (4-chloro-7H-pyrrolo[2,3-c]pyridazin-7-yl)methyl pivalate 32a (145 mg, 83%) as a brown solid. ¹HNMR (300 MHz, DMSO) δ 9.14 (s, 1H), 8.15 (d, J=3.6, 1H), 6.78 (d, J=3.6, 1H), 6.41 (s, 2H), 1.08 (s, 9H). MS (ES⁺) 268.0 (M+1).

### Example 3

### 3-(4-(7H-pyrrolo[2,3-c]pyridazin-4-yl)-1H-pyrazol-1-yl)-3-cyclopentylpropanenitrile (34j)

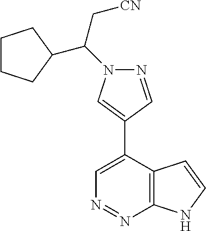

To a solution of (4-(1-(2-cyano-1-cyclopentylethyl)-1H-pyrazol-4-yl)-7H-pyrrolo[2,3-c]pyridazin-7-yl)methyl pivalate 34i (0.025 g, 0.06 mmol) in methanol (5 ml) was added 0.1 N aqueous NaOH solution (0.1 mL, 0.1 mmol) and stirred at room temperature for 3 h. The reaction mixture was concentrated in vacuo and the residue obtained was purified by flash column chromatography[silica gel 4 g, eluting with 0-100% (9:1) ethyl acetate/methanol in hexane] to furnish 3-(4-(7H-pyrrolo[2,3-c]pyridazin-4-yl)-1H-pyrazol-1-yl)-3-cyclopentylpropanenitrile 34j as a yellow semisolid; ¹H NMR (300 MHz, CDCl3) δ 12.84-11.45 (m, 1H, D₂O exchangeable), 9.09 (s, 1H), 8.17 (s, 1H), 8.14 (s, 1H), 7.79 (d, J=3.5, 1H), 6.76 (d, J=3.5, 1H), 4.37-4.25 (m, 1H), 3.16 (dd, J=8.5, 17.0, 1H), 2.98 (dd, J=3.9, 17.0, 1H), 2.64 (m, 1H), 2.04-1.94 (m, 1H), 1.82-1.52 (m, 6H), 1.34 (m, 1H). MS (ES⁺) 307.04 (M+1), (ES−) 305.00 (M−1); IR (KBr) 3431, 2954, 2250, 1598 cm⁻¹.

### Preparation of (4-(1-(2-cyano-1-cyclopentylethyl)-1H-pyrazol-4-yl)-7H-pyrrolo[2,3-c]pyridazin-7-yl)methyl pivalate (34i)

### Step 1:

To a solution of Potassium t-butoxide (1.23 g, 10.37 mmol) in THF (20 mL) at 0° C. was added diethyl cyanomethylphosphonate 34b (1.96 g, 10.87 mmol) dropwise over a period of 10 mins. The reaction mixture was allowed to warm to room temperature and stirred at room temperature for 1 h. The reaction mixture was cooled to 0° C. and added a solution of cyclopentanecarbaldehyde 34a (0.97 g, 9.88 mmol) in THF (10 mL). The reaction mixture was allowed to warm to room temperature and stirred for 48 h. The reaction was diluted with water (10 mL and extracted with ethyl acetate (3×30 ml). The ethyl acetate layers were combined and washed with brine (25 ml), dried concentrated in vacuum. The residue obtained was purified by flash column chromatography (silica gel 20 g, eluting with 0-50% ethyl acetate in hexane) to furnish 3-cyclopentylacrylonitrile 34c (0.55 g, 46%) as a colorless oil; ¹HNMR (300 MHz, DMSO) δ 6.85 (dd, J=8.1, 16.3, 0.4H), 6.66-6.51 (m, 0.6H), 5.67 (dd, J=1.2, 16.3, 0.4H), 5.56 (dd, J=0.6, 10.8, 0.6H), 2.86 (dq, J=8.1, 16.5, 0.6H), 2.60 (dt, J=8.3, 16.7, 0.4H), 1.79 (m, 2H), 1.70-1.50 (m, 4H), 1.42-1.29 (m, 2H).

### Step 2:

To a solution of pyrazole 34d (25.53 g, 375 mmol) and iodine (47.6 g, 187.5 mmol) in water (135 mL) was added 30% H₂O₂ (25.8 mL, 225 mmol). The mixture was stirred at room temperature overnight. A cold solution of 5% NaHSO₃ (100 mL) was added to the reaction mixture, affording an off-white slurry. The product was filtered and washed with water to give 4-iodo-1H-pyrazole 34e (61.9 g, 85%), as off-white solid; mp 86.8° C.; ¹H NMR (300 MHz, CDCl₃) δ 9.20 (bs, 1H), 7.63 (s, 2H); ¹³C NMR (75 MHz, CDCl₃) δ 138.75, 138.75, 56.50; Analysis: Calculated for C₃H₃IN₂: C, 18.58; H, 1.56; N, 14.44. Found: C, 18.70; H, 1.49; N, 14.41.

### Step 3:

To a solution of 4-iodo-1H-pyrazole 34e (0.72 g, 3.75 mmol) in acetonitrile (10 mL) was added 3-cyclopentylacrylonitrile 34c (0.5 g, 4.12 mmol) and DBU (0.57 g, 3.75 mmol). The reaction mixture was stirred at room temperature and concentrated in vacuum. The residue obtained was dissolved in ethyl acetate washed with 1 N aqueous HCl, brine, dried and concentrated in vacuum to furnish crude as oil. The crude was purified by flash column chromatography (silica gel 24 g, eluting with 0-50% ethyl acetate in hexane) to furnish 3-cyclopentyl-3-(4-iodo-1H-pyrazol-1-yl)propanenitrile 34f (0.845 g, 72%) as a colorless oil; ¹HNMR (300 MHz, DMSO) δ 8.06 (d, J=0.6, 1H), 7.61 (s, 1H), 4.40 (td, J=5.2, 9.0, 1H), 3.20-3.04 (m, 2H), 2.39-2.21 (m, 1H), 1.74 (m, 1H), 1.63-1.36 (m, 4H), 1.33-1.18 (m, 2H), 1.13-1.02 (m, 1H).

### Step 4:

To a degassed solution of 3-cyclopentyl-3-(4-iodo-1H-pyrazol-1-yl)propanenitrile 34f (0.43 g, 1.35 mmol) in anhydrous 1,4-dioxane (4.0 mL, 51 mmol) was added 4,4,4′,4′,5,5,5′,5′-octamethyl-2,2′-bi(1,3,2-dioxaborolane) 34 g (0.366 g, 1.43 mmol),tetrakis(triphenylphosphine)palladium(0) (47 mg, 0.041 mmol) and potassium acetate (0.41 g, 4.06 mmol) and heated at 120° C. via microwave for 3 hour. The reaction mixture was concentrated in vacuum and the residue obtained was purified by flash column chromatography (silica gel 24 g, eluting with 0-50% ethyl acetate in hexane) to furnish 3-cyclopentyl-3-(4-(4,4,5,5-tetramethyl-1,3,2-dioxaborolan-2-yl)-1H-pyrazol-1-yl)propanenitrile 34h (0.32 g) which was contaminated with 4,4,4′,4′,5,5,5′,5′-octamethyl-2,2′-bi(1,3,2-dioxaborolane) 34g. The reaction mixture was used as such for next step assuming 50% purity.

### Step 5:

To a solution of (4-chloro-7H-pyrrolo[2,3-c]pyridazin-7-yl)methyl pivalate 32a (0.21 g, 0.77 mmol) in 1,4-dioxane (5 mL), was added 3-cyclopentyl-3-(4-(4,4,5,5-tetramethyl-1,3,2-dioxaborolan-2-yl)-1H-pyrazol-1-yl)propanenitrile 34h (0.32 g, from above step) tetrakis(triphenylphosphine)palladium(0) (0.035 g, 0.031 mmol) and solid potassium carbonate (0.4 g, 3 mmol, 3.0 equiv) at room temperature. The resulting reaction mixture was degassed and heated at 100° C. for 48 h. The reaction mixture was neutralized with glacial acetic acid, diluted with water (10 ml) and ethyl acetate (10 ml). The reaction mixture was filtered to remove insoluble residues. The organic layer was separated and the aqueous layer was extracted with ethyl acetate (2×25 mL). The combined organic layers were washed with brine (25 mL), dried, filtered and concentrated in vacuum. The residue obtained was purified by flash column chromatography(silica gel 25 g, eluting with 0-100% ethyl acetate/methanol (9:1) in hexane) to furnish (4-(1-(2-cyano-1-cyclopentylethyl)-1H-pyrazol-4-yl)-7H-pyrrolo[2,3-c]pyridazin-7-yl)methyl pivalate 34i (0.025 g, 6%) as a colorless oil; ¹H NMR (300 MHz, CDCl₃) δ 9.16 (s, 1H), 8.12 (s, 1H), 8.09 (s, 1H), 7.74 (d, J=3.7, 1H), 6.73 (d, J=3.7, 1H), 6.44 (s, 2H), 4.35-4.22 (m, 1H), 3.14 (dd, J=8.5, 17.0, 1H), 2.96 (dd, J=3.9, 17.0, 1H), 2.61 (dd, J=7.4, 17.0, 1H), 1.96 (m, 1H), 1.82-1.48 (m, 6H), 1.33 (m, 1H), 1.15 (s, 9H); MS (ES+) 421.05 (M+1), 443.03 (M+23), 863.11 (2M+23), (ES−) 455.07 (M+35).

### Example 4

### (R)-3-(4-(7H-pyrrolo[2,3-e]pyridazin-4-yl)-1H-pyrazol-1-yl)-3-cyclopentyl propanenitrile (43g)

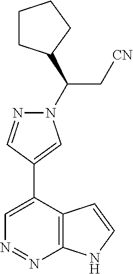

To a solution of (R)-(4-(1-(2-cyano-1-cyclopentylethyl)-1H-pyrazol-4-yl)-7H-pyrrolo[2,3-c]pyridazin-7-yl)methyl pivalate (431) (120 mg, 0.285 mmol) in methanol (3 mL) was added 1N NaOH (0.05 mL, 0.05 mmol). The reaction mixture was stirred at room temperature for 3.5 h and concentrated in vacuum to dryness. The residue obtained was purified by flash column chromatography (silica gel 12 g, eluting with methanol in chloroform 0-100%) to furnish (R)-3-(4-(7H-pyrrolo[2,3-c]pyridazin-4-yl)-1H-pyrazol-1-yl)-3-cyclopentylpropanenitrile (43g) (51 mg, 58%) as a yellow solid. ¹H NMR (300 MHz, DMSO-d₆) δ 12.40 (s, 1H, D₂O exchangeable), 9.18 (s, 1H), 8.79 (s, 1H), 8.39 (s, 1H), 7.98-7.87 (m, 1H), 6.94 (dd, J=1.5, 3.4, 1H), 4.50 (td, J=4.5, 9.3, 1H), 3.29-3.14 (m, 2H), 2.47-2.35 (m, 1H), 1.87-1.77 (m, 1H), 1.66-1.42 (m, 4H), 1.37-1.27 (m, 2H), 1.26-1.14 (m, 1H); ¹H NMR (300 MHz, CDCl₃) δ 12.66-11.07 (m, 1H), 9.14 (s, 1H), 8.19 (s, 1H), 8.16 (s, 1H), 7.87 (d, J=3.4, 1H), 6.80 (d, J=3.4, 1H), 4.37-4.26 (m, 1H), 3.17 (dd, J=8.6, 17.0, 11-1), 2.98 (dd, J=3.8, 17.0, 1H), 2.68-2.58 (m, 1H), 2.06-1.93 (m, 1H), 1.83-1.50 (m, 6H), 1.40-1.29 (m, 1H); MS (ES+) 307.12 (M+1); 329.08 (M+Na); 613.10 (2M+1); 635.07 (2M+Na); 919.25 (3M+1); 941.07 (3M+Na); (ES−) 305.02 (M−1); 340.9 (M+Cl); 611.47 (2M−1); IR (KBr) 2250 cm⁻¹; [α]_{d}=−19.4, (c=1, CHCl₃).

### Preparation of (R)-(4-(1-(2-cyano-1-cyclopentylethyl)-1H-pyrazol-4-yl)-7H-pyrrolo[2,3-c]pyridazin-7-yl)methyl pivalate (43f)

### Step 1:

To a solution of (4-(1-(1-ethoxyethyl)-1H-pyrazol-4-yl)-7H-pyrrolo[2,3-c]pyridazin-7-yl)methyl pivalate (32b) (750 mg, 2.01 mmol) in tetrahydrofuran (20 mL) was added 2N aqueous hydrochloric acid (2.52 mL, 5.04 mmol) and stirred at room temperature for 10 h. The reaction mixture was cooled with ice/water bath and the pH adjusted between 9-10 using 1N aqueous sodium hydroxide. The reaction mixture was extracted with ethyl acetate (3×50 mL). The organic extracts were combined, washed with brine (2×20 mL), dried, filtered and concentrated under vacuum to afford product (43a) as off-white solid. The solid obtained was triturated with methyl t-butylether (50 mL), heated at reflux for 20 min and cooled to room temperature. The solid obtained was collected by filtration, washed with MTBE (2×10 mL), and dried in vacuum to afford pure (4-(1H-pyrazol-4-yl)-7H-pyrrolo[2,3-c]pyridazin-7-yl)methyl pivalate (43a) (453 mg, 75%) as a tan colored solid. ¹HNMR (300 MHz, DMSO) δ13.44 (s, 1H, D₂O exchangeable), 9.33 (s, 1H), 8.69 (s, 1H), 8.37 (s, 1H), 7.99 (d, J=3.7, 1H), 7.09 (d, J=3.7, 1H), 6.40 (s, 2H), 1.09 (s, 9H); MS (ES+) 300.07 (M+1), 322.02 (M+Na); (ES−) 297.9 (M−1), 334.3 (M+Cl); Analysis: Calcd for C₁₅H₁₇N₅O₂; C, 60.08; H, 5.72; N, 23.39. Found: C, 60.03; H, 5.79; N, 23.30

### Step 2:

To a solution containing cyclopentylacrylaldehyde (43b) (prepared as given in Org. Lett., 2009, 11 (9), pp 1999-2002, 435 mg, 3.50 mmol), (2R)-2-bis[3,5-bis(trifluoromethyl)phenyl][(triethylsilyl)oxy]methylpyrrolidine (43d) (Aldrich, 42 mg, 0.07 mmol) and 4-nitrobenzoic acid (43c) (11 mg, 0.07 mmol) in anhydrous chloroform (2.0 mL, 25 mmol) which was stirred at room temperature for 10 minutes was added (4-(1H-pyrazol-4-yl)-7H-pyrrolo[2,3-c]pyridazin-7-yl)methyl pivalate (43a) (0.21 g, 0.70 mmol). The resulting mixture was stirred at room temperature overnight and concentrated in vacuum to dryness. The residue obtained was purified by flash column chromatography (silica gel 12 g, eluting with ethyl acetate in hexane 0-100%) to furnish (R)-(4-(1-(1-cyclopentyl-3-oxopropyl)-1H-pyrazol-4-yl)-7H-pyrrolo[2,3-c]pyridazin-7-yl)methyl pivalate (43e) (185 mg, 62%) as a pale yellow foam. ¹H NMR (300 MHz, CDCl₃) δ 9.73 (s, 1H), 9.14 (s, 1H), 8.05 (s, 1H), 8.03 (s, 1H), 7.75 (d, J=3.7, 1H), 6.73 (d, J=3.7, 1H), 6.43 (s, 2H), 4.55 (dt, J=12.0, 3.0 Hz, 1H), 3.51-3.41 (m, Hi), 2.95 (dd, J=18.0, 3.0 Hz, 1H), 2.59-2.43 (m, Hi), 1.88 (s, 2H), 1.67 (s, 4H), 1.53-1.42 (m, 2H), 1.15 (s, 9H).

### Step 3:

To a stirred solution of (R)-(4-(1-(1-cyclopentyl-3-oxopropyl)-1H-pyrazol-4-yl)-7H-pyrrolo[2,3-c]pyridazin-7-yl)methyl pivalate (43e) (175 mg, 0.37 mmol) in THF (5 mL) was added concentrated ammonium hydroxide (1.15, 8.0 mmol) and iodine (115 mg, 0.45 mmol). The resulting solution was stirred at room temperature for 1 h and quenched with saturated aqueous sodium thiosulfate solution (10 mL). The reaction mixture was extracted with dichloromethane (3×30 mL). The organic layers were combined washed with brine (10 mL), dried, filtered and concentrated in vacuum to dryness. The residue obtained was purified by flash column chromatography (silica gel 12 g, ethyl acetate in hexane 0-60%) to furnish (R)-(4-(1-(2-cyano-1-cyclopentylethyl)-1H-pyrazol-4-yl)-7H-pyrrolo[2,3-c]pyridazin-7-yl)methyl pivalate (431) (131 mg, 75%) as a colorless foam. ¹H NMR (300 MHz, CDCl₃) δ 9.18 (s, 1H), 8.12 (s, 1H), 8.10 (s, 1H), 7.82 (s, 1H), 6.77 (d, J=3.5, 1H), 6.43 (s, 2H), 4.28 (s, 1H), 3.14 (dd, J=8.5, 17.0, 1H), 2.96 (dd, J=18.0, 6.0, 1H), 2.68-2.52 (m, 1H), 2.04-1.93 (m, 1H), 1.79-1.53 (m, 5H), 1.37-1.21 (m, 2H), 1.16 (s, 9H); MS (ES+) 421.1 (M+1); 443.1 (M+Na, 841.2 (2M+1); 863.2 (2M+Na); (ES−) 455.2 (M+Cl).

### Example 5

### (S)-3-(4-(7H-pyrrolo[2,3-c]pyridazin-4-yl)-1H-pyrazol-1-yl)-3-cyclopentyl propanenitrile (44d)

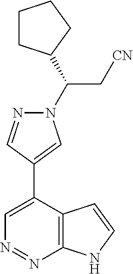

To a solution of (S)-(4-(1-(2-cyano-1-cyclopentylethyl)-1H-pyrazol-4-yl)-7H-pyrrolo[2,3-c]pyridazin-7-yl)methyl pivalate (44c) (127 mg, 0.30 mmol) in methanol (3 mL) was added 1N NaOH (0.06 mL, 0.06 mmol). The reaction mixture was stirred at room temperature for 3.5 h and concentrated in vacuum to dryness. The residue obtained was purified by flash column chromatography (silica gel 12 g, eluting with methanol in chloroform 0-100%) to furnish (S)-3-(4-(7H-pyrrolo[2,3-c]pyridazin-4-yl)-1H-pyrazol-1-yl)-3-cyclopentylpropanenitrile (44d) (50 mg, 54%) as a light yellow solid. ¹H NMR (300 MHz, DMSO) δ 12.40 (s, 1H), 9.19 (s, 1H), 8.79 (s, 1H), 8.39 (s, 1H), 7.97-7.89 (m, 1H), 6.94 (dd, J=1.5, 3.4, 1H), 4.50 (td, J=4.6, 9.4, 1H), 3.29-3.13 (m, 2H), 2.48-2.36 (m, 1H), 1.88-1.75 (m, 1H), 1.66-1.41 (m, 4H), 1.39-1.17 (m, 3H); MS (ES+) 307.08 (M+1); 613. 06 (2M+1); (ES−) 304.95 (M−1); [α]_{d}=+20.6 (c=0.98, CHCl₃).

### Preparation of (S)-(4-(1-(2-cyano-1-cyclopentylethyl)-1H-pyrazol-4-yl)-7H-pyrrolo[2,3-c]pyridazin-7-yl)methyl pivalate (44c)

### Step 1:

To a solution containing cyclopentylacrylaldehyde (43b) (prepared as given in Org. Lett., 2009, 11 (9), pp 1999-2002, 435 mg, 3.50 mmol), (2S)-2-bis[3,5-bis(trifluoromethyl)phenyl][(triethylsilyl)oxy]methylpyrrolidine (44a) (Aldrich, 42 mg, 0.07 mmol) and 4-nitrobenzoic acid (43c) (11 mg, 0.07 mmol) in anhydrous chloroform (2.0 mL, 25 mmol) which was stirred at room temperature for 10 minutes was added (4-(1H-pyrazol-4-yl)-7H-pyrrolo[2,3-c]pyridazin-7-yl)methyl pivalate (43a) (0.21 g, 0.70 mmol). The resulting mixture was stirred at room temperature overnight and concentrated in vacuum to dryness. The residue obtained was purified by flash column chromatography (silica gel 12 g, eluting with ethyl acetate in hexane 0-100%) to furnish (S)-(4-(1-(1-cyclopentyl-3-oxopropyl)-1H-pyrazol-4-yl)-7H-pyrrolo[2,3-c]pyridazin-7-yl)methyl pivalate (44b) (172 mg, 58%) as a pale yellow foam. ¹H NMR (300 MHz, CDCl₃) δ 9.73 (s, 1H), 9.14 (s, 1H), 8.05 (s, 1H), 8.04 (s, 1H), 7.77 (d, J=3.7, 1H), 6.74 (d, J=3.6, 1H), 6.43 (s, 2H), 4.55 (dt, J=3.0, 12.0, 1H), 3.52-3.40 (m, 1H), 2.96 (dd, J=3.0, 18.2, 1H), 2.59-2.44 (m, 1H), 1.94-1.85 (m, 1H), 1.75-1.54 (m, 5H), 1.53-1.40 (m, 2H), 1.15 (s, 9H).

### Step 2:

To a stirred solution of (S)-(4-(1-(1-cyclopentyl-3-oxopropyl)-1H-pyrazol-4-yl)-7H-pyrrolo[2,3-c]pyridazin-7-yl)methyl pivalate (44b) (172 mg, 0.37 mmol) in THF (5 mL) was added concentrated ammonium hydroxide (1.05, 7.3 mmol) and iodine (105 mg, 0.41 mmol). The resulting solution was stirred at room temperature for 1 h and quenched with saturated aqueous sodium thiosulfate solution (10 mL). The reaction mixture was extracted with dichloromethane (3×30 mL). The organic layers were combined washed with brine (10 mL), dried, filtered and concentrated in vacuum to dryness. The residue obtained was purified by flash column chromatography (silica gel 12 g, ethyl acetate in hexane 0-60%) to furnish (S)-(4-(1-(2-cyano-1-cyclopentylethyl)-1H-pyrazol-4-yl)-7H-pyrrolo[2,3-c]pyridazin-7-yl)methyl pivalate (44c) (131 mg, 84%) as a colorless foam. ¹H NMR (300 MHz, CDCl₃) δ 9.17 (s, 1H), 8.12 (s, 1H), 8.10 (s, 1H), 7.80 (d, J=3.6, 1H), 6.76 (d, J=3.7, 1H), 6.44 (s, 2H), 4.32-4.25 (m, 1H), 3.14 (dd, J=8.5, 17.0, 1H), 2.96 (dd, J=3.8, 17.0, 1H), 2.67-2.53 (m, 1H), 2.03-1.93 (m, 1H), 1.80-1.50 (m, 5H), 1.48-1.20 (m, 2H), 1.16 (s, 9H); MS (ES+) 421.1 (M+1); 443.1 (M+Na); 841.2 (2M+1); 863.2 (2M+Na); (ES−) 454.9 (M+Cl).

### Example 6

### tert-butyl 3-(4-(7H-pyrrolo[2,3-c]pyridazin-4-yl)-1H-pyrazol-1-yl)-3-(cyanomethyl)azetidine-1-carboxylate (45d)

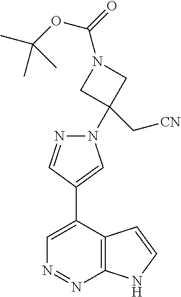

To a solution of tert-butyl 3-(cyanomethyl)-3-(4-(7-(pivaloyloxymethyl)-7H-pyrrolo[2,3-c]pyridazin-4-yl)-1H-pyrazol-1-yl)azetidine-1-carboxylate (45c) (75 mg, 0.15 mmol) in methanol (2 mL) was added 1N NaOH (0.03 mL, 0.03 mmol). The reaction mixture was stirred at room temperature for 2.5 h and concentrated in vacuum to dryness. The residue obtained was purified by flash column chromatography (silica gel 12 g, eluting with methanol in chloroform 0-10%) to furnish tert-butyl 3-(4-(7H-pyrrolo[2,3-c]pyridazin-4-yl)-1H-pyrazol-1-yl)-3-(cyanomethyl)azetidine-1-carboxylate (45d) (30 mg, 52%) as a light yellow solid. ¹HNMR (300 MHz, DMSO) δ 12.43 (s, 1H, D₂O exchangeable), 9.22 (s, 1H), 8.92 (s, 1H), 8.48 (s, 1H), 7.94 (d, J=3.4, 1H), 7.04 (d, J=3.4, 1H), 4.50 (d, J=9.0, 2H), 4.23 (d, J=9.4, 2H), 3.65 (s, 2H), 1.41 (s, 9H); MS (ES+) 380.06 (M+1), 759.11 (2M+1), (ES−) 378.28 (M−1).

### Preparation of tert-butyl 3-(cyanomethyl)-3-(4-(7-(pivaloyloxymethyl)-7H-pyrrolo[2,3-c]pyridazin-4-yl)-1H-pyrazol-1-yl)azetidine-1-carboxylate (45c)

### Step: 1

To a suspension of NaH (60% in mineral oil) (1.4 g 35 mmol) in THF (100 mL) at 0° C. was added dropwise diethyl phosphonate (6.45 mL, 41 mmol) and stirred at room temperature for 1 h. A solution of 1-boc-3-one-azetidine (45a) (5 g, 29.2 mmol) in THF (45 mL) was added to the anion at room temperature and stirred for 72 h. The reaction was quenched with water (100 mL) and extracted with ethyl acetate (3×100 mL). The combined organic layers were washed with brine (100 mL), dried, filtered and concentrated in vacuum. The residue obtained was purified by flash column chromatography (silica gel 80 g, eluting with ethyl acetate/hexanes, 0-100%) to furnish tert-butyl 3-(cyanomethylene)azetidine-1-carboxylate (45b) (3.52 g, 62%) as a white solid. ¹H NMR (300 MHz, DMSO) δ 5.84 (s, J=2.5, 1H), 4.74-4.51 (m, 4H), 1.53-1.30 (s, 9H); MS (ES−) 193.4 (M−1); IR (KBr) 2222 cm⁻¹; Analysis; Calcd for C₁₀H₁₄N₂O₂: C, 61.84; H, 7.27; N, 14.42. Found C, 61.94; H, 7.28; N, 14.38.

### Step: 2

To a solution of (4-(1H-pyrazol-4-yl)-7H-pyrrolo[2,3-c]pyridazin-7-yl)methyl pivalate (43a) (300 mg, 1.0 mmol), (E/Z)-tert-butyl 3-(cyanomethylene)azetidine-1-carboxylate (45b) (1.5 g, 5.8 mmol) in acetonitrile (5 mL) was added at room temperature DBU (0.149 mL, 1 mmol). The reaction was stirred at 50° C. for 24 h and concentrated in vacuum to dryness. The residue obtained was purified by flash column chromatography (silica gel 24 g, eluting with ethyl acetate in hexane 0-100%) to furnish tert-butyl 3-(cyanomethyl)-3-(4-(7-(pivaloyloxymethyl)-7H-pyrrolo[2,3-c]pyridazin-4-yl)-1H-pyrazol-1-yl)azetidine-1-carboxylate (45c) (383 mg, 77%). ¹H NMR (300 MHz, DMSO) δ 9.35 (s, 1H), 8.96 (s, 1H), 8.51 (s, 1H), 8.05 (d, J=3.7, 1H), 7.18 (d, J=3.7, 1H), 6.41 (s, 2H), 4.49 (d, J=9.5, 2H), 4.23 (d, J=9.5, 2H), 3.65 (s, 2H), 1.41 (s, 9H), 1.09 (s, 9H); MS (ES+) 516.0 (M+Na), (ES−) 527.9 (M+Cl).

### Example 7

### 2-(3-(4-(7H-pyrrolo[2,3-c]pyridazin-4-yl)-1H-pyrazol-1-yl)oxetan-3-yl)acetonitrile (46c)

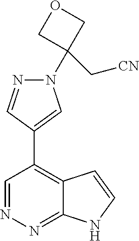

To a solution of (4-(1-(3-(cyanomethyl)oxetan-3-yl)-1H-pyrazol-4-yl)-7H-pyrrolo[2,3-c]pyridazin-7-yl)methyl pivalate (46b) (75 mg, 0.151 mmol) in methanol (2 mL) was added 1N NaOH (0.03 mL, 0.03 mmol). The reaction mixture was stirred at room temperature for 2.5 h and concentrated in vacuum to dryness. The residue obtained was purified by flash column chromatography (silica gel 12 g, eluting with methanol in chloroform 0-10%) to furnish 2-(3-(4-(7H-pyrrolo[2,3-c]pyridazin-4-yl)-1H-pyrazol-1-yl)oxetan-3-yl)acetonitrile (46c) (29 mg, 47%) as a light yellow solid. ¹H NMR (300 MHz, DMSO) δ 12.44 (s, 1H, D₂O exchangeable), 9.22 (s, 1H), 8.93 (d, J=0.4, 1H), 8.52-8.45 (m, 1H), 7.94 (d, J=3.4, 1H), 7.03 (d, J=3.5, 1H), 5.12 (t, J=9.7, 2H), 4.84 (d, J=7.4, 2H), 3.71 (s, 2H).MS (ES+) 281.10 (M+1), 561.01 (2M+1), (ES−) 279.38 (M−1), 559.48 (2M−1).

### Preparation of (4-(1-(3-(cyanomethyl)oxetan-3-yl)-1H-pyrazol-4-yl)-7H-pyrrolo[2,3-c]pyridazin-7-yl)methyl pivalate (46b)

To a solution of (4-(1H-pyrazol-4-yl)-7H-pyrrolo[2,3-c]pyridazin-7-yl)methyl pivalate (43a) (100 mg, 0.33 mmol), 2-(oxetan-3-ylidene)acetonitrile (46a) (50 mg, 0.53 mmol) in acetonitrile (3 mL) was added at room temperature DBU (50 μL, 0.33 mmol). The reaction was stirred at 50° C. for 2 h and concentrated in vacuum to dryness. The residue obtained was purified by flash column chromatography[silica gel 12 g, eluting with ethyl acetate/methanol (9:1) in hexane 0-100%] to furnish (4-(1-(3-(cyanomethyl)oxetan-3-yl)-1H-pyrazol-4-yl)-7H-pyrrolo[2,3-c]pyridazin-7-yl)methyl pivalate (46b) (99 mg, 76%). ¹HNMR (300 MHz, DMSO) δ9.36 (s, 1H), 8.98 (s, 1H), 8.52 (s, 1H), 8.05 (d, J=3.7, 1H), 7.16 (d, J=3.7, 1H), 6.41 (s, 2H), 5.14 (d, J=7.3, 2H), 4.84 (d, J=7.4, 2H), 3.71 (s, 2H), 1.09 (s, 9H); MS (ES+) 395.0 (M+1), 417.0 (M+Na), 789.0 (2M+1), 811.1 (2M+Na), (ES−) 429 (M+Cl); Analysis: Calcd for C₂₀H₂₂N₆O₃: C, 60.88; H, 5.62; N, 21.31. Found: C, 60.99; H, 5.86; N, 21.05.

### Example 8

### 3-(4-(7H-pyrrolo[2,3-c]pyridazin-4-yl)-1H-pyrazol-1-yl)-3-cyclohexylpropane nitrile (47d)

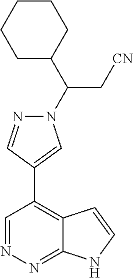

To a solution of (4-(1-(2-cyano-1-cyclohexylethyl)-1H-pyrazol-4-yl)-7H-pyrrolo[2,3-c]pyridazin-7-yl)methyl pivalate (47c) (80 mg, 0.18 mmol) in methanol (2 mL) was added 1N NaOH (30 pt, 0.03 mmol). The reaction mixture was stirred at room temperature overnight and concentrated in vacuum to dryness. The residue obtained was purified by flash column chromatography [silica gel 4 g, eluting with (ethyl acetate/methanol 9:1) in hexane 0-100%] to furnish 3-(4-(7H-pyrrolo[2,3-c]pyridazin-4-yl)-1H-pyrazol-1-yl)-3-cyclohexylpropanenitrile (47d) (39 mg, 67%) as a light yellow solid. ¹HNMR (300 MHz, DMSO) δ 12.40 (s, 1H), 9.18 (s, 1H), 8.76 (s, 1H), 8.40 (s, 1H), 7.92 (s, 1H), 6.94 (d, J=3.1, 1H), 4.47 (dd, J=7.8, 14.5, 1H), 3.26 (d, J=7.7, 2H), 1.91-1.68 (m, 3H), 1.65-1.54 (m, 2H), 1.28-0.85 (m, 6H); MS (ES+) 321.09 (M+1), 641.09 (2M+1); (ES−) 318.97 (M−1), 355.16 (M+Cl); IR (KBr) 2250 cm⁻¹.

### Preparation of intermediate compound (4-(1-(2-cyano-1-cyclohexylethyl)-1H-pyrazol-4-yl)-7H-pyrrolo[2,3-c]pyridazin-7-yl)methyl pivalate (47c)

### Step 1:

To an ice cold suspension of potassium tert-butoxide (3.39 g, 29.8 mmol) in THF (60 mL) was added dropwise a solution of diethyl cyanomethylphosphonate (5 mL, 32.2 mmol) in THF (15 mL). The reaction mixture was allowed to warm to room temperature over a period of 1 h. The anion was cooled (ice/water) and to it was added a solution of cyclohexanecarbaldehyde (47a) (3 mL, 24.8 mmol) in THF (30 mL). The reaction mixture was stirred at room temperature for 72 h and quenched with brine (60 mL). The reaction mixture was extracted with ethyl acetate (3×60 mL). The organic layers were combined washed with dried, filtered and concentrated in vacuum to dryness. The residue obtained was purified by flash column chromatography (silica gel 12 g, eluting with ethyl acetate in hexane 0-20%) to furnish (E/Z)-3-cyclohexylacrylonitrile (47b) (1.0 g, 30%) as a yellow oil. ¹H NMR (300 MHz, DMSO) δ 6.82 (dd, J=6.7, 16.6, 1H), 5.63 (dd, J=1.5, 16.6, 1H), 2.16 (d, J=8.0, 1H), 1.73-1.66 (m, 4H), 1.28-1.07 (m, 6H).

### Step 2:

To a solution of (4-(1H-pyrazol-4-yl)-7H-pyrrolo[2,3-c]pyridazin-7-yl)methyl pivalate (43a) (100 mg, 0.33 mmol), (E/Z)-3-cyclohexylacrylonitrile (47b) (1.0 g, 7.4 mmol) in acetonitrile (3 mL) was added at room temperature DBU (50 μl, 0.33 mmol). The reaction was stirred at 50° C. for 5 days and concentrated in vacuum to dryness. The residue obtained was purified by flash column chromatography [silica gel 4 g, eluting with (ethyl acetate/methanol 9:1) in hexane 0-100%] to furnish (4-(1-(2-cyano-1-cyclohexylethyl)-1H-pyrazol-4-yl)-7H-pyrrolo[2,3-c]pyridazin-7-yl)methyl pivalate (47c) (96 mg, 67%) as a sticky syrup. ¹HNMR (300 MHz, DMSO) δ 9.32 (s, 1H), 8.80 (s, 1H), 8.43 (s, 1H), 8.03 (d, J=3.7, 1H), 7.07 (d, J=3.7, 1H), 6.41 (s, 2H), 4.46 (m, 1H), 3.26 (d, J=7.1, 2H), 1.92-1.79 (m, 2H), 1.70-1.71 (m, 1H), 1.4-1.6 (m, 2H), 1.29-1.15 (m 4H), 1.08 (s, 9H), 1.08-0.90 (m, 2H).

### Example 9

### 2-(1-(4-(7H-pyrrolo[2,3-c]pyridazin-4-yl)-1H-pyrazol-1-yl)cyclopentyl)acetonitrile (48d)

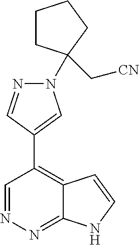

To a solution of (4-(1H-pyrazol-4-yl)-7H-pyrrolo[2,3-c]pyridazin-7-yl)methyl pivalate (43a) (100 mg, 0.33 mmol), 2-cyclopentylideneacetonitrile (48b) (89 mg, 0.83 mmol) in acetonitrile (3 mL) was added at room temperature DBU (50 μL, 0.33 mmol). The reaction was stirred at 50° C. for 72 h and concentrated in vacuum to dryness. The residue obtained was purified by flash column chromatography [silica gel 12 g, eluting with ethyl acetate/methanol (9:1) in hexane 0-100%] to furnish (4-(1-(1-(cyanomethypcyclopentyl)-1H-pyrazol-4-yl)-7H-pyrrolo[2,3-c]pyridazin-7-yl)methyl pivalate (48c) (18 mg, 13.4%) as a oil. ¹H NMR (300 MHz, CDCl₃) δ 9.18 (s, 1H), 8.16 (s, 1H), 8.12 (s, 1H), 7.83 (d, J=3.6, 1H), 6.78 (d, J=3.6, 1H), 6.43 (s, 2H), 3.06 (s, 2H), 2.64-2.53 (m, 2H), 2.29-2.19 (m, 2H), 1.97 (d, J=5.9, 4H), 1.16 (s, 9H); MS (ES+) 407.1 (M+1); and 2-(1-(4-(7H-pyrrolo[2,3-c]pyridazin-4-yl)-1H-pyrazol-1-yl)cyclopentyl)acetonitrile (48d) (16 mg, 16.5%) as a light yellow solid. ¹H NMR (300 MHz, DMSO) δ 12.39 (s, 1H, D₂O exchangeable), 9.22 (s, 1H), 8.79 (s, 1H), 8.39 (s, 1H), 7.94-7.88 (m, 1H), 7.00 (d, J=2.9, 1H), 3.32 (s, 2H), 2.61-2.54 (m, 2H), 2.04 (dd, J=6.9, 12.9, 2H), 1.84-1.75 (m, 2H), 1.73-1.65 (m, 2H); MS (ES+) 293.0 (M+1), 585.0 (2M+1), 607.0 (2M+Na), (ES−) 290.9 (M−1); IR (KBr) 2249 cm⁻¹.

### Preparation of 2-cyclopentylideneacetonitrile (48b)

To a cold suspension of NaH (60% in mineral oil, 0.88 g, 22 mmol) in THF (32 mL) was added diethyl cyanomethylphosphonate (3.6 mL, 23 mmol). The resulting mixture was stirred at room temperature for one hour before adding a solution of cyclopentanone (1.8 mL, 20 mmol) in THF (20 mL) The reaction mixture was stirred at room for 72 h and quenched with brine (40 mL) and ethyl acetate (20 mL). The aqueous phase was extracted with ethyl acetate (2×50 mL). The organic layers were combined washed with brine (100 mL), dried, filtered and concentrated in vacuum to dryness to furnish 2-cyclopentylideneacetonitrile (48b) (367 mg, 17%) as clear oil. It was used as such without further purification. ¹H NMR (300 MHz, DMSO) δ 5.68-5.35 (m, 1H), 2.66-2.30 (m, 4H), 1.85-1.52 (m, 4H).

### Example 10

### 2-(3-(4-(7H-pyrrolo[2,3-c]pyridazin-4-yl)-1H-pyrazol-1-yl)-1-(ethylsulfonyl)azetidin-3-yl)acetonitrile (49c)

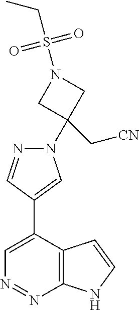

To a solution of (4-(1-(3-(cyanomethyl)-1-(ethylsulfonyl)azetidin-3-yl)-1H-pyrazol-4-yl)-7H-pyrrolo[2,3-c]pyridazin-7-yl)methyl pivalate (49b) (33 mg, 0.067 mmol) in methanol (2 mL) was added 1N NaOH (0.067 mL, 0.067 mmol). The reaction mixture was stirred at room temperature for 3.5 h and concentrated in vacuum to dryness. The residue obtained was purified by flash column chromatography [silica gel 12 g, eluting with ethyl acetate/methanol (9:1) in hexane 0-100%] to furnish 2-(3-(4-(7H-pyrrolo[2,3-c]pyridazin-4-yl)-1H-pyrazol-1-yl)-1-(ethylsulfonyl)azetidin-3-yl)acetonitrile (49c) (20 mg, 80.3%) as a light yellow solid. ¹H NMR (300 MHz, DMSO) δ 12.55-12.31 (m, 1H, D₂O exchangeable), 9.22 (s, 1H), 8.94 (s, 1H), 8.51 (s, 1H), 7.95 (d, J=3.4, 1H), 7.04 (d, J=3.4, 1H), 4.59 (d, J=9.2, 2H), 4.25 (d, J=9.2, 2H), 3.68 (s, 2H), 3.25 (q, J=7.3, 2H), 1.25 (t, J=7.3, 3H); MS (ES+) 372.00 (M+1), 394.00 (M+Na); (ES−) 369.97 (M−1), 406.20 (M+Cl); Analysis: Calcd for C₁₆H₁₇N₇O₂S: C, 51.73; H, 4.61; N, 26.39. Found: C, 51.93; H, 4.76; N, 25.88.

### Preparation of (4-(1-(3-(cyanomethyl)-1-(ethylsulfonyl)azetidin-3-yl)-1H-pyrazol-4-yl)-7H-pyrrolo[2,3-c]pyridazin-7-yl)methyl pivalate (49b)

### Step 1:

To a solution of tert-butyl 3-(cyanomethyl)-3-(4-(7-(pivaloyloxymethyl)-7H-pyrrolo[2,3-c]pyridazin-4-yl)-1H-pyrazol-1-yl)azetidine-1-carboxylate (45c) (113 mg, 0.22 mmol) in dichloromethane (10 mL) was added trifluoro acetic acid (0.38 g, 3.36 mmol) and stirred at room temperature for 24 h. The reaction mixture was neutralized with triethyl amine and concentrated in vacuum to dryness. The residue obtained was purified by flash column chromatography (silica gel 12 g, eluting with CMA-80 in chloroform 0-100%) to furnish (4-(1-(3-(cyanomethyl)azetidin-3-yl)-1H-pyrazol-4-yl)-7H-pyrrolo[2,3-c]pyridazin-7-yl)methyl pivalate (49a) (86 mg, 99%). ¹H NMR (300 MHz, DMSO) δ 9.37 (s, 1H), 9.04 (s, 1H), 8.60 (s, 1H), 8.09 (d, J=3.7, 1H), 7.17 (d, J=3.7, 1H), 6.42 (s, 2H), 4.69 (d, J=12.1, 2H), 4.45 (d, J=12.1, 2H), 3.73 (s, 2H), 3.38 (s, 1H, D₂O exchangeable), 1.09 (s, 9H); MS (ES+) 394.1 (M+1).

### Step 2:

To a cold (ice/water) solution of (4-(1-(3-(cyanomethyl)azetidin-3-yl)-1H-pyrazol-4-yl)-7H-pyrrolo[2,3-c]pyridazin-7-yl)methyl pivalate (49a) (82 mg, 0.20 mmol) in acetonitrile (3 mL) containing N,N-diisopropylethylamine (67 mg, 0.52 mmol) was added a solution of ethanesulfonyl chloride (40 mg, 31 mmol) in acetonitrile (1 mL). The reaction mixture was allowed to warm to room temperature overnight and concentrated in vacuum to dryness. The residue obtained was dissolved in ethyl acetate (50 mL) washed with brine (2×15 mL), dried, filtered and concentrated in vacuum to dryness. The residue obtained was purified by flash column chromatography [silica gel 12 g, eluting with ethyl acetate/methanol (9:1) in hexane 0-100%] to furnish (4-(1-(3-(cyanomethyl)-1-(ethylsulfonyl)azetidin-3-yl)-1H-pyrazol-4-yl)-7H-pyrrolo[2,3-c]pyridazin-7-yl)methyl pivalate (49b) (43 mg, 44%) as an oil. ¹H NMR (300 MHz, CDCl₃) δ 9.28 (s, 1H), 8.42 (s, 1H), 8.16 (s, 1H), 7.92 (d, J=3.6, 1H), 6.82 (d, J=3.6, 1H), 6.44 (s, 2H), 4.66 (d, J=9.3, 2H), 4.27 (d, J=9.4, 2H), 3.44 (s, 2H), 3.11 (q, J=7.4, 2H), 1.43 (t, J=7.4, 3H), 1.16 (s, 9H).

### Example 11

### 4-phenyl-7H-pyrrolo[2,3-c]pyridazine (50a)

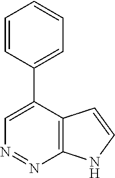

To a solution of 4-chloro-7H-pyrrolo[2,3-c]pyridazine (31i) (76 mg, 0.5 mmol) in 1,4-dioxane (2 mL) was added Phenyl boronic acid (91 mg, 0.75 mmol), potassium carbonate (276 mg, 2.0 mmol) and water (2 mL). The mixture was degassed by bubbling nitrogen gas for 15 min. Tetrakis (triphenylphosphine) Palladium (0) (23 mg, 0.02 mmol) was added and degassed for 2 min. The reaction mixture was stirred at 85° C. for 2 h, cooled to room temperature. Reaction mixture was diluted with ethyl acetate (25 L), washed with water (2×10 mL), brine (10 mL), dried, filtered and concentrated in vacuum to dryness. The residue obtained was purified by flash column chromatography [silica gel 12 g, eluting with ethyl acetate/methanol (9:1) in hexane 0 to 100%] to afford (50a) (25 mg, 26%) as a yellow tan solid. ¹HNMR (300 MHz, DMSO) δ 12.57 (s, 1H, D₂O exchangeable), 9.06 (s, 1H), 7.98 (d, J=3.4, 1H), 7.93-7.83 (m, 2H), 7.66-7.49 (m, 3H), 6.77 (d, J=3.4, 1H). MS (ES+) 196.18 (M+1), 218.14 (M+Na), 391.07 (2M+1), 413.05 (2M+Na); (ES−) 194.1 (M−1).

### Example 12

### 3-(4-(7H-pyrrolo[2,3-c]pyridazin-4-yl)-1H-pyrazol-1-yl)-4-cyclopentylbutane nitrile (51e)

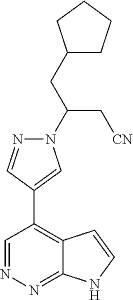

To a solution of (4-(1-(1-Cyano-3-cyclopentylpropan-2-yl)-1H-pyrazol-4-yl)-7H-pyrrolo[2,3-c]pyridazin-7-yl)methyl pivalate (51d) (80 mg, 0.184 mmol) in methanol (3 mL) was added 1N NaOH (55 μL) and stirred at room temperature for 3 h. The reaction mixture was concentrated in vacuum and the residue obtained was purified by flash column chromatography (silica gel 4 g, eluting with (9:1) ethyl acetate/methanol in hexane 0-100%) to furnish 3-(4-(7H-Pyrrolo[2,3-c]pyridazin-4-yl)-1H-pyrazol-1-yl)-4-cyclopentylbutane nitrile (51e) (25 mg, 42%) as a yellow solid. ¹HNMR (300 MHz, DMSO) δ 12.41 (s, 1H), 9.19 (s, 1H), 8.83 (s, 1H), 8.40 (s, 1H), 7.92 (d, J=3.4, 1H), 6.95 (d, J=3.4, 1H), 4.73 (m, 1H), 3.18 (d, J=6.9, 2H), 2.10 (m, 1H), 1.78 (m, 2H), 1.58-1.32 (m, 6H), 1.08 (m, 2H). MS (ES+) 641.1 (2M+1); (ES−) 319.0 (M−1), 354.8 (M+C1⁻); IR (KBr) 2249 cm⁻¹.

### Preparation of (4-(1-(1-Cyano-3-cyclopentylpropan-2-yl)-1H-pyrazol-4-yl)-7H-pyrrolo[2,3-c]pyridazin-7-yl)methyl pivalate (51d)

### Step 1:

To a solution of cyclopentylethanol (51a) (2 g, 17.5 mmol) in CH₂Cl₂ (500 mL) was added PCC (5.79 g, 26.6 mmol) and stirred at room temperature for 3 h. The reaction mixture was diluted with diethyl ether (500 mL) stirred at room temperature for 1 h, before it was filtered through a pad of celite and silica gel (1:1). The filtrate was carefully concentrated to dryness to give 2-cyclopentylacetaldehyde (51b) (2.9 g, 100%). This was pure enough to be used as such for next step.

### Step 2:

To an ice cold suspension of NaH (60% in mineral oil, 1.12 g, 28 mmol) in THF (50 mL) was added dropwise diethyl cyanomethylphosphonate (5.1 mL, 35 mmol). The mixture was stirred at room temperature for 1 h before adding a solution of 2-Cyclopentylacetaldehyde (51b) (2.9 g, 17.5 mmol) in THF (20 mL). The reaction mixture was stirred at room temperature overnight and quenched with water (100 mL) and ethyl acetate (100 mL). The organic layer was separated and the aqueous phase was washed with ethyl acetate (2×100 mL). The organic phases were combined and washed with brine (100 mL), dried over MgSO4, filtered and concentrated in vacuum. The residue obtained was purified by flash column chromatography (silica gel 40 g, eluting with ethyl acetate in hexane 0-20%) to give (E/Z)-4-cyclopentylbut-2-enenitrile (51c) (2.4 g, 100%) as a colorless oil. This was pure enough to be used as such for next step.

### Step 3:

To a solution of (4-(1H-pyrazol-4-yl)-7H-pyrrolo[2,3-c]pyridazin-7-yl)methyl pivalate (43a) (100 mg, 0.33 mmol) and (E/Z)-4-cyclopentylbut-2-enenitrile (51c) (135 μL, 0.825 mmol) in acetonitrile (3 mL) was added at room temperature DBU (50 υL, 0.33 mmol) and stirred at room temperature overnight. The reaction mixture was concentrated in vacuum and the residue obtained was purified by flash column chromatography (silica gel 4 g, eluting with ethyl acetate in hexane 0-100%) to give (4-(1-(1-cyano-3-cyclopentylpropan-2-yl)-1H-pyrazol-4-yl)-7H-pyrrolo[2,3-c]pyridazin-7-yl)methyl pivalate (51d) (80 mg, 55%) as a colorless oil. MS ES (+): 457.1, (M+Na); ES (−); 469.2, (M+Cl⁻).

### Example 13

### 3-(4-(7H-pyrrolo[2,3-c]pyridazin-4-yl)-1H-pyrazol-1-yl)-4-cyclohexylbutane nitrile (52e)

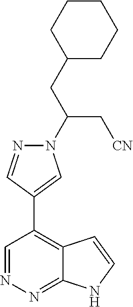

To a solution of (4-(1-(1-cyano-3-cyclohexylpropan-2-yl)-1H-pyrazol-4-yl)-7H-pyrrolo[2,3-c]pyridazin-7-yl)methyl pivalate (52d) (107 mg, 0.238 mmol) in methanol (5 mL) was added 1N NaOH (71 μL) and stirred at room temperature for 3 h. The reaction mixture was concentrated in vacuum and the residue obtained was purified by flash column chromatography (silica gel 4 g, eluting with (9:1) ethyl acetate/methanol in hexane 0-100%) to furnish 3-(4-(7H-Pyrrolo[2,3-c]pyridazin-4-yl)-1H-pyrazol-1-yl)-4-cyclohexylbutane nitrile (52e) (34 mg, 42%) as a olive colored solid. ¹HNMR (300 MHz, DMSO) δ 12.41 (s, 1H), 9.19 (s, 1H), 8.83 (s, 1H), 8.40 (s, 1H), 7.93 (d, J=3.3, 1H), 6.94 (d, J=3.3, 1H), 4.83 (m, 1H), 3.16 (d, J=6.9, 2H), 1.94 (m, 2H), 1.59 (m, 5H), 1.16-0.87 (m, 6H); IR (KBr) 2250 cm⁻¹; MS (ES+) 669.1 (2M+1); (ES−) 369.0 (M+Cl).

### Preparation of (4-(1-(1-cyano-3-cyclohexylpropan-2-yl)-1H-pyrazol-4-yl)-7H-pyrrolo[2,3-c]pyridazin-7-yl)methyl pivalate (52d)

### Step 1:

To a solution of cyclohexylethanol (52a) (3 g, 23 4 mmol) in CH₂Cl₂ (600 mL) was added PCC (7.74 g, 35.6 mmol) and stirred at room temperature for 3 h. The reaction mixture was diluted with diethyl ether (500 mL) stirred at room temperature for 1 h, before it was filtered through a pad of celite and silica gel (1:1). The filtrate was carefully concentrated to dryness to give 2-cyclopentylacetaldehyde (52b) (3.9 g, 100%). This was pure enough to be used as such for next step.

### Step 2:

To an ice cold suspension of NaH (60% in mineral oil, 1.5 g, 37.44 mmol) in THF (60 mL) was added dropwise diethyl cyanomethylphosphonate (7.4 mL, 46.8 mmol). The mixture was stirred at room temperature for 1 h before adding a solution of 2-Cyclohexylacetaldehyde (52b) (3.9 g, 23.4 mmol) in THF (20 mL). The reaction mixture was stirred at room temperature overnight and quenched with water (100 mL) and ethyl acetate (100 mL). The organic layer was separated and the aqueous phase was washed with ethyl acetate (2×100 mL). The organic phases were combined and washed with brine (100 mL), dried over MgSO4, filtered and concentrated in vacuum. The residue obtained was purified by flash column chromatography (silica gel 40 g, eluting with ethyl acetate in hexane 0-20%) to give (E/Z)-4-cyclohexylbut-2-enenitrile (52c) (3.0 g, 86%) as a colorless oil. This was pure enough to be used as such for the next step.

### Step 3:

To a solution of (4-(1H-pyrazol-4-yl)-7H-pyrrolo[2,3-c]pyridazin-7-yl)methyl pivalate (43a) (100 mg, 0.33 mmol) and (E/Z)-4-cyclohexylbut-2-enenitrile (52c) (250 μL, 0.825 mmol) in acetonitrile (3 mL) was added at room temperature DBU (50 μL, 0.33 mmol) and stirred at room temperature overnight. The reaction mixture was concentrated in vacuum and the residue obtained was purified by flash column chromatography (silica gel 4 g, eluting with ethyl acetate in hexane 0-100%) to give (4-(1-(1-cyano-3-cyclohexylpropan-2-yl)-1H-pyrazol-4-yl)-7H-pyrrolo[2,3-c]pyridazin-7-yl)methyl pivalate (52d) (107 mg, 74%). MS, ES (+) 449.2 (M+1).

### Example 14

### 3-(4-(7H-pyrrolo[2,3-c]pyridazin-4-yl)-1H-pyrazol-1-yl)-3-cyclopropylpropane nitrile (53d)

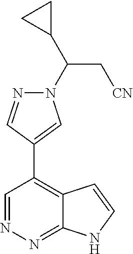

To a solution of (4-(1-(2-cyano-1-cyclopropylethyl)-1H-pyrazol-4-yl)-7H-pyrrolo[2,3-c]pyridazin-7-yl)methyl pivalate (53c) (54 mg, 0.14 mmol) in methanol (3 mL) was added 1N NaOH (41 μl). The reaction mixture was stirred at room temperature for 3 h and concentrated in vacuum to dryness. The residue obtained was purified by flash column chromatography [silica gel 4 g, eluting with ethyl acetate/methanol (9:1) in hexane 0-100%] to afford 3-(4-(7H-Pyrrolo[2,3-c]pyridazin-4-yl)-1H-pyrazol-1-yl)-3-cyclopropylpropane nitrile (53d) (26 mg, 71.9%) as a brown solid. ¹HNMR (300 MHz, DMSO, D₂O one drop) δ 9.19 (s, 1H), 8.76 (s, 1H), 8.40 (s, 1H), 7.91 (s, 1H), 6.95 (s, 1H), 4.03 (m, 1H), 3.32 (m, 2H), 1.45 (m, 1H), 0.73 (m, 1H), 0.53 (m, 3H); MS (ES+) 557.1 (2M+1), (ES−) 277.2 (M−1), 312.9 (M+Cl); IR (KBr) 2250 cm⁻¹.

### Preparation of (4-(1-(2-cyano-1-cyclopropylethyl)-1H-pyrazol-4-yl)-7H-pyrrolo[2,3-c]pyridazin-7-yl)methyl pivalate (53c)

### Step 1:

To a cold suspension of NaH (60% in mineral oil, 1.95 g, 48.8 mmol) in THF (80 mL) was added diethyl cyanomethylphosphonate (9.6 mL, 61 mmol). The resulting mixture was stirred at room temperature for one hour before adding a solution of 2-cyclopropanecarboxaldehyde (53a) (2.1 g, 30.5 mmol) in THF (30 mL). The reaction mixture was stirred at room temperature overnight and quenched with water (100 mL) and ethyl acetate (100 mL). The aqueous phase was extracted with ethyl acetate (2×100 mL). The organic layers were combined washed with brine (100 mL), dried, filtered and concentrated in vacuum to dryness. The residue obtained was purified by flash column chromatography (silica gel 40 g, eluting with ethyl acetate in hexane 0-20%) to furnish (E)-3-cyclopropylacrylonitrile (53b) (1.46 g, 51%) as colorless oil. It was pure enough to be used as such for the next step.

### Step 2:

To a solution of (4-(1H-pyrazol-4-yl)-7H-pyrrolo[2,3-c]pyridazin-7-yl)methyl pivalate (43a) (100 mg, 0.33 mmol), (E)-3-cyclopropylacrylonitrile (53b) (250 μL, 0.825 mmol) in acetonitrile (3 mL) was added at room temperature DBU (50 μL, 0.33 mmol). The reaction was stirred at 50° C. overnight and concentrated in vacuum to dryness. The residue obtained was purified by flash column chromatography (silica gel 4 g, eluting with ethyl acetate in hexane 0-100%) to furnish (4-(1-(2-cyano-1-cyclopropylethyl)-1H-pyrazol-4-yl)-7H-pyrrolo[2,3-c]pyridazin-7-yl)methyl pivalate (53c) (54 mg, 41%) as a colorless oil. ¹H NMR (300 MHz, CDCl₃) δ 9.15 (s, 1H), 8.21 (s, 1H), 8.12 (d, J=0.4, 1H), 7.74 (d, J=3.7, 1H), 6.73 (t, J=6.6, 1H), 6.44 (s, 2H), 3.81 (m, 1H), 3.22 (m, 2H), 1.69-1.41 (m, 1H), 1.18 (s, 9H), 0.95 (m, 1H), 0.87-0.77 (m, 1H), 0.67-0.48 (m, 2H); MS ES(+) 415.1 (M+Na).

### Example 15

### 3-(4-(7H-pyrrolo[2,3-c]pyridazin-4-yl)-1H-pyrazol-1-yl)-3-cyclobutylpropane nitrile (54e)

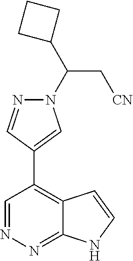

To a solution of (4-(1-(2-cyano-1-cyclobutylethyl)-1H-pyrazol-4-yl)-7H-pyrrolo[2,3-c]pyridazin-7-yl)methyl pivalate (54d) (104 mg, 0.256 mmol) in methanol (6 mL) was added 1N NaOH (77 μL). The reaction mixture was stirred at room temperature for 3 h and concentrated in vacuum to dryness. The residue obtained was purified by flash column chromatography [silica gel 4 g, eluting with ethyl acetate/methanol (9:1) in hexane 0-100%] to furnish 3-(4-(7H-pyrrolo[2,3-c]pyridazin-4-yl)-1H-pyrazol-1-yl)-3-cyclobutylpropane nitrile (54e) (18 mg, 24.2%) as a light yellow solid. ¹HNMR (300 MHz, DMSO) δ 12.45 (s, 1H), 9.22 (s, 1H), 8.80 (s, 1H), 8.42 (s, 1H), 7.95 (d, J=3.3 Hz, 1H), 6.98 (d, J=3.3 Hz, 1H), 4.71 (m, 1H), 3.13 (m, 2H), 2.88 (m, 1H), 2.08 (m, 1H), 1.79 (m, 5H); IR (KBr) 2251 cm⁻¹; MS (ES+) 585.2 (2M+1); (ES−) 326.8 (M+Cl).

### Preparation of (4-(1-(2-cyano-1-cyclobutylethyl)-1H-pyrazol-4-yl)-7H-pyrrolo[2,3-c]pyridazin-7-yl)methyl pivalate (54d)

### Step 1:

To a solution of oxalyl chloride (3.3 mL, 38.67 mmol) in dichloromethane (40 mL) cooled to −78° C. was added dropwise DMSO (5.5 mL, 70 mmol) followed by the addition of a solution of cyclobutanemethanol (54a) (3 g, 35 mmol) in dichloromethane (40 mL) at −78° C. The reaction mixture was stirred at −78° C. for 1 h, quenched with triethylamine (24.5 mL, 175 mmol) and warmed to room temperature. The reaction mixture was washed with water (50 mL), brine (50 mL), dried, filtered and concentrated in vacuum to obtain cyclobutanecarbaldehyde (54b) (1.89 g, 63%) as light yellow oil. This was pure enough to be used for the next step.

### Step 2:

To a cold suspension of NaH (60% in mineral oil, 0.94 g, 24.75 mmol) in THF (50 mL) was added diethyl cyanomethylphosphonate (4.3 mL, 27 mmol). The resulting mixture was stirred at room temperature for one hour before adding a solution of cyclobutanecarbaldehyde (54b) (1.89 g, 22.5 mmol) in THF (25 mL). The reaction mixture was stirred at room temperature overnight and quenched with water (100 mL) and ethyl acetate (100 mL). The aqueous phase was extracted with ethyl acetate (2×100 mL). The organic layers were combined washed with brine (100 mL), dried, filtered and concentrated in vacuum to dryness. The residue obtained was purified by flash column chromatography (silica gel 40 g, eluting with ethyl acetate in hexane 0-20%) to furnish (E/Z)-3-cyclobutylacrylonitrile (54c) (1.06 g, 44%) as a colorless oil. ¹HNMR (300 MHz, DMSO) δ 6.87 (ddd, J=8.2, 13.6, 20.2, 1H), 5.57 (ddd, J=1.2, 11.9, 13.6, 1H), 3.40-3.04 (m, 1H), 2.11-1.78 (m, 6H).

### Step 3:

To a solution of (4-(1H-pyrazol-4-yl)-7H-pyrrolo[2,3-c]pyridazin-7-yl)methyl pivalate (43a) (100 mg, 0.33 mmol), (E/Z)-3-cyclobutylacrylonitrile (54c) (110 μL, 0.825 mmol) in acetonitrile (3 mL) was added at room temperature DBU (50 μL, 0.33 mmol). The reaction was stirred at 50° C. overnight and concentrated in vacuum to dryness. The residue obtained was purified by flash column chromatography (silica gel 4 g, eluting with ethyl acetate in hexane 0-100%) to furnish (4-(1-(2-cyano-1-cyclobutylethyl)-1H-pyrazol-4-yl)-7H-pyrrolo[2,3-c]pyridazin-7-yl)methyl pivalate (54d) (104 mg, 77.6%) as a colorless oil. ¹HNMR (300 MHz, CDCl₃) δ 9.14 (s, 1H), 8.11 (s, 1H), 8.05 (d, J=0.5, 1H), 7.74 (d, J=3.7, 1H), 6.71 (d, J=3.7, 1H), 6.44 (s, 2H), 4.50 (m, 1H), 3.18-2.85 (m, 3H), 2.41-2.18 (m, 1H), 2.12-1.77 (m, 5H), 1.15 (s, 9H); MS (ES+) 835.2 (2M+Na).

### Example 16

### 2-(1-(4-(7H-pyrrolo[2,3-c]pyridazin-4-yl)-1H-pyrazol-1-yl)cyclobutyl)acetonitrile (55d)

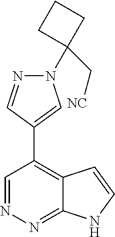

To a solution of (4-(1-(1-(cyanomethyl)cyclobutyl)-1H-pyrazol-4-yl)-7H-pyrrolo[2,3-c]pyridazin-7-yl)methyl pivalate (55c) (129 mg, 0.33 mmol) in methanol (6 mL) was added 1N NaOH (98 μL). The reaction mixture was stirred at room temperature for 3 h and concentrated in vacuum to dryness. The residue obtained was purified by flash column chromatography [silica gel 4 g, eluting with ethyl acetate/methanol (9:1) in hexane 0-100%] to furnish 2-(1-(4-(7H-pyrrolo[2,3-c]pyridazin-4-yl)-1H-pyrazol-1-yl)cyclobutyl)acetonitrile (55d) (33 mg, 36.2%) as a white solid. ¹H NMR (300 MHz, DMSO) δ 12.42 (s, 1H), 9.23 (s, 1H), 8.81 (s, 1H), 8.41 (s, 1H), 7.92 (d, J=3.3, 1H), 7.02 (d, J=3.3, 1H), 3.48 (s, 2H), 2.80 (m, 2H), 2.39 (m, 2H), 2.07 (m, 1H), 1.95 (m, 1H); IR (KBr) 2252 cm⁻¹; MS (ES+) 557.1 (2M+1), (ES−) 312.8 (M+Cl).

### Preparation of (4-(1-(1-(cyanomethyl)cyclobutyl)-1H-pyrazol-4-yl)-7H-pyrrolo[2,3-c]pyridazin-7-yl)methyl pivalate (55c)

### Step 1:

To a cold suspension of NaH (60% in mineral oil, 629 mg, 15.7 mmol) in THF (30 mL) was added diethyl cyanomethylphosphonate (2.7 mL, 17.2 mmol). The resulting mixture was stirred at room temperature for one hour before adding a solution of 2-Cyclobutanone (55a) (1 g, 14.3 mmol) in THF (15 mL). The reaction mixture was stirred at room temperature overnight and quenched with water (40 mL) and ethyl acetate (60 mL). The aqueous phase was extracted with ethyl acetate (2×40 mL). The organic layers were combined washed with brine (50 mL), dried, filtered and concentrated in vacuum to dryness. The residue obtained was purified by flash column chromatography (silica gel 40 g, eluting with ethyl acetate in hexane 0-15%) to furnish (E/Z)-2-cyclobutylideneacetonitrile (55b) (1.02 g, 38%) as colorless oil.

### Step 2:

To a solution of (4-(1H-pyrazol-4-yl)-7H-pyrrolo[2,3-c]pyridazin-7-yl)methyl pivalate (43a) (100 mg, 0.33 mmol), (E/Z)-2-cyclobutylideneacetonitrile (55b) (170 μL, 0.825 mmol) in acetonitrile (3 mL) was added at room temperature DBU (50 μL, 0.33 mmol). The reaction was stirred at 50° C. overnight and concentrated in vacuum to dryness. The residue obtained was purified by flash column chromatography (silica gel 4 g, eluting with ethyl acetate in hexane 0-100%) to furnish (4-(1-(1-(cyanomethyl)cyclobutyl)-1H-pyrazol-4-yl)-7H-pyrrolo[2,3-c]pyridazin-7-yl)methyl pivalate (55c) (129 mg, 99%). ¹HNMR (300 MHz, DMSO) δ 9.15 (s, 1H), 8.13 (s, 1H), 8.10 (s, 1H), 7.75 (d, J=3.7, 1H), 6.73 (d, J=3.7, 1H), 6.44 (s, 2H), 3.15 (s, 2H), 2.97-2.75 (m, 2H), 2.58 (m, 2H), 2.23-2.09 (m, 2H), 1.16 (s, 9H).

### Example 17

### 2-(1-(4-(7H-pyrrolo[2,3-c]pyridazin-4-yl)-1H-pyrazol-1-yl)cyclohexyl)acetonitrile (56d)

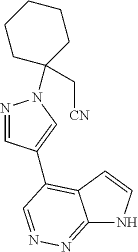

To a solution of (4-(1-(1-(cyanomethyl)cyclohexyl)-1H-pyrazol-4-yl)-7H-pyrrolo[2,3-c]pyridazin-7-yl)methyl pivalate (56c) (44 mg, 0.104 mmol) in methanol (5 mL) was added 1N NaOH (31 μL). The reaction mixture was stirred at room temperature for 3 h and concentrated in vacuum to dryness. The residue obtained was purified by flash column chromatography [silica gel 4 g, eluting with ethyl acetate/methanol (9:1) in hexane 0-100%] to furnish 2-(1-(4-(7H-pyrrolo[2,3-c]pyridazin-4-yl)-1H-pyrazol-1-yl)cyclohexyl)acetonitrile (56d) (28 mg, 88%) as an off white solid. ¹HNMR (300 MHz, DMSO) δ 12.39 (s, 1H), 9.23 (s, 1H), 8.78 (s, 1H), 8.40 (s, 1H), 7.91 (d, J=3.4, 1H), 6.99 (d, J=3.4, 1H), 3.16 (s, 2H), 2.56 (m, 1H), 1.93 (m, 2H), 1.50 (m, 7H); IR (KBr) 2246 cm⁻¹; MS (ES+) 613.1 (2M+1); (ES−) 305.4 (M−1), 340.8 (M+Cl).

### Preparation of (4-(1-(1-(cyanomethyl)cyclohexyl)-1H-pyrazol-4-yl)-7H-pyrrolo[2,3-c]pyridazin-7-yl)methyl pivalate (56c)

### Step 1:

To a cold suspension of NaH (60% in mineral oil, 880 mg, 22 mmol) in THF (40 mL) was added diethyl cyanomethylphosphonate (3.6 mL, 23 mmol). The resulting mixture was stirred at room temperature for one hour before adding a solution of 2-cyclohexanone (56a) (2.1 mL, 20 mmol) in THF (20 mL). The reaction mixture was stirred at room temperature overnight and quenched with water (40 mL) and ethyl acetate (40 mL). The aqueous phase was extracted with ethyl acetate (2×40 mL). The organic layers were combined washed with brine (50 mL), dried, filtered and concentrated in vacuum to dryness. The residue obtained was purified by flash column chromatography (silica gel 40 g, eluting with ethyl acetate in hexane 0-15%) to furnish (E/Z)-2-cyclohexylideneacetonitrile (56b) (1.05 g, 40%) as a colorless oil. ¹H NMR (300 MHz, DMSO) δ 5.39 (d, J=0.8, 1H), 2.47-2.35 (m, 2H), 2.26 (t, J=5.6, 2H), 1.65-1.49 (m, 6H).

### Step 2:

To a solution of (4-(1H-pyrazol-4-yl)-7H-pyrrolo[2,3-c]pyridazin-7-yl)methyl pivalate (43a) (100 mg, 0.33 mmol), (E/Z)-2-cyclohexylideneacetonitrile (56b) (100 μL, 0.825 mmol) in acetonitrile (3 mL) was added at room temperature DBU (50 μL, 0.33 mmol). The reaction was stirred at 50° C. overnight and concentrated in vacuum to dryness. The residue obtained was purified by flash column chromatography (silica gel 4 g, eluting with ethyl acetate in hexane 0-100%) to furnish (4-(1-(1-(cyanomethyl)cyclohexyl)-1H-pyrazol-4-yl)-7H-pyrrolo[2,3-c]pyridazin-7-yl)methyl pivalate (56c) (44 mg, 32%) as a colorless oil. ¹H NMR (300 MHz, CDCl₃) δ 9.15 (s, 1H), 8.17 (s, 1H), 8.12 (s, 1H), 7.73 (d, J=3.7, 1H), 6.72 (d, J=3.7, 1H), 6.44 (s, 2H), 2.91 (m, 2H), 2.60 (m, 2H), 2.04 (m, 2H), 1.60 (m, 6H), 1.16 (s, 9H).

### Example 18

### 3-(4-(7H-pyrrolo[2,3-c]pyridazin-4-yl)-1H-pyrazol-1-yl)-4-cyclopropylbutane nitrile (57e)

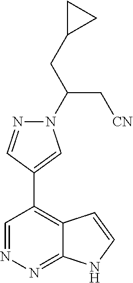

To a solution of (4-(1-(1-cyano-3-cyclopropylpropan-2-yl)-1H-pyrazol-4-yl)-7H-pyrrolo[2,3-c]pyridazin-7-yl)methyl pivalate (57d) (104 mg, 0.255 mmol) in methanol (6 mL) was added 1N NaOH (77A). The reaction mixture was stirred at room temperature for 3 h and concentrated in vacuum to dryness. The residue obtained was purified by flash column chromatography [silica gel 4 g, eluting with ethyl acetate/methanol (9:1) in hexane 0-100%] to furnish 3-(4-(7H-pyrrolo[2,3-c]pyridazin-4-yl)-1H-pyrazol-1-yl)-4-cyclopropylbutane nitrile (57e) (23 mg, 31%) as a light yellow solid. ¹HNMR (300 MHz, DMSO) δ 12.40 (s, 1H), 9.19 (s, 1H), 8.81 (s, 1H), 8.40 (s, 1H), 7.92 (d, J=3.4, 1H), 6.95 (d, J=3.4, 1H), 4.79 (m, 1H), 3.21 (dd, J=7.4, 13.1, 2H), 2.00-1.81 (m, 1H), 1.81-1.63 (m, 1H), 0.52 (m, 1H), 0.40 (m, 1H), 0.28 (m, 1H), 0.09 (m, 1H), −0.10 (m, 1H); IR (KBr) 2251 cm⁻¹; MS (ES+) 585.1 (2M+1); 607.1 (2M+Na); (ES−) 291.3 (M−1), 583.3 (2M−1).

### Preparation of (4-(1-(1-cyano-3-cyclopropylpropan-2-yl)-1H-pyrazol-4-yl)-7H-pyrrolo[2,3-c]pyridazin-7-yl)methyl pivalate (57d)

### Step 1:

To a solution of cyclopropylethanol (57a) (2.35 g, 27 mmol) in CH₂Cl₂ (100 mL) was added PCC (20% wt on aluminum, 37.9 g) and stirred at room temperature for overnight. The reaction mixture was filtered through a pad of celite and silica gel (1:1). The filtrate was carefully concentrated to dryness to give 2-cyclopropylacetaldehyde (57b) (2.2 g, 97%). ¹H NMR (300 MHz, DMSO) δ 9.84-9.24 (m, 1H), 2.19 (dd, J=1.9, 7.0, 2H), 0.94-0.70 (m, 1H), 0.45-0.35 (m, 2H), 0.07-0.01 (m, 2H). This was pure enough to be used as such for the next step.

### Step 2:

To a cold suspension of NaH (60% in mineral oil, 1.15 g, 28.6 mmol) in THF (60 mL) was added diethyl cyanomethylphosphonate (4.8 mL, 31 mmol). The resulting mixture was stirred at room temperature for one hour before adding a solution of 2-cyclopropylacetaldehyde (57b) (2.9 g, 17.5 mmol) in THF (25 mL). The reaction mixture was stirred at room temperature overnight and quenched with water (100 mL) and ethyl acetate (100 mL). The aqueous phase was extracted with ethyl acetate (2×100 mL). The organic layers were combined washed with brine (100 mL), dried, filtered and concentrated in vacuum to dryness. The residue obtained was purified by flash column chromatography (silica gel 40 g, eluting with ethyl acetate in hexane 0-20%) to furnish (E/Z)-4-cyclopropylbut-2-enenitrile (57c) (0.6 g, 32%) as a colorless oil. This was pure enough to be used as such for the next step.

### Step 3:

To a solution of (4-(1H-pyrazol-4-yl)-7H-pyrrolo[2,3-c]pyridazin-7-yl)methyl pivalate (43a) (100 mg, 0.33 mmol), (E/Z)-4-cyclopropyylbut-2-enenitrile (57c) (180 μL, 0.825 mmol) in acetonitrile (3 mL) was added at room temperature DBU (50 μL, 0.33 mmol). The reaction was stirred at 50° C. overnight and concentrated in vacuum to dryness. The residue obtained was purified by flash column chromatography (silica gel 4 g, eluting with ethyl acetate in hexane 0-100%) to furnish (4-(1-(1-cyano-3-cyclopropylpropan-2-yl)-1H-pyrazol-4-yl)-7H-pyrrolo[2,3-c]pyridazin-7-yl)methyl pivalate (57d) (104 mg, 78%); ¹H NMR (300 MHz, CDCl₃) δ 9.15 (s, 1H), 8.13 (d, J=1.3, 2H), 7.74 (d, J=3.7, 1H), 6.72 (d, J=3.7, 1H), 6.44 (s, 2H), 4.67 (tt, J=5.6, 8.0, 1H), 3.10 (m, 2H), 2.15 (m, 1H), 1.91-1.66 (m, 1H), 1.16 (s, 9H), 0.68-0.34 (m, 3H), 0.16 (m, 1H), 0.04 (m, 1H); MS ES(+) 835.2 (2M+Na).

### Example 19

### (R)-3-(4-(7H-pyrrolo[2,3-c]pyridazin-4-yl)-1H-pyrazol-1-yl)-3-cyclohexylpropanenitrile (58d)

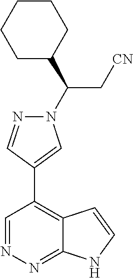

To a solution of (R)-(4-(1-(2-cyano-1-cyclohexylethyl)-1H-pyrazol-4-yl)-7H-pyrrolo[2,3-c]pyridazin-7-yl)methyl pivalate (58c) (99 mg, 0.23 mmol) in methanol (10 mL) was added 1N NaOH (68 μL). The reaction mixture was stirred at room temperature for 6 h and concentrated in vacuum to dryness. The residue obtained was purified by flash column chromatography (silica gel 4 g, eluting with CMA-80 in chloroform 0-100%) to furnish (R)-3-(4-(7H-pyrrolo[2,3-c]pyridazin-4-yl)-1H-pyrazol-1-yl)-3-cyclohexylpropanenitrile (58d) (57.5 mg, 78%) as a yellow solid. ¹HNMR (300 MHz, DMSO) δ 12.40 (s, 1H), 9.18 (s, 1H), 8.76 (s, 1H), 8.40 (s, 1H), 7.92 (s, 1H), 6.94 (s, 1H), 4.46 (s, 1H), 3.26 (d, J=6.6, 2H), 1.85 (m, 2H), 1.78-1.68 (m, 1H), 1.60 (m, 2H), 1.23 (m, 1H), 1.11 (m, 3H), 0.97 (m, 2H); IR(KBr) 2250 cm⁻¹; MS (ES+) 343.1 (M+Na), 641.2 (2M+1), 663.1 (2M+Na), MS (ES−) 321.0 (M−1); [α]_{D}=−16.0 (CHCl₃).

### Preparation of (R)-(4-(1-(2-cyano-1-cyclohexylethyl)-1H-pyrazol-4-yl)-7H-pyrrolo[2,3-c]pyridazin-7-yl)methyl pivalate (58c)

### Step 1:

To a stirred suspension of (triphenylphosphoranylidene)acetaldehyde (7.7 g, 25.3 mmol) in benzene (60 mL) was added at room temperature cyclohexanecarboxaldehyde (47a) (3 mL, 25.3 mmol). The reaction mixture was heated at reflux overnight and concentrated in vacuum to dryness. The residue obtained was purified by flash column chromatography (silica gel 40 g, eluting with ethyl acetate in hexane 0-20%) to furnish (E/Z)-3-cyclopentylacrylaldehyde (58a) (3.1 g, 89%) as a colorless oil.

### Step 2:

To a solution of (4-(1H-pyrazol-4-yl)-7H-pyrrolo[2,3-c]pyridazin-7-yl)methyl pivalate (43a) (150 mg, 0.5 mmol) in chloroform (10 mL) at room temperature was added (E/Z)-3-cyclopentylacrylaldehyde (58a) (0.435 g, 2.5 mmol) followed by (R)-α,α-Bis[3,5-bis(trifluoromethyl)phenyl]pyrrolidinemethanol trimethylsilyl ether (43d) (30 mg, 0.05 mmol), and p-nitrobenzoic acid (43c) (8.5 mg, 0.05 mmol). The resulting mixture was stirred at room temperature overnight and concentrated in vacuum to dryness. The residue obtained was purified by flash column chromatography (silica gel 12 g, eluting with ethyl acetate in hexane 0-100%) to furnish (R)-(4-(1-(1-cyclohexyl-3-oxopropyl)-1H-pyrazol-4-yl)-7H-pyrrolo[2,3-c]pyridazin-7-yl)methyl pivalate (58b) (149 mg, 68%) as a solid. ¹H NMR (300 MHz, DMSO) δ 9.63 (s, 1H), 9.28 (s, 1H), 8.74 (s, 1H), 8.32 (s, 1H), 8.01 (d, J=3.7, 1H), 7.07 (d, J=3.7, 1H), 6.39 (s, 2H), 4.71-4.64 (m 1H), 3.15 (m, 2H), 1.8-1.69 (m, 3H), 1.60 (m, 2H), 1.29-1.15 (m, 2H), 1.08 (s, 9H), 1.08-1.05 (m, 4H).

### Step 3:

To a stirred solution of (R)-(4-(1-(1-cyclohexyl-3-oxopropyl)-1H-pyrazol-4-yl)-7H-pyrrolo[2,3-c]pyridazin-7-yl)methyl pivalate (58b) (149 mg, 0.34 mmol) in THF (5 mL) was added concentrated ammonium hydroxide (0.95 mL, 13.6 mmol) and iodine (95 mg, 0.374 mmol). The resulting solution was stirred at room temperature for 1 h and quenched with saturated aqueous sodium thiosulfate solution (20 mL). The reaction mixture was extracted with dichloromethane (3×30 mL). The organic layers were combined washed with brine (30 mL), dried, filtered and concentrated in vacuum to dryness. The residue obtained was purified by flash column chromatography [silica gel 4 g, ethyl acetate/methanol (9:1) in hexane 0-100%] to furnish (R)-(4-(1-(2-cyano-1-cyclohexylethyl)-1H-pyrazol-4-yl)-7H-pyrrolo[2,3-c]pyridazin-7-yl)methyl pivalate (58c) (99 mg, 69%), MS (ES+) 435.13 (M+1).

### Example 20

### (S)-3-(4-(7H-pyrrolo[2,3-c]pyridazin-4-yl)-1H-pyrazol-1-yl)-4-cyclopentylbutanenitrile (59d)

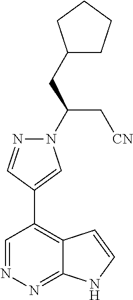

To a solution of (S)-(4-(1-(1-cyano-3-cyclopentylpropan-2-yl)-1H-pyrazol-4-yl)-7H-pyrrolo[2,3-c]pyridazin-7-yl)methyl pivalate (59c) (300 mg, 0.69 mmol) in methanol (20 mL) was added 1N NaOH (207 μL). The reaction mixture was stirred at room temperature for 6 h and concentrated in vacuum to dryness. The residue obtained was purified by flash column chromatography (silica gel 4 g, eluting with CMA-80 in chloroform 0-100%) to furnish (S)-3-(4-(7H-pyrrolo[2,3-c]pyridazin-4-yl)-1H-pyrazol-1-yl)-4-cyclopentylbutanenitrile (59d) (85 mg, 38%) as a white solid. ¹HNMR (300 MHz, DMSO) δ 12.40 (s, 1H), 9.19 (s, 1H), 8.83 (s, 1H), 8.40 (s, 1H), 7.92 (d, J=3.4, 1H), 6.95 (d, J=3.4, 1H), 4.73 (m, 1H), 3.18 (d, J=7.0, 2H), 2.10 (m, 1H), 1.78 (m, 2H), 1.62-1.35 (m, 6H), 1.24-0.93 (m, 2H). IR (KBr) 2249 cm⁻¹; MS (ES+) 321.11 (M+1).

### Preparation of (S)-(4-(1-(1-cyano-3-cyclopentylpropan-2-yl)-1H-pyrazol-4-yl)-7H-pyrrolo[2,3-c]pyridazin-7-yl)methyl pivalate (59c)

### Step 1:

To a stirred suspension of (triphenylphosphoranylidene)acetaldehyde (2.7 g, 8.93 mmol) in benzene (10 mL) was added at room temperature cyclopentanecarbaldehyde (51b) (1 g, 8.93 mmol). The reaction mixture was heated at reflux overnight and concentrated in vacuum to dryness. The residue obtained was purified by flash column chromatography (silica gel 40 g, eluting with ethyl acetate in hexane 0-20%) to furnish (E/Z)-4-cyclopentylbut-2-enal (59a) (1.1 g, 89%) as a colorless oil. ¹H NMR (300 MHz, DMSO) δ 9.50 (dd, J=4.5, 8.0, 1H), 7.02 (dt, J=7.1, 15.5, 1H), 6.09 (dd, J=8.0, 15.5, 1H), 2.33 (td, J=1.3, 7.1, 2H), 2.03-1.94 (m, 1H), 1.64-1.41 (m, 8H).

### Step 2:

To a solution of (4-(1H-pyrazol-4-yl)-7H-pyrrolo[2,3-c]pyridazin-7-yl)methyl pivalate (43a) (430 mg, 1.4 mmol) in chloroform (30 mL) at room temperature was added (E/Z)-4-cyclopentylbut-2-enal (59a) (0.967 mg, 7 mmol) followed by (R)-α,α-Bis[3,5-bis(trifluoromethyl)phen-yl]pyrrolidinemethanol trimethylsilyl ether (43d) (84 mg, 0.14 mmol), and p-nitrobenzoic acid (43c) (24 mg, 0.14 mmol). The resulting mixture was stirred at room temperature overnight and concentrated in vacuum to dryness. The residue obtained was purified by flash column chromatography (silica gel 12 g, eluting with ethyl acetate in hexane 0-100%) to furnish (S)-(4-(1-(1-cyclopentyl-4-oxobutan-2-yl)-1H-pyrazol-4-yl)-7H-pyrrolo[2,3-c]pyridazin-7-yl)methyl pivalate (59b) (390 mg, 64%) as a colorless semisolid. MS (ES+) 438.11 (M+1).

### Step 3:

To a stirred solution of (S)-(4-(1-(1-cyclopentyl-4-oxobutan-2-yl)-1H-pyrazol-4-yl)-7H-pyrrolo[2,3-c]pyridazin-7-yl)methyl pivalate (59b) (390 mg, 0.8 mmol) in THF (15 mL) was added concentrated ammonium hydroxide (2.3 mL, 32 mmol) and iodine (228 mg, 0.9 mmol). The resulting solution was stirred at room temperature for 1 h and quenched with saturated aqueous sodium thiosulfate solution (50 mL). The reaction mixture was extracted with dichloromethane (3×50 mL). The organic layers were combined washed with brine (30 mL), dried, filtered and concentrated in vacuum to dryness. The residue obtained was purified by flash column chromatography [silica gel 12 g, ethyl acetate/methanol (9:1) in hexane 0-100%] to furnish (S)-(4-(1-(1-cyano-3-cyclopentylpropan-2-yl)-1H-pyrazol-4-yl)-7H-pyrrolo[2,3-c]pyridazin-7-yl)methyl pivalate (59c) (300 mg, 86.4%). MS (ES+) 435.11 (M+1).

### Example 21

### (Z)-3-(4-(7H-pyrrolo[2,3-c]pyridazin-4-yl)-1H-pyrazol-1-yl)-3-(cyanomethyl)cyclobutanecarbonitrile (601) and (E)-3-(4-(7H-pyrrolo[2,3-c]pyridazin-4-yl)-1H-pyrazol-1-yl)-3-(cyanomethyl)cyclobutanecarbonitrile (60 g)

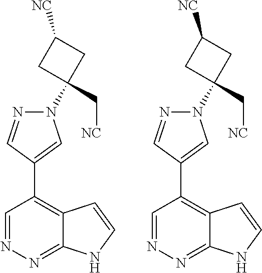

To a solution containing mixtures of (4-(1-((E)-3-cyano-1-(cyanomethyl)cyclobutyl)-1H-pyrazol-4-yl)-7H-pyrrolo[2,3-c]pyridazin-7-yl)methyl pivalate (60e) and (4-(1-((Z)-3-cyano-1-(cyanomethyl)cyclobutyl)-1H-pyrazol-4-yl)-7H-pyrrolo[2,3-c]pyridazin-7-yl)methyl pivalate (60d) (TL-908-020, 560 mg, 1.34 mmol) in methanol (50 mL) was added 1N NaOH (540 μL). The reaction mixture was stirred at room temperature for 3 h and concentrated in vacuum to dryness. The residue obtained was purified by flash column chromatography (silica gel 25 g, eluting with CMA80/CHCl₃, 0-100%) to furnish (E)-3-(4-(7H-pyrrolo[2,3-c]pyridazin-4-yl)-1H-pyrazol-1-yl)-3-(cyanomethyl)cyclobutanecarbonitrile (60 g) (isomer A, 18 mg, 4.4%)¹HNMR (300 MHz, DMSO) δ 12.42 (s, 1H), 9.21 (s, 1H), 8.90 (s, 1H), 8.47 (s, 1H), 7.93 (d, J=3.4, 1H), 7.03 (d, J=3.5, 1H), 3.57 (t, J=8.9, 1H), 3.50 (s, 2H), 3.30-3.16 (m, 2H), 3.00-2.86 (m, 2H); IR (KBr) 2235 cm⁻¹; MS (ES+) 304.2 (M+1); and (Z)-3-(4-(7H-pyrrolo[2,3-c]pyridazin-4-yl)-1H-pyrazol-1-yl)-3-(cyanomethyl)cyclobutanecarbonitrile (60f) (isomer B, 167 mg, 41%), ¹HNMR (300 MHz, DMSO) δ 12.43 (s, 1H), 9.22 (s, 1H), 8.87 (s, 1H), 8.46 (s, 1H), 7.93 (d, J=3.4, 1H), 7.04 (d, J=3.4, 1H), 3.66-3.49 (m, 3H), 3.24-3.11 (m, 2H), 2.93-2.77 (m, 2H); IR 2243 cm⁻¹; MS (ES+) 304.07 (M+1).

### Preparation of (4-(1-((E)-3-cyano-1-(cyanomethyl)cyclobutyl)-1H-pyrazol-4-yl)-7H-pyrrolo[2,3-c]pyridazin-7-yl)methyl pivalate (60e) and (4-(1-((Z)-3-cyano-1-(cyanomethyl)cyclobutyl)-1H-pyrazol-4-yl)-7H-pyrrolo[2,3-c]pyridazin-7-yl)methyl pivalate (60d)

### Step 1:

To a solution of 3-methylene cyclobutanecarbonitrile (60a) (5 g, 54.3 mmol) in water (60 mL) and 1,4-dioxane (150 mL) was added 0.2 M OsO₄ in water (1 mL) and stirred at room temperature for 5 min. Sodium periodate (24.4 g, 114 mmol) was added portion wise over a period of 30 min. The reaction mixture was stirred at room temperature for 1.5 h and extracted with dichloromethane (3×200 mL). The organic layers were combined dried, filtered and concentrated in vacuum to dryness. The residue obtained was purified by flash column chromatography (silica gel 80 g, eluting with ethyl acetate in hexane 0-50%) to furnish 3-oxocyclobutanecarbonitrile (60b) (4.15 g, 80%) as light gray solid. ¹H NMR (300 MHz, CDCl₃) δ 3.62-3.52 (m, 4H), 3.28 (m, 1H); MS (ES−): 94.1 (M−1).

### Step 2:

To an ice cold suspension of potassium tert-butoxide (1.3 g, 11.03 mmol) in THF (10 mL) was added dropwise a solution of diethyl cyanomethylphosphonate (1.9 mL, 11.55 mmol) in THF (15 mL). The reaction mixture was allowed to warm to room temperature over a period of 30 min. The anion was cooled (ice/water) and to it was added a solution of 3-oxocyclobutanecarbonitrile (60b) (1 g, 10.5 mmol) in THF (3 mL) The reaction mixture was stirred at room temperature for 2 h and quenched with water (50 mL). The reaction mixture was extracted with ethyl acetate (3×50 mL). The organic layers were combined washed with brine (100 mL), dried, filtered and concentrated in vacuum to dryness. The residue obtained was purified by flash column chromatography (silica gel 12 g, eluting with ethyl acetate in hexane 0-20%) to furnish 3-(cyanomethylene)cyclobutanecarbonitrile (60c) (774 mg, 63%) as colorless oil. ¹H NMR (300 MHz, CDCl₃) δ 5.41-5.24 (m, 4H), 3.46-3.36 (m, 1H).

### Step 3:

To a solution of (4-(1H-pyrazol-4-yl)-7H-pyrrolo[2,3-c]pyridazin-7-yl)methyl pivalate (43a) (450 mg, 1.5 mmol), 3-(cyanomethylene)cyclobutanecarbonitrile (60c) (443 mg, 3.75 mmol) in acetonitrile (20 mL) was added at room temperature DBU (225 μL, 1.5 mmol). The reaction was stirred at 50° C. overnight and concentrated in vacuum to dryness. The residue obtained was purified by flash column chromatography (silica gel 12 g, eluting with CMA80/CHCl₃, 0-100%) to furnish a mixture of (4-(1-((E)-3-cyano-1-(cyanomethyl)cyclobutyl)-1H-pyrazol-4-yl)-7H-pyrrolo[2,3-c]pyridazin-7-yl)methyl pivalate (60e) and (4-(1-((Z)-3-cyano-1-(cyanomethyl)cyclobutyl)-1H-pyrazol-4-yl)-7H-pyrrolo[2,3-c]pyridazin-7-yl)methyl pivalate (60d) (560 mg, 90%), the mixture was pure enough to be used as such for next step. MS (ES+): 418.16 (M+1).

### Example 22

### (R)-3-(4-(7H-pyrrolo[2,3-c]pyridazin-4-yl)-1H-pyrazol-1-yl)-3-cyclopentyl propan-1-ol (61b)

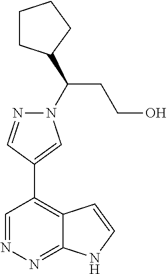

To a solution of (R)-(4-(1-(1-cyclopentyl-3-hydroxypropyl)-1H-pyrazol-4-yl)-7H-pyrrolo[2,3-c]pyridazin-7-yl)methyl pivalate (61a) (63 mg, 0.148 mmol) in methanol (3 mL) was added 1N NaOH (44 μL). The reaction mixture was stirred at room temperature for 6 h and concentrated in vacuum to dryness. The residue obtained was purified by flash column chromatography (silica gel 4 g, eluting with CMA-80 in chloroform 0-100%) to furnish (R)-3-(4-(7H-pyrrolo[2,3-c]pyridazin-4-yl)-1H-pyrazol-1-yl)-3-cyclopentyl propan-1-ol (61b) (31 mg, 67%) as an off-white solid. ¹HNMR (300 MHz, DMSO) δ 12.55-12.12 (bs, 1H), 9.16 (s, 1H), 8.64 (s, 1H), 8.30 (s, 1H), 7.88 (d, J=3.4, 1H), 6.95 (d, J=3.4, 1H), 4.51 (m, 1H), 4.22 (m, 1H), 3.32-3.23 (m, 1H), 3.07 (m, 1H), 2.38 (m, 1H), 2.06 (m, 2H), 1.84 (m, 1H), 1.55 (m, 4H), 1.37-1.09 (m, 3H); MS (ES+) 312.1 (M+1); 623.2 (2M+1).

### Preparation of (R)-(4-(1-(1-cyclopentyl-3-hydroxypropyl)-1H-pyrazol-4-yl)-7H-pyrrolo[2,3-c]pyridazin-7-yl)methyl pivalate (61a)

To a solution of (R)-(4-(1-(1-cyclopentyl-3-oxopropyl)-1H-pyrazol-4-yl)-7H-pyrrolo[2,3-c]pyridazin-7-yl)methyl pivalate (43e) (0.245 mg, 0.58 mmol) in THF (25 mL) was added NaBH₄ (22 mg) and methanol (0.5 mL). The reaction mixture was stirred at room temperature for 1 h and concentrated in vacuum to dryness. The residue obtained was purified by flash column chromatography [silica gel 12 g, eluting with (ethyl acetate/methanol 9:1) in hexane 0-100%] to furnish (R)-(4-(1-(1-cyclopentyl-3-hydroxypropyl)-1H-pyrazol-4-yl)-7H-pyrrolo[2,3-c]pyridazin-7-yl)methyl pivalate (61a) (130 mg, 48%). MS (ES+) 426.15 (M+1).

### Example 23

### (R)-4-(4-(7H-pyrrolo[2,3-e]pyridazin-4-yl)-1H-pyrazol-1-yl)-4-cyclopentyl butanenitrile (62b)

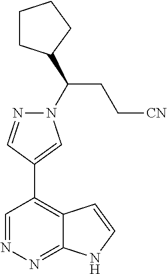

To a solution of (R)-(4-(1-(1-Cyclopentyl-3-(methylsulfonyloxy)propyl)-1H-pyrazol-4-yl)-7H-pyrrolo[2,3-c]pyridazin-7-yl)methyl pivalate (62a) (197 mg, 0.39 mmol) in DMF (5 mL) was added potassium cyanide (127 mg, 1.95 mmol), tetramethylammonium chloride (13 mg, 0.078 mmol) and 18-crown-6 (11 mg, 0.039 mmol). The reaction mixture was heated with stirring at 95° C. overnight, cooled to room temperature and quenched with water (10 mL). The reaction mixture was extracted with ethyl acetate (3×25 mL). The organic layers were combined washed with brine (10 mL), dried, filtered and concentrated in vacuum. The residue obtained was purified by flash column chromatography (silica gel 4 g, eluting with CMA-80 in chloroform 0-100%) to furnish (R)-4-(4-(7H-pyrrolo[2,3-c]pyridazin-4-yl)-1H-pyrazol-1-yl)-4-cyclopentyl butanenitrile (62b) (30 mg, 24%) as a yellow solid. ¹HNMR (300 MHz, DMSO) δ 12.36 (s, 1H), 9.17 (s, 1H), 8.72 (s, 1H), 8.34 (s, 1H), 7.90 (d, J=3.4, 1H), 6.96 (d, J=3.4, 1H), 4.13 (m, 1H), 2.26 (m, 4H), 1.89 (m, 1H), 1.56 (m, 4H), 1.26 (m, 4H). MS (ES+) 321.20 (M+1), (ES−) 319.07 (M−1).

### Preparation of (R)-(4-(1-(1-Cyclopentyl-3-(methylsulfonyloxy)propyl)-1H-pyrazol-4-yl)-7H-pyrrolo[2,3-c]pyridazin-7-yl)methyl pivalate (62a)

To a solution of (R)-(4-(1-(1-cyclopentyl-3-hydroxypropyl)-1H-pyrazol-4-yl)-7H-pyrrolo[2,3-c]pyridazin-7-yl)methyl pivalate (61a) (297 mg, 0.7 mmol) in dichloromethane (25 mL) was added TEA (39 μL, 2.8 mmol), DMAP (10 mg), and methanesulfonyl chloride (108 μL, 1.4 mmol). The reaction mixture was stirred at room temperature overnight and quenched with water (25 mL). The reaction mixture was extracted with dichloromethane (2×20 mL). The organic layers were combined washed with brine (25 mL), dried, filtered and concentrated in vacuum. The residue obtained was purified by flash column chromatography (silica gel 4 g, eluting with ethyl acetate in hexane 0-100%) to furnish (R)-(4-(1-(1-cyclopentyl-3-(methylsulfonyloxy)propyl)-1H-pyrazol-4-yl)-7H-pyrrolo[2,3-c]pyridazin-7-yl)methyl pivalate (62a) (197 mg, 56%) as a solid. ¹H NMR (300 MHz, CDCl₃) δ 9.16 (d, J=4.4, 1H), 8.09 (d, J=3.8, 1H), 8.05 (d, J=0.6, 1H), 7.73 (d, J=3.7, 1H), 6.77 (t, J=3.7, 1H), 6.44 (s, 2H), 4.36-4.02 (m, al), 3.89 (m, 1H), 2.99 (m, 3H), 2.60-2.30 (m, 4H), 1.92 (m, 1H), 1.77-1.35 (m, 6H), 1.15 (s, 9H).

### Example 24

### 2-(7H-pyrrolo[2,3-c]pyridazin-4-yl)aniline (63c)

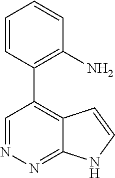

To a solution of 4-bromo-7H-pyrrolo[2,3-c]pyridazine (41a) (0.75 g, 3.8 mmol) in dioxane (18 mL)/water (2 mL) was added 2-acetamidophenylboronic acid (63b) (0.68 g, 3.8 mmol) and purged by bubbling nitrogen for 10 mins. To the reaction was added solid K₂CO₃ (2.1 g, 15.2 mmol) and tetrakis (triphenylphosphine) Palladium (0) (219 mg, 0.19 mmol). The reaction mixture was heated at 100° C. overnight. The reaction mixture was concentrated in vacuum and the residue dissolved chloroform (50 mL). The reaction mixture was filtered to remove insoluble residues and washed with saturated aqueous NaHCO₃ (25 mL), water (25 mL), brine (25 mL), dried, filtered and concentrated in vacuum. The residue obtained was purified by flash column chromatography [silica gel 12 g, eluting with ethyl acetate/methanol (9:1) in hexane 0 to 100%] to furnish 2-(7H-pyrrolo[2,3-c]pyridazin-4-yl)aniline (63c) (0.23 g, 29%) as a golden colored solid. ¹HNMR (300 MHz, DMSO) δ 12.41 (s, 1H), 8.85 (s, 1H), 7.89-7.84 (m, 1H), 7.19-7.12 (m, 2H), 6.84 (dd, J=1.1, 8.5, 1H), 6.73-6.65 (m, 1H), 6.38 (dd, J=1.5, 3.3, 1H), 4.98 (s, 21-1); MS (ES+) 233.1 (M+Na), 421.1 (2M+1), 443.0 (2M+Na), (ES−) 209.0 (M−1).

### Preparation of 4-bromo-7H-pyrrolo[2,3-c]pyridazine (41a)

### Step 1:

4-hydroxy-7H-pyrrolo[2,3-c]pyridazine-6-carboxylic acid (31 g) (4.25 g, 23.7 mmol) was added portion-wise into hot sulfolane (50 mL) at 270° C. The reaction mixture was stirred for 10 min at 270° C. and the heating stopped immediately after that. The product was isolated from the crude product by column chromatography (eluting 0-20% MeOH in DCM) to give pure 7H-pyrrolo[2,3-c]pyridazin-4-ol (31 h) (1.4 g, 44%) as a brown solid. ¹HNMR (300 MHz, DMSO-d₆) δ 13.6 (s, 11-1, D₂O exchangeable), 11.8 (s, 1H, D₂O exchangeable), 7.80-7.29 (m, 2H,), 6.59 (s, 1H), MS (ES⁺¹) 136.3 (M+1).

### Step 2:

The solution of 7H-pyrrolo[2,3-c]pyridazin-4-ol (31 h) (7.5 g, 55.5 mmol) in DMF (135 mL) was cooled to 0° C. followed by the addition of PBr₃ (10.43 mL, 111 mmol). The reaction mixture was stirred for 30 min at 0° C. then was allowed to warm to room temperature and stirred for 22 h. 1N NaHCO₃ (200 mL) was added and the product was extracted with EtOAc (4×100 mL). The combined extract was washed with brine (100 mL), dried over MgSO₄ and concentrated under reduced pressure. The residue was triturated with n-hexane to form a solid. The solid was collected by filtration to give the crude product. The crude product was purified by flash column chromatography to furnish 4-bromo-7H-pyrrolo[2,3-c]pyridazine (41a) (4.3 g, 38%) as an off-white solid. ¹HNMR (300 MHz, DMSO-d₆) δ 12.84 (s, 1H, D₂O exchangeable), 9.057 (s, 1H), 8.054 (d, 1H), 6.56 (d, 1H), MS (ES⁺¹) 196.1 (M−2).

### Example 25

### 4-(1H-pyrrol-3-yl)-7H-pyrrolo[2,3-c]pyridazine(64b)

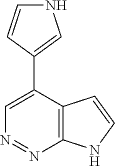

To a solution of 4-bromo-7H-pyrrolo[2,3-c]pyridazine (41a) (0.45 g, 2.27 mmol) in ethylene glycol dimethyl ether (DME, 9 mL)/water (1 mL) was added 1-(triisopropylsilyl)-1H-pyrrol-3-ylboronic acid (0.61 g, 2.27 mmol) and purged by bubbling nitrogen for 10 mins. To the reaction was added solid NaHCO₃ (0.572 g, 6.81 mmol) and bis-triphenylphosphine palladium (II) chloride (0.159 g, 0.227 mmol). The reaction mixture was heated in a microwave for 6 h at 100° C. and cooled to room temperature. The reaction mixture was filtered through celite using ethyl acetate (10 mL) for washing the celite layer. The reaction mixture was washed with water (10 mL), brine (10 mL), dried, filtered and concentrated in vacuum. The residue obtained was purified by flash column chromatography (silica gel 25 g, eluting with CMA-80 in chloroform 0-100%) to furnish 4-(1H-pyrrol-3-yl)-7H-pyrrolo[2,3-c]pyridazine (64b) (0.03 g, 7%) as a gold-yellow solid. ¹HNMR (300 MHz, DMSO).5 12.19 (s, 1H), 11.37 (s, 1H), 9.07 (s, 1H), 7.78 (s, 1H), 7.67 (d, J=2.8, 1H), 6.96 (dd, J=2.6, 4.6, 1H), 6.85 (d, J=3.3, 1H), 6.80 (d, J=1.8, 1H); MS (ES+) 185.1 (M+1); 369.0 (2M+1); (ES−) 183.0 (M−1).

### Example 26

### 7-benzyl-4-(1-(1-ethoxyethyl)-1H-pyrazol-4-yl)-7H-pyrrolo[2,3-d][1,2,3]triazine-5-carbonitrile (35d)

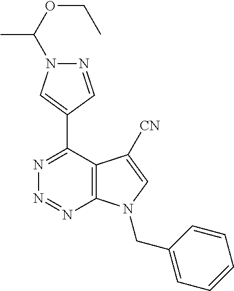

To a solution of 7-benzyl-4-hydroxy-7H-pyrrolo[2,3-d][1,2,3]triazine-5-carbonitrile (35c) (100 mg, 0.37 mmol) in 1,4-dioxane (4.5 mL) was added 1-(1-Ethoxyethyl)-4-(4,4,5,5-tetramethyl-1,3,2-dioxaborolan-2-yl)-1H-pyrazole (31k) (99 mg, 0.37 mmol), potassium carbonate (153 mg, 1.11 mmol) and water (0.5 mL). The mixture was degassed by bubbling nitrogen gas for 10 min. Tetrakis (triphenylphosphine) Palladium (0) (42 mg, 0.037 mmol) was added and degassed for 2 min. The reaction mixture was heated at 100° C. for 3 h in a microwave, cooled to room temperature and quenched with water (10 mL). The reaction mixture was extracted with ethyl acetate (2×10 mL). The ethyl acetate layers were combined washed with brine (10 mL), dried, filtered and concentrated in vacuum to dryness. The residue obtained was purified by flash column chromatography (silica gel 12 g, eluting with ethyl acetate in hexane 0 to 100%) to afford 7-benzyl-4-(1-(1-ethoxyethyl)-1H-pyrazol-4-yl)-7H-pyrrolo[2,3-d][1,2,3]triazine-5-carbonitrile (35d) (100 mg, 72%) as a tan colored solid. ¹HNMR (300 MHz, DMSO) δ 9.19 (s, 1H), 8.86 (s, 1H), 8.41 (s, 1H), 7.46-7.29 (m, 5H), 5.73 (m, 3H), 3.52 (m, 1H), 3.37 (m, 1H), 1.67 (d, J=5.9, 3H), 1.08 (t, J=7.0, 3H). MS (ES+) 374.1 (M+1), 396.0 (M+Na), 747.2 (2M+1), 769.1 (2M+Na), (ES−) 372.2 (M−1), 408.3 (M+Cl), 780.7 (2M+C1); Analysis: Calcd for C₂₀H₁₉N₇O: C, 64.33; H, 5.13; N, 26.26. Found: C, 64.18; H, 5.12; N, 26.10.

### Preparation of 7-benzyl-4-hydroxy-7H-pyrrolo[2,3-d][1,2,3]triazine-5-carbonitrile (35c)

### Step 1:

A stirred suspension of 1,2,4-triazole (5.0 g, 72 mmol) in CH₃CN (40 mL), under argon, was treated with phosphorus oxychloride (1.5 mL, 16 mmol), and then the white suspension was cooled to 0° C. Triethylamine (10 mL, 72 mmol) was added, and the mixture was allowed to stir at 0° C. for 1 h, at which time 7-benzyl-5-carboxamidopyrrolo[2,3-d][1,2,3]triazin-4-one (21e) (prepared according to the procedure given in J. Org. Chem. 2001, 66, 4776-4782, by Michael T. Migawa and Leroy B. Townsend, 0.539 g, 2.0 mmol) was added in one portion. The reaction mixture was stirred for 4.5 h at room temperature and filtered through Celite, and the filter cake was washed with CH₃CN (20 mL). The filtrate and washing were evaporated under reduced pressure, and the oily residue was dissolved in CHCl₃ (250 mL) and washed successively with sodium bicarbonate solution (2×20 mL), H₂O (20 mL), and brine (20 mL). The organic layer was dried and filtered, and the filtrate was evaporated under reduced pressure to give a brown solid, which was purified by flash chromatography (silica gel 25 g, eluting with ethyl acetate hexane 0-100%) to furnish 7-benzyl-4-(1H-1,2,4-triazol-1-yl)-7H-pyrrolo[2,3-d][1,2,3]triazine-5-carbonitrile (35a) as a tan colored solid. ¹HNMR (300 MHz, DMSO) δ 9.85 (s, 1H), 9.32 (s, 1H), 8.61 (s, 1H), 7.46-7.42 (m, 2H), 7.40-7.32 (m, 3H), 5.82 (s, 2H); ¹³CNMR (300 MHz, DMSO) δ 154.74, 149.92, 146.13, 145.06, 144.84, 135.87, 129.30, 128.77, 128.40, 114.65, 103.57, 85.59, 49.96; MS (ES+) 303 (M+1), 325 (M+Na); IR (KBr) 2236 cm⁻¹.

### Step 2:

A mixture of 7-benzyl-4-(1H-1,2,4-triazol-1-yl)-7H-pyrrolo[2,3-d][1,2,3]triazine-5-carbonitrile (35a) (0.4 g, 1.32 mmol) and potassium carbonate (0.91 g, 6.6 mmol) in DME/water (10 mL) was heated at reflux till hydrolysis is complete. DME was removed by concentration in vacuum. The aqueous layer was neutralized with glacial acetic acid and extracted with ethyl acetate (2×10 ml). The combined organic layer was washed with brine (10 ml), dried and concentrated in vacuum to furnish crude residue. Purification of the crude by flash column chromatography (silica gel 12 g, eluting with 0-100%, ethyl acetate in hexane) gave 7-benzyl-4-hydroxy-7H-pyrrolo[2,3-d][1,2,3]triazine-5-carbonitrile (35b) (0.2 g, 51%) as a pinkish white solid. ¹HNMR (300 MHz, DMSO) δ 15.20 (s, 1H), 8.61 (s, 1H), 7.41-7.29 (m, 5H), 5.63 (s, 2H); MS (ES−) 250.4 (M−1), 501.2 (2M−1).

### Step 3:

To a solution of 7-benzyl-4-hydroxy-7H-pyrrolo[2,3-d][1,2,3]triazine-5-carbonitrile (35b) (0.15 gm, 0.663 mmol), dimethylaniline (0.126 mL, 0.995 mmol) and benzyltriethylammonitun chloride (0.362 g, 1.59 mmol) in acetonitrile (5 mL) was added at room temperature POCl₃ (0.425 mL, 4.64 mmol). The reaction mixture was heated at reflux for 18 h and cooled to room temperature. The reaction mixture was concentrated in vacuum and added saturated NaHCO₃ (10 mL). The reaction mixture was extracted with ethyl acetate (10 mL); the organic layer was washed with brine (10 mL), dried, filtered and concentrated in vacuum. The residue was purified by flash column chromatography (silica gel 12 g, eluting with 0-100%, ethyl acetate in hexane) to furnish 7-benzyl-4-hydroxy-7H-pyrrolo[2,3-d][1,2,3]triazine-5-carbonitrile (35c) (0.125 g, 78%) as a pinkish tan solid. ¹HNMR (300 MHz, DMSO) δ 9.22 (s, 1H), 7.36 (m, J=6.0, 13.4, 5H), 5.78 (s, 2H); IR 2234 cm⁻¹; MS (ES−) 573.0 (2M+Cl).

### Example 27

### 7-benzyl-4-butoxy-7H-pyrrolo[2,3-d][1,2,3]triazine-5-carbonitrile (66a)

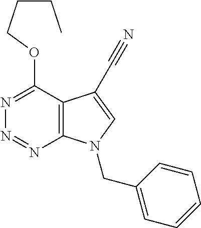

A solution of 7-benzyl-4-(1H-1,2,4-triazol-1-yl)-7H-pyrrolo[2,3-d][1,2,3]triazine-5-carbonitrile (35a) (0.15 g, 0.5 mmol) in n-BuOH (1 mL) was heated at 100° C. in a microwave for 1 h. The reaction mixture was concentrated in vacuum and the residue obtained was purified by flash column chromatography (silica gel 4 g, eluting with ethyl acetate in hexane 0-100%) to furnish 7-benzyl-4-butoxy-7H-pyrrolo[2,3-d][1,2,3]triazine-5-carbonitrile (66a) as a white solid. ¹HNMR (300 MHz, DMSO) δ 9.0 (s, 1H), 7.33 (m, 5H), 5.72 (s, 2H), 4.72 (t, 2H), 1.84 (t, 2H), 1.52 (dq, 2H), 0.97 (t, 3H); MS (ES+) 308.1 (M+1); (ES−) 342.0 (M+Cl); IR (Mk) 2236 cm⁻¹; Analysis: Calcd for C₁₇H₁₇N₅O, C, 66.43; H, 5.58; N, 22.79. Found: C, 66.37; H, 5.63; N, 22.54.

### Example 28

### (R)-3-(4-(7H-pyrrolo[2,3-c]pyridazin-4-yl)-1H-pyrazol-1-yl)-3-phenylpropane nitrile(67d)

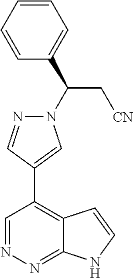

To a solution of (R)-(4-(1-(2-cyano-1-phenylethyl)-1H-pyrazol-4-yl)-7H-pyrrolo[2,3-c]pyridazin-7-yl)methyl pivalate (67c) (170 mg, 0.4 mmol) in methanol (20 mL) was added 1N NaOH (159 μL). The reaction mixture was stirred at room temperature for 6 h and concentrated in vacuum to dryness. The residue obtained was purified by flash column chromatography (silica gel 4 g, eluting with CMA-80 in chloroform 0-100%) to furnish (R)-3-(4-(7H-pyrrolo[2,3-c]pyridazin-4-yl)-1H-pyrazol-1-yl)-3-phenylpropane nitrile (67d) (24 mg, 19%) as a yellow solid. ¹HNMR (300 MHz, DMSO) δ 12.42 (s, 1H), 9.18 (s, 1H), 8.90 (s, 1H), 8.44 (s, 1H), 7.93 (d, J=3.4 Hz, 1H), 7.44-7.35 (m, 5H), 6.93 (d, J=3.4 Hz, 1H), 6.05 (dd, J=9.6, 5.8 Hz, 1H), 3.79 (dd, J=16.9, 9.6 Hz, 1H), 3.61 (dd, J=16.8, 5.8 Hz, 1H); MS (ES+): 315.07 (M+1).

### Preparation of (R)-(4-(1-(2-cyano-1-phenylethyl)-1H-pyrazol-4-yl)-7H-pyrrolo[2,3-c]pyridazin-7-yl)methyl pivalate (67c)

### Step 1:

To a solution of (4-(1H-pyrazol-4-yl)-7H-pyrrolo[2,3-c]pyridazin-7-yl)methyl pivalate (43a) (430 mg, 1.4 mmol) in chloroform (30 mL) at room temperature was added trans-cinnamaldehyde (67a) (881 4, 7 mmol) followed by (R)-α,α-Bis[3,5-bis(trifluoromethyl)phenyl]pyrrolidinemethanol trimethylsilyl ether (43d) (84 mg, 0.14 mmol), and p-nitrobenzoic acid (43c) (24 mg, 0.14 mmol). The resulting mixture was stirred at room temperature overnight and concentrated in vacuum to dryness. The residue obtained was purified by flash column chromatography (silica gel 25 g, eluting with ethyl acetate in hexane 0-100%) to furnish (R)-(4-(1-(3-oxo-1-phenylpropyl)-1H-pyrazol-4-yl)-7H-pyrrolo[2,3-c]pyridazin-7-yl)methyl pivalate (67b) (340 mg, 56%) as a solid. ¹H NMR (300 MHz, DMSO)S 9.71 (s, 1H), 9.29 (d, J=3.2, 1H), 8.88 (s, 1H), 8.37 (s, 1H), 8.01 (d, J=3.7, 1H), 7.40-7.34 (m, 5H), 7.07 (d, J=3.7, 1H), 6.39 (s, 2H), 6.14 (dd, J=5.1, 9.3, 1H), 3.80 (dd, J=10.1, 17.3, 1H), 3.41 (dd, J=5.5, 17.8, 1H), 1.08 (s, 9H); MS (ES+) 464.05 (M+CH₃OH+1).

### Step 2:

To a stirred solution of (R)-(4-(1-(3-oxo-1-phenylpropyl)-1H-pyrazol-4-yl)-7H-pyrrolo[2,3-c]pyridazin-7-yl)methyl pivalate (67b) (300 mg, 0.67 mmol) in THF (15 mL) was added concentrated ammonium hydroxide (2.0 mL, 28 mmol) and iodine (196 mg, 0.9 mmol). The resulting solution was stirred at room temperature for 1 h and quenched with saturated aqueous sodium thiosulfate solution (50 mL). The reaction mixture was extracted with dichloromethane (3×50 mL). The organic layers were combined washed with brine (30 mL), dried, filtered and concentrated in vacuum to dryness. The residue obtained was purified by flash column chromatography [silica gel 12 g, ethyl acetate/methanol (9:1) in hexane 0-100%] to furnish (R)-(4-(1-(2-cyano-1-phenylethyl)-1H-pyrazol-4-yl)-7H-pyrrolo[2,3-c]pyridazin-7-yl)methyl pivalate (67c) (170 mg, 58%) as a solid. ¹H NMR (300 MHz, CDCl₃) δ 9.09 (s, 1H), 8.16 (s, 1H), 7.97 (s, 1H), 7.72 (d, J=3.7, 1H), 7.52-7.33 (m, 5H), 6.65 (d, J=3.7, 1H), 6.42 (s, 2H), 5.73 (dd, J=6.5, 7.8, 1H), 3.67 (dd, J=8.0, 16.8, 1H), 3.33 (dd, J=6.4, 16.8, 1H), 1.15 (s, 9H); MS (ES⁺) 429.21 (M+1).

### Example 29

### (R)-3-(4-(7H-pyrrolo[2,3-c]pyridazin-4-yl)-1H-pyrazol-1-yl)-3-(3-hydroxy phenyl)propanenitrile (68f)

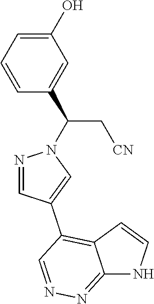

To a solution of (R)-(4-(1-(2-cyano-1-(3-hydroxyphenyl)ethyl)-1H-pyrazol-4-yl)-7H-pyrrolo[2,3-c]pyridazin-7-yl)methyl pivalate (68e) (45 mg, 0.32 mmol) in methanol (20 mL) was added 1N NaOH (128 μL). The reaction mixture was stirred at room temperature for 6 h. The reaction mixture was concentrated in vacuum and the residue obtained was purified by flash column chromatography [silica gel 4 g, eluting with ethyl acetate/methanol (9:1) in hexane 0-100%] to furnish (R)-3-(4-(7H-pyrrolo[2,3-c]pyridazin-4-yl)-1H-pyrazol-1-yl)-3-(3-hydroxy phenyl)propanenitrile (681) (18 mg, 19%) as a light yellow solid. ¹HNMR (300 MHz, DMSO) δ12.43 (s, 1H), 9.59 (s, 1H), 9.18 (s, 1H), 8.88 (s, 1H), 8.44 (s, 1H), 7.93 (d, J=3.4 Hz, 1H), 7.18 (t, J=7.9, 1H), 6.94 (d, J=3.4 Hz, 1H), 6.85 (d, J=7.7, 1H), 6.73 (dd, J=5.5, 12.7, 2H), 5.94 (dd, J=9.5, 5.7 Hz, 1H), 3.73 (dd, J=16.8, 9.7 Hz, 1H), 3.55 (dd, J=16.9, 5.7 Hz, 1H); MS (ES+) 331.1 (M+1).

### Preparation of (R)-(4-(1-(2-cyano-1-(3-hydroxyphenyl)ethyl)-1H-pyrazol-4-yl)-7H-pyrrolo[2,3-c]pyridazin-7-yl)methyl pivalate (68e)

### Step 1:

A mixture of 3-hydroxybenzaldehyde (68a) (5 g, 41 mmol) and vinyl acetate (68b) (4.15 mL, 45.1 mmol) in acetonitrile (15 mL) was added at room temperature to the suspension of potassium carbonate (6.8 g, 49.2 mmol) in acetonitrile (50 mL) and water (0.2 mL). The reaction mixture was refluxed for 40 h and cooled to room temperature. The reaction mixture was diluted with water (50 mL) and ethyl acetate (50 mL). The aqueous layer was separated and extracted with ethyl acetate (2×50 mL). The combined organic layers were dried, filtered and concentrated in vacuum to dryness. The residue obtained was purified by flash column chromatography (silica gel 40 g, eluting with ethyl acetate in hexane 0-50%) to furnish (E)-3-(3-hydroxyphenyl)acrylaldehyde (68c) (1.54 g, 25%) as a light yellow solid. ¹H NMR (300 MHz, DMSO) δ 9.72 (s, 1H), 9.66 (d, J=7.8, 1H), 7.67 (d, J=15.9, 1H), 7.28 (t, J=7.8, 1H), 7.18 (dd, J=1.2, 6.5, 1H), 7.12-7.06 (m, 1H), 6.89 (ddd, J=1.0, 2.4, 8.0, 1H), 6.75 (dd, J=7.8, 15.9, 1H); MS (ES+) 319.6 (2M+1).

### Step 2:

To a solution of (4-(1H-pyrazol-4-yl)-7H-pyrrolo[2,3-c]pyridazin-7-yl)methyl pivalate (43a) (430 mg, 1.4 mmol) in chloroform (30 mL) at room temperature was added (E/Z)-3-(3-hydroxyphenyl)acrylaldehyde (68c) (725 mg, 4.9 mmol), followed by (R)-α,α-Bis[3,5-bis(trifluoromethyl)phen-yl]pyrrolidinemethanol trimethylsilyl ether (43d) (84 mg, 0.14 mmol), and p-nitrobenzoic acid (43c) (24 mg, 0.14 mmol). The resulting mixture was stirred at room temperature overnight and concentrated in vacuum to dryness. The residue obtained was purified by flash column chromatography [silica gel 25 g, eluting with ethyl acetate/methanol (9:1) in hexane 0-100%] to furnish (R)-(4-(1-(1-(3-hydroxyphenyl)-3-oxopropyl)-1H-pyrazol-4-yl)-7H-pyrrolo[2,3-c]pyridazin-7-yl)methyl pivalate (68d) (393 mg, 63%) as a solid. MS (ES+) 480.07 (M+CH₃OH+1).

### Step-3:

To a stirred solution of give (R)-(4-(1-(1-(3-hydroxyphenyl)-3-oxopropyl)-1H-pyrazol-4-yl)-7H-pyrrolo[2,3-c]pyridazin-7-yl)methyl pivalate (68d) (300 mg, 0.67 mmol) in THF (15 mL) was added concentrated ammonium hydroxide (2.0 mL, 28 mmol) and iodine (196 mg, 0.9 mmol). The resulting solution was stirred at room temperature for 1 h and quenched with saturated aqueous sodium thiosulfate solution (50 mL). The reaction mixture was extracted with dichloromethane (3×50 mL). The organic layers were combined washed with brine (30 mL), dried, filtered and concentrated in vacuum to dryness. The residue obtained was purified by flash column chromatography [silica gel 12 g, ethyl acetate/methanol (9:1) in hexane 0-100%] to furnish (R)-(4-(1-(2-cyano-1-(3-hydroxyphenyl)ethyl)-1H-pyrazol-4-yl)-7H-pyrrolo[2,3-c]pyridazin-7-yl)methyl pivalate (68e) (145 mg, 49%) as a solid, which was pure enough to be used as such for next step. MS (ES+) 445.06 (M+1).

### Example 30

### (4-bromo-7H-pyrrolo[2,3-c]pyridazin-7-yl)methyl pivalate (69a)

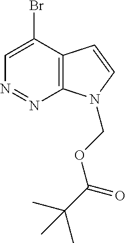

To a solution of 4-Bromo-7H-pyrrolo[2,3-c]pyridazine (41a) (3.5 g, 17.7 mmol) in dichloromethane (100 mL) was added at room temperature triethylamine (25 mL, 180 mmol), DMAP (100 mg), and chloromethyl pivalate (10.2 mL, 70 mmol). The reaction mixture was stirred at room temperature overnight and quenched with water (200 mL). The reaction mixture was extracted with dichloromethane (2×150 mL). The organic layers were combined dried, filtered and concentrated in vacuum to dryness. The residue obtained was purified by flash column chromatography (silica gel 120 g, eluting with ethyl acetate in hexane 0-100%) to furnish. (4-bromo-7H-pyrrolo[2,3-c]pyridazin-7-yl)methyl pivalate (69a) (2.6 g, 47%) as white solid. ¹HNMR (300 MHz, DMSO) δ 9.22 (s, 1H), 8.16 (dd, J=2.0, 3.6, 1H), 6.69 (d, J=3.6, 1H), 6.41 (s, 2H), 1.09 (s, 9H). ¹³C NMR (300 MHz, DMSO) δ 176.99, 150.21, 144.91, 136.65, 124.16, 117.88, 99.93, 67.47, 38.24; MS (ES+): 646.8 (2M+Na).

### Example 31

### 4-hydroxy-7H-pyrrolo[2,3-d][1,2,3]triazine-5-carboxamide (70c)

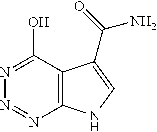

A stirred mixture of 2-amino-pyrrole-3,4-dicarboxamide (70b) (0.672 g, 4 mmol), AcOH (glacial, 40 mL), and H2O (20 mL) was cooled to 0° C. (ice bath), and tert-butylnitrite (1.151 mL, 9.6 mmol) was added over a 5 min period. The reaction was allowed to stir at 0° C. for 15 min and then at room temperature for 90 min. At that time, the flask was covered and allowed to stand for 16 h. The resultant mixture was then reduced to one-half of its original volume and cooled at 10° C. for 1 h, and the precipitate was collected by filtration, washed with H₂O (30 mL), and dried under reduced pressure at 78° C. for 24 h to give 4-hydroxy-7H-pyrrolo[2,3-d][1,2,3]triazine-5-carboxamide (70c) (0.4 g, 56%) as a purple solid. ¹HNMR (300 MHz, DMSO) δ 15.11 (s, 1H), 13.60 (s, 1H), 9.21 (s, 1H), 8.06 (s, 1H), 7.47 (s, 1H); ¹HNMR (300 MHz, DMSO/D₂O) δ 8.07 (s, 1H); ¹³CNMR (300 MHz, DMSO) δ 162.72, 156.85, 145.46, 130.57, 114.76, 105.43; MS (ES+) 180.2 (M+1); (ES−) 178.1 (M−1).

### Preparation of 2-amino-pyrrole-3,4-dicarboxamide (70b)

A stirred mixture of isopropyl alcohol (600 mL), commercial grade Raney nickel (50 g), and 2-amino-5-(methylthio)pyrrole-3,4-dicarboxamide (70a) (prepared as given in Gewalt, V. K.; Kleinert, M.; Thiele, B.; Hentschel, M. J Prakt.Chem. 1972, 2, 303-314, 15 g, 70.0 mmol) was heated at reflux temperature for 24 h. The reaction mixture was filtered (hot) through Celite. The Celite was resuspended in 2-propanol (500 mL) and then filtered through another bed of Celite. The solvent portions were combined and evaporated under reduced pressure, and the resultant solid was triturated with isopropyl alcohol and collected by filtration. The solid was dried in vacuum to afford first crop of 2-amino-pyrrole-3,4-dicarboxamide (70b) 2.64 g (22%) as a purple solid. A second crop was obtained by dissolving the solid from the reaction in hot water (50 mL) and filtering it through celite to remove Raney nickel. The filtrate was concentrated in vacuum, and the solid obtained was collected by filtration dried in vacuum to furnish 2-amino-pyrrole-3,4-dicarboxamide (70b) (2.305 g, 20%) as purple needles: mp>210° C. (dec). ¹HNMR (300 MHz, DMSO) δ 10.48 (s, 1H), 10.01-9.64 (bs, 1H), 7.49 (bs, 1H), 6.96 (d, J=2.7, 1H), 6.93-6.83 (bs, 1H), 6.50-6.25 (bs, 1H), 6.09 (s, 2H). ¹³CNMR (300 MHz, DMSO) δ 168.45, 168.20, 147.64, 116.19, 113.55, 93.10; MS (ES+) 169.2 (M+1), 191.1 (M+Na).

### Example 32

### 7H-pyrrolo[2,3-c]pyridazin-4-N,N′-di(trimethyl)silylamine (76b)

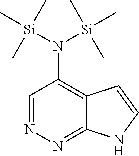

To a solution of 4-bromo-7H-pyrrolo[2,3-c]pyridazine (41a) (99 mg, 0.50 mmol) in 1,4-dioxane (12 mL) was added chloro(2-dicyclohexylphosphino-2′,6′-dimethoxy-1, 1′-biphenyl)[2-(2-aminoethylphenyl)]-Pd(II) (0.05 mmol) and bubbled with nitrogen for 15 min. To the solution was added LiHMDS (1 M in THF, 2 mL), bubbled with nitrogen again for 5 min and heated at reflux for 14 h. The reaction mixture was cooled to room temperature and quenched with saturated aqueous NH₄Cl (6 mL), diluted with water (20 mL) and extracted with ethyl acetate (2×50 mL). The organic layers were combined washed with brine (30 mL), dried, and concentrated in vacuum to dryness. The residue obtained was purified by flash column chromatography [silica gel 4 g, eluting with ethyl acetate/methanol (9:1) in hexane 1:0 to 1:1, followed by chloroform/methanol 1:0 to 4:1(R_{f}=0.48 with hexanes/ethyl acetate/methanol=1:1:0.1)] to furnish 7H-pyrrolo[2,3-c]pyridazin-4-N,N′-di(trimethyl)silylamine (76b) (61 mg, 44%) as a light brown solid and 7H-pyrrolo[2,3-c]pyridazin-4-amine (76c) (12 mg, 18%, R_{f}=0.24 with chloroform/methanol=4:1). ¹H NMR (300 MHz, DMSO-d₆): δ 12.17 (s, 1H), 8.33 (s, 1H), 7.67 (d, J=3.4, 1H), 6.36 (d, J=3.4, 1H),-0.00 (s, 18H); MS (ES+): 279.1 (M+1).

### Example 33

### 7H-pyrrolo[2,3-c]pyridazin-4-amine (76c)

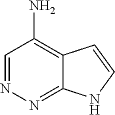

To a solution of 7H-pyrrolo[2,3-c]pyridazin-4-N,N′-di(trimethyl)silylamine (76b) (48 mg, 0.17 mmol) in methanol (4 mL) was added 4 N HCl in dioxane (1 mL) and stirred at room temperature for 2 h. The reaction mixture was concentrated to dryness and the residue obtained was purified by flash column chromatography (silica gel 4 g, eluting chloroform/methanol, 1:0 to 4:1, R_{f}=0.24 with chloroform/methanol=4:1) to afford 7H-pyrrolo[2,3-c]pyridazin-4-amine (76c) (18 mg, 79%) as a white solid. ¹HNMR (300 MHz, DMSO-d₆): δ 11.62 (s, 1H), 8.15 (s, 1H), 7.33 (d, J=3.4, 1H), 6.51 (d, J=3.4, 1H), 6.41 (s, 2H); MS (ES⁺): 135.2 (M+1).

### Example 34

### 2-(4-(7H-pyrrolo[2,3-c]pyridazin-4-yl)-1H-pyrazol-1-yl)cyclopentane carbonitrile (78e)

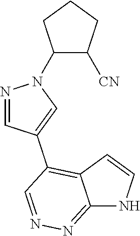

To a solution of (4-(1-(2-cyanocyclopentyl)-1H-pyrazol-4-yl)-7H-pyrrolo[2,3-c]pyridazin-7-yl)methyl pivalate (78d) (123 mg, 0.31 mmol) in methanol (10 mL) was added 1N NaOH (94 μL). The reaction mixture was stirred at room temperature for 6 h and concentrated in vacuum to dryness. The residue obtained was purified by flash column chromatography (silica gel 4 g, eluting with CMA-80 in chloroform 0-100%) to furnish 2-(4-(7H-pyrrolo[2,3-c]pyridazin-4-yl)-1H-pyrazol-1-yl)cyclopentanecarbonitrile (78e) (42 mg, 48%) as a yellow solid. ¹HNMR (300 MHz, DMSO) δ 12.41 (s, 1H), 9.18 (s, 1H), 8.80 (s, 1H), 8.40 (s, 1H), 7.92 (d, J=3.4, 1H), 6.95 (d, J=3.4, 1H), 5.09 (q, J=8.2, 1H), 3.53 (q, J=8.6, 1H), 2.39-2.24 (m, 2H), 2.18-2.04 (m, 1H), 2.03-1.83 (m, 3H); MS (ES+) 279.15 (M+1); (ES−) 217.0 (M−1).

### Preparation of (4-(1-(2-cyanocyclopentyl)-1H-pyrazol-4-yl)-7H-pyrrolo[2,3-c]pyridazin-7-yl)methyl pivalate (78d)

### Step 1:

To an acidic solution of sodium periodate (60 g, 0.28 mol) in water (500 mL) was added the solution of 1,2-cyclohexanediol (78a) (25 g, 0.215 mol) in ethyl ether (300 mL). The mixture was stirred vigorously for 0.5 hour at room temperature. After addition of KOH aqueous solution (20%, 80 ml), the reaction mixture was stirred for an additional 1 hour. The mixture was extracted with ethyl ether (2×250 mL). The organic layers were combined and dried. The solvent was removed to give cyclopent-1-enecarbaldehyde (78b) as a yellow oil (yield: 18.3 g, 88%).

### Step 2:

To a solution of (4-(1H-pyrazol-4-yl)-7H-pyrrolo[2,3-c]pyridazin-7-yl)methyl pivalate (43a) (750 mg, 2 5 mmol) in chloroform (50 mL) at room temperature was added cyclopent-1-enecarbaldehyde (78b) (2.4 mL, 25 mmol) followed by (R)-α,α-Bis[3,5-bis(trifluoromethyl)phenyl]pyrrolidinemethanol trimethylsilyl ether (43d) (224 mg, 0.375 mmol), and p-nitrobenzoic acid (43c) (63 mg, 0.375 mmol). The resulting mixture was stirred at room temperature overnight and concentrated in vacuum to dryness. The residue obtained was purified by flash column chromatography [silica gel 25 g, eluting with (9:1) ethyl acetate/methanol in hexane 0-100%) to furnish (4-(1-(2-formylcyclopentyl)-1H-pyrazol-4-yl)-7H-pyrrolo[2,3-c]pyridazin-7-yl)methyl pivalate (78c) (840 mg, 85%) as a white solid. ¹HNMR (300 MHz, CDCl₃) δ 9.82 (s, 1H), 9.12 (d, J=3.8 Hz, 1H), 8.06 (s, 1H), 8.02 (d, J=0.5 Hz, 1H), 7.73 (d, J=3.7 Hz, 1H), 6.71 (d, J=3.7 Hz, 1H), 6.43 (s, 2H), 5.15 (dd, J=14.1, 7.6 Hz, 1H), 3.45 (t, J=5.1 Hz, 1H), 2.35-2.26 (m, 2H), 2.12-1.95 (m, 3H), 1.83-1.70 (m, 1H), 1.16 (s, 9H); MS (ES+) 428.16 (M+MeOH+1).

### Step 3:

To a stirred solution of (4-(1-(2-formylcyclopentyl)-1H-pyrazol-4-yl)-7H-pyrrolo[2,3-c]pyridazin-7-yl)methyl pivalate (78c) (158 mg, 0.4 mmol) in THF (10 mL) was added concentrated ammonium hydroxide (1.2 mL, 1.6 mmol) and iodine (112 mg, 0.44 mmol). The resulting solution was stirred at room temperature for 1 h and quenched with saturated aqueous sodium thiosulfate solution (20 mL). The reaction mixture was extracted with dichloromethane (3×30 mL). The organic layers were combined washed with brine (30 mL), dried, filtered and concentrated in vacuum to dryness. The residue obtained was purified by flash column chromatography [silica gel 4 g, ethyl acetate/methanol (9:1) in hexane 0-100%] to furnish (4-(1-(2-cyanocyclopentyl)-1H-pyrazol-4-yl)-7H-pyrrolo[2,3-c]pyridazin-7-yl)methyl pivalate (78d) (123 mg, 80%) as a white solid, ¹H NMR (300 MHz, CDCl₃) δ 9.14 (s, 1H), 8.10 (s, 1H), 8.07 (s, 1H), 7.75 (d, J=3.7 Hz, 1H), 6.71 (d, J=3.7 Hz, 1H), 6.44 (s, 2H), 4.91 (q, J=7.8 Hz, 1H), 3.43 (dd, J=16.4, 8.3 Hz, 1H), 2.48-2.29 (m, 3H), 2.20-1.96 (m, 3H), 1.16 (s, 9H).MS (ES+) 393.08 (M+1), 807.15 (2M+1).

### Example 35

### (2-(4-(7H-pyrrolo[2,3-c]pyridazin-4-yl)-1H-pyrazol-1-yl)cyclopentyl)methanol (781)

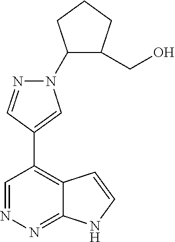

To a solution of (4-(1-(2-(hydroxymethyl)cyclopentyl)-1H-pyrazol-4-yl)-7H-pyrrolo[2,3-c]pyridazin-7-yl)methyl pivalate (78g) (60 mg, 0.15 mmol) in methanol (5 mL) was added 1N NaOH (60 μL). The reaction mixture was stirred at room temperature for 6 h and concentrated in vacuum to dryness. The residue obtained was purified by flash column chromatography (silica gel 4 g, eluting with CMA-80 in chloroform 0-100%) to furnish (2-(4-(7H-pyrrolo[2,3-c]pyridazin-4-yl)-1H-pyrazol-1-yl)cyclopentyl)methanol (78f) (23 mg, 54%) as an off-white solid. ¹H NMR (300 MHz, DMSO) δ 12.35 (s, 1H), 9.15 (s, 1H), 8.64 (s, 1H), 8.28 (s, 1H), 7.88 (d, J=3.4 Hz, 1H), 6.93 (d, J=3.4 Hz, 1H), 4.67 (t, J=5.2 Hz, 1H), 4.56 (q, J=7.5 Hz, 1H), 3.49-3.34 (m, 2H), 2.47-2.38 (m, 1H), 2.18-2.01 (m, 2H), 1.98-1.88 (m, 1H), 1.87-1.79 (m, 1H), 1.74-1.63 (m, 1H), 1.59-1.48 (m, 1H). MS (ES+) 284.2 (M+1); 567.2 (2M+1); (ES−) 282 (M−1), 565.1 (2M−1).

### Preparation of (4-(1-(2-(hydroxymethyl)cyclopentyl)-1H-pyrazol-4-yl)-7H-pyrrolo[2,3-c]pyridazin-7-yl)methyl pivalate (78g)

To a solution of ((4-(1-(2-formylcyclopentyl)-1H-pyrazol-4-yl)-7H-pyrrolo[2,3-c]pyridazin-7-yl)methyl pivalate (78c) (0.51 mg, 1.28 mmol) in THF (50 mL) was added NaBH₄ (48 mg, 1.28 mmol) and methanol (1 mL). The reaction mixture was stirred at room temperature for 1 h and concentrated in vacuum to dryness. The residue obtained was purified by flash column chromatography [silica gel 12 g, eluting with (ethyl acetate/methanol 9:1) in hexane 0-100%] to furnish (4-(1-(2-(hydroxymethyl)cyclopentyl)-1H-pyrazol-4-yl)-7H-pyrrolo[2,3-c]pyridazin-7-yl)methyl pivalate (78g) (298 mg, 58%) as a off white solid; ¹H NMR (300 MHz, CDCl₃) δ 9.12 (s, 1H), 8.06 (s, 1H), 8.04 (s, 1H), 7.72 (d, J=3.7 Hz, 1H), 6.70 (d, J=3.7 Hz, 1H), 6.43 (s, 2H), 4.57 (q, J=7.9 Hz, 1H), 3.73 (m, 2H), 2.62-2.50 (m, 1H), 2.43-2.31 (m, 1H), 2.29-2.20 (m, 1H), 2.13-1.93 (m, 2H), 1.87-1.75 (m, 1H), 1.61-1.49 (m, 1H), 1.60-1.49 (m, 1H), 1.16 (s, 9H). MS (ES+) 398.19 (M+1).

### Example 36

### (4-(1-(2-((methylsulfonyloxy)methyl)cyclopentyl)-1H-pyrazol-4-yl)-7H-pyrrolo[2,3-c]pyridazin-7-yl)methyl pivalate (78h)

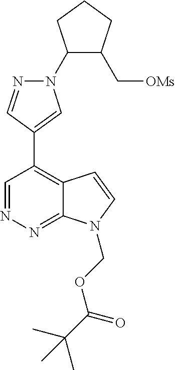

To a solution of (4-(1-(2-(hydroxymethyl)cyclopentyl)-1H-pyrazol-4-yl)-7H-pyrrolo[2,3-c]pyridazin-7-yl)methyl pivalate (78g) (210 mg, 0.528 mmol) in dichloromethane (20 mL) was added TEA (295 μL. 2.11 mmol), DMAP (7 mg), and methanesulfonyl chloride (123 μL. 1.58 mmol). The reaction mixture was stirred at room temperature overnight and quenched with water (25 mL). The reaction mixture was extracted with dichloromethane (2×20 mL). The organic layers were combined washed with brine (25 mL), dried, filtered and concentrated in vacuum. The residue obtained was purified by flash column chromatography (silica gel 4 g, eluting with ethyl acetate in hexane 0-100%) to furnish ((4-(1-(2-((methylsulfonyloxy)methyl)cyclopentyl)-1H-pyrazol-4-yl)-7H-pyrrolo[2,3-c]pyridazin-7-yl)methyl pivalate (78h) (197 mg, 78%) as a off-white solid. ¹HNMR (300 MHz, CDCl₃) δ 9.18 (s, 1H), 8.11 (s, 1H), 8.08 (s, 1H), 7.78 (d, J=3.7, 1H), 6.79 (d, J=3.7, 1H), 6.42 (s, 2H), 4.34-4.26 (m, 2H), 3.00 (s, 3H), 2.90-2.75 (m, 1H), 2.30 (dd, J=7.6, 15.2, 2H), 2.19-1.96 (m, 3H), 1.91-1.60 (m, 2H), 1.16 (s, 9H); MS (ES+) 476.03 (M+1).

### Example 37

### 2-(2-(4-(7H-pyrrolo[2,3-c]pyridazin-4-yl)-1H-pyrazol-1-yl)cyclopentyl)acetonitrile (78i)

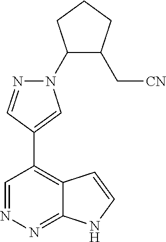

To a solution of ((4-(1-(2-((methylsulfonyloxy)methyl)cyclopentyl)-1H-pyrazol-4-yl)-7H-pyrrolo[2,3-c]pyridazin-7-yl)methyl pivalate (78h) (189 mg, 0.4 mmol) in DMF (5 mL) was added potassium cyanide (129 mg, 1.99 mmol), tetraethylammonium chloride (13 mg, 0.078 mmol) and 18-crown-6 (11 mg, 0.039 mmol). The reaction mixture was heated with stirring at 95° C. for 3 h, cooled to room temperature and quenched with water (10 mL). The reaction mixture was extracted with ethyl acetate (3×25 mL). The organic layers were combined washed with brine (10 mL), dried, filtered and concentrated in vacuum. The residue obtained was purified by flash column chromatography (silica gel 4 g, eluting with CMA-80 in chloroform 0-100%) to furnish 2-(2-(4-(7H-pyrrolo[2,3-c]pyridazin-4-yl)-1H-pyrazol-1-yl)cyclopentyl)acetonitrile (78i) (37 mg, 31%) as a yellow solid. ¹HNMR (300 MHz, DMSO) δ 12.37 (s, 1H), 9.16 (s, 1H), 8.72 (s, 1H), 8.33 (s, 1H), 7.89 (d, J=3.4, 1H), 6.96 (d, J=3.4, 1H), 4.50 (d, J=8.0, 1H), 2.74-2.58 (m, 3H), 2.23 (m, 1H), 2.18 (m, 2H), 1.96-1.71 (m, 2H), 1.55 (m, 1H); MS (ES+) 292.338 (M+1).

### Example 38

### 3-(4-methyl-3-(methyl(6-oxo-6,7-dihydro-5H-pyrrolo[2,3-d]pyrimidin-4-yl)amino)piperidin-1-yl)-3-oxopropanenitrile (79d)

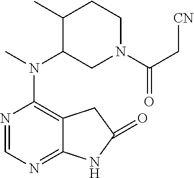

To a solution of 4-(methyl(4-methylpiperidin-3-yl)amino)-5H-pyrrolo[2,3-d]pyrimidin-6(7H)-one (79c) (10 mg, 0.038 mmol) in DMF (1.5 mL) was added 2-cyanoacetic acid (5 mg, 0.058 mmol), N,N-diiospropylethylamine (DIEA, 0.015 mL, 0.086 mmol) and cooled with ice/water. The cold mixture was treated with 2-(1H-7-azabenzotriazol-1-yl) —1,1,3,3-tetramethyl uronium hexafluorophosphate methanamini (HATU, 22 mg, 0.058 mmol) and warmed up slowly to room temperature. The reaction mixture was diluted with chloroform/methanol (3:1, mL) and washed with water (5 mL). The aqueous phase was separated and extracted with chloroform/methanol (3:1, 2×10 mL). The organic layers were combined dried, filtered, and concentrated in vacuum. The residue obtained was and purified by flash column chromatography [silica gel 4 g, eluting with chloroform/methanol (1:0 to 95:5), R_{f}=0.22 with chloroform/methanol=95:5)] to afford 3-(4-methyl-3-(methyl(6-oxo-6,7-dihydro-5H-pyrrolo[2,3-d]pyrimidin-4-yl)amino)piperidin-1-yl)-3-oxopropanenitrile (79d) (10 mg, 80%) as a colorless semisolid. ¹H NMR (at 350.2 K, 300 MHz, DMSO-d₆): δ 10.68 (s, 1H), 8.13 (s, 1H), 4.64-4.50 (m, 1H), 4.04-3.22 (m, 8H), 3.07 (s, 3H), 2.32-2.18 (m, 1H), 1.82-1.46 (m, 2H), 0.99 (d, J=7.1 Hz, 3H); MS (ES⁺): 329.1 (M+1).

### Preparation of 4-(methyl(4-methylpiperidin-3-yl)amino)-5H-pyrrolo[2,3-d]pyrimidin-6(7H)-one (79c)

### Step 1:

### Preparation of Compound (79a)

To a stirred solution of potassium tert-butoxide (64.85 g, 577.95 mmol) in tetrahydrofuran (160 mL) was added dimethyl carbonate (36.41 g, 404.56 mmol) by maintaining the temperature below 30° C. To this mixture a solution of 3-amino-4-methylpyridine (25 g, 231.18 mmol) in tetrahydrofuran (100 mL) was added at a rate that maintained the temperature below 30° C. The viscous reaction mixture was diluted with tetrahydrofuran (250 mL) and stirred for 18 h. The reaction was quenched with water (200 mL); the organic layer was separated and washed with brine (100 mL). The aqueous layers were extracted with ethyl acetate (200 mL); washed with water (100 mL) and brine (50 mL). The organic layers were combined dried and concentrated in vacuum. The crude residue obtained was recrystallized from dichloromethane (100 mL) and hexanes (400 mL) to give pure methyl 4-methylpyridin-3-ylcarbamate (34.8 g, 90.5%) as a cream color solid. ¹H NMR (300 MHz, DMSO-d₆) δ 9.11 (s, 1H, D₂O exchangeable), 8.49 (s, 1H), 8.22 (d, J=4.9, 1H), 7.23 (d, J=4.9, 1H), 3.67 (s, 3H), 2.22 (s, 3H); MS (ES+) 167.2 (M+1), 189.2 (M+23). Analysis: Calculated for C₈H₁₀N₂O₂: C, 57.82; H, 6.06; N, 16.85. Found: C, 57.70; H, 6.12; N, 16.79.

A solution of above methyl 4-methylpyridin-3-ylcarbamate (34 g, 204.60 mmol) in acetic acid (400 mL) was degassed for 2 h by bubbling with nitrogen gas. To the solution was added Rhodium on carbon (5%, 50% wet, 5 g) and hydrogenated (150 psi, Hydrogen) at 100° C. (external jacket temperature) for 72 h. The reaction mixture was filtered through celite and concentrated in vacuum. The residue obtained was azeotroped with toluene to furnish crude methyl 4-methylpiperidin-3-ylcarbamate as an acetate salt (57 g). ¹H NMR (300 MHz, DMSO-d₆) δ 6.87 (d, J=9.0, 1H, D₂O exchangeable), 3.53 (m, 4H, 1H D₂O exchangeable), 2.86-2.78 (m, 1H), 2.74 (dd, J=3.4, 13.0, 1H), 2.59 (dd, J=2.7, 12.8, 1H), 2.42 (dt, J=7.9, 21.3, 2H), 1.78-1.60 (m, 1H), 1.34-1.19 (m, 2H), 0.78 (d, J=6.8, 3H); MS (ES+) 173.3 (M+1).

To a stirred solution of above methyl 4-methylpiperidin-3-ylcarbamate (56.17 g, 326.59 mmol) and acetic acid (20 mL) in toluene (500 mL) was added benzaldehyde (51.98 g, 489.89 mmol) at 20° C. The reaction was stirred at the same temperature for 2.5 h. The imine obtained was added to a stirred solution of sodium triacetoxyborohydride (103.82 g, 489.89 mmol) in toluene (300 mL) at 20° C. The reaction was stirred for 18 h at the same temperature and pH was adjusted between 7.0 and 7.5 using aqueous sodium hydroxide (2N). The aqueous layer was separated and extracted with toluene (2×200 mL). The toluene layers were combined, added conc. HCl (70 mL) and heated to 80° C. for about 2 h. The solution was concentrated to dryness and the residue obtained was triturated with toluene. The solid obtained was collected by filtration and dried to afford methyl 1-benzyl-4-methylpiperidin-3-ylcarbamate hydrochloride (36.5 g, 60% from methyl 4-methylpyridin-3-ylcarbamate) as a colorless crystalline solid. ¹H NMR (300 MHz, CDCl₃) δ 12.31 (s, 1H, D₂O exchangeable), 7.62-7.52 (m, 3H), 7.48-7.42 (m, 2H), 4.33-4.14 (m, 2H), 4.06 (d, J=12.9, 1H), 3.65 (s, 3H), 3.52 (d, J=10.8, 1H), 3.31 (d, J=11.5, 1H), 2.91-2.60 (m, 2H), 2.28 (d, J=13.6, 1H), 1.83 (s, 1H), 1.66 (d, J=15.1, 1H), 0.97 (d, J=6.5, 3H); MS (ES+) 263.2 (M+1).

To a stirred suspension of methyl 1-benzyl-4-methylpiperidin-3-ylcarbamate hydrochloride from above (45 g, 150 mmol) in tetrahydrofuran (190 mL) was added a solution of lithium aluminum hydride (1 M solution in THF, 225 mL, 225 mmol) at −15° C. The reaction mixture was refluxed for 2 h and cooled to 0° C. The reaction mixture was carefully quenched by adding water and the inorganic salt obtained were filtered off and washed with tetrahydrofuran (100 mL). The filtrate was concentrated in vacuum to afford cis-1-benzyl-N,4-dimethylpiperidin-3-amine (79a) (33 g) as a colorless oil. ¹H NMR (300 MHz, DMSO-d₆) δ 7.35-7.27 (m, 5H), 7.26-7.21 (m, 1H), 3.52-3.38 (m, 2H), 3.34 (s, 1H), 2.32 (s, 1H), 2.18 (s, 3H), 2.08 (d, J=12.5, 2H), 1.66 (s, 1H), 1.47-1.27 (m, 3H), 0.88-0.82 (m, 3H).

### Step 2:

To a suspension of 4-chloro-5H-pyrrolo[2,3-d]pyrimidin-6(7H)-one (100 (100 mg, 0.59 mmol) in 2-propanol (1.4 mL) was added 1-benzyl-N,4-dimethylpiperidin-3-amine (79a) (cis, racemic, 390 mg, 1.79 mmol) and N,N-diiospropylethylamine (0.55 mL, 3.16 mmol). The reaction mixture was heated in a microwave for 5 h (power set: 300W; temperature set: 160° C.). The reaction mixture was concentrated in vacuum and the residue obtained was purified by flash column chromatography (silica gel 12 g, eluting with (hexanes/10% methanol in ethyl acetate=1:0 to 1:3), to afford 4-((1-benzyl-4-methylpiperidin-3-yl)(methyl)amino)-5H-pyrrolo[2,3-d]pyrimidin-6(7H)-one (79b) (62 mg) as a light brown gum. MS (ES+) 352.2 (M+1).

### Step 3:

A solution of above product 4-((1-benzyl-4-methylpiperidin-3-yl)(methyl)amino)-5H-pyrrolo[2,3-d]pyrimidin-6(7H)-one (79b) (59 mg, 0.17 mmol) in methanol (15 mL) was added TFA (26 μl, 0.33 mmol), palladium hydroxide (55 mg, 20%) and hydrogenated at ˜50 psi for 6 h. The reaction mixture was filtered and concentrated in vacuum. The residue obtained was purified by flash column chromatography [silica gel 4 g, eluting with chloroform/CMA 80 (1:0 to 1:1), R_{f}=0.14 with chloroform/CMA 80=1:1] to afford 4-(methyl(4-methylpiperidin-3-yl)amino)-5H-pyrrolo[2,3-d]pyrimidin-6(7H)-one (79c) (11 mg, 7.5% for two steps). ¹H NMR (300 MHz, MeOH-d₄): δ 8.15 (s, 1H), 4.50-4.60 (m 1H), 3.12-3.25 (m, 2H), 3.14 (s, 3H), 2.92-2.71 (m, 4H), 2.44-2.25 (m, 1H), 2.01-1.82 (m, 1H), 1.50-1.60 (m, 1H), 1.10 (d, J=7.2 Hz, 3H); MS (ES+) 262.2 (M+1).

### Example 39

The following illustrate representative pharmaceutical dosage forms, containing a compound of formula I (‘Compound X’), for therapeutic or prophylactic use in humans.

| (i) Tablet 1 | mg/tablet |
|---|---|
| Compound X = | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X = | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X = | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X = (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/ml) | mg/ml |
|---|---|
| Compound X = (free acid form) | 10.0 |
| Monobasic sodium phosphate | 0.3 |
| Dibasic sodium phosphate | 1.1 |
| Polyethylene glycol 400 | 200.0 |
| 01N Sodium hydroxide solution | q.s. |
| (pH adjustment to 7.0-7.5) | |
| Water for injection | q.s. ad 1 mL |

| (vi) Aerosol | mg/can |
|---|---|
| Compound X = | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

TABLE I
Activity for Representative Compounds of
the Invention for JAK Family of Enzymes

| Compound | Activity |
|---|---|
| 31m | IC₅₀ > 10 uM |
| 32c | IC₅₀ > 10 uM |
| 34j | IC₅₀ < 5 uM |
| 43g | IC₅₀ < 5 uM |
| 44d | IC₅₀ < 5 uM |
| 45d | IC₅₀ > 10 uM |
| 46c | IC₅₀ < 10 uM |
| 47d | IC₅₀ < 5 uM |
| 48d | IC₅₀ < 5 uM |
| 49c | IC₅₀ < 5 uM |
| 50a | IC₅₀ > 10 uM |
| 51e | IC₅₀ < 5 uM |
| 52e | IC₅₀ < 5 uM |
| 53d | IC₅₀ < 5 uM |
| 54e | IC₅₀ < 5 uM |
| 55d | IC₅₀ < 5 uM |
| 56d | IC₅₀ < 5 uM |
| 57e | IC₅₀ < 5 uM |
| 58d | IC₅₀ < 5 uM |
| 59d | IC₅₀ < 5 uM |
| 60f | IC₅₀ < 5 uM |
| 60g | IC₅₀ < 5 uM |
| 62b | IC₅₀ < 5 uM |
| 67d | IC₅₀ < 5 uM |
| 68f | IC₅₀ < 5 uM |
| 70c | IC₅₀ > 10 uM |
| 43a | IC₅₀ > 10 uM |

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A compound of formula I: 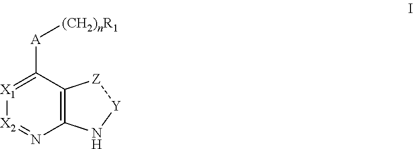
wherein:
A is CR₂R₃, NR₃, O or S; or when R₁ is other than H, A can also be absent;
X₁ is N or CR₄;
X₂ is N or CR₅;
Y is CR₆R₇, C═O or C═S, and Z is CR₈R₉, NR₁₀, O, S, C═O, C═S;
or Y is O, S or NR₁₁, and Z is CR₁₂R₁₃, C═O or C═S;
or Y is CR₆ and Z is CR₈ when X₁ is N or CR₄ and X₂ is N;
the bond represented by-is a single bond; or when X₁ is N or CR₄, X₂ is N, Y is CR₆ and Z is CR₈ the bond represented by-is a double bond;
n is 0 or 1;
R₁ is H, halogen, alkyl, cycloalkyl, heterocycle, heteroaryl, aryl or a bridged ring group; wherein any aryl or heteroaryl of R₁ is optionally substituted with one or more Rₐ groups; and wherein any alkyl, cycloalkyl, heterocycle or bridged ring group of R₁ is optionally substituted with one or more groups selected from Rₐ, oxo and ═NOR_{z}; or R₁ is halogen when A is CR₂R₃ or absent; or R₁ is —Oalkyl when A is CR₂R₃, NR₃ or absent; wherein —Oalkyl is optionally substituted with one or more groups selected from Rₐ, oxo and ═NOR_{z};
R₂ is H, alkyl or cycloalkyl;
R₃ is H, CN, —C(O)alkyl, —C(O)alkenyl, —C(O)alkynyl, —C(O)cycloalkyl, —C(O)aryl, —C(═O)C(═O)NHlower alkyl, —CONR_{b}R_{c}, alkyl, alkenyl, heterocycle, heteroaryl or aryl; wherein any aryl, —C(O)aryl or heteroaryl of R₃ is optionally substituted with one or more R_{d} groups; and wherein any alkyl, alkenyl, heterocycle, —C(O)alkyl, —C(O)alkenyl, —C(O)alkynyl, —C(O)cycloalkyl or —C(═O)C(═O)NHlower alkyl of R₃ is optionally substituted with one or more groups selected from R_{d}, oxo and ═NOR_{z}; and R₄ is H, halogen, alkyl, cycloalkyl, alkenyl, alkynyl, aryl, heteroaryl, heterocycle, NO₂, CN, OH, —ORₑ, —NR_{f}R_{g}, N₃, —SH, —SRₑ, —C(O)alkyl, —C(O)alkenyl, —C(O)alkynyl, —C(O)cycloalkyl, —C(O)aryl, —C(O)heteroaryl, —C(O)heterocycle, —C(O)ORₕ, —C(O)NR_{f}R_{g}, —C(═NR_{f})NR_{f}R_{g}, —NR_{f}CORₑ, —NR_{f}C(O)ORₑ, —NR_{f}S(O)₂Rₑ, —NR_{f}CONR_{f}R_{g}, —OC(O)NR_{f}R_{g}, —S(O)Rₑ, —S(O)NR_{f}R_{g}, —S(O)₂Rₑ, —S(O)₂OH, —S(O)₂NR_{f}R_{g} or —C(═O)C(═O)NHlower alkyl; wherein any aryl, heteroaryl, —C(O)aryl or —C(O)heteroaryl of R₄ is optionally substituted with one or more Rᵢ M groups; and wherein any alkyl, cycloalkyl, alkenyl, alkynyl, heterocycle, —C(O)alkyl, —C(O)alkenyl, —C(O)alkynyl, —C(O)cycloalkyl, —C(O)heterocycle or —C(═O)C(═O)NHlower alkyl of R₄ is optionally substituted with one or more groups selected from Rᵢ, oxo and ═NOR_{z};
or R₃ and R₄ together with the atoms to which they are attached form a five-membered heterocycle or a five-membered heteroaryl; wherein the five-membered heterocycle is optionally substituted with one or more groups selected from oxo or alkyl; and wherein the five-membered heteroaryl is optionally substituted with —OR₁₆ or —NHR₁₇;
R₅ is H, halogen, alkyl, cycloalkyl, alkenyl, alkynyl, aryl, heteroaryl, heterocycle, NO₂, CN, —OH, —ORⱼ, —NRₖRₘ, N₃, SH, —SRⱼ, —C(O)Rₙ, —C(O)ORₙ, —C(O)NRₖRₘ, —C(═NRₖ)NRₖRₘ, —NRₖCORⱼ, —NRₖC(O)ORⱼ, —NRₖS(O)₂Rⱼ, —NRₖCONRₖRₘ, —OC(O)NRₖRₘ, —S(O)Rⱼ, —S(O)NRₖRₘ, —S(O)₂Rⱼ, —S(O)₂OH, or —S(O)₂NRₖRₘ; wherein any aryl or heteroaryl of R₅ is optionally substituted with one or more Rₚ groups; and wherein any alkyl, cycloalkyl, alkenyl, alkynyl or heterocycle of R₅ is optionally substituted with one or more groups selected from Rₚ, oxo and ═NOR_{z};
R₆ is H, OH, —CN, NO₂, CO₂R_{q}, —C(O)R_{q}, —NR_{q}COR_{q}, —NR_{q}Rᵣ, halogen, lower alkyl, CONR_{q}Rᵣ, or alkenyl; wherein lower alkyl or alkenyl is optionally substituted with one or more Rₛ groups;
R₇ is H, OH, NO₂, CO₂H, —NR_{q}Rᵣ, halogen or lower alkyl; which lower alkyl is optionally substituted with one or more Rₛ groups;
R₈ is H, OH, —CN, NO₂, CO₂R_{q}, —C(O)R_{q}, —NR_{q}COR_{q}, —NR_{q}Rᵣ, halogen, lower alkyl, CONR_{q}Rᵣ, or alkenyl; wherein lower alkyl or alkenyl is optionally substituted with one or more Rₛ groups;
R₉ is H, OH, NO₂, CO₂H, —NR_{q}Rᵣ, halogen or lower alkyl; which lower alkyl is optionally substituted with one or more Rₛ groups;
R₁₀ is H or alkyl;
R₁₁ is H or alkyl;
R₁₂ is H or alkyl;
R₁₃ is H or alkyl;
R₁₆ is H or alkyl;
R₁₇ is H, —C(O)alkyl, —C(O)alkenyl, —C(O)alkynyl, —C(O)cycloalkyl, —C(O)aryl, —C(O)heteroaryl, —C(O)heterocycle, or —C(═O)C(═O)NHR₁₈;
R₁₈ is lower alkyl or cycloalkyl; wherein lower alkyl or cycloalkyl is optionally substituted with one or more —Olower alkyl;
each Rₐ is independently selected from halogen, aryl, heteroaryl, heterocycle, alkyl, alkenyl, alkynyl, cycloalkyl, OH, CN, —OR_{z}, —Oaryl, —Oheterocycle, —Oheteroaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2}, —C(O)heterocycle, —C(O)aryl, —C(O)heteroaryl and —C(O)C(O)R_{z}; wherein any aryl, heteroaryl, —Oaryl, —Oheteroaryl, —Saryl, —Sheteroaryl, —S(O)aryl, —S(O)heteroaryl, —S(O)₂aryl, —S(O)₂heteroaryl, —NHCOaryl, —NHCOheteroaryl, —NHS(O)₂aryl, —C(O)aryl or —C(O)heteroaryl of Rₐ is optionally substituted with one or more R_{y} groups; and wherein any heterocycle, —Oheterocycle, alkyl, alkenyl, alkynyl, cycloalkyl or —C(O)heterocycle of Rₐ is optionally substituted with one or more groups selected from R_{y}, oxo, ═NOR_{z}, ═NOH and ═CR_{z3}R_{z4};
R_{b} and R_{c}, are each independently selected from H, alkyl, alkenyl, alkynyl, cycloalkyl, heterocycle, aryl and heteroaryl; or R_{b} and R_{c} together with the nitrogen to which they are attached form a pyrrolidino, piperidino, piperazino, azetidino, morpholino, or thiomorpholino;
each R_{d} is independently selected from halogen, aryl, heteroaryl, heterocycle, R_{z}, OH, CN, —OR_{z}, —Oaryl, —OC(O)R_{z}, —OC(O)NR_{z1}R_{z2}, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z}; wherein any aryl, heteroaryl, heterocycle, —Oaryl, —Saryl, —Sheteroaryl, —S(O)aryl, —S(O)heteroaryl, —S(O)₂aryl, —S(O)₂heteroaryl, —NHCOaryl, —NHCOheteroaryl or —NHS(O)₂aryl of R_{d} is optionally substituted with one or more R_{y} groups;
each Rₑ is independently alkyl, alkenyl, alkynyl, cycloalkyl, heterocycle, heteroaryl or aryl;
R_{f} and R_{g} are each independently selected from H, alkyl, alkenyl, alkynyl, cycloalkyl, heterocycle, aryl and heteroaryl; or R_{f} and R_{g} together with the nitrogen to which they are attached form a pyrrolidino, piperidino, piperazino, azetidino, morpholino, or thiomorpholino;
each Rₕ is independently H, alkyl, alkenyl, alkynyl, cycloalkyl, heterocycle, heteroaryl or aryl;
each Rᵢ is independently selected from halogen, aryl, heteroaryl, heterocycle, R_{z}, OH, CN, —OR_{z}, —Oaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z}; wherein any aryl, heteroaryl, heterocycle, —Oaryl, —Saryl, —Sheteroaryl, —S(O)aryl, —S(O)heteroaryl, —S(O)₂aryl, —S(O)₂heteroaryl, —NHCOaryl or —NHCOheteroaryl of Rᵢ is optionally substituted with one or more R_{y} groups;
each Rⱼ is independently alkyl, alkenyl, alkynyl, cycloalkyl, heterocycle, heteroaryl or aryl;
Rₖ and Rₘ are each independently selected from H, alkyl, alkenyl, alkynyl, cycloalkyl, heterocycle, aryl and heteroaryl; or Rₖ and Rₘ together with the nitrogen to which they are attached form a pyrrolidino, piperidino, piperazino, azetidino, morpholino, or thiomorpholino;
each Rₙ is independently H, alkyl, alkenyl, alkynyl, cycloalkyl, heterocycle, heteroaryl or aryl;
each Rₚ is independently selected from halogen, aryl, heteroaryl, heterocycle, R_{z}, OH, CN, —OR_{z}, —Oaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z}; wherein any aryl, heteroaryl, heterocycle, —Oaryl, —Saryl, —Sheteroaryl, —S(O)aryl, —S(O)heteroaryl, —S(O)₂aryl, —S(O)₂heteroaryl, —NHCOaryl, —NHCOheteroaryl or —NHS(O)₂aryl of Rₚ is optionally substituted with one or more R_{y} groups;
R_{q} and Rᵣ are each independently selected from H, alkyl, alkenyl, alkynyl, cycloalkyl, heterocycle and heteroaryl; or R_{q} and Rᵣ together with the nitrogen to which they are attached form a pyrrolidino, piperidino, piperazino, azetidino, morpholino, or thiomorpholino ring;
each Rₛ is independently selected from halogen, aryl, heteroaryl, heterocycle, R_{z}, OH, CN, —OR_{z}, —Oaryl, —OC(O)R_{z}, —OC(O)NR_{z1}R_{z2}, oxo, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R₂, —NHS(O)₂R_{z}, —NHS(O)₂aryl, —NHS(O)₂NH₂, NO₂, ═NOR_{z}, —CHO, —C(O)R_{z}, —C(O)OH, —C(O)OR_{z}, —C(O)NR_{z1}R_{z2} and —C(O)C(O)R_{z}; wherein any aryl, heteroaryl, heterocycle, —Oaryl, —Saryl, —Sheteroaryl, —S(O)aryl, —S(O)heteroaryl, —S(O)₂aryl, —S(O)₂heteroaryl, —NHCOaryl, —NHCOheteroaryl or —NHS(O)₂aryl of Rₛ is optionally substituted with one or more R_{y} groups;
each Rₜ is independently selected from halogen, CF₃, —OCF₃, CN, OH, —NH₂, —Olower alkyl, —Oaryl, —NHlower alkyl, —N(lower alkyl)₂, —C(O)NHlower alkyl, —C(O)N(lower alkyl)₂, aryl, heterocycle and heteroaryl; wherein any aryl, —Oaryl, heteroaryl or heterocycle of Rₜ is optionally substituted with one or more groups selected from aryl and alkyl; and wherein any —Olower alkyl, —NHlower alkyl, N(lower alkyl)₂, —C(O)NHlower alkyl or —C(O)N(lower alkyl)₂, of Rₜ is optionally substituted with one or more NH₂ groups;
each R_{y} is independently halogen, R_{z}, OH, CN, —OR_{z}, —Oaryl, —Oheteroaryl, —OC(O)R_{z}, —OC(O)OR_{z}, —OC(O)NR_{z1}R_{z2}, SH, SR_{z}, —Saryl, —Sheteroaryl, —S(O)R_{z}, —S(O)aryl, —S(O)heteroaryl, —S(O)₂OH, —S(O)₂R_{z}, —S(O)₂OR_{z}, —S(O)₂Oaryl, —OS(O)₂R_{z}, —S(O)₂aryl, —OS(O)₂aryl, —S(O)₂heteroaryl, —OS(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, CHO, —C(O)R_{z}, —C(O)OH, —C(O)OR_{z}, —C(O)Oaryl, —C(O)NR_{z1}R_{z2}, —C(O)aryl, —OC(O)aryl, —C(O)heteroaryl, —OC(O)heteroaryl, —C(O)C(O)R_{z}, —C(═NCN)NH₂, aryl, heterocycle or heteroaryl; wherein any —Oaryl, —Oheteroaryl, —Saryl, —Sheteroaryl, —S(O)aryl, —S(O)heteroaryl, —S(O)₂Oaryl, —S(O)₂aryl, —OS(O)₂aryl, —S(O)₂heteroaryl, —OS(O)₂heteroaryl, —NHCOaryl, —NHCOheteroaryl, —NHS(O)₂aryl, —C(O)Oaryl, —C(O)aryl, —OC(O)aryl, —C(O)heteroaryl, —OC(O)heteroaryl, aryl or heteroaryl of R_{y} is optionally substituted with one or more halogen, OH, SH, R_{z}, —OR_{z}, —SR_{z}, CN, —NR_{z1}R_{z2}, —NO₂, —CHO, —Oaryl, —Oheteroaryl, —C(O)R_{z}, —C(O)OR_{z}, —C(O)OH, —NHCOR_{z}, —NHS(O)₂R_{z}, —NHS(O)₂aryl, —C(O)NR_{z1}R_{z2}, —NHCONR_{z1}R_{z2}, —NHCOheteroaryl, —NHCOaryl, —NHC(O)OR_{z}, —(C₂-C₆)alkynyl, —S(O)R_{z}, —S(O)₂R_{z}, —S(O)aryl, —S(O)₂aryl, —S(O)₂NR_{z1}R_{z2}, —Saryl, —Sheteroaryl, aryl or heteroaryl; wherein —Oaryl, —Oheteroaryl, —NHS(O)₂aryl, —NHCOheteroaryl, —NHCOaryl, —S(O)aryl, —S(O)₂aryl, —Saryl, —Sheteroaryl, aryl or heteroaryl is optionally substituted with one or more groups selected from halogen, CN, —CF₃, NO₂ and (C₁-C₃)alkyl; and wherein any heterocycle of R_{y} is optionally substituted with one or more groups selected from halogen, CN, NO₂, oxo, OH, SH, R_{z}, —OR_{z}, —S(O)₂R_{z}, —S(O)₂aryl, —S(O)₂heteroaryl, —C(O)R_{z}, —C(O)aryl, —C(O)heteroaryl or heteroaryl; wherein —S(O)₂aryl, —S(O)₂heteroaryl, —C(O)aryl, —C(O)heteroaryl or heteroaryl is optionally substituted with one or more groups selected from halogen, CN, —CF₃, NO₂ and (C₁-C₃)alkyl;
each R_{z} is independently lower alkyl or cycloalkyl; wherein any lower alkyl of R_{z} is optionally substituted with one or more groups selected from halogen, CN, —SCN, OH, −NH₂, —Olower alkyl, —NHlower alkyl, —N(lower alkyl)₂, —C(O)NHlower alkyl, —C(O)N(lower alkyl)₂, —C(O)lower alkyl, heterocycle, cycloalkyl, aryl, heteroaryl, —S(O)₂aryl, —S(O)aryl, —Saryl, —Sheteroaryl, —Oaryl and —Oheteroaryl, wherein aryl, heterocycle, heteroaryl, —S(O)₂aryl, —S(O)aryl, —Saryl, —Sheteroaryl, —Oaryl or —Oheteroaryl is optionally substituted with one or more lower alkyl, CN, —O(C₁-C₆)alkyl, NH₂, —NHheteroaryl or —NHS(O)₂(C₁-C₆)alkyl; and wherein any cycloalkyl of R_{z} is optionally substituted with one or more groups selected from (C₁-C₆)alkyl, halogen, CN, OH, —NH₂, —Olower alkyl, —NHlower alkyl, —C(O)NHlower alkyl, —C(O)N(lower alkyl)₂, heterocycle, cycloalkyl, aryl and heteroaryl, wherein aryl, heterocycle or heteroaryl is optionally substituted with one or more lower alkyl; and wherein (C₁-C₆)alkyl is optionally substituted with OH, —NHC(O)aryl or —O(C₁-C₆)alkyl;
R_{z1} and R_{z2} are each independently selected from H, alkyl, alkenyl, alkynyl, lower cycloalkyl, aryl, heterocycle and heteroaryl; wherein any alkyl, alkenyl or alkynyl of R_{z1} or R_{z2} is optionally substituted with one or more Rₜ or groups; and wherein any lower cycloalkyl, aryl, heterocycle or heteroaryl of R_{z1} or R_{z2} is optionally substituted with one or more groups selected from Rₜ or (C₁-C₆)alkyl; or R_{z1} and R_{z2} together with the nitrogen to which they are attached form a cyclic amino; wherein the cyclic amino is optionally substituted with one or more groups selected from Rₜ, oxo and alkyl; and
R_{z3} and R_{z4} are each independently selected from H and CN; or R_{z3} and R_{z4} together with the atom to which they are attached form a cycloalkyl;
or a salt thereof;
provided that when X₁ is CR₄, X₂ is CR₅, Z is C═O and Y is O; then R₅ is H; and that when X₁ is N, X₂ is CR₅, Y is CR₆R₇ and Z is O; then R₅ is H.

2-4. (canceled)

5. The compound of claim 1, which is a compound of formula Ib: 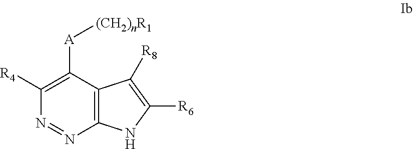
or a salt thereof.

6. The compound of claim 1, which is a compound of formula Ic: 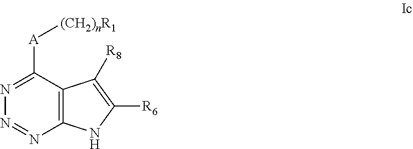
or a salt thereof.

7-10. (canceled)

11. The compound of claim 1, wherein R₄ is:

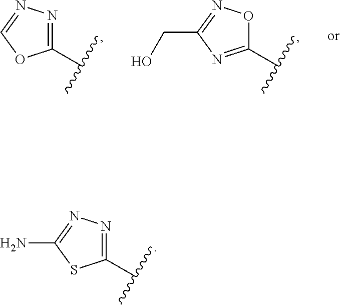

12-22. (canceled)

23. The compound of claim 1, wherein A is absent.

24-34. (canceled)

35. The compound of claim 1, wherein R₁ is:

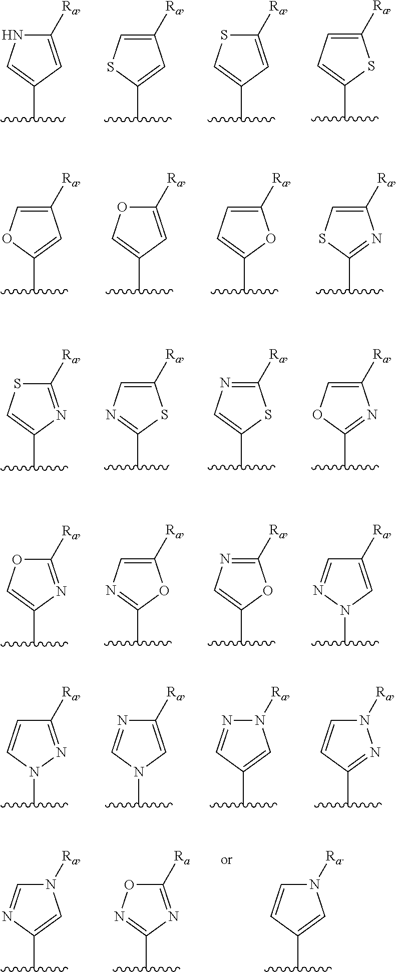

36. (canceled)

37. The compound of claim 1, wherein R₁ is:

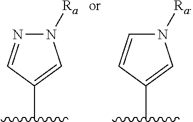

38-47. (canceled)

48. The compound of claim 1, wherein Rₐ is:

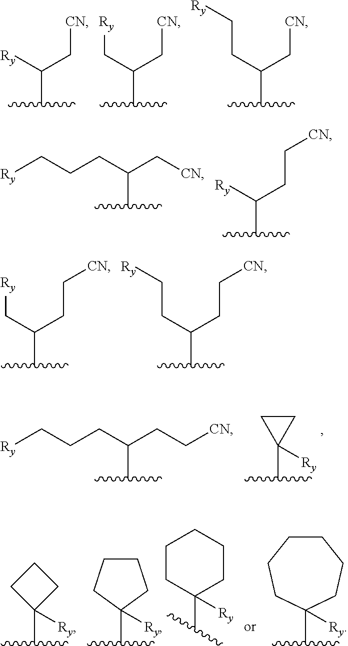

49-55. (canceled)

56. The compound of claim 1, wherein Rₐ is:

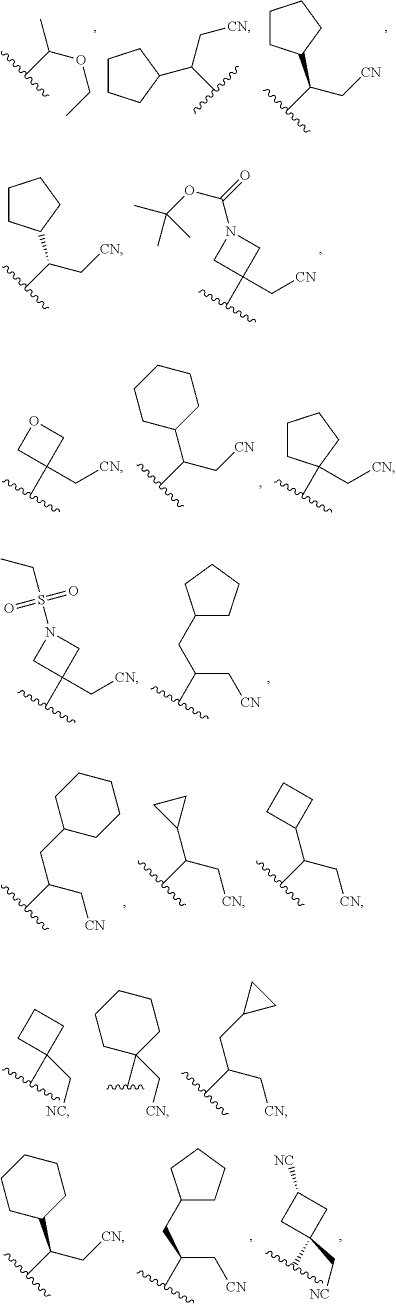

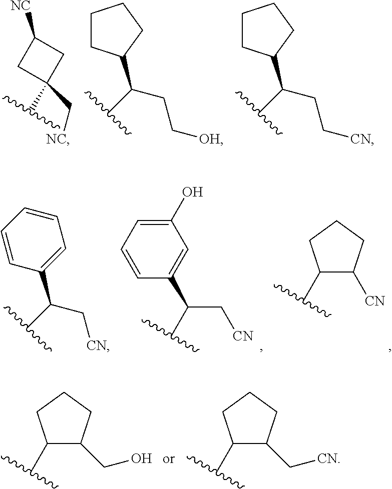

57. The compound of claim 1, wherein Rₐ is:

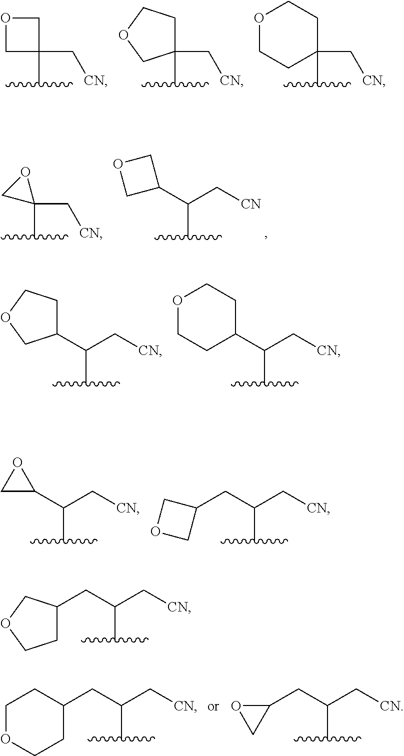

58. The compound of claim 1, wherein Rₐ is: 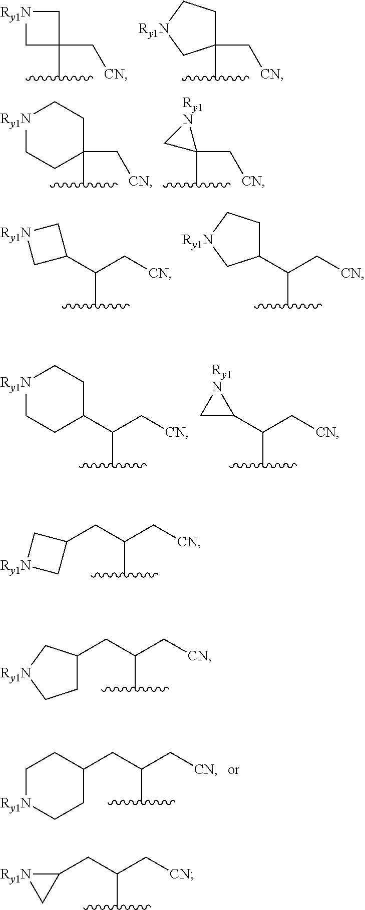
wherein each R_{y1} is independently R_{z}, —S(O)₂R_{z}, —S(O)₂aryl, —S(O)₂heteroaryl, —C(O)R_{z}, —C(O)aryl, —C(O)heteroaryl, or heteroaryl; wherein any aryl or hetereoaryl of R_{y1} is optionally substituted with one or more halogen or (C₁-C₃)alkyl.

59. The compound of claim 1, wherein Rₐ is:

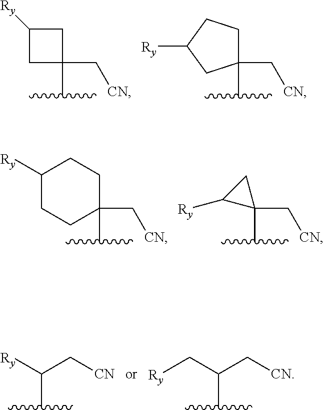

60. (canceled)

61. (canceled)

62. The compound of claim 1, wherein Rₐ is:

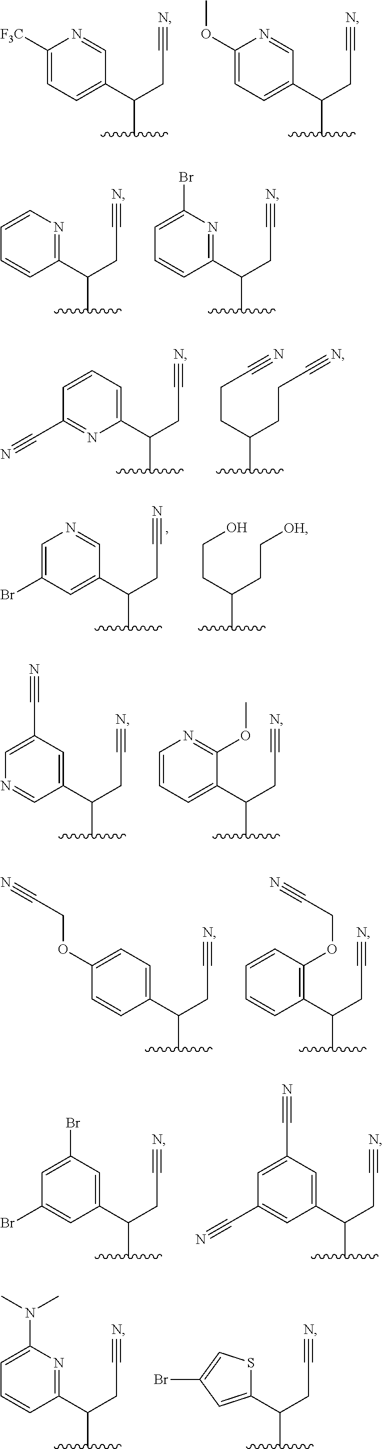

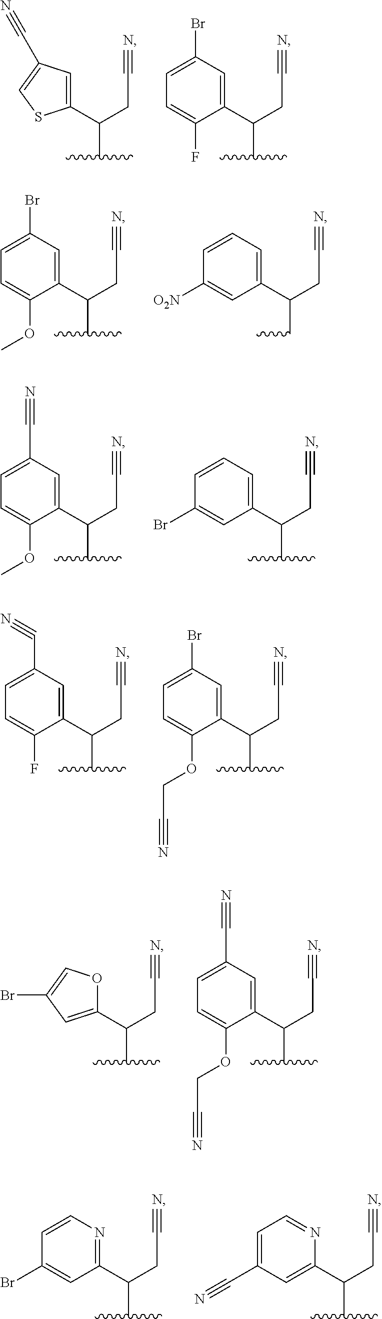

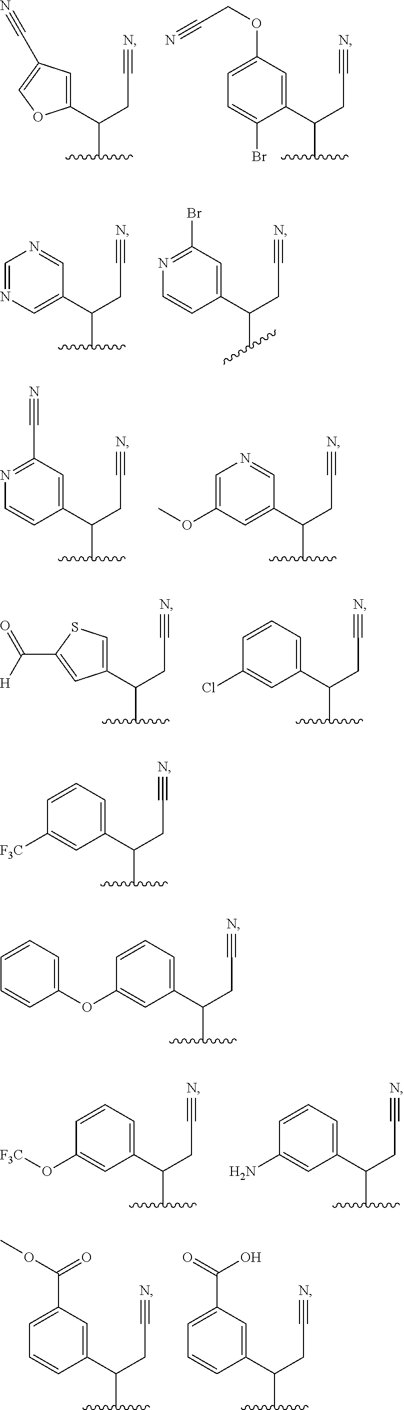

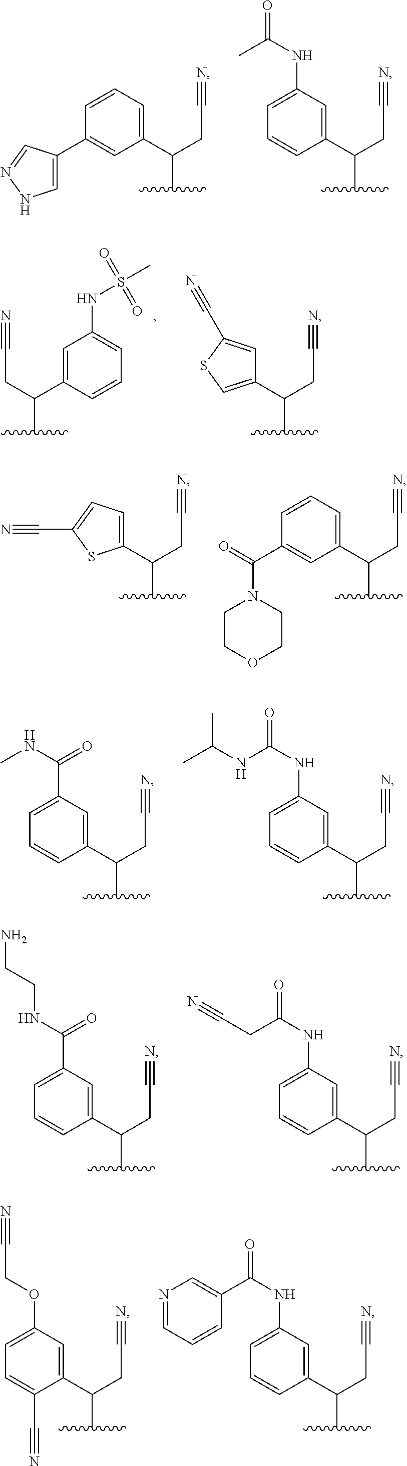

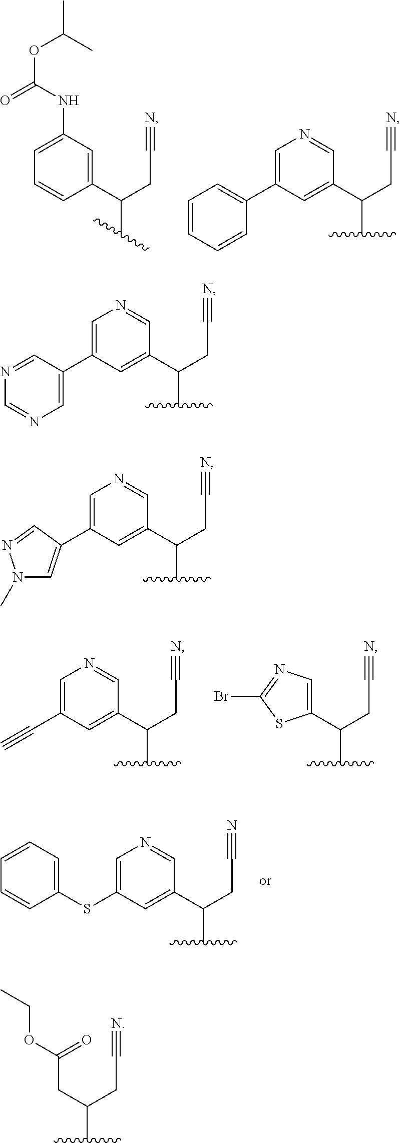

63. The compound of claim 1, wherein R₁ is:

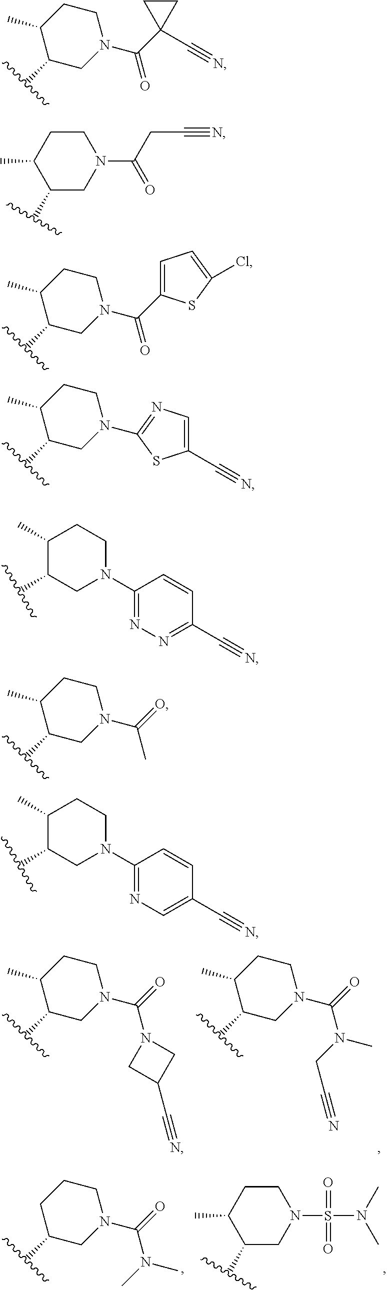

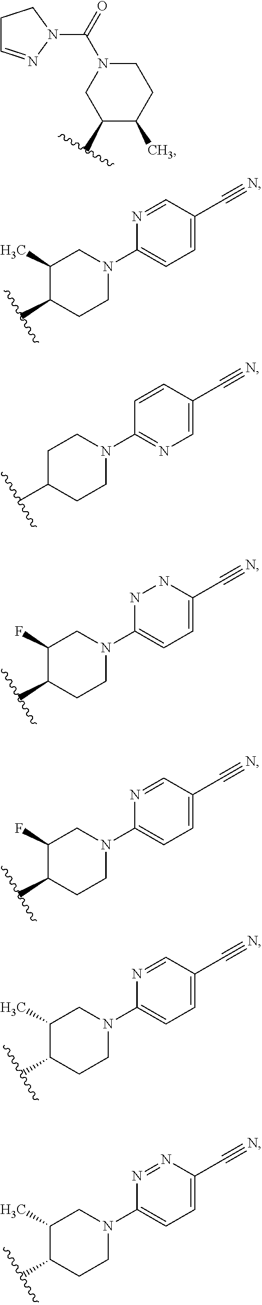

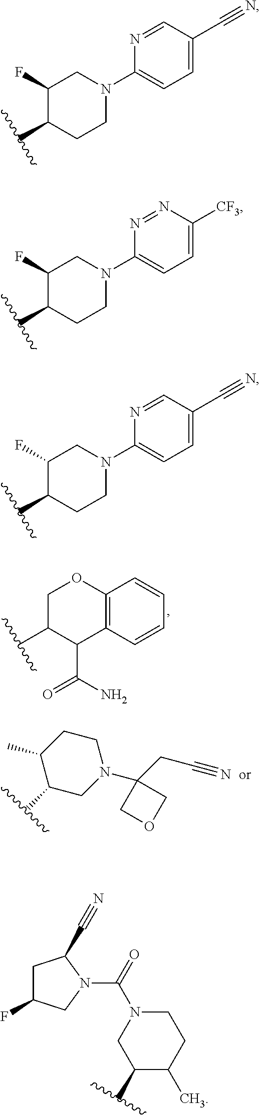

64. The compound of claim 1, wherein R₁ is:

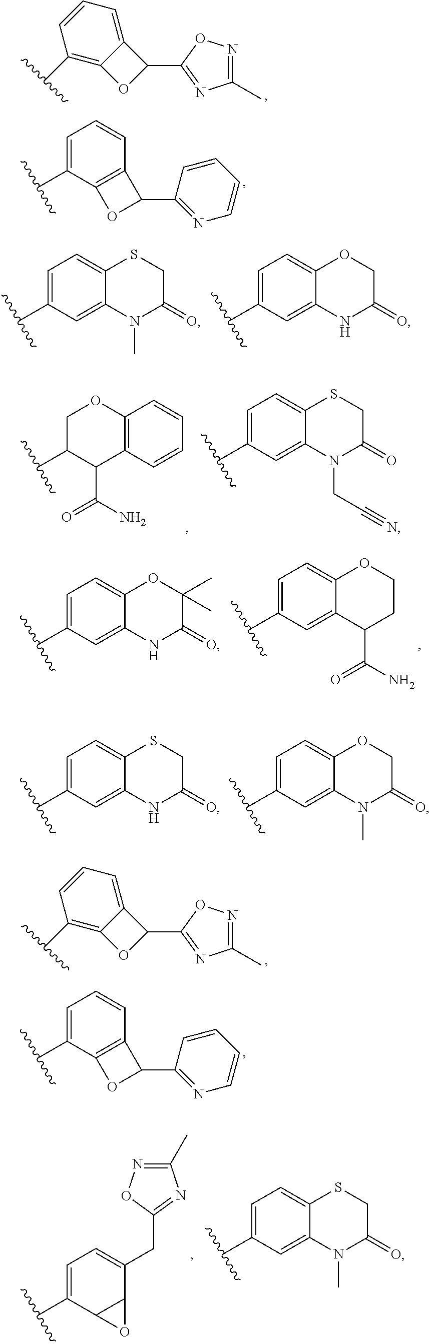

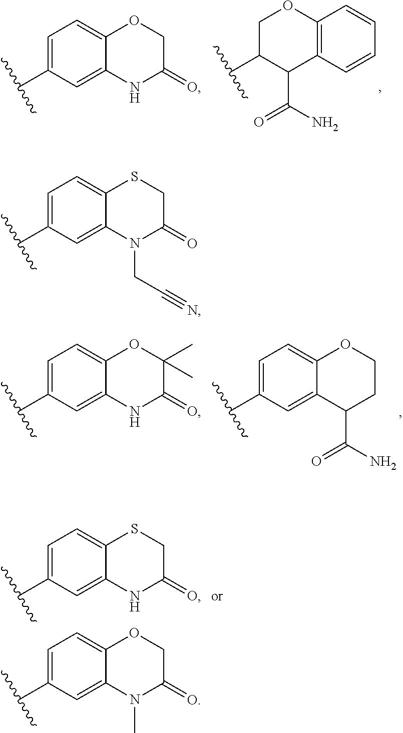

65. The compound of claim 1, wherein R₁ is:

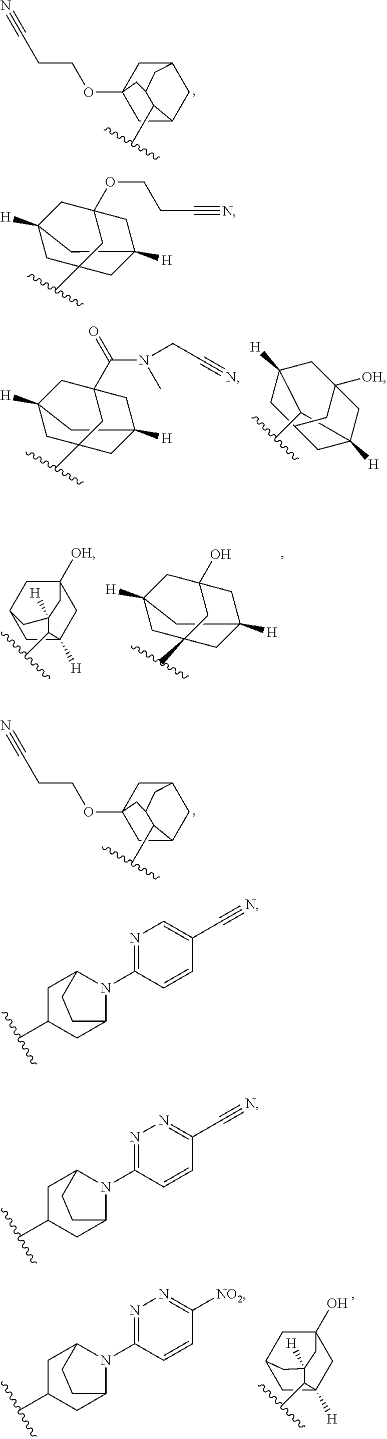

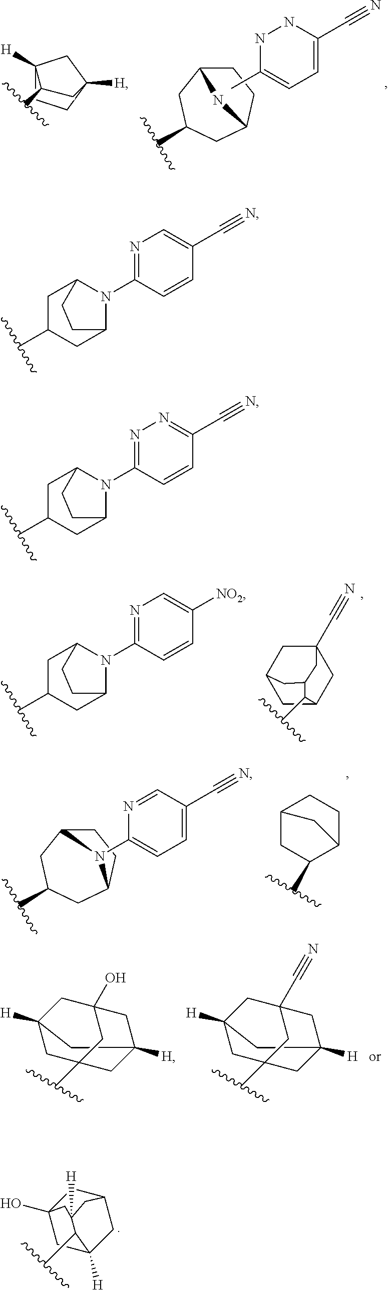

66. The compound of claim 1, wherein R₁ is:

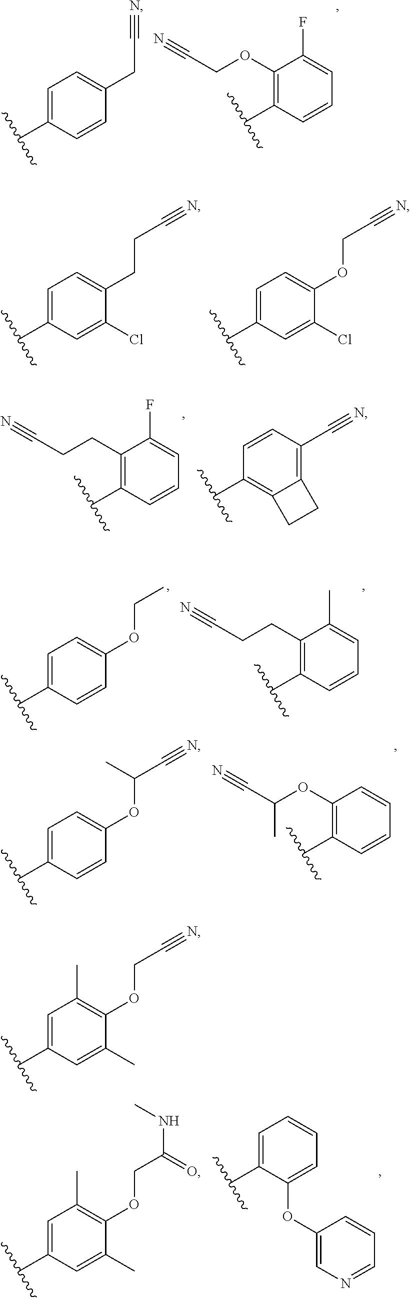

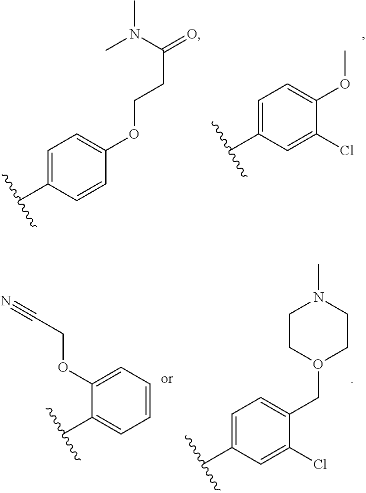

67. The compound of claim 1, wherein R₁ is:

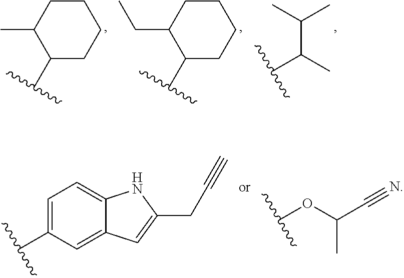

68. The compound of claim 1, wherein R₁ is:

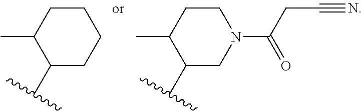

69. The compound of claim 1, wherein R₁ is:

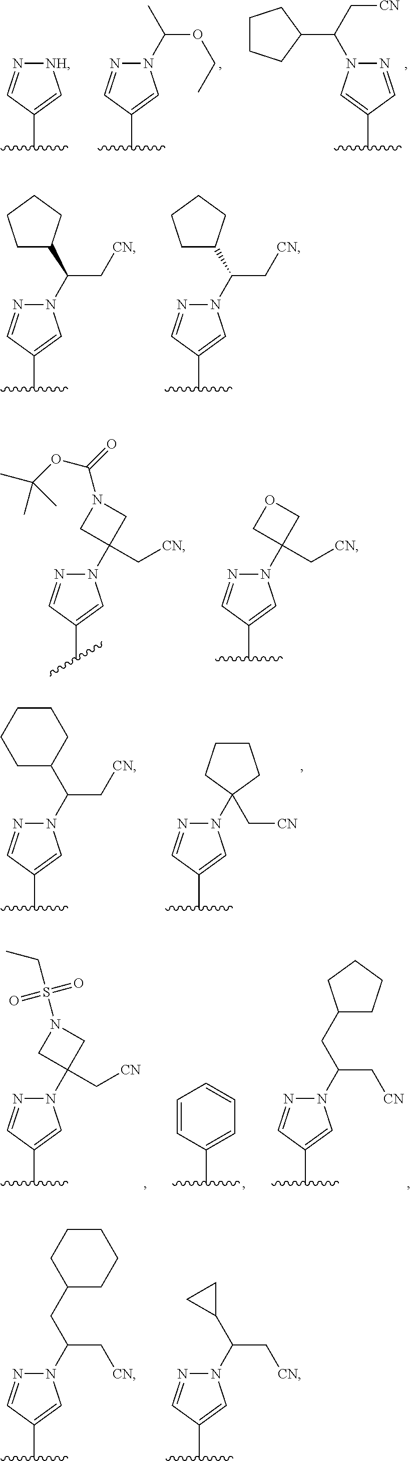

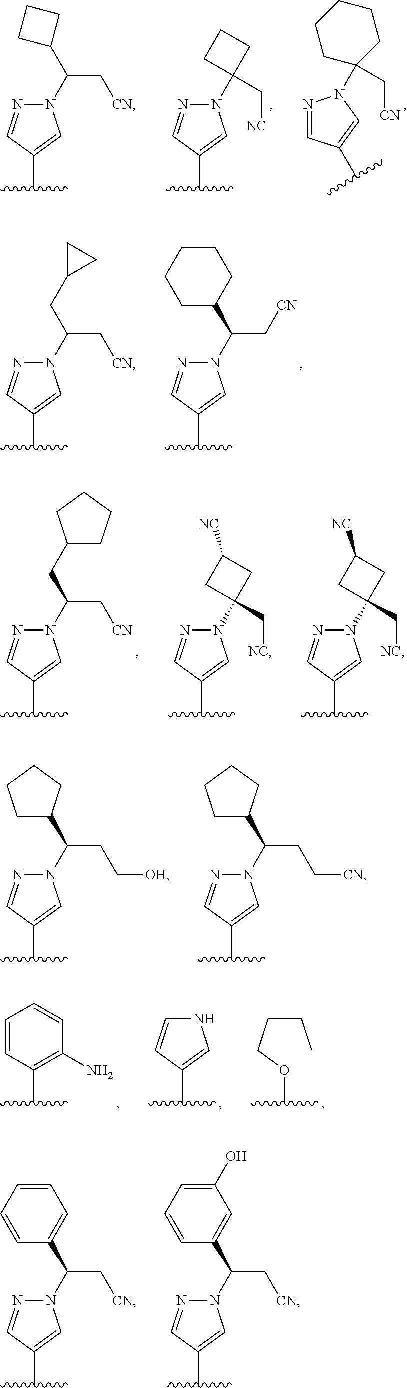

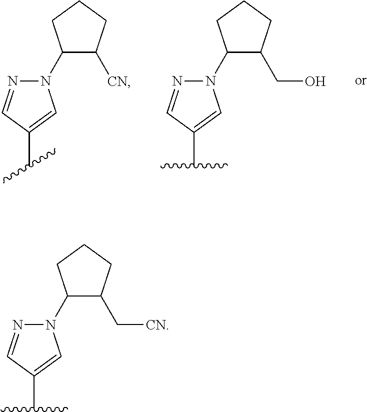

70-72. (canceled)

73. The compound of claim 1, which is a compound of formula: 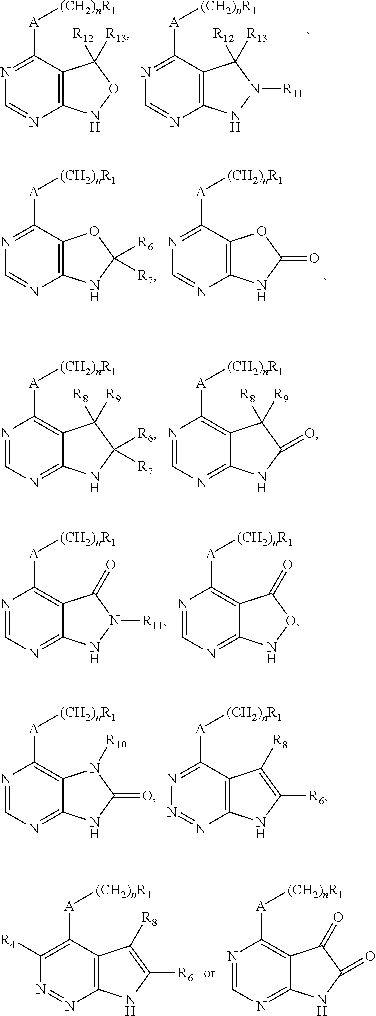
or a salt thereof.

74-77. (canceled)

78. The compound of claim 1, wherein n is 0.

79. The compound of claim 1, which is: 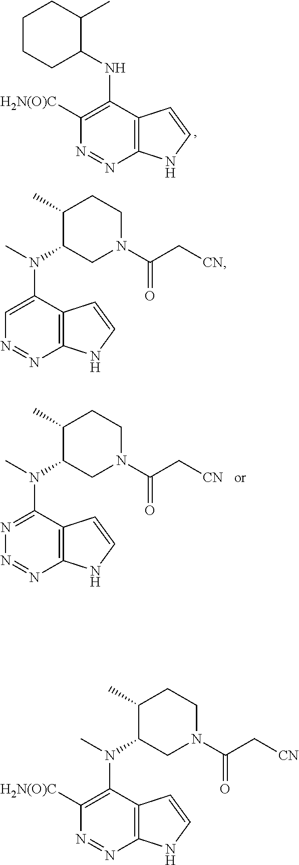
or a salt thereof.

80. The compound of claim 1, which is: 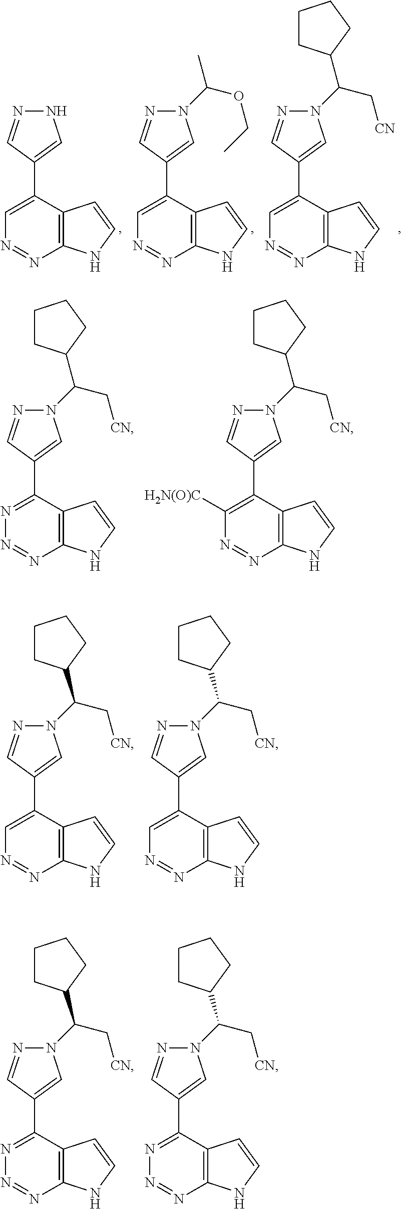 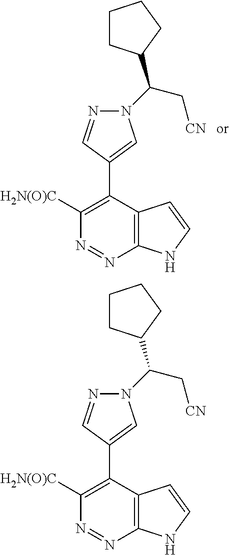
or a salt thereof.

81. The compound of claim 1, which is:
4-(1H-pyrazol-4-yl)-7H-pyrrolo[2,3-c]pyridazine;
4-(1-(1-ethoxyethyl)-1H-pyrazol-4-yl)-7H-pyrrolo[2,3-c]pyridazine;
3-(4-(7H-pyrrolo[2,3-c]pyridazin-4-yl)-1H-pyrazol-1-yl)-3-cyclopentylpropanenitrile;
(R)-3-(4-(7H-pyrrolo[2,3-c]pyridazin-4-yl)-1H-pyrazol-1-yl)-3-cyclopentyl propanenitrile;
(S)-3-(4-(7H-pyrrolo[2,3-c]pyridazin-4-yl)-1H-pyrazol-1-yl)-3-cyclopentyl propanenitrile;
tert-butyl 3-(4-(7H-pyrrolo[2,3-c]pyridazin-4-yl)-1H-pyrazol-1-yl)-3-(cyanomethyl)azetidine-1-carboxylate;
2-(3-(4-(7H-pyrrolo[2,3-c]pyridazin-4-yl)-1H-pyrazol-1-yl)oxetan-3-yl)acetonitrile;
3-(4-(7H-pyrrolo[2,3-c]pyridazin-4-yl)-1H-pyrazol-1-yl)-3-cyclohexylpropane nitrile;
2-(1-(4-(7H-pyrrolo[2,3-c]pyridazin-4-yl)-1H-pyrazol-1-yl)cyclopentyl)acetonitrile;
2-(3-(4-(7H-pyrrolo[2,3-c]pyridazin-4-yl)-1H-pyrazol-1-yl)-1-(ethylsulfonyl)azetidin-3-yl)acetonitrile;
4-phenyl-7H-pyrrolo[2,3-c]pyridazine;
3-(4-(7H-pyrrolo[2,3-c]pyridazin-4-yl)-1H-pyrazol-1-yl)-4-cyclopentylbutane nitrile;
3-(4-(7H-pyrrolo[2,3-c]pyridazin-4-yl)-1H-pyrazol-1-yl)-4-cyclohexylbutane nitrile;
3-(4-(7H-pyrrolo[2,3-c]pyridazin-4-yl)-1H-pyrazol-1-yl)-3-cyclopropylpropane nitrile;
3-(4-(7H-pyrrolo[2,3-c]pyridazin-4-yl)-1H-pyrazol-1-yl)-3-cyclobutylpropane nitrile;
2-(1-(4-(7H-pyrrolo[2,3-c]pyridazin-4-yl)-1H-pyrazol-1-yl)cyclobutyl)acetonitrile;
2-(1-(4-(7H-pyrrolo[2,3-c]pyridazin-4-yl)-1H-pyrazol-1-yl)cyclohexyl)acetonitrile;
3-(4-(7H-pyrrolo[2,3-c]pyridazin-4-yl)-1H-pyrazol-1-yl)-4-cyclopropylbutane nitrile;
(R)-3-(4-(7H-pyrrolo[2,3-c]pyridazin-4-yl)-1H-pyrazol-1-yl)-3-cyclohexylpropane nitrile;
(S)-3-(4-(7H-pyrrolo[2,3-c]pyridazin-4-yl)-1H-pyrazol-1-yl)-4-cyclopentylbutane nitrile;
(E)-3-(4-(7H-pyrrolo[2,3-c]pyridazin-4-yl)-1H-pyrazol-1-yl)-3-(cyanomethyl)cyclobutanecarbonitrile;
(Z)-3-(4-(7H-pyrrolo[2,3-c]pyridazin-4-yl)-1H-pyrazol-1-yl)-3-(cyanomethyl)cyclobutanecarbonitrile;
(R)-3-(4-(7H-pyrrolo[2,3-c]pyridazin-4-yl)-1H-pyrazol-1-yl)-3-cyclopentyl propan-1-ol;
(R)-4-(4-(7H-pyrrolo[2,3-c]pyridazin-4-yl)-1H-pyrazol-1-yl)-4-cyclopentylbutanenitrile;
2-(7H-pyrrolo[2,3-c]pyridazin-4-yl)aniline;
4-(1H-pyrrol-3-yl)-7H-pyrrolo[2,3-c]pyridazine;
(R)-3-(4-(7H-pyrrolo[2,3-c]pyridazin-4-yl)-1H-pyrazol-1-yl)-3-phenylpropane nitrile;
(R)-3-(4-(7H-pyrrolo[2,3-c]pyridazin-4-yl)-1H-pyrazol-1-yl)-3-(3-hydroxyphenyl);
4-hydroxy-7H-pyrrolo[2,3-d][1,2,3]triazine-5-carboxamide;
2-(4-(7H-pyrrolo[2,3-c]pyridazin-4-yl)-1H-pyrazol-1-yl)cyclopentane carbonitrile;
(2-(4-(7H-pyrrolo[2,3-c]pyridazin-4-yl)-1H-pyrazol-1-yl)cyclopentyl)methanol;
2-(2-(4-(7H-pyrrolo[2,3-c]pyridazin-4-yl)-1H-pyrazol-1-yl)cyclopentyl)acetonitrile; or
3-(4-methyl-3-(methyl(6-oxo-6,7-dihydro-5H-pyrrolo[2,3-d]pyrimidin-4-yl)amino)piperidin-1-yl)-3-oxopropanenitrile;
or a salt thereof.

82. The compound of claim h which is:
2S)-2-(4-(7H-pyrrolo[2,3-c]pyridazin-4-yl)-1H-pyrazol-1-yl)cyclopentanecarbonitrile;
2S)-2-(4-(7H-pyrrolo[2,3-c]pyridazin-4-yl)-1H-pyrazol-1-yl)cyclopentanecarbonitrile;
2R)-2-(4-(7H-pyrrolo[2,3-c]pyridazin-4-yl)-1H-pyrazol-1-yl)cyclopentanecarbonitrile;
2R)-2-(4-(7H-pyrrolo[2,3-c]pyridazin-4-yl)-1H-pyrazol-1-yl)cyclopentanecarbonitrile;
((1S,2S)-2-(4-(7H-pyrrolo[2,3-c]pyridazin-4-yl)-1H-pyrazol-1-yl)cyclopentyl)methanol;
((1R,2S)-2-(4-(7H-pyrrolo[2,3-c]pyridazin-4-yl)-1H-pyrazol-1-yl)cyclopentyl)methanol;
((1R,2R)-2-(4-(7H-pyrrolo[2,3-c]pyridazin-4-yl)-1H-pyrazol-1-yl)cyclopentyl)methanol;
((1 S,2R)-2-(4-(7H-pyrrolo[2,3-c]pyridazin-4-yl)-1H-pyrazol-1-yl)cyclopentyl)methanol;
2-(2-(4-(7H-pyrrolo[2,3-c]pyridazin-4-yl)-1H-pyrazol-1-yl)cyclopentyl)acetonitrile;
2-((1R,2S)-2-(4-(7H-pyrrolo[2,3-c]pyridazin-4-yl)-1H-pyrazol-1-yl)cyclopentyl)acetonitrile;
2-((1S,2S)-2-(4-(7H-pyrrolo[2,3-c]pyridazin-4-yl)-1H-pyrazol-1-yl)cyclopentyl)acetonitrile;
2-((1S,2R)-2-(4-(7H-pyrrolo[2,3-c]pyridazin-4-yl)-1H-pyrazol-1-yl)cyclopentyl)acetonitrile;
2-((1R,2R)-2-(4-(7H-pyrrolo[2,3-c]pyridazin-4-yl)-1H-pyrazol-1-yl)cyclopentyl)acetonitrile;
(S)-3-(4-(7H-pyrrolo[2,3-c]pyridazin-4-yl)-1H-pyrazol-1-yl)-3-cyclohexylpropanenitrile;
(R)-3-(4-(7H-pyrrolo[2,3-c]pyridazin-4-yl)-1H-pyrazol-1-yl)-4-cyclopentylbutanenitrile;
(S)-3-(4-(7H-pyrrolo[2,3-c]pyridazin-4-yl)-1H-pyrazol-1-yl)-4-cyclohexylbutanenitrile;
(R)-3-(4-(7H-pyrrolo[2,3-c]pyridazin-4-yl)-1H-pyrazol-1-yl)-4-cyclohexylbutanenitrile;
(R)-3-(4-(7H-pyrrolo[2,3-c]pyridazin-4-yl)-1H-pyrazol-1-yl)-3-cyclopropylpropanenitrile;
(S)-3-(4-(7H-pyrrolo[2,3-c]pyridazin-4-yl)-1H-pyrazol-1-yl)-3-cyclopropylpropanenitrile;
(R)-3-(4-(7H-pyrrolo[2,3-c]pyridazin-4-yl)-1H-pyrazol-1-yl)-3-cyclobutylpropanenitrile;
(S)-3-(4-(7H-pyrrolo[2,3-c]pyridazin-4-yl)-1H-pyrazol-1-yl)-3-cyclobutylpropanenitrile;
(R)-3-(4-(7H-pyrrolo[2,3-c]pyridazin-4-yl)-1H-pyrazol-1-yl)-4-cyclopropylbutanenitrile;
(S)-3-(4-(7H-pyrrolo[2,3-c]pyridazin-4-yl)-1H-pyrazol-1-yl)-4-cyclopropylbutanenitrile;
3-(4-(7H-pyrrolo[2,3-c]pyridazin-4-yl)-1H-pyrazol-1-yl)-3-cyclopentylpropan-1-ol;
(S)-3-(4-(7H-pyrrolo[2,3-c]pyridazin-4-yl)-1H-pyrazol-1-yl)-3-cyclopentylpropan-1-ol;
4-(4-(7H-pyrrolo[2,3-c]pyridazin-4-yl)-1H-pyrazol-1-yl)-4-cyclopentylbutanenitrile;
(S)-4-(4-(7H-pyrrolo[2,3-c]pyridazin-4-yl)-1H-pyrazol-1-yl)-4-cyclopentylbutanenitrile;
3-(4-(7H-pyrrolo[2,3-c]pyridazin-4-yl)-1H-pyrazol-1-yl)-3-phenylpropanenitrile;
(S)-3-(4-(7H-pyrrolo[2,3-c]pyridazin-4-yl)-1H-pyrazol-1-yl)-3-phenylpropanenitrile;
3-(4-(7H-pyrrolo[2,3-c]pyridazin-4-yl)-1H-pyrazol-1-yl)-3-(3-hydroxyphenyl)propanenitrile;
(S)-3-(4-(7H-pyrrolo[2,3-c]pyridazin-4-yl)-1H-pyrazol-1-yl)-3-(3-hydroxyphenyl)propanenitrile;
3-(4-(7H-pyrrolo[2,3-c]pyridazin-4-yl)-1H-pyrazol-1-yl)-3-(2-hydroxyphenyl)propanenitrile;
(S)-3-(4-(7H-pyrrolo[2,3-c]pyridazin-4-yl)-1H-pyrazol-1-yl)-3-(2-hydroxyphenyl)propanenitrile;
(R)-3-(4-(7H-pyrrolo[2,3-c]pyridazin-4-yl)-1H-pyrazol-1-yl)-3-(2-hydroxyphenyl)propanenitrile;
3-(4-(7H-pyrrolo[2,3-c]pyridazin-4-yl)-1H-pyrazol-1-yl)-3-(4-hydroxyphenyl)propanenitrile;
(S)-3-(4-(7H-pyrrolo[2,3-c]pyridazin-4-yl)-1H-pyrazol-1-yl)-3-(4-hydroxyphenyl)propanenitrile;
(R)-3-(4-(7H-pyrrolo[2,3-c]pyridazin-4-yl)-1H-pyrazol-1-yl)-3-(4-hydroxyphenyl)propanenitrile;
2-((1S,2S)-2-(4-(7H-pyrrolo[2,3-c]pyridazin-4-yl)-1H-pyrazol-1-yl)cyclopentyl)acetonitrile;
2-((1R,2S)-2-(4-(7H-pyrrolo[2,3-c]pyridazin-4-yl)-1H-pyrazol-1-yl)cyclopentyl)acetonitrile;
2-((1S,2R)-2-(4-(7H-pyrrolo[2,3-c]pyridazin-4-yl)-1H-pyrazol-1-yl)cyclopentyl)acetonitrile; or
2-((1R,2R)-2-(4-(7H-pyrrolo[2,3-c]pyridazin-4-yl)-1H-pyrazol-1-yl)cyclopentyl)acetonitrile;
or a salt thereof.

83. A pharmaceutical composition, comprising a compound of claim 1,
or a pharmaceutically acceptable salt thereof, and a pharmaceutically acceptable diluent or carrier.

84. (canceled)

85. A method for treating a disease or condition associated with pathologic Janus kinase (JAKE activation in a mammal, comprising administering to a mammal in need thereof an effective amount of a compound of claim 1, or a pharmaceutically acceptable salt thereof.

86. (canceled)

87. (canceled)

88. The method of claim 85, wherein the disease or condition associated with pathologic JAK activation is cancer.

89. (canceled)

90. A method for suppressing an immune response in a mammal, comprising administering to a mammal in need thereof an effective amount of a compound of claim 1, or a pharmaceutically acceptable salt thereof.

91. (canceled)

92. (canceled)
